SEQUENCE LISTING (341 sequences)

SEQ ID NO: 1 - DNA, 960 bp, Bacillus subtilis
  CDS (1)..(960)
atgcaactattcgatctgccgctcgaccaattgcaaacatataagcctgaaaaaacagcaccgaaagatttttctgagttttggaaattgtctttggaggaacttgcaaaagtccaagcagaacctgatttacagccggttgactatcctgctgacggagtaaaagtgtaccgtctcacatataaaagcttcggaaacgcccgcattaccggatggtacgcggtgcctgacaagcaaggcccgcatccggcgatcgtgaaatatcatggctacaatgcaagctatgatggtgagattcatgaaatggtaaactgggcactccatggctacgccgcattcggcatgcttgtccgcggccagcagagcagcgaggatacgagtatttcactgcacggtcacgctttgggctggatgacgaaaggaattcttgataaagatacatactattaccgcggtgtttatttggacgccgtccgcgcgcttgaggtcatcagcagcttcgacgaggttgacgaaacaaggatcggtgtgacaggaggaagccaaggcggaggtttaaccattgccgcagcagcgctgtcagacattccaaaagccgcggttgccgattatccttatttaagcaacttcgaacgggccattgatgtggcgcttgaacagccgtaccttgaaatcaattccttcttcagaagaaatggcagcccggaaacagaagtgcaggcgatgaagacactttcatatttcgatattatgaatctcgctgaccgagtgaaggtgcctgtcctgatgtcaatcggcctgattgacaaggtcacgccgccgtccaccgtgtttgccgcctacaatcatttggaaacagagaaagagctgaaggtgtaccgctacttcggacatgagtatatccctgcttttcaaacggaaaaacttgctttctttaagcagcatcttaaaggctgataa

SEQ ID NO: 2 - protein, 318 aa, Bacillus subtilis
MQLFDLPLDQLQTYKPEKTAPKDFSEFWKLSLEELAKVQAEPDLQPVDYPADGVKVYRLTYKSFGNARITGWYAVPDKQGPHPAIVKYHGYNASYDGEIHEMVNWALHGYAAFGMLVRGQQSSEDTSISLHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEVQAMKTLSYFDIMNLADRVKVPVLMSIGLIDKVTPPSTVFAAYNHLETEKELKVYRYFGHEYIPAFQTEKLAFFKQHLKG

SEQ ID NO: 3 - DNA, 957 bp, Bacillus subtilis
atgcaactattcgatctgccgctcgaccaattgcaaacatataagcctgaaaaaacagcaccgaaagatttttctgagttttggaaattgtctttggaggaacttgcaaaagtccaagcagaacctgatttacagccggttgactatcctgctgacggagtaaaagtgtaccgtctcacatataaaagcttcggaaacgcccgcattaccggatggtacgcggtgcctgacaaggaaggcccgcatccggcgatcgtgaaatatcatggctacaatgcaagctatgatggtgagattcatgaaatggtaaactgggcactccatggctacgccacattcggcatgcttgtccgcggccagcagagcagcgaggatacgagtatttcaccgcacggtcacgctttgggctggatgacgaaaggaattcttgataaagatacatactattaccgcggtgtttatttggacgccgtccgcgcgcttgaggtcatcagcagcttcgacgaggttgacgaaacaaggatcggtgtgacaggaggaagccaaggcggaggtttaaccattgccgcagcagcgctgtcagacattccaaaagccgcggttgccgattatccttatttaagcaacttcgaacgggccattgatgtggcgcttgaacagccgtaccttgaaatcaattccttcttcagaagaaatggcagcccggaaacagaagtgcaggcgatgaagacactttcatatttcgatattatgaatctcgctgaccgagtgaaggtgcctgtcctgatgtcaatcggcctgattgacaaggtcacgccgccgtccaccgtgtttgccgcctacaatcatttggaaacaaagaaagagctgaaggtgtaccgctacttcggacatgagtatatccctgcttttcaaactgaaaaacttgctttctttaagcagcatcttaaaggctga

SEQ ID NO: 4 - protein, 318 aa, Bacillus subtilis
MQLFDLPLDQLQTYKPEKTAPKDFSEFWKLSLEELAKVQAEPDLQPVDYPADGVKVYRLTYKSFGNARITGWYAVPDKEGPHPAIVKYHGYNASYDGEIHEMVNWALHGYATFGMLVRGQQSSEDTSISPHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEVQAMKTLSYFDIMNLADRVKVPVLMSIGLIDKVTPPSTVFAAYNHLETKKELKVYRYFGHEYIPAFQTEKLAFFKQHLKG

SEQ ID NO: 5 - DNA, 957 bp, Bacillus subtilis
atgcaactattcgatctgccgctcgaccaattgcaaacgtataagcctgaaaaaacaacaccgaacgatttttctgagttttggaaatcgtctttggacgaacttgcgaaagtcaaagcagcacctgatttacagctggttgattatcctgctgatggagtcaaggtgtaccgcctcacatataaaagcttcggaaacgcccgcattaccggatggtacgcagtgcctgacaaggaaggaccgcatccggcgatcgtcaaatatcatggctacaacgctagctatgacggtgagattcatgaaatggtaaactgggcgctccacggttacgccgcattcggcatgctagtccgcggccagcagagcagcgaggatacgagtatttctccacatggccatgctttgggctggatgacgaaaggaatccttgataaagatacatactattaccggggcgtttatttggacgctgtccgcgcgcttgaggtcatcagcagctttgacgaagttgacgaaacaagaatcggtgtgacaggcggaagccaaggaggcggcttaaccattgccgcagccgctctgtcagacattccaaaagccgcggttgccgattatccttatttaagcaactttgaacgggccattgatgtggcgcttgaacagccgtaccttgaaatcaattccttctttagaagaaatggaagcccggaaacggaagagaaggcgatgaagacactttcatatttcgatattatgaatctcgctgaccgagtgaaggtccctgtcctgatgtcgatcggtctgattgacaaggtcacgccgccgtccaccgtgtttgccgcatacaaccacttggagacagagaaagagctcaaagtgtaccgctacttcgggcatgagtatatccctgcctttcaaacagaaaaacttgctttctttaagcagcatcttaaaggctga

SEQ ID NO: 6 - protein, 318 aa, Bacillus subtilis
MQLFDLPLDQLQTYKPEKTTPNDFSEFWKSSLDELAKVKAAPDLQLVDYPADGVKVYRLTYKSFGNARITGWYAVPDKEGPHPAIVKYHGYNASYDGEIHEMVNWALHGYAAFGMLVRGQQSSEDTSISPHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEEKAMKTLSYFDIMNLADRVKVPVLMSIGLIDKVTPPSTVFAAYNHLETEKELKVYRYFGHEYIPAFQTEKLAFFKQHLKG

SEQ ID NO: 7 - DNA, 957 bp, Bacillus licheniformis
atgcagcagccttatgatatgccgcttgaacagctttatcagtataaacctgaacggacggcaccggccgattttaaagagttctggaagggttcattggaggaattggcaaatgaaaaagcgggaccgcagcttgaaccgcatgaatatccggctgacggggtaaaagtctactggcttacatacagaagcatcgggggagcgcgaattaaaggctggtacgcagtacccgaccgccaagggcctcatcctgcgatcgtcaaataccacggctataacgcaagctatgacggagacattcacgatattgtcaattgggctcttcacggctatgcggcattcggtatgctggtccgcggacagaacagcagtgaagatacagagatctctcatcacggacatgtacccggctggatgacaaaaggaatcctcgatccgaaaacatattactacagaggggtctatttagatgccgtacgagcagtcgaagtggtcagcggttttgctgaagtcgatgaaaagcggatcggggtgatcggggcaagccaaggaggcgggctggccgtcgcggtttcggcgctgtccgatattccaaaagcagccgtgtcagaatacccttatttaagcaattttcaacgagcgatcgatacagcgatcgaccagccatatctcgaaatcaactcctttttcagaagaaacaccagtccggatattgagcaggcggccatgcataccctgtcttatttcgatgtcatgaaccttgcccaattggtcaaagcgaccgtactcatgtcgatcggactggttgacaccatcactccgccatccaccgtctttgcggcttacaatcacttggaaacggataaagaaataaaagtgtaccgttattttggacacgaatacatcccgccgttccaaaccgaaaagctggcgtttctgagaaagcatctgaaataa

SEQ ID NO: 8 - protein, 318 aa, Bacillus licheniformis
MQQPYDMPLEQLYQYKPERTAPADFKEFWKGSLEELANEKAGPQLEPHEYPADGVKVYWLTYRSIGGARIKGWYAVPDRQGPHPAIVKYHGYNASYDGDIHDIVNWALHGYAAFGMLVRGQNSSEDTEISHHGHVPGWMTKGILDPKTYYYRGVYLDAVRAVEVVSGFAEVDEKRIGVIGASQGGGLAVAVSALSDIPKAAVSEYPYLSNFQRAIDTAIDQPYLEINSFFRRNTSPDIEQAAMHTLSYFDVMNLAQLVKATVLMSIGLVDTITPPSTVFAAYNHLETDKEIKVYRYFGHEYIPPFQTEKLAFLRKHLK

SEQ ID NO: 9 - DNA, 963 bp, Bacillus pumilis
atgcaattgttcgatttatcactagaagagctaaaaaaatataaaccaaagaaaacagcacgtcctgatttctcagacttttggaagaaatcgctcgaagaactgcgccaagtggaggcagagccaacacttgaatcttatgactatccagtgaaaggcgtcaaggtgtaccgcctgacgtatcaaagctttggacattctaaaattgaaggcttttatgctgtgcctgatcaaactggtccgcatccagcgctcgttcgttttcatggctataatgccagctatgacggcggcattcacgacatcgtcaactgggcgctgcacggctatgcaacatttggtatgctcgtccgcggtcaaggtggcagtgaagacacatcagtgacaccaggcgggcatgcattagggtggatgacaaaaggcattttatcgaaagatacgtactattatcgaggcgtttatctagatgctgttcgtgcacttgaagtcattcagtctttccccgaagtagatgaacaccgtatcggcgtgatcggtggaagtcaggggggtgcgttagcgattgcggccgcagccctttcagacattccaaaagtcgttgtggcagactatccttacttatcaaattttgagcgtgcagttgatgttgccttggagcagccttatttagaaatcaattcatactttcgcagaaacagtgatccgaaagtggaggaaaaggcatttgagacattaagctattttgatttaatcaatttagctggatgggtgaaacagccaacattgatggcgatcggtctgattgacaaaataaccccaccatctactgtgtttgcggcatacaaccatttagaaacagataaagacctgaaagtatatcgctattttggacacgagtttatccctgcttttcaaacagagaagctgtcctttttacaaaagcatttgcttctatcaacataa

SEQ ID NO: 10 - protein, 320 aa, Bacillus pumilis
MQLFDLSLEELKKYKPKKTARPDFSDFWKKSLEELRQVEAEPTLESYDYPVKGVKVYRLTYQSFGHSKIEGFYAVPDQTGPHPALVRFHGYNASYDGGIHDIVNWALHGYATFGMLVRGQGGSEDTSVTPGGHALGWMTKGILSKDTYYYRGVYLDAVRALEVIQSFPEVDEHRIGVIGGSQGGALAIAAAALSDIPKVVVADYPYLSNFERAVDVALEQPYLEINSYFRRNSDPKVEEKAFETLSYFDLINLAGWVKQPTLMAIGLIDKITPPSTVFAAYNHLETDKDLKVYRYFGHEFIPAFQTEKLSFLQKHLLLST

SEQ ID NO: 11 - DNA, 963 bp, Clostridium thermocellum
atggcacaattatatgatatgcctttggaggaattaaaaaaatataagcctgcgcttacaaaacagaaagattttgatgagttttgggaaaaaagccttaaagagctggctgaaattcctttaaaatatcaacttataccttatgattttccggcccggagggtaaaagttttcagagttgaatatcttggttttaaaggtgcaaatattgaagggtggcttgccgttcccgagggagaagggttgtatcccgggcttgtacagtttcacggatacaactgggcgatggatggatgtgttcccgatgtggtaaattgggctttgaatggatatgccgcatttcttatgcttgttcggggacagcagggaagaagcgtggacaatattgtgcccggcagcggtcatgctttgggatggatgtcgaaaggtattttgtcaccggaggaatattattatagaggagtatatatggatgcggttcgtgctgttgaaattttggcttcgcttccttgtgtggatgaatcgagaataggagtgacagggggcagccagggtggaggacttgcactggcggtggctgctctgtccggcataccgaaagttgcagccgtgcattatccgtttctggcacattttgagcgtgccattgacgttgcgccggacggcccttatcttgaaattaacgaatatttaagaagaaacagcggtgaagaaatagaaagacaggtaaagaaaaccctttcctattttgatatcatgaatcttgctccccgtataaaatgccgtacttggatttgcactggtcttgtggatgagattactcctccgtcaacggtttttgcagtgtacaatcacctcaaatgcccaaaggaaatttcggtattcagatattttgggcatgaacatatgccaggaagcgttgaaatcaagctgaggatacttatggatgagctgaatccgtaa

SEQ ID NO: 12 - protein, 320 aa, Clostridium thermocellum
MAQLYDMPLEELKKYKPALTKQKDFDEFWEKSLKELAEIPLKYQLIPYDFPARRVKVFRVEYLGFKGANIEGWLAVPEGEGLYPGLVQFHGYNWAMDGCVPDVVNWALNGYAAFLMLVRGQQGRSVDNIVPGSGHALGWMSKGILSPEEYYYRGVYMDAVRAVEILASLPCVDESRIGVTGGSQGGGLALAVAALSGIPKVAAVHYPFLAHFERAIDVAPDGPYLEINEYLRRNSGEEIERQVKKTLSYFDIMNLAPRIKCRTWICTGLVDEITPPSTVFAVYNHLKCPKEISVFRYFGHEHMPGSVEIKLRILMDELNP

SEQ ID NO: 13 - DNA, 978 bp, Thermotoga neapolitana
atggccttcttcgatatgccccttgaggaactgaaaaagtaccggcctgaaaggtacgaggagaaagatttcgatgagttctggagggaaacacttaaagaaagcgaaggattccctctggatcccgtctttgaaaaggtggactttcatctcaaaacggttgaaacgtacgatgttactttctctggatacagggggcagagaataaagggctggcttcttgttccgaagttggcggaagaaaagcttccatgcgtcgtgcagtacataggttacaatggtggaaggggttttccacacgactggctgttctggccgtcaatgggttacatctgttttgtcatggacaccagggggcagggaagcggctggatgaagggagacacaccggattaccctgagggtccagtcgatccacagtaccccggattcatgacgaggggcattctggatccgggaacctattactacaggcgagtcttcgtggatgcggtcagggcggtggaagcagccatttccttcccgagagtggattccaggaaggtggtggtggccggaggcagtcagggtgggggaatcgcccttgcggtgagtgccctgtcgaacagggtgaaggctctgctctgcgatgtgccgtttctgtgccacttcagaagggccgtgcaacttgtcgacacacacccatacgtggagatcaccaacttcctcaaaacccacagggacaaagaggagattgttttcagaacactttcctacttcgatggtgtgaactttgcagcaagggcaaaggtgcccgccctgttttccgttgggctcatggacaccatctgtcctccctcgacggtcttcgccgcttacaaccactacgccggtccaaaggagatcagaatctatccgtacaacaaccacgaaggtggaggttctttccaggcaattgagcaggtgaaattcttgaagagactatttgaggaaggctag

SEQ ID NO: 14 - protein, 325 aa, Thermotoga neapolitana
MAFFDMPLEELKKYRPERYEEKDFDEFWRETLKESEGFPLDPVFEKVDFHLKTVETYDVTFSGYRGQRIKGWLLVPKLAEEKLPCVVQYIGYNGGRGFPHDWLFWPSMGYICFVMDTRGQGSGWMKGDTPDYPEGPVDPQYPGFMTRGILDPGTYYYRRVFVDAVRAVEAAISFPRVDSRKVVVAGGSQGGGIALAVSALSNRVKALLCDVPFLCHFRRAVQLVDTHPYVEITNFLKTHRDKEEIVFRTLSYFDGVNFAARAKVPALFSVGLMDTICPPSTVFAAYNHYAGPKEIRIYPYNNHEGGGSFQAIEQVKFLKRLFEEG

SEQ ID NO: 15 - DNA, 978 bp, Thermotoga maritima
atggccttcttcgatttaccactcgaagaactgaagaaatatcgtccagagcggtacgaagagaaagacttcgatgagttctgggaagagacactcgcagagagcgaaaagttccccttagaccccgtcttcgagaggatggagtctcacctcaaaacagtcgaagcgtacgatgtcaccttctccggatacaggggacagaggatcaaagggtggctccttgttccaaaactggaagaagaaaaacttccctgcgttgtgcagtacataggatacaacggtggaagaggattccctcacgactggctgttctggccttctatgggttacatatgtttcgtcatggatactcgaggtcagggaagcggctggctgaaaggagacacaccggattaccctgagggtcccgttgaccctcagtatccaggattcatgacaagaggaatactggatcccagaacttactactacagacgagtcttcacggacgctgtcagagccgttgaagctgctgcttcttttcctcaggtagatcaagaaagaatcgtgatagctggaggcagtcagggtggcggaatagcccttgcggtgagcgctctctcaaagaaagcaaaggctcttctgtgcgatgtgccgtttctgtgtcacttcagaagagcagtacagcttgtggatacgcatccatacgcggagatcacgaactttctaaagacccacagagacaaggaagaaatcgtgttcaggactctttcctatttcgatggagtgaacttcgcagccagagcgaagatccctgcgctgttttctgtgggtctcatggacaacatttgtcctccttcaacggttttcgctgcctacaattactacgctggaccgaaggaaatcagaatctatccgtacaacaaccacgagggaggaggctctttccaagcggttgaacaggtgaaattcttgaaaaaactatttgagaaaggctaa

SEQ ID NO: 16 - protein, 325 aa, Thermotoga maritima
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNICPPSTVFAAYNYYAGPKEIRIYPYNNHEGGGSFQAVEQVKFLKKLFEKG

SEQ ID NO: 17 - DNA, 963 bp, Thermoanaerobacterium sp.
atgggacttttcgacatgccattacaaaaacttagagaatacactggtacaaatccatgccctgaagatttcgatgagtattggaatagggctttagatgagatgaggtcagttgatcctaaaattgaattgaaagaaagtagctttcaagtatcctttgcagaatgctatgacttgtactttacaggtgttcgtggtgccagaattcatgcaaagtatataaaacctaagacagaagggaaacatccagcgttgataagatttcatggatattcgtcaaattcaggcgactggaacgacaaattaaattacgtggcggcaggcttcaccgttgtggctatggatgtaagaggtcaaggagggcagtctcaagatgttggcggtgtaactgggaatactttaaatgggcatattataagagggctagacgatgatgctgataatatgcttttcaggcatattttcttagacactgcccaattggctggaatagttatgaacatgccagaagttgatgaagatagagtgggagtcatgggaccttctcaaggcggagggctgtcgttggcgtgtgctgcattggagccaagggtacgcaaagtagtatctgaatatccttttttatctgactacaagagagtttgggacttagaccttgcaaaaaacgcctatcaagagattacggactatttcaggctttttgacccaaggcatgaaagggagaatgaggtatttacaaagcttggatatatagacgttaaaaaccttgcgaaaaggataaaaggcgatgtcttaatgtgcgttgggcttatggaccaagtatgtccgccatcaactgtttttgcagcctacaacaacatacagtcaaaaaaagatataaaagtgtatcctgattatggacatgaacctatgagaggatttggagatttagcgatgcagtttatgttggaactatattcataa

SEQ ID NO: 18 - protein, 320 aa, Thermoanaerobacterium sp.
MGLFDMPLQKLREYTGTNPCPEDFDEYWNRALDEMRSVDPKIELKESSFQVSFAECYDLYFTGVRGARIHAKYIKPKTEGKHPALIRFHGYSSNSGDWNDKLNYVAAGFTVVAMDVRGQGGQSQDVGGVTGNTLNGHIIRGLDDDADNMLFRHIFLDTAQLAGIVMNMPEVDEDRVGVMGPSQGGGLSLACAALEPRVRKVVSEYPFLSDYKRVWDLDLAKNAYQEITDYFRLFDPRHERENEVFTKLGYIDVKNLAKRIKGDVLMCVGLMDQVCPPSTVFAAYNNIQSKKDIKVYPDYGHEPMRGFGDLAMQFMLELYS

SEQ ID NO: 19 - DNA, 978 bp, Bacillus sp.
  CDS (1)..(978)
atgaacctttttgatatgccccttgaggagctgcagcattacaagcctgcccagaccaggcaggatgattttgagtcattctggaaaaagcggattgaggagaacagtcaatatccgctgaatatagaagtaatggagcgggtttatccggttccgggagtgagagtatatgatatttattttgacgggttccggaattcccgcatccatggggtgtatgttactccagaaactccgggagcggacactcctgcggcagtgatttttcacggctataactggaacacgctgcagccgcattacagcttcaagcacgtgattcaggggattcctgtactgatggtggaggtgcggggacaaaatctcttgtctccagatagaaatcattatgggaatggaggtccgggaggctggatgacactcggcgtgatggatcccgatcaatattattacagcctggtatatatggactgcttccgcagcattgatgctgtcagggaactgtcgaggaagagaagtgtgtttgtggaaggcggaagccagggaggtgcactggcgattgccgcagccgccctgcaggatgacatcctgcttgcactcgccgacatcccttttctcacccatttcaagcgttccgtggagctttcctcggatggaccgtatcaggagatttcccactacttcaaagttcatgatcctcttcatcaaacggaagagcaggtatatcagacgctcagctatgtggactgcatgaacatggccagcatggttgaatgtccagtccttctttcagccggtctggaagacatcgtttgtcccccgtccagtgcatttgcactgttcaaccatctcggcgggccaaaagaaatacgggcctatccggaatacgcccatgaagtaccggctgtccatgaagaggaaaagctgaagtttatatcttcaaggctaaaaaatagagaaaagaggtgccggccatga

SEQ ID NO: 20 - protein, 325 aa, Bacillus sp.
MNLFDMPLEELQHYKPAQTRQDDFESFWKKRIEENSQYPLNIEVMERVYPVPGVRVYDIYFDGFRNSRIHGVYVTPETPGADTPAAVIFHGYNWNTLQPHYSFKHVIQGIPVLMVEVRGQNLLSPDRNHYGNGGPGGWMTLGVMDPDQYYYSLVYMDCFRSIDAVRELSRKRSVFVEGGSQGGALAIAAAALQDDILLALADIPFLTHFKRSVELSSDGPYQEISHYFKVHDPLHQTEEQVYQTLSYVDCMNMASMVECPVLLSAGLEDIVCPPSSAFALFNHLGGPKEIRAYPEYAHEVPAVHEEEKLKFISSRLKNREKRCRP

SEQ ID NO: 21 - DNA, 960 bp, Bacillus halodurans
ttagagatcagataaaaattgaaaaatccgatcacgatggcctggcaaatcttcgtgagcaaagtctggatataactcgatactttttgtcgtcgtgagtttgttatacatggcaaattgtgtagacggcgggcaaaccgtatccattaacccaacagcaagtaagacttctccctttacgagtggagcaagatgctgaatatcaatatagcctagcttcgtaaagatttcagcctcacgtcggtgctgtggatcaaagcgacgaaaatacgtttgcaattcgtcataagctttctcggctaaatccatctcccatacgcgttggtaatcgctaaggaaaggataaacaggagctacctttttaattttcggttccaaagccgcacaagcaatcgctaaggcccctccttgtgaccaacctgtcactgccacgcgctcttcatcgacttcaggaaggttcatcacaatgttggcaagctgagccgtatcaagaaacacatgacggaacaataattgatcagcattatcatcgagtccgcgtattatatgaccggaatgagtattccccttcacgcctcctgtgtcttcagacaagcctccttgcccgcgaacgtccattgcaagaacagaatatccgagggctgcgtaatgaagtaaacccgtccattcccccgcattcatcgtatatccgtgaaaatgaataaccgccgggtgtgtcccgctcgtgtgtcttgggcgcacgtattttgcgtgaattctagcacccctaacccctgtaaaatataggtggaagcattctgcatacgtggtttgaaaatcactcggtatgagctctacgtttggatttacctttctcatctcttgtaaagcacgatcccaatactcagtaaagtcatctggctttggattacgtcccatgtactcttttaattcggttaacggcatgtctattagtggcat

SEQ ID NO: 22 - protein, 319 aa, Bacillus halodurans
MPLIDMPLTELKEYMGRNPKPDDFTEYWDRALQEMRKVNPNVELIPSDFQTTYAECFHLYFTGVRGARIHAKYVRPRHTSGTHPAVIHFHGYTMNAGEWTGLLHYAALGYSVLAMDVRGQGGLSEDTGGVKGNTHSGHIIRGLDDNADQLLFRHVFLDTAQLANIVMNLPEVDEERVAVTGWSQGGALAIACAALEPKIKKVAPVYPFLSDYQRVWEMDLAEKAYDELQTYFRRFDPQHRREAEIFTKLGYIDIQHLAPLVKGEVLLAVGLMDTVCPPSTQFAMYNKLTTTKSIELYPDFAHEDLPGHRDRIFQFLSDL

SEQ ID NO: 23 - DNA, 954 bp, Bacillus clausii
atgccattagtcgatatgccgttgcgcgagttgttagcttatgaaggaataaaccctaaaccagcagattttgaccaatactggaaccgggccaaaacggaaattgaagcgattgatcccgaagtcactctagtcgaatcttctttccagtgttcgtttgcaaactgttaccatttctattatcgaagcgctggaaatgcaaaaatccatgcgaaatacgtacagccaaaagcaggggagaagacgccagcagtttttatgttccatgggtatggggggcgttcagccgaatggagcagcttgttaaattatgtagcggcgggtttttctgttttctatatggacgtgcgtggacaaggtggaacttcagaggatcctgggggcgtaagggggaatacatataggggccacattattcgcggcctcgatgccgggccagacgcacttttttaccgcagcgttttcttggacaccgtccaattggttcgtgctgctaaaacattgcctcacatcgataaaacacggcttatggccacagggtggtcgcaagggggcgccttaacgcttgcctgtgctgcccttgttcctgaaatcaagcgtcttgctccagtatacccgtttttaagcgattacaagcgagtgtggcaaatggatttagcggttcgttcgtataaagaattggctgattatttccgttcatacgatccgcaacataaacgccatggcgaaatttttgaacgccttggctacatcgatgtccagcatcttgctgaccggattcaaggagatgtcctaatgggagttggtttaatggatacagaatgcccgccgtctacccaatttgctgcttataataaaataaaggctaaaaaatcgtatgagctctatcctgattttggccatgagcaccttccaggaatgaacgatcatatttttcgctttttcactagttga

SEQ ID NO: 24 - protein, 317 aa, Bacillus clausii
MPLVDMPLRELLAYEGINPKPADFDQYWNRAKTEIEAIDPEVTLVESSFQCSFANCYHFYYRSAGNAKIHAKYVQPKAGEKTPAVFMFHGYGGRSAEWSSLLNYVAAGFSVFYMDVRGQGGTSEDPGGVRGNTYRGHIIRGLDAGPDALFYRSVFLDTVQLVRAAKTLPHIDKTRLMATGWSQGGALTLACAALVPEIKRLAPVYPFLSDYKRVWQMDLAVRSYKELADYFRSYDPQHKRHGEIFERLGYIDVQHLADRIQGDVLMGVGLMDTECPPSTQFAAYNKIKAKKSYELYPDFGHEHLPGMNDHIFRFFTS

SEQ ID NO: 25 - DNA, 960 bp, Bacillus subtilis
  CDS (1)..(960)
atgcaactattcgatctgccgctcgaccaattgcaaacatataagcctgaaaaaacagcaccgaaagatttttctgagttttggaaattgtctttggaggaacttgcaaaagtccaagcagaacctgatctacagccggttgactatcctgctgacggagtaaaagtgtaccgtctcacatataaaagcttcggaaacgcccgcattaccggatggtacgcggtgcctgacaagcaaggcccgcatccggcgatcgtgaaatatcatggctacaatgcaagctatgatggtgagattcatgaaatggtaaactgggcactccatggctacgccgcattcggcatgcttgtccgcggccagcagagcagcgaggatacgagtatttcaccgcacggtcacgctttgggctggatgacgaaaggaattcttgataaagatacatactattaccgcggtgtttatttggacgccgtccgcgcgcttgaggtcatcagcagcttcgacgaggttgacgaaacaaggatcggtgtgacaggaggaagccaaggcggaggtttaaccattgccgcagcagcgctgtcagacattccaaaagccgcggttgccgattatccttatttaagcaacttcgaacgggccattgatgtggcgcttgaacagccgtaccttgaaatcaattccttcttcagaagaaatggcagcccggaaacagaagtgcaggcgatgaagacactttcatatttcgatattatgaatctcgctgaccgagtgaaggtgcctgtcctgatgtcaatcggcctgattgacaaggtcacgccgccatccaccgtgtttgccgcctacaatcatttggaaacagagaaagagctgaaggtgtaccgctacttcggacatgagtatatccctgcttttcaaacggaaaaacttgctttctttaagcagcatcttaaaggctgataa

SEQ ID NO: 26 - protein, 318 aa, Bacillus subtilis
MQLFDLPLDQLQTYKPEKTAPKDFSEFWKLSLEELAKVQAEPDLQPVDYPADGVKVYRLTYKSFGNARITGWYAVPDKQGPHPAIVKYHGYNASYDGEIHEMVNWALHGYAAFGMLVRGQQSSEDTSISPHGHALGWMTKGILDKDTYYYRGVYLDAVRALEVISSFDEVDETRIGVTGGSQGGGLTIAAAALSDIPKAAVADYPYLSNFERAIDVALEQPYLEINSFFRRNGSPETEVQAMKTLSYFDIMNLADRVKVPVLMSIGLIDKVTPPSTVFAAYNHLETEKELKVYRYFGHEYIPAFQTEKLAFFKQHLKG

SEQ ID NO: 27 - protein, 325 aa, Thermotoga neapolitana
  MISC_FEATURE (277)..(277): Xaa is Ala, Val, Ser, or Thr.
MAFFDMPLEELKKYRPERYEEKDFDEFWRETLKESEGFPLDPVFEKVDFHLKTVETYDVTFSGYRGQRIKGWLLVPKLAEEKLPCVVQYIGYNGGRGFPHDWLFWPSMGYICFVMDTRGQGSGWMKGDTPDYPEGPVDPQYPGFMTRGILDPGTYYYRRVFVDAVRAVEAAISFPRVDSRKVVVAGGSQGGGIALAVSALSNRVKALLCDVPFLCHFRRAVQLVDTHPYVEITNFLKTHRDKEEIVFRTLSYFDGVNFAARAKVPALFSVGLMDTIXPPSTVFAAYNHYAGPKEIRIYPYNNHEGGGSFQAIEQVKFLKRLFEEG

SEQ ID NO: 28 - protein, 325 aa, Thermotoga maritima
  MISC_FEATURE (277)..(277): Xaa is Ala, Val, Ser, or Thr.
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNIXPPSTVFAAYNYYAGPKEIRIYPYNNHEGGGSFQAVEQVKFLKKLFEKG

SEQ ID NO: 29 - protein, 326 aa, Thermotoga lettingae
  MISC_FEATURE (277)..(277): Xaa is Ala, Val, Ser, or Thr.
MVYFDMPLEDLRKYLPQRYEEKDFDDFWKQTIHETRGYFQEPILKKVDFYLQNVETFDVTFSGYRGQKIKGWLILPKFRNGKLPCVVEFVGYGGGRGFPYDWLLWSAAGYAHFIMDTRGQGSNWMKGDTPDYEDNPSDPQYPGFLTKGVLNPETYYYRRVFMDAFMAVETISQLEQIDSQTIILSGASQGGGIALAVSALSSKVMALLCDVPFLCHYKRAVQITDSMPYAEITRYCKTHIDKIQTVFRTLSYFDGVNFAARAKCPALFSVGLMDDIXPPSTVFAAYNYYAGEKDIRIYPYNNHEGGGSFHTLEKLKFVKKTISMRE

SEQ ID NO: 30 - protein, 325 aa, Thermotoga petrophilia
  MISC_FEATURE (277)..(277): Xaa is Ala, Val, Ser, or Thr.
MAFFDLPLEELKKYRPERYEEKDFDEFWEGTLAENEKFPLDPVFERMESHLKTVEAYDVTFSGYMGQRIKGWLLVPKLEEEKLPCVVQYIGYNGGRGFPHDWLFWPSMGYICFVMDTRGQGSGWMKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNIXPPSTVFAAYNHYAGPKEIRIYPYNNHEGGGSFQAIEQVKFLKRLFEKG

SEQ ID NO: 31 - protein, 325 aa, Thermotoga sp. RQ2a
  MISC_FEATURE (277)..(277): Xaa is Ala, Val, Ser, or Thr.
MAFFDLPLEELKKYRPERYEEKDFDEFWKETLAESEKFPLDPVFERMESHLKTVEVYDVTFSGYRGQRIKGWLLVPKLEEEKLPCVVQYIGYNGGRGFPHDWLFWPSMGYICFVMDTRGQGSGWLKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNIXPPSTVFAAYNHYAGPKEIRIYPYNNHEGGGSFQAIEQVKFLKRLFEKG

SEQ ID NO: 32 - protein, 329 aa, Thermotoga sp. RQ2b
  MISC_FEATURE (278)..(278): Xaa is Ala, Val, Ser, or Thr.
MALFDMPLEKLRSYLPDRYEEEDFDLFWKETLEESRKFPLDPIFERVDYLLENVEVYDVTFSGYRGQRIKAWLILPVVKKEERLPCIVEFIGYRGGRGFPFDWLFWSSAGYAHFVMDTRGQGTSRVKGDTPDYCDEPINPQFPGFMTRGILDPRTYYYRRVFTDAVRAVETASSFPGIDPERIAVVGTSQGGGIALAVAALSEIPKALVSNVPFLCHFRRAVQITDNAPYSEIVNYLKVHRDKEEIVFRTLSYFDGVNFAARAKIPALFSVALMDKTXPPSTVFAAYNHYAGPKEIKVYPFNEHEGGESFQRMEELRFMKRILKGEFKA

SEQ ID NO: 33 - protein, 326 aa, Thermotoga lettingae
MVYFDMPLEDLRKYLPQRYEEKDFDDFWKQTIHETRGYFQEPILKKVDFYLQNVETFDVTFSGYRGQKIKGWLILPKFRNGKLPCVVEFVGYGGGRGFPYDWLLWSAAGYAHFIMDTRGQGSNWMKGDTPDYEDNPSDPQYPGFLTKGVLNPETYYYRRVFMDAFMAVETISQLEQIDSQTIILSGASQGGGIALAVSALSSKVMALLCDVPFLCHYKRAVQITDSMPYAEITRYCKTHIDKIQTVFRTLSYFDGVNFAARAKCPALFSVGLMDDICPPSTVFAAYNYYAGEKDIRIYPYNNHEGGGSFHTLEKLKFVKKTISMRE

SEQ ID NO: 34 - protein, 325 aa, Thermotoga petrophilia
MAFFDLPLEELKKYRPERYEEKDFDEFWEGTLAENEKFPLDPVFERMESHLKTVEAYDVTFSGYMGQRIKGWLLVPKLEEEKLPCVVQYIGYNGGRGFPHDWLFWPSMGYICFVMDTRGQGSGWMKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNICPPSTVFAAYNHYAGPKEIRIYPYNNHEGGGSFQAIEQVKFLKRLFEKG

SEQ ID NO: 35 - protein, 325 aa, Thermotoga sp. RQ2a
MAFFDLPLEELKKYRPERYEEKDFDEFWKETLAESEKFPLDPVFERMESHLKTVEVYDVTFSGYRGQRIKGWLLVPKLEEEKLPCVVQYIGYNGGRGFPHDWLFWPSMGYICFVMDTRGQGSGWLKGDTPDYPEDPVDPQYPGFMTRGILDPRTYYYRRVFTDAVRAVEAAASFPRVDHERIVIAGGSQGGGIALAVSALSKKAKALLCDVPFLCHFRRAVQLVDTHPYAEITNFLKTHRDKEEIVFRTLSYFDGVNFAVRAKIPALFSVGLMDNICPPSTVFAAYNHYAGPKEIRIYPYNNHEGGGSFQAIEQVKFLKRLFEKG

SEQ ID NO: 36 - protein, 329 aa, Thermotoga sp. RQ2b
MALFDMPLEKLRSYLPDRYEEEDFDLFWKETLEESRKFPLDPIFERVDYLLENVEVYDVTFSGYRGQRIKAWLILPVVKKEERLPCIVEFIGYRGGRGFPFDWLFWSSAGYAHFVMDTRGQGTSRVKGDTPDYCDEPINPQFPGFMTRGILDPRTYYYRRVFTDAVRAVETASSFPGIDPERIAVVGTSQGGGIALAVAALSEIPKALVSNVPFLCHFRRAVQITDNAPYSEIVNYLKVHRDKEEIVFRTLSYFDGVNFAARAKIPALFSVALMDKTCPPSTVFAAYNHYAGPKEIKVYPFNEHEGGESFQRMEELRFMKRILKGEFKA

SEQ ID NO: 37 - DNA, 960 bp, Thermoanaerobacterium saccharolyticum
atgggtctgttcgatatgccactgcaaaaactgcgtgaatataccggtaccaacccatgtcctgaggatttcgatgaatactgggatcgcgcactggacgaaatgcgtagcgttgatcctaaaatcaagatgaagaagagctcctttcaagttccgttcgcggaatgttacgatctgtattttaccggcgttcgtggtgcccgcattcacgcgaaatacattcgtccgaaaaccgaaggcaaacacccggcgctgattcgcttccatggttactccagcaactctggtgattggaacgacaagctgaactacgttgcggctggttttaccgtagtagcgatggacgctcgtggccagggtggccaatctcaggacgtcggcggtgttaatggcaacaccctgaacggtcacatcatccgtggcctggacgatgatgcagataacatgctgttccgtcatattttcctggacaccgcgcagctggctggtatcgttatgaacatgccggaaatcgatgaggaccgcgtagctgttatgggtccgtcccagggcggcggtctgtccctggcgtgtgcggctctggaacctaaaatccgtaaagtagtgtccgaatatccgttcctgagcgactacaagcgtgtgtgggatctggatctggccaaaaatgcgtaccaagaaatcactgactatttccgtctgttcgacccacgccacgaacgtgagaacgaggtttttactaaactgggttacattgacgtaaagaacctggcgaaacgtatcaaaggtgatgttctgatgtgcgtgggcctgatggatcaggtctgcccgccgagcaccgtatttgcagcatacaacaacatccagtccaagaaggacatcaaagtctacccggactatggtcacgaaccgatgcgtggcttcggtgacctggctatgcagttcatgctggaactgtattct

SEQ ID NO: 38 - protein, 320 aa, Thermoanaerobacterium saccharolyticum
MGLFDMPLQKLREYTGTNPCPEDFDEYWDRALDEMRSVDPKIKMKKSSFQVPFAECYDLYFTGVRGARIHAKYIRPKTEGKHPALIRFHGYSSNSGDWNDKLNYVAAGFTVVAMDARGQGGQSQDVGGVNGNTLNGHIIRGLDDDADNMLFRHIFLDTAQLAGIVMNMPEIDEDRVAVMGPSQGGGLSLACAALEPKIRKVVSEYPFLSDYKRVWDLDLAKNAYQEITDYFRLFDPRHERENEVFTKLGYIDVKNLAKRIKGDVLMCVGLMDQVCPPSTVFAAYNNIQSKKDIKVYPDYGHEPMRGFGDLAMQFMLELYS

SEQ ID NO: 39 - DNA, 939 bp, Lactococcus lactis
atgacaaaaataaacaattggcaagattatcaaggaagttcacttaaaccagaggattttgataaattttgggatgaaaaaattaatttggtttcaaatcatcaatttgaatttgaattaatagaaaaaaatctttcctctaaggtagttaacttttatcatttgtggtttacagctattgatggagctaaaattcatgctcagttaattgttcccaagaatttgaaagagaaatacccagccatcttacaatttcatggttatcattgcgatagtggggattgggtcgataaaatagggatagttgccgaagggaatgtagttcttgcgcttgattgtcgaggacaaggtggtttaagtcaagataatattcaaactatggggatgacaatgaagggactcattgttcgaggaattgatgaagggtatgaaaatctctattacgttcgccaatttatggacttaataactgcaaccaaaattttatccgagtttgattttgttgatgaaacaaatataagtgcacaaggtgcttctcaaggtggagcgcttgccgttgcttgcgccgcactttctcctcttataaaaaaggtgactgccacttacccctttctttcagattatcgcaaagcttatgagcttggtgccgaggaatctgctttcgaagaacttccatattggtttcagtttaaagatccacttcatctaagagaagactggttttttaatcagttggaatacattgatattcaaaatttagcaccaagaattaaggctgaggtcatttggatcctaggcggcaaagatactgttgttcctccgattacgcaaatggcggcttacaataaaatacaaagtaaaaaatctctctatgtcttacctgaatacggccatgaatatcttcctaaaattagcgactggttaagagagaatcaataa

SEQ ID NO: 40 - protein, 312 aa, Lactococcus lactis
MTKINNWQDYQGSSLKPEDFDKFWDEKINLVSNHQFEFELIEKNLSSKVVNFYHLWFTAIDGAKIHAQLIVPKNLKEKYPAILQFHGYHCDSGDWVDKIGIVAEGNVVLALDCRGQGGLSQDNIQTMGMTMKGLIVRGIDEGYENLYYVRQFMDLITATKILSEFDFVDETNISAQGASQGGALAVACAALSPLIKKVTATYPFLSDYRKAYELGAEESAFEELPYWFQFKDPLHLREDWFFNQLEYIDIQNLAPRIKAEVIWILGGKDTVVPPITQMAAYNKIQSKKSLYVLPEYGHEYLPKISDWLRENQ

SEQ ID NO: 41 - DNA, 972 bp, Mesorhizobium loti
atgccgttcccggatctgatccagcccgaactgggcgcttatgtcagcagtgtcggcatgccggacgactttgcccaattctggacgtcgaccatcgccgaggctcgccaggccggcggtgaggtcagtatcgtgcaggcgcagacgacactgaaggcggtccagtccttcgatgtcacgtttccaggatacggcggtcatccaatcaaaggatggctgatcttgccgacgcaccacaaggggcggcttcccctcgtcgtgcagtatatcggctatggcggcggccgcggcttggcgcatgagcaactgcattgggcggcgtcaggctttgcctatttccgaatggatacacgcgggcagggaagcgactggagcgtcggtgagaccgccgatcccgtcggctcgacctcgtccattcccggctttatgacgcgtggcgtgctggacaagaatgactactattaccggcgcctgttcaccgatgccgtgagggcgatagatgctctgctcggactggacttcgtcgatcccgaacgcatcgcggtttgcggtgacagtcagggaggcggtatttcgctcgccgttggcggcatcgacccgcgcgtcaaggccgtaatgcccgacgttccatttctgtgcgactttccgcgcgctgtgcagactgccgtgcgcgatccctatttggaaatcgttcgctttctggcccagcatcgcgaaaagaaggcggcagtctttgaaacgctcaactatttcgactgcgtcaacttcgcccggcggtccaaggcgccggcgctgttttcggtggccctgatggacgaagtctgcccgccctctaccgtgtatggcgcattcaatgcctatgcaggcgaaaagaccatcacagagtacgaattcaacaatcatgaaggcgggcaaggctatcaagagcgccaacagatgacgtggctcagcaggctgttcggtgtcggctga

SEQ ID NO: 42 - protein, 323 aa, Mesorhizobium loit
MPFPDLIQPELGAYVSSVGMPDDFAQFWTSTIAEARQAGGEVSIVQAQTTLKAVQSFDVTFPGYGGHPIKGWLILPTHHKGRLPLVVQYIGYGGGRGLAHEQLHWAASGFAYFRMDTRGQGSDWSVGETADPVGSTSSIPGFMTRGVLDKNDYYYRRLFTDAVRAIDALLGLDFVDPERIAVCGDSQGGGISLAVGGIDPRVKAVMPDVPFLCDFPRAVQTAVRDPYLEIVRFLAQHREKKAAVFETLNYFDCVNFARRSKAPALFSVALMDEVCPPSTVYGAFNAYAGEKTITEYEFNNHEGGQGYQERQQMTWLSRLFGVG

SEQ ID NO: 43 - DNA, 990 bp, Geobacillus stearothermophilus
atgttcgatatgccgttagcacaattacagaaatacatggggacaaatccgaagccggctgattttgctgacttttggagtcgagcgttggaggaattatctgcccaatcgttgcattatgagctgattccggcaacatttcaaacgacagtggcgagttgctaccatttgtatttcacgggagtcggcggggctagagtccattgtcagttagtaaaaccgagagagcagaagcagaaaggcccggggttggtatggtttcatggctaccatacgaatagcggcgattgggtcgataaactggcatatgctgcggcaggttttactgtattggcgatggattgccgcggccaaggaggaaaatcagaggataatttgcaagtgaaaggcccaacattgaagggccatattattcgcggaattgaggatccaaatcctcatcatctttattatcgaaatgtttttttagatacagttcaggcggtaagaattttatgctctatggatcatattgatcgtgaacgaattggtgtatatggcgcttcccaaggaggagcgttggcattagcgtgtgctgctctggaaccatcggtggtgaaaaaagcggttgtgctctatccatttttatcggattataagcgggcgcaagagttggatatgaaaaataccgcgtatgaggaaattcattattattttcgatttttagatcccacacatgagcgggaagaagaagtattttacaaactaggctatattgatattcaactcttagccgatcggatttgtgccgatgttttatgggctgttgcgctagaagaccatatttgtcccccgtccacacaatttgctgtttataataaaattaagtcaaaaaaagacatggttttgttttacgagtatggtcatgagtatttaccgactatgggagaccgtgcttatctgtttttttgcccgatcttctttccaatccaaaagagaaacgttaagtaa

SEQ ID NO: 44 - protein, 329 aa, Geobacillus stearothermophilus
MFDMPLAQLQKYMGTNPKPADFADFWSRALEELSAQSLHYELIPATFQTTVASCYHLYFTGVGGARVHCQLVKPREQKQKGPGLVWFHGYHTNSGDWVDKLAYAAAGFTVLAMDCRGQGGKSEDNLQVKGPTLKGHIIRGIEDPNPHHLYYRNVFLDTVQAVRILCSMDHIDRERIGVYGASQGGALALACAALEPSVVKKAVVLYPFLSDYKRAQELDMKNTAYEEIHYYFRFLDPTHEREEEVFYKLGYIDIQLLADRICADVLWAVALEDHICPPSTQFAVYNKIKSKKDMVLFYEYGHEYLPTMGDRAYLFFCPIFFPIQKRNVK

SEQ ID NO: 45 - DNA, 978 bp, artificial sequence
  synthetic construct
atggcgttcttcgacctgcctctggaagaactgaagaaataccgtccagagcgttacgaagagaaggacatcgacgagttctgggaggaaactctggcggagaccgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgacctggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacgacatcagccctccttctaccgttttcgcggcatataactattatgcgggtccgaaagaaatccgtatctatccgtacaacaaccacgaaggcggtggtggctttcaggctgttgaacaagtgaaatccctgaagaaactgtttgagaagggctaa

SEQ ID NO: 46 - protein, 325 aa, artificial sequence
  synthetic construct
MAFFDLPLEELKKYRPERYEEKDIDEFWEETLAETEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDLERIVIAGGSQGGGIALAVSALSKKAKALLCDVPFLCHFRRAVQLVDTHPYAEITNFLKTHRDKEEIVFRTLSYFDGVNFAARAKIPALFSVGLMDDISPPSTVFAAYNYYAGPKEIRIYPYNNHEGGGGFQAVEQVKSLKKLFEKG

SEQ ID NO: 47 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacgacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 48 - protein, 325 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDDISPPSTVFAAYNYYAGPKEIRIYPYNNHEGGGSFQAVEQVKFLKKLFEKG

SEQ ID NO: 49 - DNA, 978 bp, Artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaatccctgaagaaactgtttgagaagggctaa

SEQ ID NO: 50 - protein, 325 aa, Artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKSLKKLFEKG

SEQ ID NO: 51 - DNA, 978 bp, Artificial sequence
  synthetic construct
atggcgttcttcgacctgcctctggaagaactgaagaaataccgtccagagcgttacgaagagaaggacttcgacgagttctgggaggaaactctggcggagaccgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 52 - protein, 325 aa, Artificial sequence
  synthetic construct
MAFFDLPLEELKKYRPERYEEKDFDEFWEETLAETEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 53 - DNA, 978 bp, Artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgacctggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 54 - protein, 325 aa, Artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDLERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 55 - DNA, 978 bp, artificial sequence
  synthetic construct
atggcgttcttcgacctgcctcgggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggcagaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctctggttgaccaggagcgtattgatatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttatccgcaccctgtcctatttcgacggcgttaacttcgcggctcgtgcaaaaattccggcactgttctctgttggtctgatggacaacatcagccctccttctaccgttttcgcggcatataactattatgcgggtctgaaagaaatccgtatctatccgtacaacaaccacgaaggcggtggtagctttcaggctgttgaacaagtgaaattcctgaagaaactgtttgagaagggctaa

SEQ ID NO: 56 - protein, 325 aa, artificial sequence
  Synthetic Construct
MAFFDLPREELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWQKGDTPDYPEGPVDPQYPGFMTRGILDPRTYYYRRVFTDAVRAVEAAASFPLVDQERIDIAGGSQGGGIALAVSALSKKAKALLCDVPFLCHFRRAVQLVDTHPYAEITNFLKTHRDKEEIVIRTLSYFDGVNFAARAKIPALFSVGLMDNISPPSTVFAAYNYYAGLKEIRIYPYNNHEGGGSFQAVEQVKFLKKLFEKG

SEQ ID NO: 57 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccggaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggaactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 58 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPELEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPELFSVGLMDNISPPSTVFAAYNYYAGPKEIRIYPYNNHEGGGSFQAVEQVKFLKKLFEKG

SEQ ID NO: 59 - DNA, 978 bp, artificial sequence
  synthetic construct
atggcgttcttcgacctgcctctggaagaactgaagaaataccgtccagagcgttacgaagagaaggacttcgacgagtactgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtgttctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggtactgttctctgttggtctgatggacaacatcagccctccttctaccgttttcgcggcatataactattatgcgggtccgaaagaaacccgtatctatccgtacaacagccacgaaggcggtggtagctttcaggctgttgaacaagtgaaattcctgaagaaactgtttgagaagggctaa

SEQ ID NO: 60 - protein, 325 aa, artificial sequence
  Synthetic Construct
MAFFDLPLEELKKYRPERYEEKDFDEYWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGVLDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPVLFSVGLMDNISPPSTVFAAYNYYAGPKETRIYPYNSHEGGGSFQAVEQVKFLKKLFEKG

SEQ ID NO: 61 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgcccaggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 62 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIAQAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 63 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggcttcatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 64 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGFICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 65 - protein, 12 aa, artificial sequence
  Synthetic construct
RVPNKTVTVDGA

SEQ ID NO: 66 - protein, 12 aa, artificial sequence
  Synthetic construct
DRHKSKYSSTKS

SEQ ID NO: 67 - protein, 12 aa, artificial sequence
  Synthetic construct
KNFPQQKEFPLS

SEQ ID NO: 68 - protein, 12 aa, artificial sequence
  Synthetic construct
QRNSPPAMSRRD

SEQ ID NO: 69 - protein, 12 aa, artificial sequence
  Synthetic construct
TRKPNMPHGQYL

SEQ ID NO: 70 - protein, 12 aa, artificial sequence
  Synthetic construct
KPPHLAKLPFTT

SEQ ID NO: 71 - protein, 12 aa, artificial sequence
  Synthetic construct
NKRPPTSHRIHA

SEQ ID NO: 72 - protein, 12 aa, artificial sequence
  Synthetic construct
NLPRYQPPCKPL

SEQ ID NO: 73 - protein, 12 aa, artificial sequence
  Synthetic construct
RPPWKKPIPPSE

SEQ ID NO: 74 - protein, 12 aa, artificial sequence
  Synthetic construct
RQRPKDHFFSRP

SEQ ID NO: 75 - protein, 12 aa, artificial sequence
  Synthetic construct
SVPNKXVTVDGX

SEQ ID NO: 76 - protein, 12 aa, artificial sequence
  Synthetic construct
TTKWRHRAPVSP

SEQ ID NO: 77 - protein, 12 aa, artificial sequence
  Synthetic construct
WLGKNRIKPRAS

SEQ ID NO: 78 - protein, 12 aa, artificial sequence
  Synthetic construct
SNFKTPLPLTQS

SEQ ID NO: 79 - protein, 12 aa, artificial sequence
  Synthetic construct
SVSVGMKPSPRP

SEQ ID NO: 80 - protein, 7 aa, artificial sequence
  Synthetic construct
DLHTVYH

SEQ ID NO: 81 - protein, 7 aa, artificial sequence
  Synthetic construct
HIKPPTR

SEQ ID NO: 82 - protein, 7 aa, artificial sequence
  Synthetic construct
HPVWPAI

SEQ ID NO: 83 - protein, 7 aa, artificial sequence
  Synthetic construct
MPLYYLQ

SEQ ID NO: 84 - protein, 26 aa, artificial sequence
  Synthetic construct
HLTVPWRGGGSAVPFYSHSQITLPNH

SEQ ID NO: 85 - protein, 41 aa, artificial sequence
  Synthetic construct
GPHDTSSGGVRPNLHHTSKKEKRENRKVPFYSHSVTSRGNV

SEQ ID NO: 86 - protein, 7 aa, artificial sequence
  Synthetic construct
KHPTYRQ

SEQ ID NO: 87 - protein, 7 aa, artificial sequence
  Synthetic construct
HPMSAPR

SEQ ID NO: 88 - protein, 7 aa, artificial sequence
  Synthetic construct
MPKYYLQ

SEQ ID NO: 89 - protein, 7 aa, artificial sequence
  Synthetic construct
MHAHSIA

SEQ ID NO: 90 - protein, 12 aa, artificial sequence
  Synthetic construct
AKPISQHLQRGS

SEQ ID NO: 91 - protein, 12 aa, artificial sequence
  Synthetic construct
APPTPAAASATT

SEQ ID NO: 92 - protein, 12 aa, artificial sequence
  Synthetic construct
DPTEGARRTIMT

SEQ ID NO: 93 - protein, 12 aa, artificial sequence
  Synthetic construct
LDTSFPPVPFHA

SEQ ID NO: 94 - protein, 12 aa, artificial sequence
  Synthetic construct
LDTSFHQVPFHQ

SEQ ID NO: 95 - protein, 11 aa, artificial sequence
  Synthetic construct
LPRIANTWSPS

SEQ ID NO: 96 - protein, 12 aa, artificial sequence
  Synthetic construct
RTNAADHPAAVT

SEQ ID NO: 97 - protein, 12 aa, artificial sequence
  Synthetic construct
SLNWVTIPGPKI

SEQ ID NO: 98 - protein, 12 aa, artificial sequence
  Synthetic construct
TDMQAPTKSYSN

SEQ ID NO: 99 - protein, 12 aa, artificial sequence
  Synthetic construct
TIMTKSPSLSCG

SEQ ID NO: 100 - protein, 12 aa, artificial sequence
  Synthetic construct
TPALDGLRQPLR

SEQ ID NO: 101 - protein, 12 aa, artificial sequence
  Synthetic construct
TYPASRLPLLAP

SEQ ID NO: 102 - protein, 12 aa, artificial sequence
  Synthetic construct
AKTHKHPAPSYS

SEQ ID NO: 103 - protein, 12 aa, artificial sequence
  Synthetic construct
TDPTPFSISPER

SEQ ID NO: 104 - protein, 12 aa, artificial sequence
  Synthetic construct
SQNWQDSTSYSN

SEQ ID NO: 105 - protein, 12 aa, artificial sequence
  Synthetic construct
WHDKPQNSSKST

SEQ ID NO: 106 - protein, 12 aa, artificial sequence
  Synthetic construct
LDVESYKGTSMP

SEQ ID NO: 107 - protein, 7 aa, artificial sequence
  Synthetic construct
NTPKENW

SEQ ID NO: 108 - protein, 7 aa, artificial sequence
  Synthetic construct
NTPASNR

SEQ ID NO: 109 - protein, 7 aa, artificial sequence
  Synthetic construct
PRGMLST

SEQ ID NO: 110 - protein, 7 aa, artificial sequence
  Synthetic construct
PPTYLST

SEQ ID NO: 111 - protein, 12 aa, artificial sequence
  Synthetic construct
TIPTHRQHDYRS

SEQ ID NO: 112 - protein, 7 aa, artificial sequence
  Synthetic construct
TPPTHRL

SEQ ID NO: 113 - protein, 7 aa, artificial sequence
  Synthetic construct
LPTMSTP

SEQ ID NO: 114 - protein, 7 aa, artificial sequence
  Synthetic construct
LGTNSTP

SEQ ID NO: 115 - protein, 12 aa, artificial sequence
  Synthetic construct
TPLTGSTNLLSS

SEQ ID NO: 116 - protein, 7 aa, artificial sequence
  Synthetic construct
TPLTKET

SEQ ID NO: 117 - protein, 7 aa, artificial sequence
  Synthetic construct
KQSHNPP

SEQ ID NO: 118 - protein, 7 aa, artificial sequence
  Synthetic construct
QQSHNPP

SEQ ID NO: 119 - protein, 7 aa, artificial sequence
  Synthetic construct
TQPHNPP

SEQ ID NO: 120 - protein, 12 aa, artificial sequence
  Synthetic construct
STNLLRTSTVHP

SEQ ID NO: 121 - protein, 12 aa, artificial sequence
  Synthetic construct
HTQPSYSSTNLF

SEQ ID NO: 122 - protein, 7 aa, artificial sequence
  Synthetic construct
SLLSSHA

SEQ ID NO: 123 - protein, 12 aa, artificial sequence
  Synthetic construct
QQSSISLSSHAV

SEQ ID NO: 124 - protein, 7 aa, artificial sequence
  Synthetic construct
NASPSSL

SEQ ID NO: 125 - protein, 7 aa, artificial sequence
  Synthetic construct
HSPSSLR

SEQ ID NO: 126 - protein, 7 aa, artificial sequence
  Synthetic construct
KXSHHTH

SEQ ID NO: 127 - protein, 7 aa, artificial sequence
  Synthetic construct
EXSHHTH

SEQ ID NO: 128 - protein, 12 aa, artificial sequence
  Synthetic construct
SHHTHYGQPGPV

SEQ ID NO: 129 - protein, 7 aa, artificial sequence
  Synthetic construct
LESTSLL

SEQ ID NO: 130 - protein, 7 aa, artificial sequence
  Synthetic construct
DLTLPFH

SEQ ID NO: 131 - protein, 8 aa, artificial sequence
  Synthetic construct
RTNAADHP

SEQ ID NO: 132 - protein, 12 aa, artificial sequence
  Synthetic construct
IPWWNIRAPLNA

SEQ ID NO: 133 - protein, 18 aa, artificial sequence
  Synthetic construct
EQISGSLVAAPWEGEGER

SEQ ID NO: 134 - protein, 12 aa, artificial sequence
  synthetic hair-binding peptide
TPPELLHGAPRS

SEQ ID NO: 135 - protein, 18 aa, artificial sequence
  Synthetic construct
LDTSFHQVPFHQKRKRKD

SEQ ID NO: 136 - protein, 18 aa, artificial sequence
  Synthetic construct
EQISGSLVAAPWKRKRKD

SEQ ID NO: 137 - protein, 18 aa, artificial sequence
  Synthetic construct
TPPELLHGDPRSKRKRKD

SEQ ID NO: 138 - protein, 13 aa, artificial sequence
  Synthetic construct
NTSQLSTEGEGED

SEQ ID NO: 139 - protein, 13 aa, artificial sequence
  Synthetic construct
TPPELLHGDPRSC

SEQ ID NO: 140 - protein, 20 aa, artificial sequence
  synthetic hair-binding peptide
HINKTNPHQGNHHSEKTQRQ

SEQ ID NO: 141 - protein, 15 aa, artificial sequence
  Synthetic construct
HAHKNQKETHQRHAA

SEQ ID NO: 142 - protein, 15 aa, artificial sequence
  Synthetic construct
HEHKNQKETHQRHAA

SEQ ID NO: 143 - protein, 20 aa, artificial sequence
  Synthetic construct
HNHMQERYTEPQHSPSVNGL

SEQ ID NO: 144 - protein, 17 aa, artificial sequence
  Synthetic construct
THSTHNHGSPRHTNADA

SEQ ID NO: 145 - protein, 20 aa, artificial sequence
  synthetic hair-binding peptide
GSCVDTHKADSCVANNGPAT

SEQ ID NO: 146 - protein, 20 aa, artificial sequence
  synthetic hair-binding peptide
AQSQLPDKHSGLHERAPQRY

SEQ ID NO: 147 - protein, 20 aa, artificial sequence
  Synthetic construct
AQSQLPAKHSGLHERAPQRY

SEQ ID NO: 148 - protein, 20 aa, artificial sequence
  Synthetic construct
AQSQLPEKHSGLHERAPQRY

SEQ ID NO: 149 - protein, 20 aa, artificial sequence
  synthetic hair-binding peptide
TDMMHNHSDNSPPHRRSPRN

SEQ ID NO: 150 - protein, 20 aa, artificial sequence
  synthetic hair-binding peptide
TPPELAHTPHHLAQTRLTDR

SEQ ID NO: 151 - protein, 12 aa, artificial sequence
  Synthetic construct
RLLRLLRLLRLL

SEQ ID NO: 152 - protein, 12 aa, artificial sequence
  Synthetic construct
TPPELLHGEPRS

SEQ ID NO: 153 - protein, 12 aa, artificial sequence
  Synthetic construct
TPPELLHGAPRS

SEQ ID NO: 154 - protein, 12 aa, artificial sequence
  Synthetic construct
EQISGSLVAAPW

SEQ ID NO: 155 - protein, 12 aa, artificial sequence
  Synthetic construct
NEVPARNAPWLV

SEQ ID NO: 156 - protein, 13 aa, artificial sequence
  Synthetic construct
NSPGYQADSVAIG

SEQ ID NO: 157 - protein, 12 aa, artificial sequence
  Synthetic construct
AKPISQHLQRGS

SEQ ID NO: 158 - protein, 12 aa, artificial sequence
  Synthetic construct
LDTSFPPVPFHA

SEQ ID NO: 159 - protein, 12 aa, artificial sequence
  Synthetic construct
SLNWVTIPGPKI

SEQ ID NO: 160 - protein, 12 aa, artificial sequence
  Synthetic construct
TQDSAQKSPSPL

SEQ ID NO: 161 - protein, 12 aa, artificial sequence
  Synthetic construct
KELQTRNVVQRE

SEQ ID NO: 162 - protein, 12 aa, artificial sequence
  Synthetic construct
QRNSPPAMSRRD

SEQ ID NO: 163 - protein, 12 aa, artificial sequence
  Synthetic construct
TPTANQFTQSVP

SEQ ID NO: 164 - protein, 12 aa, artificial sequence
  Synthetic construct
AAGLSQKHERNR

SEQ ID NO: 165 - protein, 12 aa, artificial sequence
  Synthetic construct
ETVHQTPLSDRP

SEQ ID NO: 166 - protein, 12 aa, artificial sequence
  Synthetic construct
KNFPQQKEFPLS

SEQ ID NO: 167 - protein, 12 aa, artificial sequence
  Synthetic construct
LPALHIQRHPRM

SEQ ID NO: 168 - protein, 12 aa, artificial sequence
  Synthetic construct
QPSHSQSHNLRS

SEQ ID NO: 169 - protein, 12 aa, artificial sequence
  Synthetic construct
RGSQKSKPPRPP

SEQ ID NO: 170 - protein, 12 aa, artificial sequence
  Synthetic construct
THTQKTPLLYYH

SEQ ID NO: 171 - protein, 12 aa, artificial sequence
  Synthetic construct
TKGSSQAILKST

SEQ ID NO: 172 - protein, 7 aa, artificial sequence
  Synthetic construct
TAATTSP

SEQ ID NO: 173 - protein, 7 aa, artificial sequence
  Synthetic construct
LGIPQNL

SEQ ID NO: 174 - protein, 20 aa, artificial sequence
  Synthetic construct
THSTHNHGSPRHTNADAGNP

SEQ ID NO: 175 - protein, 20 aa, artificial sequence
  Synthetic construct
QQHKVHHQNPDRSTQDAHHS

SEQ ID NO: 176 - protein, 15 aa, artificial sequence
  Synthetic construct
HHGTHHNATKQKNHV

SEQ ID NO: 177 - protein, 15 aa, artificial sequence
  Synthetic construct
STLHKYKSQDPTPHH

SEQ ID NO: 178 - protein, 12 aa, artificial sequence
  Synthetic construct
SVSVGMKPSPRP

SEQ ID NO: 179 - protein, 12 aa, artificial sequence
  synthetic hair-binding peptide
TPPTNVLMLATK

SEQ ID NO: 180 - protein, 12 aa, artificial sequence
  Synthetic construct
TPPELLHGDPRS

SEQ ID NO: 181 - protein, 7 aa, artificial sequence
  synthetic hair-binding peptide
NTSQLST

SEQ ID NO: 182 - protein, 15 aa, artificial sequence
  Synthetic construct
STLHKYKSQDPTPHH

SEQ ID NO: 183 - protein, 12 aa, artificial sequence
  synthetic hair-binding peptide
GMPAMHWIHPFA

SEQ ID NO: 184 - protein, 15 aa, artificial sequence
  synthetic hair-binding peptide
HDHKNQKETHQRHAA

SEQ ID NO: 185 - protein, 20 aa, artificial sequence
  Synthetic construct
HNHMQERYTDPQHSPSVNGL

SEQ ID NO: 186 - protein, 20 aa, artificial sequence
  synthetic hair-binding peptide
TAEIQSSKNPNPHPQRSWTN

SEQ ID NO: 187 - protein, 21 aa, artificial sequence
  Dyed-hair-bindg peptide
SSADFASFGFFGFSAASADSR

SEQ ID NO: 188 - protein, 23 aa, artificial sequence
  Dyed-hair-bindg peptide
SSFAEAWSRAWPRAEVFFPSRGY

SEQ ID NO: 189 - protein, 17 aa, artificial sequence
  Dyed-hair-bindg peptide
SSFSVNEPHAWMAPLSR

SEQ ID NO: 190 - protein, 17 aa, artificial sequence
  Dyed-hair-bindg peptide
SSFSWVYGHGGLGFASR

SEQ ID NO: 191 - protein, 17 aa, artificial sequence
  Dyed-hair-bindg peptide
SSFVSWSPYKSPPELSR

SEQ ID NO: 192 - protein, 21 aa, artificial sequence
  Dyed-hair-bindg peptide
SSFYGSSAFVSSGVSVAYGSR

SEQ ID NO: 193 - protein, 21 aa, artificial sequence
  Dyed-hair-bindg peptide
SSGSVAVSAEASWFSGVAASR

SEQ ID NO: 194 - protein, 15 aa, artificial sequence
  Dyed-hair-bindg peptide
SSHDEHYQYHYYSSR

SEQ ID NO: 195 - protein, 15 aa, artificial sequence
  Dyed-hair-bindg peptide
SSHYYYNDYDHQSSR

SEQ ID NO: 196 - protein, 17 aa, artificial sequence
  Dyed-hair-bindg peptide
SSLFNMYGHQSVLGPSR

SEQ ID NO: 197 - protein, 17 aa, artificial sequence
  Dyed-hair-bindg peptide
SSLFSDVHYGSNKALSR

SEQ ID NO: 198 - protein, 17 aa, artificial sequence
  Dyed-hair-bindg peptide
SSLLSDFHYGDMWDASR

SEQ ID NO: 199 - protein, 15 aa, artificial sequence
  Dyed-hair-bindg peptide
SSNYNYNYNYQYSSR

SEQ ID NO: 200 - protein, 21 aa, artificial sequence
  synethetic construct
SSNYNYNYNYQYSSREGEGER

SEQ ID NO: 201 - protein, 21 aa, ARTIFICIAL SEQUENCE
  Synthetic construct
SSNYNYNYNYQYSSRKRKRKD

SEQ ID NO: 202 - protein, 15 aa, artificial sequence
  Dyed-hair-bindg peptide
SSQYYQDYQYYHSSR

SEQ ID NO: 203 - protein, 23 aa, artificial sequence
  Dyed-hair-bindg peptide
SSSCMGSHNPRMSVEESTRNCSR

SEQ ID NO: 204 - protein, 23 aa, artificial sequence
  Dyed-hair-bindg peptide
SSSCNNNWFYSSTLPGGDHACSR

SEQ ID NO: 205 - protein, 23 aa, artificial sequence
  Dyed-hair-bindg peptide
SSSCYDVECSSFVAWMRGPSSSR

SEQ ID NO: 206 - protein, 21 aa, artificial sequence
  Dyed-hair-bindg peptide
SSSFAASSAFSFLVDAVAWSR

SEQ ID NO: 207 - protein, 17 aa, artificial sequence
  Dyed-hair-bindg peptide
SSSFAYLVPDDGWLSSR

SEQ ID NO: 208 - protein, 21 aa, artificial sequence
  Dyed-hair-bindg peptide
SSSGAVFSSGGADAGWGVWSR

SEQ ID NO: 209 - protein, 23 aa, artificial sequence
  Dyed-hair-bindg peptide
SSSSADAAYGHCCGAGFSTFSSR

SEQ ID NO: 210 - protein, 23 aa, artificial sequence
  Dyed-hair-bindg peptide
SSSSDVHNSIIGWDFYHSRGSSR

SEQ ID NO: 211 - protein, 21 aa, artificial sequence
  Dyed-hair-bindg peptide
SSSSLDFFSYSAFSGGVAESR

SEQ ID NO: 212 - protein, 23 aa, artificial sequence
  Dyed-hair-bindg peptide
SSSSNDSNVSWFHYYASGLTSSR

SEQ ID NO: 213 - protein, 21 aa, artificial sequence
  Dyed-hair-bindg peptide
SSVDYEVPLAVAAEWGFSVSR

SEQ ID NO: 214 - protein, 15 aa, artificial sequence
  Dyed-hair-bindg peptide
SSYHYDYDHYYESSR

SEQ ID NO: 215 - protein, 15 aa, artificial sequence
  Dyed-hair-bindg peptide
SSYYNYHYQYQDSSR

SEQ ID NO: 216 - protein, 15 aa, artificial sequence
  Dyed-hair-bindg peptide
SSYYYDYYQQDYSSR

SEQ ID NO: 217 - protein, 12 aa, artificial sequence
  Synthetic construct
KRGRHKRPKRHK

SEQ ID NO: 218 - protein, 7 aa, artificial sequence
  Synthetic construct
RLLRLLR

SEQ ID NO: 219 - protein, 12 aa, artificial sequence
  Synthetic construct
HKPRGGRKKALH

SEQ ID NO: 220 - protein, 18 aa, artificial sequence
  Synthetic construct
KPRPPHGKKHRPKHRPKK

SEQ ID NO: 221 - protein, 18 aa, artificial sequence
  Synthetic construct
RGRPKKGHGKRPGHRARK

SEQ ID NO: 222 - protein, 12 aa, artificial sequence
  Synthetic construct
TPFHSPENAPGS

SEQ ID NO: 223 - protein, 13 aa, artificial sequence
  Synthetic construct
TPFHSPENAPGSK

SEQ ID NO: 224 - protein, 16 aa, artificial sequence
  Synthetic construct
TPFHSPENAPGSGGGS

SEQ ID NO: 225 - protein, 17 aa, artificial sequence
  Synthetic construct
TPFHSPENAPGSGGGSS

SEQ ID NO: 226 - protein, 15 aa, artificial sequence
  Synthetic construct
TPFHSPENAPGSGGG

SEQ ID NO: 227 - protein, 7 aa, artificial sequence
  Synthetic construct
FTQSLPR

SEQ ID NO: 228 - protein, 12 aa, artificial sequence
  Synthetic construct
KQATFPPNPTAY

SEQ ID NO: 229 - protein, 12 aa, artificial sequence
  Synthetic construct
HGHMVSTSQLSI

SEQ ID NO: 230 - protein, 7 aa, artificial sequence
  Synthetic construct
LSPSRMK

SEQ ID NO: 231 - protein, 7 aa, artificial sequence
  Synthetic construct
LPIPRMK

SEQ ID NO: 232 - protein, 7 aa, artificial sequence
  Synthetic construct
HQRPYLT

SEQ ID NO: 233 - protein, 7 aa, artificial sequence
  Synthetic construct
FPPLLRL

SEQ ID NO: 234 - protein, 7 aa, artificial sequence
  Synthetic construct
QATFMYN

SEQ ID NO: 235 - protein, 11 aa, artificial sequence
  Synthetic construct
VLTSQLPNHSM

SEQ ID NO: 236 - protein, 7 aa, artificial sequence
  Synthetic construct
HSTAYLT

SEQ ID NO: 237 - protein, 12 aa, artificial sequence
  Synthetic construct
APQQRPMKTFNT

SEQ ID NO: 238 - protein, 12 aa, artificial sequence
  Synthetic construct
APQQRPMKTVQY

SEQ ID NO: 239 - protein, 7 aa, artificial sequence
  Synthetic construct
PPWLDLL

SEQ ID NO: 240 - protein, 7 aa, artificial sequence
  Synthetic construct
PPWTFPL

SEQ ID NO: 241 - protein, 7 aa, artificial sequence
  Synthetic construct
SVTHLTS

SEQ ID NO: 242 - protein, 7 aa, artificial sequence
  Synthetic construct
VITRLTS

SEQ ID NO: 243 - protein, 12 aa, artificial sequence
  Synthetic construct
DLKPPLLALSKV

SEQ ID NO: 244 - protein, 12 aa, artificial sequence
  Synthetic construct
SHPSGALQEGTF

SEQ ID NO: 245 - protein, 12 aa, artificial sequence
  Synthetic construct
FPLTSKPSGACT

SEQ ID NO: 246 - protein, 12 aa, artificial sequence
  Synthetic construct
DLKPPLLALSKV

SEQ ID NO: 247 - protein, 7 aa, artificial sequence
  Synthetic construct
PLLALHS

SEQ ID NO: 248 - protein, 7 aa, artificial sequence
  Synthetic construct
VPISTQI

SEQ ID NO: 249 - protein, 12 aa, artificial sequence
  Synthetic construct
YAKQHYPISTFK

SEQ ID NO: 250 - protein, 7 aa, artificial sequence
  Synthetic construct
HSTAYLT

SEQ ID NO: 251 - protein, 12 aa, artificial sequence
  Synthetic construct
STAYLVAMSAAP

SEQ ID NO: 252 - protein, 12 aa, artificial sequence
  Synthetic construct
SVSVGMKPSPRP

SEQ ID NO: 253 - protein, 12 aa, artificial sequence
  Synthetic construct
TMGFTAPRFPHY

SEQ ID NO: 254 - protein, 12 aa, artificial sequence
  Synthetic construct
NLQHSVGTSPVW

SEQ ID NO: 255 - protein, 15 aa, artificial sequence
  Synthetic construct
QLSYHAYPQANHHAP

SEQ ID NO: 256 - protein, 12 aa, artificial sequence
  Synthetic construct
NQAASITKRVPY

SEQ ID NO: 257 - protein, 14 aa, artificial sequence
  Synthetic construct
SGCHLVYDNGFCDH

SEQ ID NO: 258 - protein, 14 aa, artificial sequence
  Synthetic construct
ASCPSASHADPCAH

SEQ ID NO: 259 - protein, 14 aa, artificial sequence
  Synthetic construct
NLCDSARDSPRCKV

SEQ ID NO: 260 - protein, 12 aa, artificial sequence
  Synthetic construct
NHSNWKTAADFL

SEQ ID NO: 261 - protein, 12 aa, artificial sequence
  Synthetic construct
GSSTVGRPLSYE

SEQ ID NO: 262 - protein, 12 aa, artificial sequence
  Synthetic construct
SDTISRLHVSMT

SEQ ID NO: 263 - protein, 12 aa, artificial sequence
  Synthetic construct
SPLTVPYERKLL

SEQ ID NO: 264 - protein, 12 aa, artificial sequence
  Synthetic construct
SPYPSWSTPAGR

SEQ ID NO: 265 - protein, 12 aa, artificial sequence
  Synthetic construct
VQPITNTRYEGG

SEQ ID NO: 266 - protein, 12 aa, artificial sequence
  Synthetic construct
WPMHPEKGSRWS

SEQ ID NO: 267 - protein, 14 aa, artificial sequence
  Synthetic construct
DACSGNGHPNNCDR

SEQ ID NO: 268 - protein, 14 aa, artificial sequence
  Synthetic construct
DHCLGRQLQPVCYP

SEQ ID NO: 269 - protein, 14 aa, artificial sequence
  Synthetic construct
DWCDTIIPGRTCHG

SEQ ID NO: 270 - protein, 12 aa, artificial sequence
  Synthetic construct
ALPRIANTWSPS

SEQ ID NO: 271 - protein, 12 aa, artificial sequence
  Synthetic construct
YPSFSPTYRPAF

SEQ ID NO: 272 - protein, 8 aa, Artificial Sequence
  Synthetic construct - caspace 3 cleavable linker
LESGDEVD

SEQ ID NO: 273 - protein, 37 aa, Artificial Sequence
  synthetic construct
TSTSKASTTTTSSKTTTTSSKTTTTTSKTSTTSSSST

SEQ ID NO: 274 - protein, 22 aa, Artificial Sequence
  synthetic construct
GQGGYGGLGSQGAGRGGLGGQG

SEQ ID NO: 275 - protein, 10 aa, Artificial Sequence
  synthetic construct
GPGGYGPGQQ

SEQ ID NO: 276 - protein, 9 aa, Artificial Sequence
  synthetic construct
GGSGPGSGG

SEQ ID NO: 277 - protein, 5 aa, Artificial Sequence
  synthetic construct
GGPKK

SEQ ID NO: 278 - protein, 5 aa, Artificial Sequence
  synthetic construct
GPGVG

SEQ ID NO: 279 - protein, 7 aa, Artificial Sequence
  synthetic construct
GGGCGGG

SEQ ID NO: 280 - protein, 4 aa, Artificial Sequence
  synthetic construct
GGGC

SEQ ID NO: 281 - protein, 14 aa, Artificial Sequence
  synthetic construct
PHMASMTGGQQMGS

SEQ ID NO: 282 - protein, 16 aa, Artificial Sequence
  synthetic construct
GPEEAAKKEEAAKKPA

SEQ ID NO: 283 - protein, 14 aa, Artificial Sequence
  synthetic construct
GSGGGGSGSGGGGS

SEQ ID NO: 284 - protein, 37 aa, Artificial Sequence
  synthetic construct
GPEPEPEPEPIPEPPKEAPVVIEKPKPKPKPKPKPPA

SEQ ID NO: 285 - protein, 18 aa, artificial sequence
  synthetic construct
GPGSGGAGSPGSAGGPGS

SEQ ID NO: 286 - DNA, 1293 bp, artificial sequence
  synthetic construct
atggcattcttcgacctgccactggaggagctgaagaaatatcgtcctgagcgctatgaagaaaaggattttgatgagttctgggaggaaactctggcagaaagcgagaaattcccgctggacccggtgtttgaacgtatggagagccatttgaaaaccgtggaagcctacgatgtgacctttagcggctatcgtggtcaacgcattaaaggttggctgctggtgcctaagctggaagaagagaagttgccttgcgtggtgcaatacattggttacaacggtggtcgtggttttccgcacgattggttgttctggccgagcatgggttacatttgctttgtgatggatacccgcggtcaaggtagcggttggctgaaaggcgacaccccggattacccggagggtccagtcgacccacagtacccgggttttatgacccgtggtatccttgacccgcgtacctactactaccgtcgtgtgttcaccgacgcggtacgtgcagttgaggcagccgcgtccttcccacaggttgaccaggaacgcatcgtgattgcgggtggctcgcaaggtggtggtatcgcattggcggttagcgctctgtccaagaaagcaaaagcactgctgtgcgacgtgccgtttctgtgtcacttccgtcgtgcagttcagctggttgatacgcacccttacgccgaaattaccaactttctgaaaacgcaccgcgataaggaagaaatcgtgttccgcaccctgagctattttgacggcgtcaatttcgcagcgcgtgcgaagattccagcgttgttcagcgttggtctgatggataacatttccccgccttctaccgttttcgcggcctacaactactacgcaggcccgaaagagattcgcatctacccatataacaaccatgagggtggcggtagcttccaggcagttgagcaagttaagttcctgaagaagctgttcgaaaagggtggtccgggttcgggtggtgcgggcagcccgggtagcgccggtggccctggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 287 - DNA, 1305 bp, artificial sequence
  synthetic construct
atggcattcttcgacctgccactggaggagctgaagaaatatcgtcctgagcgctatgaagaaaaggattttgatgagttctgggaggaaactctggcagaaagcgagaaattcccgctggacccggtgtttgaacgtatggagagccatttgaaaaccgtggaagcctacgatgtgacctttagcggctatcgtggtcaacgcattaaaggttggctgctggtgcctaagctggaagaagagaagttgccttgcgtggtgcaatacattggttacaacggtggtcgtggttttccgcacgattggttgttctggccgagcatgggttacatttgctttgtgatggatacccgcggtcaaggtagcggttggctgaaaggcgacaccccggattacccggagggtccagtcgacccacagtacccgggttttatgacccgtggtatccttgacccgcgtacctactactaccgtcgtgtgttcaccgacgcggtacgtgcagttgaggcagccgcgtccttcccacaggttgaccaggaacgcatcgtgattgcgggtggctcgcaaggtggtggtatcgcattggcggttagcgctctgtccaagaaagcaaaagcactgctgtgcgacgtgccgtttctgtgtcacttccgtcgtgcagttcagctggttgatacgcacccttacgccgaaattaccaactttctgaaaacgcaccgcgataaggaagaaatcgtgttccgcaccctgagctattttgacggcgtcaatttcgcagcgcgtgcgaagattccagcgttgttcagcgttggtctgatggataacatttccccgccttctaccgttttcgcggcctacaactactacgcaggcccgaaagagattcgcatctacccatataacaaccatgagggtggcggtagcttccaggcagttgagcaagttaagttcctgaagaagctgttcgaaaagggtggtccgggttcgggtggtgcgggcagcccgggtagcgccggtggccctggatccgcgcaaagccaactgccggacaaacatagcggtctgcacgagcgtgcgccgcagcgttacggtagcggtacggcggaaattcaatccagcaagaacccgaacccgcacccgcagcgcagctggaccaatggcagcggtcataatcacatgcaagagcgttacacggacccgcagcacagcccgagcgttaatggtttgggtagcggccacgaccataagaatcagaaagaaacccatcaacgccacgcggcgtccagccaccaccaccatcaccac

SEQ ID NO: 288 - protein, 431 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 289 - protein, 435 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 290 - protein, 88 aa, artificial sequence
  synthetic construct
PSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 291 - protein, 92 aa, artificial sequence
  synthetic construct
AQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 292 - DNA, 6368 bp, artificial sequence
  synthetic construct
agatctcgatcccgcgaaattaatacgactcactatagggagaccacaacggtttccctctagaaataattttgtttaactttaagaaggagatatacatatgcacactccagaacatatcaccgcagtagtacagcgttttgtggcagctctgaacgcgggcgagctggaaggtattgtggcgctgttcgcggaagaagccaccgtggaagaaccggtgggttctgaaccgcgttccggcaccgcagcctgccgtgaattttacgcaaacagcctgaagctgccgctggcggttgaactgacccaagaatgtcgtgcggtggctaacgaagccgctttcgcgttcaccgtgtccttcgaataccagggtcgtaagaccgttgtggcgccatgcgaacactttcgtttcaacggcgcaggcaaagtggtttccatccgcgcactgttcggtgaaaagaacatccatgcttgtcagggatccgatccgactccgccgacgaatgtactgatgctggcaaccaaaggcggtggtacgcattccacgcacaaccatggcagcccgcgccacacgaatgctgacgcaggcaatccgggcggcggcaccccaccaaccaatgtcctgatgctggctactaaaggcggcggcacgcattctacccacaaccatggtagcccgcgccatactaatgcagatgccggcaacccgggcggtggtaccccgccaaccaacgttctgatgctggcgacgaaaggtggcggtacccattccacgcataatcatggcagccctcgccacaccaacgctgatgctggtaatcctggtggcggtaagaagaaataataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 293 - protein, 325 aa, Thermotoga maritima
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKG

SEQ ID NO: 294 - protein, 431 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGGTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 295 - protein, 435 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGGTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 296 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgtcctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 297 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALSSVGLMDNITPPSTVFAAYNYYAGPKEIRIYPYNNHEGGGSFQAVEQVKFLKKLFEKG

SEQ ID NO: 298 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttctgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaactcctgaagaaactgtttgagaagggctaa

SEQ ID NO: 299 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFCRAVQLVDTHPYAEITNFLKTHRDKEEIVFRTLSYFDGVNFAARAKIPALFSVGLMDNITPPSTVFAAYNYYAGPKEIRIYPYNNHEGGGSFQAVEQVKLLKKLFEKG

SEQ ID NO: 300 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagataccctaccgtacgcggagattgctaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgtgggtccgaaagaaatccgtatctatccgtacaacaaccacgaaggcggtggtagctttcaggctgttgaacaagtgaaattcctgaagaaactgtttgagaagggctaa

SEQ ID NO: 301 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTLPYAEIANFLKTHRDKEEIVFRTLSYFDGVNFAARAKIPALFSVGLMDNITPPSTVFAAYNYYVGPKEIRIYPYNNHEGGGSFQAVEQVKFLKKLFEKG

SEQ ID NO: 302 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcggcggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 303 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFGGVNFAARAKIPALFSVGLMDNITPPSTVFAAYNYYAGPKEIRIYPYNNHEGGGSFQAVEQVKFLKKLFEKG

SEQ ID NO: 304 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctagtgcaaaatttccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 305 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAASAKFPALFSVGLMDNITPPSTVFAAYNYYAGPKEIRIYPYNNHEGGGSFQAVEQVKFLKKLFEKG

SEQ ID NO: 306 - DNA, 978 bp, artificial sequence
  synthetic construct
atggcgttcttcgacctgcctctggaagaactgaagaaataccgtccagagcgttacgaagagaaggacttcgacgagttctgtgaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgtcctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 307 - protein, 325 aa, artificial sequence
  Synthetic Construct
MAFFDLPLEELKKYRPERYEEKDFDEFCEETLAESEKFPLDPVFERMESHLKTVEAYDVTFSGYRGQRIKGWLLVPKLEEEKLPCVVQYIGYNGGRGFPHDWLSWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNITPPSTVFAAYNYYAGPKEIRIYPYNNHEGGGSFQAVEQVKFLKKLFEKG

SEQ ID NO: 308 - DNA, 978 bp, artificial sequence
  synthetic construct
atggcgttcttcgacctgcctctggaagaactgaagaaataccgtccagagcgttacgaagagaaggacttcgacgagttctgggaggaaactctggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggatccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccgccactgttctctgttggtctgatggacaacatcagccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 309 - protein, 325 aa, artificial sequence
  Synthetic Construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPPLFSVGLMDNISPPSTVFAAYNYYAGPKEIRIYPYNNHEGGGSFQAVEQVKFLKKLFEKG

SEQ ID NO: 310 - DNA, 978 bp, artificial sequence
  synthetic construct
atggcgttcttcgacctgcctctggaagaactgaagaaataccgtccagagcgttacgaagagaaggacttcgacgagttctgtgaggaaactccggcggagagcgaaaagtttccgctggacccagtgttcgagcgtatggaatctcacctgaaaaccgtggaggcatatgacgttactttttctggttaccgtggccagcgtatcaaaggctggctgctggttccgaaactggaggaagaaaaactgccgtgcgtagttcagtacatcggttacaacggtggccgtggctttccgcacgattggctgttctggccgtctatgggctacatttgcttcgtcatggatactcgtggtcagggttccggctggctgaaaggcgatactccggattatccggagggcccggtagacccgcagtaccctggcttcatgacgcgtggtattctggaaccgcgtacctattactatcgccgcgtttttaccgatgcagttcgtgccgtagaggccgcggcttctttccctcaggttgaccaggagcgtattgttatcgctggtggctcccagggtggcggcatcgccctggcggtatctgcgctgagcaagaaagctaaggcactgctgtgtgacgtcccgttcctgtgtcacttccgtcgcgctgttcagctggtagatacccatccgtacgcggagattactaacttcctgaaaactcaccgcgacaaagaagaaatcgttttccgcaccctgtcctatttcgacggcgttaacttcgcggctcgtgcaaaaattccggcactgttctctgttggtctgatggacaacatcacccctccttctaccgttttcgcggcatataactattatgcgggtccgaaagaaatccgtatctatccgtacaacaaccacgaaggcggtggtagctttcaggctgttgaacaagtgaaattcctgaagaaactgtttgagaagggctaa

SEQ ID NO: 311 - protein, 325 aa, artificial sequence
  Synthetic Construct
MAFFDLPLEELKKYRPERYEEKDFDEFCEETPAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILEPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNITPPSTVFAAYNYYAGPKEIRIYPYNNHEGGGSFQAVEQVKFLKKLFEKG

SEQ ID NO: 312 - protein, 88 aa, artificial sequence
  synthetic construct
PSAQSQLPDRHSGLHERAPQRYGPEPEPEPEPIPEPPREAPVVIERPRPRPRPRPRPPAHDHRNQRETHQRHAAGSGGGGSPHHHHHH

SEQ ID NO: 313 - protein, 16 aa, artificial sequence
  synthetic construct
GKGKGKGKGKHHHHHH

SEQ ID NO: 314 - protein, 216 aa, Mycobacterium smegmatis
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 315 - protein, 272 aa, Pseudomonas fluorescens
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKR

SEQ ID NO: 316 - DNA, 1278 bp, artificial sequence
  synthetic construct
atgcagctgttcgatttgagcctggaagaattgaaaaagtacaaaccgaaaaagacggcgcgtccggacttttctgatttttggaaaaagtctctggaggaactgcgtcaggtcgaggcggagccgaccctggaaagctacgactaccctgtcaagggcgttaaggtgtaccgcctgacctaccagagcttcggtcatagcaaaatcgagggtttctatgcggtgccggaccaaaccggtccgcacccggcactggttcgtttccacggttataacgccagctatgatggcggtatccatgacatcgtcaattgggcactgcatggttacgcaacgtttggcatgctggttcgcggccaaggcggtagcgaggataccagcgttaccccgggtggccacgcgctgggctggatgaccaagggtattctgtccaaggatacctattactaccgtggtgtatacttggatgcagttcgtgcgctggaggtcattcaaagctttccggaagttgacgagcatcgtatcggtgtgattggtggtagccagggtggcgcgctggcgattgcagctgccgcgttgagcgatattccgaaagtcgtggttgcggactatccgtatctgtcgaactttgagcgcgctgtcgacgtggcactggaacaaccgtacctggagattaacagctacttccgccgtaatagcgacccgaaagtggaggagaaggcgtttgaaactctgagctattttgatctgatcaatctggcgggttgggtgaaacaaccgaccctgatggccattggcctgatcgataagatcactccgccgtctacggtgttcgcggcatataaccacctggaaaccgacaaagacttgaaagtttaccgttatttcggccacgagtttatcccagccttccagacggaaaagttgagcttcctgcagaaacacctgctgctgagcacgggtccgggcagcggcggtgctggttcccctggcagcgccggtggtccaggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 317 - protein, 426 aa, artificial sequence
  synthetic construct
MQLFDLSLEELKKYKPKKTARPDFSDFWKKSLEELRQVEAEPTLESYDYPVKGVKVYRLTYQSFGHSKIEGFYAVPDQTGPHPALVRFHGYNASYDGGIHDIVNWALHGYATFGMLVRGQGGSEDTSVTPGGHALGWMTKGILSKDTYYYRGVYLDAVRALEVIQSFPEVDEHRIGVIGGSQGGALAIAAAALSDIPKVVVADYPYLSNFERAVDVALEQPYLEINSYFRRNSDPKVEEKAFETLSYFDLINLAGWVKQPTLMAIGLIDKITPPSTVFAAYNHLETDKDLKVYRYFGHEFIPAFQTEKLSFLQKHLLLSTGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 318 - DNA, 1254 bp, artificial sequence
  synthetic construct
atgaccaaaatcaacaattggcaagactaccaaggtagctctctgaaaccggaggacttcgataagttttgggacgagaaaatcaatctggtgagcaatcatcagtttgagtttgagctgatcgaaaagaacctgagcagcaaggttgtgaatttctatcatctgtggttcaccgcaattgacggtgcaaaaatccacgcgcaattgatcgtcccgaaaaacctgaaagaaaagtatcctgccatcctgcaatttcacggttatcactgcgatagcggcgactgggttgacaaaattggcatcgtggcggaaggcaacgtagtgctggcactggattgtcgcggtcagggtggcctgagccaagacaatatccagacgatgggtatgactatgaaaggtctgattgttcgcggcattgacgagggttatgagaacctgtactacgtccgtcaattcatggatctgatcaccgcgacgaagattctgagcgaattcgattttgtcgatgaaaccaacatcagcgcgcagggcgccagccaaggtggtgcgctggcggttgcgtgcgcggcactgagcccgctgattaagaaggtcacggctacgtacccgttcttgtccgactaccgtaaagcgtacgaactgggtgccgaggaaagcgcctttgaggagctgccatattggttccagtttaaagacccgttgcacttgcgtgaggattggttcttcaaccagctggaatacatcgacattcagaatctggctccgcgtattaaggcagaggttatttggatcttgggcggtaaagataccgtggtgccgccgattacccaaatggctgcgtacaacaagattcagtccaagaaaagcctgtatgttctgcctgaatacggccacgagtatctgccgaagatttcggattggctgcgcgaaaatcagggtccgggtagcggcggtgcgggttctccgggcagcgcaggcggtccgggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 319 - protein, 418 aa, artificial sequence
  synthetic construct
MTKINNWQDYQGSSLKPEDFDKFWDEKINLVSNHQFEFELIEKNLSSKVVNFYHLWFTAIDGAKIHAQLIVPKNLKEKYPAILQFHGYHCDSGDWVDKIGIVAEGNVVLALDCRGQGGLSQDNIQTMGMTMKGLIVRGIDEGYENLYYVRQFMDLITATKILSEFDFVDETNISAQGASQGGALAVACAALSPLIKKVTATYPFLSDYRKAYELGAEESAFEELPYWFQFKDPLHLREDWFFNQLEYIDIQNLAPRIKAEVIWILGGKDTVVPPITQMAAYNKIQSKKSLYVLPEYGHEYLPKISDWLRENQGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 320 - DNA, 1287 bp, artificial sequence
  synthetic construct
atgccgtttccggatctgatccagccggagctgggcgcatacgtcagctccgtcggtatgccggacgatttcgctcaattctggaccagcaccattgccgaagctcgtcaggcaggtggcgaggttagcatcgtccaagctcagacgactctgaaagcagtccagagcttcgacgttaccttcccgggctacggcggtcacccgatcaagggttggctgatcctgccaacccaccacaaaggtcgcctgccgctggtggtacagtacattggttatggcggtggtcgtggcttggcgcatgaacagttgcactgggcagcatccggctttgcgtacttccgcatggacacccgtggtcaaggtagcgattggtcggttggtgagactgccgacccggttggtagcaccagcagcatcccgggctttatgacccgtggtgtgctggataagaatgactactattaccgtcgcttgttcacggacgcggtccgtgctattgatgcgctgctgggtctggactttgtggacccggagcgcattgccgtctgcggtgacagccagggtggcggtatcagcctggcggttggcggcatcgatccgcgtgttaaagcggttatgccggatgtgccgttcctgtgtgattttccgcgtgccgtccagacggccgttcgcgacccgtacctggagattgtgcgctttttggcacaacaccgtgaaaagaaagcagcggtgttcgaaaccctgaactattttgactgtgtgaattttgcgcgtcgtagcaaagcgcctgcgctgtttagcgtggcgctgatggatgaagtgtgcccgccatctaccgtttatggtgccttcaacgcgtatgcgggcgaaaagaccattacggagtacgagttcaataaccacgagggtggccaaggctatcaagaacgtcagcaaatgacgtggctgtctcgcctgttcggtgtcggcggtccgggtagcggtggtgcgggcagccctggcagcgcaggtggtccgggatcccctagcgcacaaagccaactgccggacaagcatagcggcctgcacgaacgtgctccgcagcgttacggtccggaaccggaaccggaaccggagccgatcccagaaccgccgaaagaggccccagttgttattgaaaagccgaagccgaaaccgaagccgaagccgaagccgcctgcgcatgatcataagaatcagaaggaaacccatcagcgtcacgccgctggttcgggcggtggtggtagcccgcaccatcaccaccaccac

SEQ ID NO: 321 - protein, 429 aa, artificial sequence
  synthetic construct
MPFPDLIQPELGAYVSSVGMPDDFAQFWTSTIAEARQAGGEVSIVQAQTTLKAVQSFDVTFPGYGGHPIKGWLILPTHHKGRLPLVVQYIGYGGGRGLAHEQLHWAASGFAYFRMDTRGQGSDWSVGETADPVGSTSSIPGFMTRGVLDKNDYYYRRLFTDAVRAIDALLGLDFVDPERIAVCGDSQGGGISLAVGGIDPRVKAVMPDVPFLCDFPRAVQTAVRDPYLEIVRFLAQHREKKAAVFETLNYFDCVNFARRSKAPALFSVALMDEVCPPSTVYGAFNAYAGEKTITEYEFNNHEGGQGYQERQQMTWLSRLFGVGGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 322 - DNA, 966 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccccttccgcgcagagccagctgccggacaaacactccggtctgcacgagcgcgcaccgcagcgttacggtccggagccagagccggaaccggagccgatcccggagccgccgaaagaagcgccggttgttatcgagaagccgaaaccgaagccaaagcctaagccgaagccaccggcccatgaccacaaaaatcaaaaggaaacccatcagcgtcacgcagcgggttctggtggtggcggcagcccgcatcaccaccatcaccat

SEQ ID NO: 323 - protein, 322 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 324 - DNA, 966 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccccgagcgcgcaatcccagttgccggatcgccacagcggtctgcatgagcgtgccccgcaacgttacggtccagagccggagccggaaccggagccgattccggaaccaccgcgcgaggctccggtggttatcgaacgtccacgtccacgcccgcgcccgcgtccgcgtccaccggcgcacgaccaccgtaatcaacgtgaaacccaccagcgccacgcagccggcagcggtggtggtggtagcccgcatcatcaccatcatcac

SEQ ID NO: 325 - protein, 322 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSPSAQSQLPDRHSGLHERAPQRYGPEPEPEPEPIPEPPREAPVVIERPRPRPRPRPRPPAHDHRNQRETHQRHAAGSGGGGSPHHHHHH

SEQ ID NO: 326 - DNA, 978 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccgcgcaaagccaactgccggacaaacatagcggtctgcacgagcgtgcgccgcagcgttacggtagcggtacggcggaaattcaatccagcaagaacccgaacccgcacccgcagcgcagctggaccaatggcagcggtcataatcacatgcaagagcgttacacggacccgcagcacagcccgagcgttaatggtttgggtagcggccacgaccataagaatcagaaagaaacccatcaacgccacgcggcgtccagccaccaccaccatcaccac

SEQ ID NO: 327 - protein, 326 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 328 - DNA, 750 bp, artificial sequence
  synthetic construct
atggcgaaacgcattctgtgcttcggcgacagcctgacctggggttgggttccggtcgaggatggcgcaccgacggaacgttttgcgccggatgtgcgttggacgggtgtgctggctcagcaactgggtgccgattttgaggtcatcgaagagggtctggtcgcacgtacgaccaacattgatgacccgaccgacccgcgtctgaacggcgcaagctatttgccgagctgtctggcgacccacctgccgctggatctggtgattatcatgttgggcaccaatgataccaaagcttatttccgccgcaccccgctggacatcgcgctgggcatgagcgtcttggtgacgcaggttctgactagcgctggcggtgtcggtactacgtaccctgcgccgaaagtcctggtggttagcccgccaccgctggcgccgatgccgcacccgtggttccaactgatttttgaaggcggtgagcaaaagacgaccgagttggcccgtgtttacagcgcgttggcgagctttatgaaagttccgtttttcgacgcgggcagcgttattagcaccgatggcgtggacggtatccatttcaccgaagcaaataaccgtgacctgggtgtggccctggctgaacaagtgcgcagcctgctgggtccgggctccggtggtgccggttcgccgggtagcgcaggcggtcctggatccggcaaaggcaagggtaagggtaaaggtaaacatcatcaccaccaccac

SEQ ID NO: 329 - protein, 250 aa, artificial sequence
  synthetic construct
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLLGPGSGGAGSPGSAGGPGSGKGKGKGKGKHHHHHH

SEQ ID NO: 330 - DNA, 1134 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccccgagcgcccaaagccagctgcctgataagcacagcggtctgcatgagcgtgctcctcaacgttatggtccggagccggaaccggaaccagagccgatcccggaaccgccgaaggaagccccggtcgtgattgaaaaaccgaaaccgaagccgaaaccaaagccgaagccgccagcgcatgaccacaagaatcagaaagaaacgcaccaacgtcacgccgctggctctggcggtggcggttcgccgcatcatcaccaccaccat

SEQ ID NO: 331 - protein, 378 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 332 - DNA, 1134 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccccgagcgcgcaatcccagttgccggatcgccacagcggtctgcatgagcgtgccccgcaacgttacggtccagagccggagccggaaccggagccgattccggaaccaccgcgcgaggctccggtggttatcgaacgtccacgtccacgcccgcgcccgcgtccgcgtccaccggcgcacgaccaccgtaatcaacgtgaaacccaccagcgccacgcagccggcagcggtggtggtggtagcccgcatcatcaccatcatcac

SEQ ID NO: 333 - protein, 378 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSPSAQSQLPDRHSGLHERAPQRYGPEPEPEPEPIPEPPREAPVVIERPRPRPRPRPRPPAHDHRNQRETHQRHAAGSGGGGSPHHHHHH

SEQ ID NO: 334 - DNA, 1146 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccgcgcaaagccaactgccggacaaacatagcggtctgcacgagcgtgcgccgcagcgttacggtagcggtacggcggaaattcaatccagcaagaacccgaacccgcacccgcagcgcagctggaccaatggcagcggtcataatcacatgcaagagcgttacacggacccgcagcacagcccgagcgttaatggtttgggtagcggccacgaccataagaatcagaaagaaacccatcaacgccacgcggcgtccagccaccaccaccatcaccac

SEQ ID NO: 335 - protein, 382 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSAQSQLPDKHSGLHERAPQRYGSGTAEIQSSKNPNPHPQRSWTNGSGHNHMQERYTDPQHSPSVNGLGSGHDHKNQKETHQRHAASSHHHHHH

SEQ ID NO: 336 - DNA, 918 bp, artificial sequence
  synthetic construct
atgtccaccttcgttgcgaaagatggcacccagatttactttaaagactggggcagcggcaagccggttctgtttagccacggctggccgctggacgcggatatgtgggagtatcagatggagtacctgagcagccgtggttaccgtaccatcgccttcgatcgccgtggttttggtcgcagcgatcaaccgtggaccggcaatgattatgacacgttcgcagatgacattgcccagctgatcgagcacctggacctgaaagaggttaccctggtcggtttcagcatgggcggtggtgacgtcgcgcgctacattgcgcgtcatggttccgctcgtgtggcgggtctggtcctgctgggtgctgtaacgccactgtttggtcaaaagccggattatccgcagggtgtgccgttggatgtgtttgcgcgcttcaaaaccgagttgctgaaagaccgtgcgcaattcatcagcgacttcaacgcaccgttttacggtatcaacaaaggccaagttgtcagccagggcgttcaaacgcagacgctgcagattgcgctgctggcaagcctgaaggcgaccgttgactgcgtgacggcttttgcggaaactgattttcgtccggacatggcgaagattgatgttccgaccttggtgattcacggtgacggcgatcagatcgtgccgttcgaaaccaccggtaaggttgcggccgagctgatcaaaggtgcggagctgaaagtgtacaaggacgcgcctcacggcttcgcagtcactcatgcacagcaactgaacgaggacttgctggccttcttgaaacgcggtccgggctccggtggcgcaggcagcccgggtagcgcaggtggtccgggatccggcaaaggcaagggtaagggtaaaggtaaacatcatcaccaccaccac

SEQ ID NO: 337 - protein, 306 aa, artificial sequence
  synthetic construct
MSTFVAKDGTQIYFKDWGSGKPVLFSHGWPLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKRGPGSGGAGSPGSAGGPGSGKGKGKGKGKHHHHHH

SEQ ID NO: 338 - protein, 216 aa, Mycobacterium smegmatis
MAKRILCFGDSLTWGWVPVEDGAPTERFAPDVRWTGVLAQQLGADFEVIEEGLSARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 339 - protein, 272 aa, Pseudomonas fluorescens
MSTFVAKDGTQIYFKDWGSGKPVLFSHGWLLDADMWEYQMEYLSSRGYRTIAFDRRGFGRSDQPWTGNDYDTFADDIAQLIEHLDLKEVTLVGFSMGGGDVARYIARHGSARVAGLVLLGAVTPLFGQKPDYPQGVPLDVFARFKTELLKDRAQFISDFNAPFYGINKGQVVSQGVQTQTLQIALLASLKATVDCVTAFAETDFRPDMAKIDVPTLVIHGDGDQIVPFETTGKVAAELIKGAELKVYKDAPHGFAVTHAQQLNEDLLAFLKR

SEQ ID NO: 340 - DNA, 1293 bp, artificial sequence
  synthetic construct
atggcattcttcgacctgcctctggaagaactgaaaaagtaccgcccagagcgttacgaagagaaagacttcgacgagttttgggaagaaacgctggcggagagcgagaagttcccattggacccggtttttgaacgcatggagagccacctgaaaaccgtggaggcgtatgacgtgaccttctctggttatcgtggccagcgcattaagggttggttgctggtcccgaaactggaagaagaaaagctgccgtgcgtggtccaatacatcggttacaatggtggccgtggttttccgcacgattggctgttctggccgagcatgggttacatttgctttgtcatggacacccgtggccaaggcagcggctggctgaagggtgatacgccggattatccggagggtccggtcgatccgcagtatccgggcttcatgacccgtggtatcttggacccgcgtacctactattatcgtcgtgtgttcacggacgcggttcgtgcggttgaggcagcagcgagctttccgcaggtcgaccaagagcgtatcgtgatcgccggtggctcccagggtggcggcattgcgctggctgttagcgctctgtccaagaaagccaaagcgctgctgtgtgacgttccgtttctgtgtcatttccgtcgcgctgtccagctggtggatacgcatccgtacgcggaaattaccaattttctgaaaacgcaccgtgataaagaagagatcgtctttcgcaccctgagctacttcgatggtgttaacttcgcagcacgcgcgaagattccggcgttgttcagcgtgggtctgatggataacatttctccaccgagcaccgtttttgccgcctacaactattacgcaggtccgaaagagatccgcatctacccgtataacaatcacgagggcggtggcagctttcaggcggttgagcaagtgaaattcctgaagaagctgtttgagaagggtggtccgggctctggcggtgcgggcagcccgggttccgcaggcggtccgggatccccgagcgcgcaatcccagttgccggatcgccacagcggtctgcatgagcgtgccccgcaacgttacggtccagagccggagccggaaccggagccgattccggaaccaccgcgcgaggctccggtggttatcgaacgtccacgtccacgcccgcgcccgcgtccgcgtccaccggcgcacgaccaccgtaatcaacgtgaaacccaccagcgccacgcagccggcagcggtggtggtggtagcccgcatcatcaccatcatcac

SEQ ID NO: 341 - protein, 431 aa, artificial sequence
  synthetic construct
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNISPPSTVFAAYNYYAGPKEIRIYPYNNHEGGGSFQAVEQVKFLKKLFEKGGPGSGGAGSPGSAGGPGSPSAQSQLPDRHSGLHERAPQRYGPEPEPEPEPIPEPPREAPVVIERPRPRPRPRPRPPAHDHRNQRETHQRHAAGSGGGGSPHHHHHH

(19) United States
(12) Patent Application Publication
(10) Pub. No.: US 2012/0317733 A1
(43) Pub. Date: Dec. 20, 2012

(54) ENZYMATIC PERACID GENERATION FOR USE IN HAIR CARE PRODUCTS

(75) Inventors: Dexter A. Chisholm, Unionville, PA (US); Xiumin Cui, Swarthmore, PA (US); Scott D. Cunningham, Chadds Ford, PA (US); Robert DiCosimo, Chadds Ford, PA (US); Stephen R. Fahnestock, Wilmington, DE (US); Tanja Maria Gruber, Media, PA (US); Yongqing Huang, Wilmington, DE (US); Xueping Jiang, Wilmington, DE (US); Anju Parthasarathy, Glenmoore, PA (US); Mark S. Payne, Wilmington, DE (US); Pierre E. Rouviere, Wilmington, DE (US); Hong Wang, Kennett Square, PA (US)

(73) Assignee: E.I. DU PONT DE NEMOURS AND COMPANY, Wilmington, DE (US)

(21) Appl. No.: 13/330,105

(22) Filed: Dec. 19, 2011

Related U.S. Application Data
(60) Provisional application No. 61/424,847, filed on Dec. 20, 2010.

(51) Int. Cl. A61K 8/66; A61K 8/92; A61Q 9/04; C12N 9/96

(52) U.S. Cl. 8/161; 435/188

(57) ABSTRACT

Disclosed herein are compositions and methods to treat hair with a peracid-based benefit agent. The peracid benefit agent can be used for hair bleaching, hair weakening, hair removal, hair waiving, hair straightening or any combination thereof. The peracid may be enzymatically generated from a carboxylic acid ester substrate using an enzyme having perhydrolytic activity (perhydrolase) in the presence of a source of peroxygen. A fusion protein comprising the perhydrolase coupled to a hair-binding domain, either directly or through an optional linker, may be used to target the perhydrolytic activity to the hair surface.

### CROSS-REFERENCE TO RELATED APPLICATIONS

This application claims benefit of U.S. Provisional Patent Application No. 61/424,847 filed Dec. 20, 2010, which is incorporated by reference herein in its entirety.

### FIELD OF THE INVENTION

This invention relates to the field of personal care products comprising at least one peracid as hair care benefit agent. The peracid may be enzymatically produced in the presence of at least one suitable carboxylic acid ester substrate and a source of peroxygen. Specifically, at least one enzyme catalyst comprising an enzyme having perhydrolytic activity is used to produce a peracid benefit agent for use in a hair care product. The perhydrolytic enzyme may be in the form a fusion protein engineered to contain at least one peptidic component having affinity for hair.

### BACKGROUND OF THE INVENTION

Peroxycarboxylic acids (“peracids”) are effective antimicrobial agents. Methods to clean, disinfect, and/or sanitize hard surfaces, food products, living plant tissues, and medical devices against undesirable microbial growth have been described (e.g., U.S. Pat. No. 6,545,047; U.S. Pat. No. 6,183,807; U.S. Pat. No. 6,518,307; U.S. Pat. No. 5,683,724; and U.S. Patent Application Publication No. 2003-0026846 A1). Peracids have also been reported to be useful in preparing bleaching compositions for laundry detergent applications (e.g., U.S. Pat. No. 3,974,082; U.S. Pat. No. 5,296,161; and U.S. Pat. No. 5,364,554).

It has also been reported that peracids may oxidize keratinous materials such as hair, skin and nails. For example, United Kingdom published patent specification GB 692,478(A) to Alexander, P., et al. describes a method of oxidizing the disulfide bonds of keratinous materials to sulphydryl or sulphonic acids using an aqueous solution of saturated peraliphatic acids not having more than 4 carbon atoms at a temperature below 100° C., such that the oxidized material is readily soluble in dilute alkali. Lillie et al. (J. Histochem. Cytochem., (1954) 95-102) discloses oxidation-induced basophilia of keratinous structures. U.S. Pat. No. 6,270,791 to Van Dyke et al. discloses a method to obtain water soluble peptides from a keratin-containing source, such as hair, comprising oxidizing a keratin-containing material in an aqueous solution for form water soluble peptides. The oxidizing agent may include peracetic acid.

Hair care compositions and methods describing the use of a peracid have been reported. Chinese Patent Application Publication CN 101440575 A to Zheng, Y., discloses a method of treating hair with peracetic acid and a catalase followed by treating hair with a protease. US2002-0053110 A1; U.S. Pat. No. 6,022,381; U.S. Pat. No. 6,004,355; WO97/24106; and WO97/24108 to Dias et al. describe hair coloring compositions comprising a peroxyacid oxidizing agent and an oxidative hair coloring agent. U.S. Pat. No. 3,679,347 to Brown, F., describes dyeing human hair with a peroxy compound and a reactive dyestuff. United Kingdom patent GB1560399 A to Clark et al. describes compositions for hair treatment comprising an organic peracid component and an aqueous foam-forming solution containing an organic surfactant and a C10-C21 fatty acid amide. German patent application publication DE19733841 A1 to Till et al. discloses an agent for oxidative treatment of human hair comprising magnesium monoperphthalate.

Hahn, F. et al. (Leder (1967) 18(8):184-192) discloses a method of unhairing by oxidizing hair keratin with peracetic acid, Na₂O₂, and CAROAT® or ClO₂; followed by dissolving the oxidized hair with alkali. U.S. Pat. No. 3,479,127 to Hahn et al. discloses a process for unhairing of skins (calfskins, goatskins, sheepskin) and cowhides with peracids (3 hour treatment of 0.5 to 5 wt % peracetic acid, pH 2 to 5.5) followed by treatment with neutral salts or weak or strong alkaline acting salts or bases. The use of peracid in formulation suitable for use in a personal care depilatory product is not described.

Skin whitening compositions comprising a peracid have also been disclosed. US2007-1066339 A1 to Gupta, S., discloses skin whitening compositions comprising an anionic zeolite cage complex coupled to an active oxygen donor agent, such as peracetic acid. US2006-0161121 A1 to Klaveness et al. discloses peroxide compositions for skin bleaching comprising 2 to 6 wt % of a peroxide bleaching agent. The peroxide bleaching agents described included organic peracids and salts thereof.

The inclusion of specific variant subtilisin Carlsberg proteases having perhydrolytic activity in a body care product is disclosed in U.S. Pat. No. 7,510,859 to Wieland et al. Perhydrolytic enzymes beyond the specific variant proteases are not described nor are there any working examples demonstrating the enzymatic production of peracid as a personal care benefit agent.

U.S. Patent Application Publication Nos. 2008-0176783 A1; 2008-0176299 A1; 2009-0005590 A1; and 2010-0041752 A1 to DiCosimo et al. disclose enzymes structurally classified as members of the CE-7 family of carbohydrate esterases (i.e., cephalosporin C deacetylases [CAHs] and acetyl xylan esterases [AXEs]) that are characterized by significant perhydrolytic activity for converting carboxylic acid ester substrates (in the presence of a suitable source of peroxygen, such as hydrogen peroxide) into peroxycarboxylic acids at concentrations sufficient for use as a disinfectant and/or a bleaching agent. Some members of the CE-7 family of carbohydrate esterases have been demonstrated to have perhydrolytic activity sufficient to produce 4000-5000 ppm peracetic acid from acetyl esters of alcohols, diols, and glycerols in 1 minute and up to 9000 ppm between 5 minutes and 30 minutes once the reaction components were mixed (DiCosimo et al., U.S. 2009-0005590 A1). U.S. Patent application publication No. 2010-0087529 A1 describes variant CE-7 enzymes having improved perhydrolytic activity. Although the CE-7 perhydrolases have exceptional perhydrolytic activity, their use in cosmetic personal care products has not been disclosed. As such, a problem to be solved is to provide personal care compositions and methods comprising the use of at least one CE-7 perhydrolase for the production of a peracid benefit agent.

Peracids are strong oxidizing agents that may be reactive towards a variety of materials, including materials not targeted for the desired benefit. As such, certain personal care applications may benefit from the ability to target/focus the peracid benefit agent to the desired body surface by localizing peracid production on or near the desired target body surface. Enzymatic peracid production may benefit by targeting the perhydrolase to the body surface.

The use of antibodies, antibody fragments (F_{ab}), single chain fused variable region antibodies (scFc), Camelidae antibodies, and large scaffold display proteins as peptidic affinity materials may not be suitable for some personal care applications due to their size and cost. As such, there remains a need in certain low cost cosmetic applications to use shorter, less expensive peptidic affinity materials for targeted delivery of a benefit agent.

The use of shorter, biopanned peptides to target a cosmetic benefit agent to a body surface has been described (U.S. Pat. Nos. 7,220,405; 7,309,482; 7,285,264 and 7,807,141; U.S. Patent Application Publication Nos. 2005-0226839 A1; 2007-0196305 A1; 2006-0199206 A1; 2007-0065387 A1; 2008-0107614 A1; 2007-0110686 A1; 2006-0073111 A1; 2010-0158846; 2010-0158847; and 2010-0247589; and published PCT applications WO2008/054746; WO2004/048399, and WO2008/073368). The use of a peptidic material having affinity for hair to couple an active perhydrolytic enzyme (i.e., “targeted perhydrolases”) for the production of a peracid benefit agent has not been described.

As such, an additional problem to be solved is to provide compositions and methods suitable to target enzymatic peracid production to hair.

### SUMMARY OF THE INVENTION

Compositions and methods are provided to enzymatically produce a peracid benefit agent that may be used in applications such as hair removal (depilatory agent), a decrease in hair tensile strength, a hair pretreatment used to enhance other depilatory products (such as thioglycolate-based hair removal products), hair bleaching, hair dye pretreatment (oxidative hair dyes), hair curling, and hair conditioning.

In one embodiment, a method to provide a peracid-based benefit agent preferentially to hair and not skin is provided comprising:
a) providing a composition comprising a population of enzymes having perhydrolytic activity; said enzymes having at least one binding domain having affinity for hair;
b) contacting a body surface comprising hair and skin with the composition of step a), whereby a first fraction of the population of enzymes binds durably to hair and a second fraction of the population of enzymes does not durably bind to hair;
c) rinsing the body surface to remove the second fraction of enzymes not durably bound to hair;
d) optionally drying the rinsed body surface;
e) contacting said enzymes durably bound to hair with an aqueous solution comprising hydrogen peroxide and at least one carboxylic acid ester substrate; whereby a peracid benefit agent is generated, preferentially providing a peracid-based benefit to hair and not skin; and
f) optionally repeating steps (a) through (e).

In another embodiment, the peracid-based benefit is selected from group consisting of hair removal, hair weakening, hair bleaching, hair styling, hair curling, hair conditioning, hair pretreating prior to application of a non-peracid-based benefit agent, and combinations thereof. In a preferred aspect, the peracid-based benefit is hair removal or hair weakening.

A single step method of combining and contacting the peracid benefit to hair is also provided. In one embodiment, a method to provide a peracid benefit agent to hair is provided comprising:
a) providing a set of reaction components comprising:
   1) at least one enzyme having perhydrolytic activity;
   2) a source of peroxygen; and
   3) a substrate selected form the group consisting of
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have solubility in water of at least 5 ppm at 25° C.;
ii) glycerides having the structure

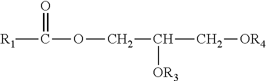

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

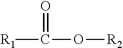

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides; and
v) mixtures thereof;
b) contacting a body surface comprising hair with an effective amount of an enzymatically generated peracid is obtained by combining the set of reaction components; whereby a peracid-based benefit is provided to the body surface comprising hair;
c) rinsing the body surface;
d) optionally drying the rinsed body surface;
e) optionally repeating steps (a) through (d).

The perhydrolytic enzyme may be targeted to the hair by using fusion proteins comprising the perhydrolytic enzyme coupled through an optional peptide spacer to a peptidic component having affinity for hair. In a preferred aspect, the enzyme having perhydrolytic activity is a fusion protein having the following general structure:

PAH-[L]_{y}-HSBD

or

HSBD-[L]_{y}-PAH

wherein
PAH is the enzyme having perhydrolytic activity;
HSBD is a peptidic component having affinity for hair;
L is an optional peptide linker ranging from 1 to 100 amino acids in length; and
y is 0 or 1.

In a further embodiment, a hair care product is provided comprising:

a) an enzyme catalyst having perhydrolytic activity,

b) at least one substrate selected from the group consisting of:
1) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have solubility in water of at least 5 ppm at 25° C.;
2) glycerides having the structure

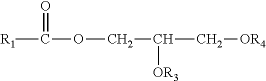

wherein R₁=C1 to C7 straight chain or branched chain alkyl optionally substituted with an hydroxyl or a C1 to C4 alkoxy group and R₃ and R₄ are individually H or R₁C(O);
3) one or more esters of the formula

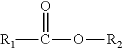

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and 3) acetylated saccharides selected from the group consisting of acetylated monosaccharides, acetylated disaccharides, and acetylated polysaccharides;

c) a source of peroxygen; and

d) a dermally acceptable carrier medium suitable for use in a hair care product.

A method of removing or weakening the tensile strength of hair using an a composition comprising 0.001 wt % to 4 wt % peracetic acid is also provided comprising
a) providing a composition comprising 0.001 to 4 wt % peracetic acid;
b) contacting a body surface comprising hair with said peracetic acid under suitable conditions to form peracid-treated hair;
c) optionally rinsing the peracetic acid-treated hair with an aqueous solution;
d) optionally drying the hair after step (b) or step (c); and
e) repeating steps (a) through (d) until the hair is removed from the body surface.

In a preferred aspect, the above peracetic acid composition applied to the body surface is an aqueous composition. In a further preferred aspect, the peracetic acid composition applied to the body surface does not include, by proviso, substantially a non-aqueous composition (i.e., comprising essentially of one or more organic solvents).

Hydrolytic enzymes having perhydrolytic activity are used to enzymatically produce the peracid providing a benefit to hair. In one embodiment, the perhydrolytic enzyme is selected from the group lipases, proteases, esterases, acyl transferases, aryl esterases, carbohydrate esterases, and combinations thereof. In a preferred aspect, the perhydrolytic aryl esterase comprises an amino acid sequence having at least 95% identify to SEQ ID NO: 314.

In another preferred aspect, the perhydrolytic enzyme is structurally classified as a carbohydrate esterase. In a preferred aspect, the carbohydrate esterases are CE-7 carbohydrate esterases, each having a CE-7 signature motif that aligns with a reference sequence SEQ ID NO: 2 using CLUSTALW, said signature motif comprising:

a) an RGQ motif at positions corresponding to positions 118-120 of SEQ ID NO: 2;

b) a GXSQG motif at positions corresponding to positions 179-183 of SEQ ID NO: 2; and

c) an HE motif at positions corresponding to positions 298-299 of SEQ ID NO: 2.

In yet another embodiment, the enzyme having perhydrolytic activity comprises an amino acid sequence having at least 95% identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

In another embodiment, a hair care product having a set of reaction components is provided comprising:
a) 0.1 to 50 wt % of at least one substrate selected from the group consisting of:
   1) esters having the structure

[X]ₘR₅
wherein X=an ester group of the formula R₆C(O)O;
R₆=a C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group;
wherein R₅ optionally comprises one or more ether linkages;
m is an integer ranging from 1 to the number of carbon atoms in R₅; and
wherein said esters have solubility in water of at least 5 ppm at 25° C.;
2) glycerides having the structure

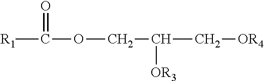

wherein R₁=C1 to C7 straight chain or branched chain alkyl optionally substituted with an hydroxyl or a C1 to C4 alkoxy group and R₃ and R₄ are individually H or R₁C(O);
3) one or more esters of the formula

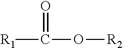

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
4) acetylated saccharides selected from the group consisting of acetylated monosaccharides, acetylated disaccharides, and acetylated polysaccharides;

b) 0.1 to 15 wt % a source of hydrogen peroxide;

c) 0.001 to 1 wt % of an enzyme catalyst comprising at least one enzyme having perhydrolytic activity; and

d) up to 98 wt % of the balance of the hair care composition comprising a dermally acceptable carrier medium; whereby upon mixing the reaction components a peracid is produced.

In a preferred aspect of the above method, the enzyme having perhydrolytic activity is a CE-7 carbohydrate esterase, an aryl esterase, or a combination thereof.

In another embodiment, a method for the production of a fusion protein comprising a perhydrolytic enzyme coupled to at least one hair binding domain is provided comprising:

a) providing a recombinant microbial host cell comprising an expressible genetic construct encoding a fusion protein, said fusion protein comprising an enzyme having perhydrolytic activity coupled to a peptidic component having affinity for hair;

b) growing the recombinant microbial host cell under suitable conditions whereby the fusion protein is produced; and

c) optionally recovering the fusion protein.

### BRIEF DESCRIPTION OF THE BIOLOGICAL SEQUENCES

The following sequences comply with 37 C.F.R. §§1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NO: 1 is the nucleic acid sequence encoding a cephalosporin C deacetylase from Bacillus subtilis ATCC® 31954™.

SEQ ID NO: 2 is the amino acid sequence of a cephalosporin C deacetylase from Bacillus subtilis ATCC® 31954™.

SEQ ID NO: 3 is the nucleic acid sequence encoding a cephalosporin C deacetylase from Bacillus subtilis subsp. subtilis strain 168.

SEQ ID NO: 4 is the amino acid sequence of a cephalosporin C deacetylase from Bacillus subtilis subsp. subtilis strain 168.

SEQ ID NO: 5 is the nucleic acid sequence encoding a cephalosporin C deacetylase from B. subtilis ATCC® 6633™.

SEQ ID NO: 6 is the acid sequence of a cephalosporin C deacetylase from B. subtilis ATCC® 6633™.

SEQ ID NO: 7 is the nucleic acid sequence encoding a cephalosporin C deacetylase from B. licheniformis ATCC® 14580™.

SEQ ID NO: 8 is the deduced amino acid sequence of a cephalosporin C deacetylase from B. licheniformis ATCC® 14580™.

SEQ ID NO: 9 is the nucleic acid sequence encoding an acetyl xylan esterase from B. pumilus PS213.

SEQ ID NO: 10 is the deduced amino acid sequence of an acetyl xylan esterase from B. pumilus PS213.

SEQ ID NO: 11 is the nucleic acid sequence encoding an acetyl xylan esterase from Clostridium thermocellum ATCC® 27405™.

SEQ ID NO: 12 is the deduced amino acid sequence of an acetyl xylan esterase from Clostridium thermocellum ATCC® 27405™.

SEQ ID NO: 13 is the nucleic acid sequence encoding an acetyl xylan esterase from Thermotoga neapolitana.

SEQ ID NO: 14 is the amino acid sequence of an acetyl xylan esterase from Thermotoga neapolitana.

SEQ ID NO: 15 is the nucleic acid sequence encoding an acetyl xylan esterase from Thermotoga maritima MSB8.

SEQ ID NO: 16 is the amino acid sequence of an acetyl xylan esterase from Thermotoga maritima MSB8.

SEQ ID NO: 17 is the nucleic acid sequence encoding an acetyl xylan esterase from Thermoanaerobacterium sp. JW/SL Y5485.

SEQ ID NO: 18 is the deduced amino acid sequence of an acetyl xylan esterase from Thermoanaerobacterium sp. JW/SL YS485.

SEQ ID NO: 19 is the nucleic acid sequence of a cephalosporin C deacetylase from Bacillus sp. NRRL B-14911. It should be noted that the nucleic acid sequence encoding the cephalosporin C deacetylase from Bacillus sp. NRRL B-14911 as reported in GENBANK® Accession number ZP_{—}01168674 appears to encode a 15 amino acid N-terminal addition that is likely incorrect based on sequence alignments with other cephalosporin C deacetylases and a comparison of the reported length (340 amino acids) versus the observed length of other CAH enzymes (typically 318-325 amino acids in length; see U.S. Patent Application Publication No. US-2010-0087528-A1; herein incorporated by reference). As such, the nucleic acid sequence as reported herein encodes the cephalosporin C deacetylase sequence from Bacillus sp. NRRL B-14911 without the N-terminal 15 amino acids reported under GENBANK® Accession number ZP_{—}01168674.

SEQ ID NO: 20 is the deduced amino acid sequence of the cephalosporin C deacetylase from Bacillus sp. NRRL B-14911 encoded by the nucleic acid sequence of SEQ ID NO: 19.

SEQ ID NO: 21 is the nucleic acid sequence encoding a cephalosporin C deacetylase from Bacillus halodurans C-125.

SEQ ID NO: 22 is the deduced amino acid sequence of a cephalosporin C deacetylase from Bacillus halodurans C-125.

SEQ ID NO: 23 is the nucleic acid sequence encoding a cephalosporin C deacetylase from Bacillus clausii KSM-K16.

SEQ ID NO: 24 is the deduced amino acid sequence of a cephalosporin C deacetylase from Bacillus clausii KSM-K16.

SEQ ID NO: 25 is the nucleic acid sequence encoding a Bacillus subtilis ATCC® 29233™ cephalosporin C deacetylase (CAH).

SEQ ID NO: 26 is the deduced amino acid sequence of a Bacillus subtilis ATCC® 29233™ cephalosporin C deacetylase (CAH).

SEQ ID NO: 27 is the deduced amino acid sequence of a Thermotoga neapolitana acetyl xylan esterase variant from U.S. Patent Application Publication No. 2010-0087529 (incorporated herein by reference in its entirety), where the Xaa residue at position 277 is Ala, Val, Ser, or Thr.

SEQ ID NO: 28 is the deduced amino acid sequence of a Thermotoga maritima MSB8 acetyl xylan esterase variant from U.S. Patent Application Publication No. 2010-0087529, where the Xaa residue at position 277 is Ala, Val, Ser, or Thr.

SEQ ID NO: 29 is the deduced amino acid sequence of a Thermotoga lettingae acetyl xylan esterase variant from U.S. Patent Application Publication No. 2010-0087529, where the Xaa residue at position 277 is Ala, Val, Ser, or Thr.

SEQ ID NO: 30 is the deduced amino acid sequence of a Thermotoga petrophila acetyl xylan esterase variant from U.S. Patent Application Publication No. 2010-0087529, where the Xaa residue at position 277 is Ala, Val, Ser, or Thr.

SEQ ID NO: 31 is the deduced amino acid sequence of a Thermotoga sp. RQ2 acetyl xylan esterase variant derived from “RQ2(a)” from U.S. Patent Application Publication No. 2010-0087529, where the Xaa residue at position 277 is Ala, Val, Ser, or Thr.

SEQ ID NO: 32 is the deduced amino acid sequence of a Thermotoga sp. RQ2 acetyl xylan esterase variant derived from “RQ2(b)” from U.S. Patent Application Publication No. 2010-0087529, where the Xaa residue at position 278 is Ala, Val, Ser, or Thr.

SEQ ID NO: 33 is the deduced amino acid sequence of a Thermotoga lettingae acetyl xylan esterase.

SEQ ID NO: 34 is the deduced amino acid sequence of a Thermotoga petrophila acetyl xylan esterase.

SEQ ID NO: 35 is the deduced amino acid sequence of a first acetyl xylan esterase from Thermotoga sp. RQ2 described herein as “RQ2(a)”.

SEQ ID NO: 36 is the deduced amino acid sequence of a second acetyl xylan esterase from Thermotoga sp. RQ2 described herein as “RQ2(b)”.

SEQ ID NO: 37 is the codon optimized nucleic acid sequence encoding a Thermoanearobacterium saccharolyticum cephalosporin C deacetylase.

SEQ ID NO: 38 is the deduced amino acid sequence of a Thermoanearobacterium saccharolyticum cephalosporin C deacetylase.

SEQ ID NO: 39 is the nucleic acid sequence encoding the acetyl xylan esterase from Lactococcus lactis (GENBANK® accession number EU255910).

SEQ ID NO: 40 is the amino acid sequence of the acetyl xylan esterase from Lactococcus lactis (GENBANK® accession number ABX75634.1).

SEQ ID NO: 41 is the nucleic acid sequence encoding the acetyl xylan esterase from Mesorhizobium loti (GENBANK® accession number NC_{—}002678.2).

SEQ ID NO: 42 is the amino acid sequence of the acetyl xylan esterase from Mesorhizobium loti (GENBANK® accession number BAB53179.1).

SEQ ID NO: 43 is the nucleic acid sequence encoding the acetyl xylan esterase from Geobacillus stearothermophilus (GENBANK® accession number AF038547.2).

SEQ ID NO: 44 is the amino acid sequence of the acetyl xylan esterase from Geobacillus stearothermophilus (GENBANK® accession number AAF70202.1).

SEQ ID NO: 45 is the nucleic acid sequence encoding a variant acetyl xylan esterase (a.k.a. variant “A3”) having the following substitutions relative to the wild-type Thermotoga maritima acetyl xylan esterase amino acid sequence: (F24I/S35T/Q179L/N275D/C277S/S308G/F317S).

SEQ ID NO: 46 is the amino acid sequence of the “A3” variant acetyl xylan esterase.

SEQ ID NO: 47 is the nucleic acid sequence encoding the N275D/C277S variant acetyl xylan esterase.

SEQ ID NO: 48 is the amino acid sequence of the N275D/C277S variant acetyl xylan esterase.

SEQ ID NO: 49 is the nucleic acid sequence encoding the C277S/F317S variant acetyl xylan esterase.

SEQ ID NO: 50 is the amino acid sequence of the C277S/F317S variant acetyl xylan esterase.

SEQ ID NO: 51 is the nucleic acid sequence encoding the S35T/C277S variant acetyl xylan esterase.

SEQ ID NO: 52 is the amino acid sequence of the S35T/C277S variant acetyl xylan esterase.

SEQ ID NO: 53 is the nucleic acid sequence encoding the Q179L/C277S variant acetyl xylan esterase.

SEQ ID NO: 54 is the amino acid sequence of the Q179L/C277S variant acetyl xylan esterase.

SEQ ID NO: 55 is the nucleic acid sequence encoding the variant acetyl xylan esterase 843H9 having the following substitutions relative to the wild-type Thermotoga maritima acetyl xylan esterase amino acid sequence: (L8R/L125Q/Q176L/V183D/F247I/C277S/P292L).

SEQ ID NO: 56 is the amino acid sequence of the 843H9 variant acetyl xylan esterase.

SEQ ID NO: 57 is the nucleic acid sequence encoding the variant acetyl xylan esterase 843F12 having the following substitutions relative to the wild-type Thermotoga maritima acetyl xylan esterase amino acid sequence: K77 E/A266E/C277S.

SEQ ID NO: 58 is the amino acid sequence of the 843F12 variant acetyl xylan esterase.

SEQ ID NO: 59 is the nucleic acid sequence encoding the variant acetyl xylan esterase 843C12 having the following substitutions relative to the wild-type Thermotoga maritima acetyl xylan esterase amino acid sequence: F27Y/I149V/A266V/C277S/I295T/N3025.

SEQ ID NO: 60 is the amino acid sequence of the 843C12 variant acetyl xylan esterase.

SEQ ID NO: 61 is the nucleic acid sequence encoding the variant acetyl xylan esterase 842H3 having the following substitutions relative to the wild-type Thermotoga maritima acetyl xylan esterase amino acid sequence: L195Q/C277S.

SEQ ID NO: 62 is the amino acid sequence of the 842H3 variant acetyl xylan esterase.

SEQ ID NO: 63 is the nucleic acid sequence encoding the variant acetyl xylan esterase 841A7 having the following substitutions relative to the wild-type Thermotoga maritima acetyl xylan esterase amino acid sequence: Y110F/C277S.

SEQ ID NO: 64 is the amino acid sequence of the 841A7 variant acetyl xylan esterase.

SEQ ID NOs: 65-221, 271, 290, and 291 are a non-limiting list of amino acid sequences of peptides having affinity for hair.

SEQ ID NO: 217-269 are the amino acid sequences of peptides having affinity for skin.

SEQ ID NOs: 270-271 are the amino acid sequences of peptides having affinity for nail.

SEQ ID NOs: 272-285 are the amino acid sequences peptide linkers/spacers.

SEQ ID NO: 286 is the nucleic acid sequence encoding fusion peptide C277S-HC263.

SEQ ID NO: 287 is the nucleic acid sequence encoding the fusion construct C277S-HC1010.

SEQ ID ON: 288 is the amino acid sequence of fusion peptide C277S-HC263.

SEQ ID NO: 289 is the amino acid sequence of fusion peptide C277S-HC1010.

SEQ ID ON: 290 is the amino acid of hair-binding domain HC263.

SEQ ID NO: 291 is the amino acid sequence of hair-binding domain HC1010.

SEQ ID ON: 292 if the nucleic acid sequence of expression plasmid pLD001.

SEQ ID NO: 293 is the amino acid sequence of T. maritima variant C277S.

SEQ ID NO: 294 is the amino acid sequence of fusion peptide C277S-HC263 further comprising a D128G substitution (“CPAH-HC263”).

SEQ ID NO: 295 is the amino acid sequence of fusion peptide C277S-HC1010 further comprising a D128G substitution (“CPAH-HC1010”).

SEQ ID NO: 296 is the nucleic acid sequence encoding the variant acetyl xylan esterase 006A10 (U.S. Provisional Patent Appl. No. 61/425,561; hereby incorporated by reference) having the following substitutions relative to the wild-type Thermotoga maritima acetyl xylan esterase amino acid sequence: (F268S/C277T).

SEQ ID NO: 297 is the amino acid sequence of the 006A10 variant acetyl xylan esterase.

SEQ ID NO: 298 is the nucleic acid sequence encoding the variant acetyl xylan esterase 006E10 (U.S. Provisional Patent Appl. No. 61/425,561) having the following substitutions relative to the wild-type Thermotoga maritima acetyl xylan esterase amino acid sequence: (R218C/C277T/F317L).

SEQ ID NO: 299 is the amino acid sequence of the 006E10 variant acetyl xylan esterase.

SEQ ID NO: 300 is the nucleic acid sequence encoding the variant acetyl xylan esterase 006E12 (U.S. Provisional Patent Appl. No. 61/425,561) having the following substitutions relative to the wild-type Thermotoga maritima acetyl xylan esterase amino acid sequence: (H227L/T233A/C277T/A290V).

SEQ ID NO: 301 is the amino acid sequence of the 006E12 variant acetyl xylan esterase.

SEQ ID NO: 302 is the nucleic acid sequence encoding the variant acetyl xylan esterase 006G11 (U.S. Provisional Patent Appl. No. 61/425,561) having the following substitutions relative to the wild-type Thermotoga maritima acetyl xylan esterase amino acid sequence: (D254G/C277T).

SEQ ID NO: 303 is the amino acid sequence of the 006G11 variant acetyl xylan esterase.

SEQ ID NO: 304 is the nucleic acid sequence encoding the variant acetyl xylan esterase 006F12 (U.S. Provisional Patent Appl. No. 61/425,561) having the following substitutions relative to the wild-type Thermotoga maritima acetyl xylan esterase amino acid sequence: (R261S/I264F/C277T).

SEQ ID NO: 305 is the amino acid sequence of the 006F12 variant acetyl xylan esterase.

SEQ ID NO: 306 is the nucleic acid sequence encoding the variant acetyl xylan esterase 006B12 (U.S. Provisional Patent Appl. No. 61/425,561) having the following substitutions relative to the wild-type Thermotoga maritima acetyl xylan esterase amino acid sequence: (W28C/F104S/C277T).

SEQ ID NO: 307 is the amino acid sequence of the 006B12 variant acetyl xylan esterase.

SEQ ID NO: 308 is the nucleic acid sequence encoding the variant acetyl xylan esterase 87464 (U.S. Provisional Patent Appl. No. 61/425,561; hereby incorporated by reference) having the following substitutions relative to the wild-type Thermotoga maritima acetyl xylan esterase amino acid sequence: (A266P/C277S).

SEQ ID NO: 309 is the amino acid sequence of the 873B4 variant acetyl xylan esterase.

SEQ ID NO: 310 is the nucleic acid sequence encoding the variant acetyl xylan esterase 006D10 (U.S. Provisional Patent Appl. No. 61/425,561; hereby incorporated by reference) having the following substitutions relative to the wild-type Thermotoga maritima acetyl xylan esterase amino acid sequence: (W28C/L32P/D151E/C277T).

SEQ ID NO: 311 is the amino acid sequence of the 006D10 variant acetyl xylan esterase.

SEQ ID NO: 312 is the amino acid sequence of hair-binding domain “HC263KtoR”, a variant of hair binding domain “HC263” (SEQ ID NO: 290) in which 10 lysine residues have been replaced by 10 arginine residues.

SEQ ID NO: 313 is the amino acid sequence of the charged peptide (GK)₅-H6.

SEQ ID NO: 314 is the amino acid sequence of the S54V variant of the aryl esterase from Mycobacterium smegmatis.

SEQ ID NO: 315 is the amino acid sequence of the L29P variant of the hydrolase from Pseudomonas fluorescens.

SEQ ID NO: 316 is the nucleotide sequence of the synthetic gene encoding the acetyl xylan esterase from Bacillus pumilus fused at its C-terminus to the hair binding domain HC263 via a flexible linker.

SEQ ID NO: 317 is the amino acid sequence of the acetyl xylan esterase from Bacillus pumilus fused at its C-terminus to the hair binding domain HC263 via a flexible linker.

SEQ ID NO: 318 is the nucleotide sequence of the synthetic gene encoding the acetyl xylan esterase from Lactococcus lactis fused at its C-terminus to the hair binding domain HC263 via a flexible linker.

SEQ ID NO: 319 is the amino acid sequence of the acetyl xylan esterase from Lactococcus lactis fused at its C-terminus to the hair binding domain HC263 via a flexible linker.

SEQ ID NO: 320 is the nucleotide sequence of the synthetic gene encoding the acetyl xylan esterase from Mesorhizobium loti fused at its C-terminus to the hair binding domain HC263 via a flexible linker.

SEQ ID NO: 321 is the amino acid sequence of the acetyl xylan esterase from Mesorhizobium loti fused at its C-terminus to the hair binding domain HC263 via a flexible linker.

SEQ ID NO: 322 is the nucleotide sequence of the synthetic gene encoding the S54V variant of the aryl esterase from Mycobacterium smegmatis fused at its C-terminus to the hair binding domain HC263 via a flexible linker.

SEQ ID NO: 323 is the amino acid sequence of the S54V variant of the aryl esterase from Mycobacterium smegmatis fused at its C-terminus to the hair binding domain HC263 via a flexible linker.

SEQ ID NO: 324 is the nucleotide sequence of the synthetic gene encoding the S54V variant of the aryl esterase from Mycobacterium smegmatis fused at its C-terminus to the hair binding domain HC263KtoR via a flexible linker.

SEQ ID NO: 325 is the amino acid sequence of the S54V variant of the aryl esterase from Mycobacterium smegmatis fused at its C-terminus to the hair binding domain HC263KtoR via a flexible linker.

SEQ ID NO: 326 is the nucleotide sequence of the synthetic gene encoding the S54V variant of the aryl esterase from Mycobacterium smegmatis fused at its C-terminus to the hair binding domain HC1010 (SEQ ID NO: 291) via a flexible linker.

SEQ ID NO: 327 is the amino acid sequence of the S54V variant of the aryl esterase from Mycobacterium smegmatis fused at its C-terminus to the hair binding domain HC1010 via a flexible linker.

SEQ ID NO: 328 is the nucleotide sequence of the synthetic gene encoding the S54V variant of the aryl esterase from Mycobacterium smegmatis fused at its C-terminus to the charged peptide (GK)₅-His6 via a flexible linker.

SEQ ID NO: 329 is the amino acid sequence of the S54V variant of the aryl esterase from Mycobacterium smegmatis fused at its C-terminus to the charged peptide (GK)₅-His6 via a flexible linker.

SEQ ID NO: 330 is the nucleotide sequence of the synthetic gene encoding the L29P variant of the hydrolase from Pseudomonas fluorescens fused at its C-terminus to the hair binding domain HC263 via a flexible linker.

SEQ ID NO: 331 is the amino acid sequence of the L29P variant of the hydrolase from Pseudomonas fluorescens fused at its C-terminus to the hair binding domain HC263 via a flexible linker.

SEQ ID NO: 332 is the nucleotide sequence of the synthetic gene encoding the L29P variant of the hydrolase from Pseudomonas fluorescens fused at its C-terminus to the hair binding domain HC263KtoR via a flexible linker.

SEQ ID NO: 333 is the amino acid sequence of the L29P variant of the hydrolase from Pseudomonas fluorescens fused at its C-terminus to the hair binding domain HC263FtoR via a flexible linker.

SEQ ID NO: 334 is the nucleotide sequence of the synthetic gene encoding the L29P variant of the hydrolase from Pseudomonas fluorescens fused at its C-terminus to the hair binding domain HC1010 (SEQ ID NO: 291) via a flexible linker.

SEQ ID NO: 335 is the amino acid sequence of the L29P variant of the hydrolase from Pseudomonas fluorescens fused at its C-terminus to the hair binding domain HC1010 via a flexible linker.

SEQ ID NO: 336 is the nucleotide sequence of the synthetic gene encoding the L29P variant of the hydrolase from Pseudomonas fluorescens fused at its C-terminus to the charged peptide (GK)₅-His6 via a flexible linker.

SEQ ID NO: 337 is the amino acid sequence of the L29P variant of the hydrolase from Pseudomonas fluorescens fused at its C-terminus to the charged peptide (GK)₅-His6 via a flexible linker.

SEQ ID NO: 338 is the amino acid sequence of the wild type Mycobacterium smegmatis aryl esterase.

SEQ ID NO: 339 is the amino acid sequence of the wild type Pseudomonas fluorescens esterase.

SEQ ID NO: 340 is the nucleotide sequence of the synthetic gene encoding the C277S variant of the perhydrolase from Thermotoga maritima fused at its C-terminus to the hair binding domain HC263KtoR via a flexible linker.

SEQ ID NO: 341 is the amino acid sequence of the C277S variant of the perhydrolase from Thermotoga maritima fused at its C-terminus to the hair binding domain HC263FtoR via a flexible linker.

### DETAILED DESCRIPTION OF THE INVENTION

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, “contacting” refers to placing a composition in contact with the target body surface for a period of time sufficient to achieve the desired result (target surface binding, peracid based effects, etc.). In one embodiment, “contacting” may refer to placing a composition comprising (or capable of producing) an efficacious concentration of peracid in contact with a target body surface for a period of time sufficient to achieve the desired result. In another embodiment, “contacting” may also refer to the placing at least one component of a personal care composition, such as one or more of the reaction components used to enzymatic perhydrolysis, in contact with a target body surface. Contacting includes spraying, treating, immersing, flushing, pouring on or in, mixing, combining, painting, coating, applying, affixing to and otherwise communicating a peracid solution or a composition comprising an efficacious concentration of peracid, a solution or composition that forms an efficacious concentration of peracid or a component of the composition that forms an efficacious concentration of peracid with the body surface.

As used herein, the terms “substrate”, “suitable substrate”, and “carboxylic acid ester substrate” interchangeably refer specifically to:
(a) one or more esters having the structure

[X]ₘR₅
wherein
X is an ester group of the formula R₆C(O)O;
R₆ is a C1 to C7 linear, branched or cyclic hydrocarbyl moiety, optionally substituted with a hydroxyl group or C1 to C4 alkoxy group, wherein R₆ optionally comprises one or more ether linkages where R₆ is C2 to C7;
R₅ is a C1 to C6 linear, branched, or cyclic hydrocarbyl moiety or a cyclic five-membered heteroaromatic or six-membered cyclic aromatic or heteroaromatic moiety optionally substituted with a hydroxyl group; wherein each carbon atom in R₅ individually comprises no more than one hydroxyl group or no more than one ester or carboxylic acid group, and wherein R₅ optionally comprises one or more ether linkages;
m is 1 to the number of carbon atoms in R₅,
said one or more esters having solubility in water of at least 5 ppm at 25° C.; or
(b) one or more glycerides having the structure

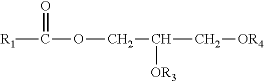

wherein R₁ is a C1 to C7 straight chain or branched chain alkyl optionally substituted with an hydroxyl or a C1 to C4 alkoxy group and R₃ and R₄ are individually H or R₁C(O); or
(c) one or more esters of the formula

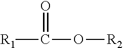

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; or
(d) one or more acetylated monosaccharides, acetylated disaccharides, or acetylated polysaccharides; or
(e) any combination of (a) through (d).

As used herein, the term “peracid” is synonymous with peroxyacid, peroxycarboxylic acid, peroxy acid, percarboxylic acid and peroxoic acid.

As used herein, the term “peracetic acid” is abbreviated as “PAA” and is synonymous with peroxyacetic acid, ethaneperoxoic acid and all other synonyms of CAS Registry Number 79-21-0.

As used herein, the term “monoacetin” is synonymous with glycerol monoacetate, glycerin monoacetate, and glyceryl monoacetate.

As used herein, the term “diacetin” is synonymous with glycerol diacetate; glycerin diacetate, glyceryl diacetate, and all other synonyms of CAS Registry Number 25395-31-7.

As used herein, the term “triacetin” is synonymous with glycerin triacetate; glycerol triacetate; glyceryl triacetate, 1,2,3-triacetoxypropane; 1,2,3-propanetriol triacetate and all other synonyms of CAS Registry Number 102-76-1.

As used herein, the term “monobutyrin” is synonymous with glycerol monobutyrate, glycerin monobutyrate, and glyceryl monobutyrate.

As used herein, the term “dibutyrin” is synonymous with glycerol dibutyrate and glyceryl dibutyrate.

As used herein, the term “tributyrin” is synonymous with glycerol tributyrate, 1,2,3-tributyrylglycerol, and all other synonyms of CAS Registry Number 60-01-5.

As used herein, the term “monopropionin” is synonymous with glycerol monopropionate, glycerin monopropionate, and glyceryl monopropionate.

As used herein, the term “dipropionin” is synonymous with glycerol dipropionate and glyceryl dipropionate.

As used herein, the term “tripropionin” is synonymous with glyceryl tripropionate, glycerol tripropionate, 1,2,3-tripropionylglycerol, and all other synonyms of CAS Registry Number 139-45-7.

As used herein, the terms “acetylated sugar” and “acetylated saccharide” refer to mono-, di- and polysaccharides comprising at least one acetyl group. Examples include, but are not limited to glucose pentaacetate; xylose tetraacetate; acetylated xylan; acetylated xylan fragments; β-D-ribofuranose-1,2,3,5-tetraacetate; tri-O-acetyl-D-galactal; and tri-O-acetyl-glucal.

As used herein, the terms “hydrocarbyl”, “hydrocarbyl group”, and “hydrocarbyl moiety” is meant a straight chain, branched or cyclic arrangement of carbon atoms connected by single, double, or triple carbon to carbon bonds and/or by ether linkages, and substituted accordingly with hydrogen atoms. Such hydrocarbyl groups may be aliphatic and/or aromatic. Examples of hydrocarbyl groups include methyl, ethyl, propyl, isopropyl, butyl, isobutyl, t-butyl, cyclopropyl, cyclobutyl, pentyl, cyclopentyl, methylcyclopentyl, hexyl, cyclohexyl, benzyl, and phenyl. In a preferred embodiment, the hydrocarbyl moiety is a straight chain, branched or cyclic arrangement of carbon atoms connected by single carbon to carbon bonds and/or by ether linkages, and substituted accordingly with hydrogen atoms.

As used herein, the terms “monoesters” and “diesters” of 1,2-ethanediol; 1,2-propanediol; 1,3-propanediol; 1,2-butanediol; 1,3-butanediol; 2,3-butanediol; 1,4-butanediol; 1,2-pentanediol; 2,5-pentanediol; 1,5-pentanediol; 1,6-pentanediol; 1,2-hexanediol; 2,5-hexanediol; 1,6-hexanediol; and mixtures thereof, refer to said compounds comprising at least one ester group of the formula RC(O)O, wherein R is a C1 to C7 linear hydrocarbyl moiety. In one embodiment, the carboxylic acid ester substrate is selected from the group consisting of propylene glycol diacetate (PGDA), ethylene glycol diacetate (EDGA), and mixtures thereof.

As used herein, the term “propylene glycol diacetate” is synonymous with 1,2-diacetoxypropane, propylene diacetate, 1,2-propanediol diacetate, and all other synonyms of CAS Registry Number 623-84-7.

As used herein, the term “ethylene glycol diacetate” is synonymous with 1,2-diacetoxyethane, ethylene diacetate, glycol diacetate, and all other synonyms of CAS Registry Number 111-55-7.

As used herein, the terms “suitable enzymatic reaction mixture”, “components suitable for in situ generation of a peracid”, “suitable reaction components”, “suitable aqueous reaction mixture”, “reaction mixture”, and “peracid-generating components” refer to the materials and water in which the reactants and the perhydrolytic enzyme catalyst come into contact. In one embodiment, the peracid-generating components will include at least one perhydrolase, preferably in the form of a fusion protein comprising a binding domain having affinity for a body surface such as hair, at least one suitable carboxylic acid ester substrate, a source of peroxygen, and water (e.g., aqueous solution comprising a source of peroxygen, such as hydrogen peroxide). In a preferred aspect, the perhydrolase is a CE-7 perhydrolase, preferable in the form of a fusion protein targeted to a body surface, such as hair.

As used herein, the term “perhydrolysis” is defined as the reaction of a selected substrate with peroxide to form a peracid. Typically, inorganic peroxide is reacted with the selected substrate in the presence of a catalyst to produce the peroxycarboxylic acid. As used herein, the term “chemical perhydrolysis” includes perhydrolysis reactions in which a substrate (a peroxycarboxylic acid precursor) is combined with a source of hydrogen peroxide wherein peroxycarboxylic acid is formed in the absence of an enzyme catalyst. As used herein, the term “enzymatic perhydrolysis” includes perhydrolysis reactions in which a carboxylic acid ester substrate (a peracid precursor) is combined with a source of hydrogen peroxide and water whereby the enzyme catalyst catalyzes the formation of peracid.

As used herein, the term “perhydrolase activity” refers to the catalyst activity per unit mass (for example, milligram) of protein, dry cell weight, or immobilized catalyst weight.

As used herein, “one unit of enzyme activity” or “one unit of activity” or “U” is defined as the amount of perhydrolase activity required for the production of 1 μmol of peroxycarboxylic acid product per minute at a specified temperature.

As used herein, the terms “enzyme catalyst” and “perhydrolase catalyst” refer to a catalyst comprising an enzyme having perhydrolysis activity and may be in the form of a whole microbial cell, permeabilized microbial cell(s), one or more cell components of a microbial cell extract, partially purified enzyme, or purified enzyme. The enzyme catalyst may also be chemically modified (such as by pegylation or by reaction with cross-linking reagents). The perhydrolase catalyst may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997.

As used herein, “acetyl xylan esterases” refers to an enzyme (E.C. 3.1.1.72; AXEs) that catalyzes the deacetylation of acetylated xylans and other acetylated saccharides. As illustrated herein, several enzymes classified as acetyl xylan esterases are provided having significant perhydrolytic activity.

As used herein, the terms “cephalosporin C deacetylase” and “cephalosporin C acetyl hydrolase” refer to an enzyme (E.C. 3.1.1.41) that catalyzes the deacetylation of cephalosporins such as cephalosporin C and 7-aminocephalosporanic acid (Mitsushima et al., (1995) Appl. Env. Microbiol. 61(6):2224-2229). The amino acid sequences of several cephalosporin C deacetylases having significant perhydrolytic activity are provided herein.

As used herein, the term “Bacillus subtilis ATCC® 31954™” refers to a bacterial cell deposited to the American Type Culture Collection (ATCC) having international depository accession number ATCC® 31954™. As described herein, an enzyme having significant perhydrolase activity from B. subtilis ATCC® 31954™ is provided as SEQ ID NO: 2 (see United States Patent Application Publication No. 2010-0041752). The amino acid sequence of the isolated enzyme has 100% amino acid identity to the cephalosporin C deacetylase provided by GENBANK® Accession No. BAA01729.1 (Mitsushima et al., supra).

As used herein, the term “Thermotoga maritima MSB8” refers to a bacterial cell reported to have acetyl xylan esterase activity (GENBANK® NP_{—}227893.1; see U.S. Patent Application Publication No. 2008-0176299). The amino acid sequence of the enzyme having perhydrolase activity from Thermotoga maritima MSB8 is provided as SEQ ID NO: 16.

As used herein, an “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

For example, it is well known in the art that alterations in a gene which result in the production of a chemically equivalent amino acid at a given site, but do not affect the functional properties of the encoded protein are common. For the purposes of the present invention substitutions are defined as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, and Trp.
   Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

As used herein, the terms “signature motif” and “diagnostic motif” refer to conserved structures shared among a family of enzymes having a defined activity. The signature motif can be used to define and/or identify the family of structurally-related enzymes having similar enzymatic activity for a defined family of substrates. The signature motif can be a single contiguous amino acid sequence or a collection of discontiguous, conserved motifs that together form the signature motif. Typically, the conserved motif(s) is represented by an amino acid sequence. In one embodiment, the perhydrolytic enzyme comprises a CE-7 carbohydrate esterase signature motif.

As used herein, the term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

As used herein, “synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as pertaining to a DNA sequence, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequences to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein, “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As used herein, the term “sequence analysis software” refers to any computer algorithm or software program that is useful for the analysis of nucleotide or amino acid sequences. “Sequence analysis software” may be commercially available or independently developed. Typical sequence analysis software will include, but is not limited to, the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990)), and DNASTAR (DNASTAR, Inc. 1228 S. Park St. Madison, Wis. 53715 USA), CLUSTALW (for example, version 1.83; Thompson et al., Nucleic Acids Research, 22(22):4673-4680 (1994)), and the FASTA program incorporating the Smith-Waterman algorithm (VV. R. Pearson, Comput. Methods Genome Res., [Proc. Int. Symp.] (1994), Meeting Date 1992, 111-20. Editor(s): Suhai, Sandor. Publisher: Plenum, New York, N.Y.), Vector NTI (Informax, Bethesda, Md.) and Sequencher v. 4.05. Within the context of this application it will be understood that where sequence analysis software is used for analysis, that the results of the analysis will be based on the “default values” of the program referenced, unless otherwise specified. As used herein “default values” will mean any set of values or parameters set by the software manufacturer that originally load with the software when first initialized.

As used herein, the term “body surface” refers to any surface of the human body that may serve as the target for a benefit agent, such as a peracid benefit agent. Typical body surfaces include but are not limited to hair, skin, nails, teeth, and gums. The present methods and compositions are directed to hair care applications and personal care products. As such, the body surface comprises hair. In one embodiment, the body surface is human hair.

As used herein, the term “body wash” refers to a water-based composition designed to clean the skin and comprising one or more surfactants to provide cleansing action and foaming. Additionally, the body wash formulation may contain a wide variety of other ingredients such as thickeners, humectants, emollients, buffers, fragrances, glycerol, dyes, preservatives, proteins and stabilizing agents.

The terms moisturizer, a lotion or a body lotion refer to a low to medium-viscosity emulsion of oil and water, most often oil-in-water but possibly water-in-oil with the primary benefit in a skin care application to hydrate the skin or to reduce its water loss. Nearly all moisturizer contain a combination of emollients, occlusives, and humectants. Emollients, which are mainly lipids and oils, hydrate and improve the appearance of the skin. Occlusives such as petrolatum, lanolin and bee wax reduce transepidermal water loss by creating hydrophobic barrier over the skin. Humectants such as glycerol and urea able to attract water from the external environment and enhance water absorption from the dermis into the epidermis. In addition, the moisturizer formulations may contain emulsifiers to maintain stability of emulsions, and use thickeners to achieve desired viscosity and skin feel. A wide variety of other ingredients such as fragrances, dyes, preservatives, therapeutic agents, proteins and stabilizing agents are commonly added for other consumer preferred attributes.

As used herein, “pharmaceutically-acceptable” means that drugs, medicaments and/or inert ingredients which the term describes are suitable for use in contact with the tissues of humans and other animals without undue toxicity, incompatibility, instability, irritation, allergic response, and the like, commensurate with a reasonable benefit/risk ratio.

As used herein, “personal care products” means products used in the cleaning, bleaching and/or disinfecting of hair, skin, scalp, and teeth, including, but not limited to shampoos, body lotions, shower gels, topical moisturizers, toothpaste, toothgels, mouthwashes, mouthrinses, anti-plaque rinses, and/or other topical cleansers. In some particularly preferred embodiments, these products are utilized on humans, while in other embodiments, these products find use with non-human animals (e.g., in veterinary applications). In a preferred embodiment, the term “personal care products” refers to hair care products. In one embodiment, the hair care product is in the form of a powder, paste, gel, liquid, oil, ointment, spray, foam, tablet, a hair shampoo, a hair conditioner rinse or any combination thereof.

As used herein, the term “biological contaminants” refers to one or more unwanted and/or pathogenic biological entities including, but not limited to, microorganisms, spores, viruses, prions, and mixtures thereof. In one embodiment, a process is provided to enzymatically produce an efficacious concentration of at least one peracid useful to reduce and/or eliminate the presence of the biological contaminants.

As used herein, the term “disinfect” refers to the process of destruction of or prevention of the growth of biological contaminants. As used herein, the term “disinfectant” refers to an agent that disinfects by destroying, neutralizing, or inhibiting the growth of biological contaminants. Typically, disinfectants are used to treat inanimate objects or surfaces. As used herein, the term “disinfection” refers to the act or process of disinfecting. As used herein, the term “antiseptic” refers to a chemical agent that inhibits the growth of disease-carrying microorganisms. In one aspect, the biological contaminants are pathogenic microorganisms.

As used herein, the term “sanitary” means of or relating to the restoration or preservation of health, typically by removing, preventing or controlling an agent that may be injurious to health. As used herein, the term “sanitize” means to make sanitary. As used herein, the term “sanitizer” refers to a sanitizing agent. As used herein the term “sanitization” refers to the act or process of sanitizing.

As used herein, the term “biocide” refers to a chemical agent, typically broad spectrum, which inactivates or destroys microorganisms. A chemical agent that exhibits the ability to inactivate or destroy microorganisms is described as having “biocidal” activity. Peracids can have biocidal activity. Typical alternative biocides known in the art may include, but are not limited to chlorine, chlorine dioxide, chloroisocyanurates, hypochlorites, ozone, acrolein, amines, chlorinated phenolics, copper salts, organo-sulphur compounds, and quaternary ammonium salts.

As used herein, the phrase “minimum biocidal concentration” refers to the minimum concentration of a biocidal agent that, for a specific contact time, will produce a desired lethal, irreversible reduction in the viable population of the targeted microorganisms. The effectiveness can be measured by the log₁₀ reduction in viable microorganisms after treatment. In one aspect, the targeted reduction in viable microorganisms after treatment is at least a 3-log₁₀ reduction, more preferably at least a 4-log₁₀ reduction, and most preferably at least a 5-log₁₀ reduction. In another aspect, the minimum biocidal concentration is at least a 6-log₁₀ reduction in viable microbial cells.

As used herein, the terms “peroxygen source” and “source of peroxygen” refer to compounds capable of providing hydrogen peroxide at a concentration of about 1 mM or more when in an aqueous solution including, but not limited to, hydrogen peroxide, hydrogen peroxide adducts (e.g., urea-hydrogen peroxide adduct (carbamide peroxide)), perborates, and percarbonates. As described herein, the concentration of the hydrogen peroxide provided by the peroxygen compound in the aqueous reaction formulation is initially at least 0.1 mM or more upon combining the reaction components. In one embodiment, the hydrogen peroxide concentration in the aqueous reaction formulation is at least 0.5 mM. In another embodiment, the hydrogen peroxide concentration in the aqueous reaction formulation is at least 10 mM. In another embodiment, the hydrogen peroxide concentration in the aqueous reaction formulation is at least 100 mM. In another embodiment, the hydrogen peroxide concentration in the aqueous reaction formulation is at least 200 mM. In another embodiment, the hydrogen peroxide concentration in the aqueous reaction formulation is 500 mM or more. In yet another embodiment, the hydrogen peroxide concentration in the aqueous reaction formulation is 1000 mM or more. The molar ratio of the hydrogen peroxide to enzyme substrate, e.g., triglyceride, (H₂O₂:substrate) in the aqueous reaction formulation may be from about 0.002 to 20, preferably about 0.1 to 10, and most preferably about 0.5 to 5.

As used herein, the term “oligosaccharide” refers to compounds containing between 2 and at least 24 monosaccharide units linked by glycosidic linkages. The term “monosaccharide” refers to a compound of empirical formula (CH₂O)ₙ, where n≧3, the carbon skeleton is unbranched, each carbon atom except one contains a hydroxyl group, and the remaining carbon atom is an aldehyde or ketone at carbon atom 2. The term “monosaccharide” also refers to intracellular cyclic hemiacetal or hemiketal forms.

As used herein, the term “excipient” refers to inactive substance used as a carrier for active ingredients in a formulation. The excipient may be used to stabilize the active ingredient in a formulation, such as the storage stability of the active ingredient. Excipients are also sometimes used to bulk up formulations that contain active ingredients. As described herein, the “active ingredient” may be an enzyme having perhydrolytic activity, a peracid produced by the perhydrolytic enzyme under suitable reaction conditions, or a combination thereof.

The term “substantially free of water” will refer to a concentration of water in a formulation that does not adversely impact the storage stability of the enzyme or an enzyme powder when present in the carboxylic acid ester. The carboxylic acid ester may contain a very low concentration of water, for example, triacetin typically has between 180 ppm and 300 ppm of water. In one embodiment, the perhydrolytic enzyme is stored in the carboxylic acid ester substrate that is substantially free of water. In a further embodiment, “substantially free of water” may mean less than 2000 ppm, preferably less than 1000 ppm, more preferably less than 500 ppm, and even more preferably less than 250 ppm of water in the formulation comprising the enzyme (or enzyme powder) and the carboxylic acid ester. In one embodiment, the perhydrolytic enzyme may be stored in an aqueous solution if the generation system is designed such that the enzyme is stable in the aqueous solution (for example, a solution that does not contain a significant concentration of a carboxylic acid ester substrate capable of being hydrolyzed by the enzyme during storage). In one embodiment, the perhydrolytic enzyme may be stored in a mixture comprising the carboxylic acid ester substrate that is substantially free of water and one or more buffers (e.g., sodium and/or potassium salts of bicarbonate, citrate, acetate, phosphate, pyrophosphate, methylphosphonate, succinate, malate, fumarate, tartrate, and maleate).

As used herein, the term “effective amount” will refer to the amount of a given condition and/or composition subjected to a target material to obtain a desired effect (e.g., subjecting hair to a peracid for hair removal or tensile strength modification).

As used herein, the term “population of enzymes” refers to a plurality of perhydrolytic enzymes, wherein the population may be comprised of different perhydrolytic enzymes. On one embodiment, the population of enzymes may be comprises of fusion proteins comprising a perhydrolytic enzyme coupled to a peptidic component having affinity for a target material, such as a hair. The population of enzymes may comprise a subpopulation or fraction which is capable of binding durably to a target surface, such as hair, through the inclusion of at least one binding domain coupled to the perhydrolytic enzyme(s). A treatment, such as washing or rinsing, may be used to differentiate the subpopulation that bind durably to a target material and the subpopulation that do not bind durably to the target material.

As used herein, the term “binds durably to hair” will refer to perhydrolytic enzymes, typically in the form of a fusion protein, that (after being contacted and bound to hair) remain bound to hair after being subject to a subsequent washing/rinsing. The washing or rinsing step used to remove the subpopulation of perhydrolytic enzymes not durably bound to hair will typically be comprised of water, such as tap water, typically used when rinsing hair in a shower. In one embodiment, the perhydrolytic enzymes which bind durably to hair may be defined by those enzymes (typically targeted perhydrolases) which are capable of remaining bound to hair after washing/rinsing the hair using the following: 4 rinses at 25° C. using 50 mM phosphate buffer, pH 7.2, containing 1% TWEEN®-20 followed by 2 rinses at 25° C. using 50 mM phosphate buffer. The use of peptidic component having strong affinity for hair when design the targeted perhydrolase(s) in combination with a rinsing step facilitates preferential production of the peracid-based benefit agent on hair and not skin.

### Enzymes Having Perhydrolytic Activity

Enzymes having perhydrolytic activity may include some enzymes classified as lipases, proteases, esterases, acyl transferases, aryl esterases, carbohydrate esterases, and combinations so long as the enzyme has perhydrolytic activity for one or more of the present substrates. Examples may include, but are not limited to perhydrolytic proteases (subtilisin Carlsberg variant; U.S. Pat. No. 7,510,859), perhydrolytic aryl esterases (Pseudomonas fluorescens; SEQ ID NO: 315 [L29P variant] and SEQ ID NO: 339 [wild type]; U.S. Pat. No. 7,384,787), a perhydrolytic aryl esterase from Mycobacterium smegmatis (SEQ ID NO: 314 [S54V variant] and SEQ ID NO: 338 [wild type]; U.S. Pat. No. 7,754,460; WO2005/056782; and EP1689859 B1), and perhydrolytic carbohydrate esterases. In a preferred aspect, the perhydrolytic carbohydrate esterase is a CE-7 carbohydrate esterase.

In one embodiment, suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to any of the amino acid sequences encoding an enzyme having perhydrolytic activity as reported herein.

In another embodiment, the suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

In one embodiment, the suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 314, 315, 338, and 339.

In another embodiment, substantially similar perhydrolytic enzymes may include those encoded by polynucleotide sequences that hybridize under highly stringent hybridization conditions (0.1×SSC, 0.1% SDS, 65° C. and washed with 2×SSC, 0.1% SDS followed by a final wash of 0.1×SSC, 0.1% SDS, 65° C.) to the polynucleotide sequences encoding any of the present perhydrolytic enzymes.

In a preferred embodiment, the perhydrolases may be in the form of fusion proteins having at least one peptidic component having affinity for at least one body surface. In one embodiment, all alignments used to determine if a targeted perhydrolase (fusion protein) comprises a substantially similar sequence to any of the perhydrolases described herein are based on the amino acid sequence of the perhydrolytic enzyme without the peptidic component having the affinity for a body surface.

### CE-7 Perhydrolases

In a preferred embodiment, the present personal care compositions and methods comprise enzymes having perhydrolytic activity that are structurally classified as members of the carbohydrate family esterase family 7 (CE-7 family) of enzymes (see Coutinho, P. M., Henrissat, B. “Carbohydrate-active enzymes: an integrated database approach” in Recent Advances in Carbohydrate Bioengineering, H. J. Gilbert, G. Davies, B. Henrissat and B. Svensson eds., (1999) The Royal Society of Chemistry, Cambridge, pp. 3-12.). The CE-7 family of enzymes has been demonstrated to be particularly effective for producing peroxycarboxylic acids from a variety of carboxylic acid ester substrates when combined with a source of peroxygen (WO2007/070609 and U.S. Patent Application Publication Nos. 2008-0176299, 2008-176783, 2009-0005590, 2010-0041752, and 2010-0087529, as well as U.S. patent application Ser. No. 12/571,702 and U.S. Provisional Patent Application No. 61/318,016 to DiCosimo et al.; each incorporated herein by reference).

Members of the CE-7 family include cephalosporin C deacetylases (CAHs; E.C. 3.1.1.41) and acetyl xylan esterases (AXEs; E.G. 3.1.1.72). Members of the CE-7 esterase family share a conserved signature motif (Vincent et al., J. Mol. Biol., 330:593-606 (2003)). Perhydrolases comprising the CE-7 signature motif (“CE-7 perhydrolases”) and/or a substantially similar structure are suitable for use in the compositions and methods described herein. Means to identify substantially similar biological molecules are well known in the art (e.g., sequence alignment protocols, nucleic acid hybridizations and/or the presence of a conserved signature motif). In one aspect, the perhydrolase includes an enzyme comprising the CE-7 signature motif and at least 20%, preferably at least 30%, more preferably at least 33%, more preferably at least 40%, more preferably at least 42%, more preferably at least 50%, more preferably at least 60%, more preferably at least 70%, more preferably at least 80%, more preferably at least 90%, and most preferably at least 90%, 91%, 92%, 93%, 94%, 95%, 96%, 97%, 98%, or 99% amino acid identity to one of the sequences provided herein.

As used herein, the phrase “enzyme is structurally classified as a CE-7 enzyme”, “CE-7 perhydrolase” or “structurally classified as a carbohydrate esterase family 7 enzyme” will be used to refer to enzymes having perhydrolysis activity which are structurally classified as a CE-7 carbohydrate esterase. This family of enzymes can be defined by the presence of a signature motif (Vincent et al., supra). The signature motif for CE-7 esterases comprises three conserved motifs (residue position numbering relative to reference sequence SEQ ID NO: 2; the CE-7 perhydrolase from B. subtilis ATCC® 31954™):

a) Arg118-Gly119-Gln120;

b) Gly179-Xaa180-Ser181-Gln182-Gly183; and

c) His298-Glu299.

Typically, the Xaa at amino acid residue position 180 is glycine, alanine, proline, tryptophan, or threonine. Two of the three amino acid residues belonging to the catalytic triad are in bold. In one embodiment, the Xaa at amino acid residue position 180 is selected from the group consisting of glycine, alanine, proline, tryptophan, and threonine.

Further analysis of the conserved motifs within the CE-7 carbohydrate esterase family indicates the presence of an additional conserved motif (LXD at amino acid positions 267-269 of SEQ ID NO: 2) that may be used to further define a perhydrolase belonging to the CE-7 carbohydrate esterase family. In a further embodiment, the signature motif defined above may include an additional (fourth) conserved motif defined as:

Leu267-Xaa268-Asp269.

The Xaa at amino acid residue position 268 is typically isoleucine, valine, or methionine. The fourth motif includes the aspartic acid residue (bold) belonging to the catalytic triad (Ser181-Asp269-His298).

The CE-7 perhydrolases may be in the form of fusion proteins having at least one peptidic component having affinity for at least one body surface. In one embodiment, all alignments used to determine if a targeted perhydrolase (fusion protein) comprises the CE-7 signature motif will be based on the amino acid sequence of the perhydrolytic enzyme without the peptidic component having the affinity for a body surface.

A number of well-known global alignment algorithms (i.e., sequence analysis software) may be used to align two or more amino acid sequences representing enzymes having perhydrolase activity to determine if the enzyme is comprised of the present signature motif. The aligned sequence(s) are compared to the reference sequence (SEQ ID NO: 2) to determine the existence of the signature motif. In one embodiment, a CLUSTAL alignment (such as CLUSTALW) using a reference amino acid sequence (as used herein the perhydrolase sequence (SEQ ID NO: 2) from the Bacillus subtilis ATCC® 31954™) is used to identify perhydrolases belonging to the CE-7 esterase family. The relative numbering of the conserved amino acid residues is based on the residue numbering of the reference amino acid sequence to account for small insertions or deletions (for example, typically five amino acids of less) within the aligned sequence.

Examples of other suitable algorithms that may be used to identify sequences comprising the present signature motif (when compared to the reference sequence) include, but are not limited to, Needleman and Wunsch (J. Mol. Biol. 48, 443-453 (1970); a global alignment tool) and Smith-Waterman (J. Mol. Biol. 147:195-197 (1981); a local alignment tool). In one embodiment, a Smith-Waterman alignment is implemented using default parameters. An example of suitable default parameters include the use of a BLOSUM62 scoring matrix with GAP open penalty=10 and a GAP extension penalty=0.5.

A comparison of the overall percent identity among perhydrolases indicates that enzymes having as little as 33% amino acid identity to SEQ ID NO: 2 (while retaining the signature motif) exhibit significant perhydrolase activity and are structurally classified as CE-7 carbohydrate esterases. In one embodiment, suitable perhydrolases include enzymes comprising the CE-7 signature motif and at least 20%, preferably at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 2.

Examples of suitable CE-7 carbohydrate esterases having perhydrolytic activity include, but are not limited to, enzymes having an amino acid sequence such as SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 299, 301, 303, 305, 307, 309, and 311. In one embodiment, the enzyme comprises an amino acid sequence selected from the group consisting of 14, 16, 27, 28, 29, 30, 31, 32, 33, 34, 35, 36, 46, 48, 50, 52, 54, 56, 58, 60, 62, and 64. In a further preferred embodiment, the CE-7 carbohydrate esterase is derived from the Thermotoga maritima CE-7 carbohydrate esterase (SEQ ID NO: 18).

As used herein, the term “CE-7 variant”, “variant perhydrolase” or “variant” will refer to CE-7 perhydrolases having a genetic modification that results in at least one amino acid addition, deletion, and/or substitution when compared to the corresponding enzyme (typically the wild type enzyme) from which the variant was derived; so long as the CE-7 signature motif and the associated perhydrolytic activity are maintained. CE-7 variant perhydrolases may also be used in the present compositions and methods. Examples of CE-7 variants are provided as SEQ ID NOs: 27, 28, 29, 30, 31, 32, 48, 50, 52, 54, 56, 58, 60, 62, 64, 293, 297, 299, 301, 303, 305, 307, 309, and 311. In one embodiment, the variants may include SEQ ID NOs: 27, 28, 50, 52, 54, 56, 58, 60, 62, and 64.

The skilled artisan recognizes that substantially similar CE-7 perhydrolase sequences (retaining the signature motifs) may also be used in the present compositions and methods. In one embodiment, substantially similar sequences are defined by their ability to hybridize, under highly stringent conditions with the nucleic acid molecules associated with sequences exemplified herein. In another embodiment, sequence alignment algorithms may be used to define substantially similar enzymes based on the percent identity to the DNA or amino acid sequences provided herein.

As used herein, a nucleic acid molecule is “hybridizable” to another nucleic acid molecule, such as a cDNA, genomic DNA, or RNA, when a single strand of the first molecule can anneal to the other molecule under appropriate conditions of temperature and solution ionic strength. Hybridization and washing conditions are well known and exemplified in Sambrook, J. and Russell, D., T. Molecular Cloning: A Laboratory Manual, Third Edition, Cold Spring Harbor Laboratory Press, Cold Spring Harbor (2001). The conditions of temperature and ionic strength determine the “stringency” of the hybridization. Stringency conditions can be adjusted to screen for moderately similar molecules, such as homologous sequences from distantly related organisms, to highly similar molecules, such as genes that duplicate functional enzymes from closely related organisms. Post-hybridization washes typically determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of conditions uses higher temperatures in which the washes are identical to those above except for the temperature of the final two 30 min washes in 0.2×SSC, 0.5% SDS was increased to 60° C. Another preferred set of highly stringent hybridization conditions is 0.1×SSC, 0.1% SDS, 65° C. and washed with 2×SSC, 0.1% SDS followed by a final wash of 0.1×SSC, 0.1% SDS, 65° C.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (Sambrook and Russell, supra). For hybridizations with shorter nucleic acids, i.e., oligonucleotides, the position of mismatches becomes more important, and the length of the oligonucleotide determines its specificity (Sambrook and Russell, supra). In one aspect, the length for a hybridizable nucleic acid is at least about 10 nucleotides. Preferably, a minimum length for a hybridizable nucleic acid is at least about 15 nucleotides in length, more preferably at least about 20 nucleotides in length, even more preferably at least 30 nucleotides in length, even more preferably at least 300 nucleotides in length, and most preferably at least 800 nucleotides in length. Furthermore, the skilled artisan will recognize that the temperature and wash solution salt concentration may be adjusted as necessary according to factors such as length of the probe.

As used herein, the term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs. Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, Md.), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chema et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment is preferred. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5.

In one aspect, suitable isolated nucleic acid molecules encode a polypeptide having an amino acid sequence that is at least about 20%, preferably at least 30%, 33%, 40%, 50%, 60%, 70%, 80%, 85%, 90%, 91%, 92%, 93%, 94%, 95%, 96%, 97%, 98%, or 99% identical to the amino acid sequences reported herein. In another aspect, suitable isolated nucleic acid molecules encode a polypeptide having an amino acid sequence that is at least about 20%, preferably at least 30%, 33%, 40%, 50%, 60%, 70%, 80%, 85%, 90%, 91%, 92%, 93%, 94%, 95%, 96%, 97%, 98%, or 99% identical to the amino acid sequences reported herein; with the proviso that the polypeptide retains the CE-7 signature motif. Suitable nucleic acid molecules not only have the above homologies, but also typically encode a polypeptide having about 210 to 340 amino acids in length, about 300 to about 340 amino acids, preferably about 310 to about 330 amino acids, and most preferably about 318 to about 325 amino acids in length wherein each polypeptide is characterized as having perhydrolytic activity.

### Targeted Perhydrolases

As used herein, the term “targeted perhydrolase” and “targeted enzyme having perhydrolytic activity” will refer to a fusion proteins comprising at least one perhydrolytic enzyme (wild type or variant thereof) fused/coupled to at least one peptidic component having affinity for a target surface, preferably a targeted body surface. The perhydrolytic enzyme within the targeted perhydrolase may be any perhydrolytic enzyme and may include lipases, proteases, esterases, acyl transferases, aryl esterases, carbohydrate esterases, and combinations so long as the enzyme has perhydrolytic activity for one or more of the present substrates. Examples may include, but are not limited to perhydrolytic proteases (subtilisin variant; U.S. Pat. No. 7,510,859), perhydrolytic esterase (Pseudomonas fluorescens; U.S. Pat. No. 7,384,787; SEQ ID NO: 315 [L29P variant] and SEQ ID NO: 339 [wild type]), a perhydrolytic aryl esterase (Mycobacterium smegmatis; U.S. Pat. No. 7,754,460; WO2005/056782; and EP1689859 B1; SEQ ID NOs: 314 [S54V variant] and 338 [wild type]).

As used herein the terms “at least one binding domain having affinity for hair”, “peptidic component having affinity for a body surface”, “peptidic component having affinity for hair”, and “HSBD” will refer to a peptidic component of a fusion protein that is not part of the perhydrolytic enzyme comprising at least one polymer of two or more amino acids joined by a peptide bond; wherein the component has affinity for hair, preferably human hair.

In one embodiment, the peptidic component having affinity for a body surface may be an antibody, an F_{ab} antibody fragment, a single chain variable fragment (scFv) antibody, a Camelidae antibody (Muyldermans, S., Rev. Mol. Biotechnol. (2001) 74:277-302), a non-antibody scaffold display protein (Hosse et al., Prot. Sci. (2006) 15(1): 14-27 and Binz, H. et al. (2005) Nature Biotechnology 23, 1257-1268 for a review of various scaffold-assisted approaches) or a single chain polypeptide lacking an immunoglobulin fold. In another aspect, the peptidic component having affinity for a body surface is a single chain peptide lacking an immunoglobulin fold (i.e., a body surface-binding peptide or a body surface-binding domain comprising at least one body surface-binding peptide having affinity for hair). In a preferred embodiment, the peptidic component is a single chain peptide comprising one or more body surface-binding peptides having affinity for hair.

The peptidic component having affinity for hair may be separated from the perhydrolytic enzyme by an optional peptide linker. Certain peptide linkers/spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

In one embodiment, the peptidic component having affinity for hair may include one or more hair-binding peptide, each optionally and independently separated by a peptide spacer of 1 to 100 amino acids in length. Examples of hair-binding peptides and/or hair-binding domains comprising a hair-binding peptide may include, but are not limited to SEQ ID NOs: 65-221, 271, 290, 291, 312, and 313. Examples of peptide linkers/spacer may include, but are not limited to SEQ ID NOs: 272 through 285.

Peptides previously identified as having affinity for one body surface may have affinity for the hair as well. As such, the fusion peptide may comprise at least one previously reported to have affinity for another body surface, such as skin (SEQ ID NOs: 217-269) or nail (SEQ ID NOs: 270-271). In another embodiment, the fusion peptide may include any body surface-binding peptide designed to have electrostatic attraction to the target body surface (e.g., a body surface-binding peptide engineered to electrostatically bind to the target body surface).

### Targeted CE-7 Perhydrolases

In a preferred embodiment, the “targeted perhydrolase” is a targeted CE-7 carbohydrate esterase having perhydrolytic activity. As used herein, the terms “targeted CE-7 perhydrolase” and “targeted CE-7 carbohydrate esterase” will refer to fusion proteins comprising at least one CE-7 perhydrolase (wild type or variant perhydrolase) fused/coupled to at least one peptidic component having affinity for a targeted surface, preferably hair. The peptidic component having affinity for a body surface may be any of those describe above. In a preferred aspect, the peptidic component in a targeted CE-7 perhydrolase is a single chain peptide lacking an immunoglobulin fold (i.e., a body surface-binding peptide or a body surface-binding domain comprising at least one body surface-binding peptide having affinity for hair). In a preferred embodiment, the peptidic component is a single chain peptide comprising one or more body surface-binding peptides having affinity for hair.

The peptidic component having affinity for hair/hair surface may be separated from the CE-7 perhydrolase by an optional peptide linker. Certain peptide linkers/spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

As such, examples of targeted CE-7 perhydrolases may include, but are not limited to, any of the CE-7 perhydrolases having an amino acid sequence selected from the group consisting of SEQ ID NOs 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 301, 303, 305, 307, 309, and 311 coupled to a peptidic component having affinity for hair. In a preferred embodiment, examples of targeted perhydrolases may include, but are not limited to, any of CE-7 perhydrolases having an amino acid sequence selected from the group consisting of SEQ ID NOs 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 301, 303, 305, 307, 309, and 311 coupled to one or more body surface-binding peptides having affinity for hair (optionally through a peptide spacer).

In another embodiment, targeted CE-7 perhydrolases may comprise peptides previously identified as having affinity for one body surface may have affinity for an oral care surface as well. As such, the fusion peptide may comprise at least one previously reported to have affinity for another body surface, such as skin (SEQ ID NOs: 217-269) or nail (SEQ ID NOs: 270-271). In one embodiment, the CE-7 fusion peptide comprises at least one hair-binding peptide from the group comprising SEQ ID NOs: 65-221, 271, 290, and 291. In another embodiment, the CE-7 perhydrolase fusion peptide may include any body surface-binding peptide designed to have electrostatic attraction to the target body surface (e.g., a body surface-binding peptide engineered to electrostatically bind to the target body surface).

In another embodiment, examples of targeted CE-7 perhydrolases may include, but are not limited to SEQ ID NOs 288, 289, 294, 295, 317, 319, and 321.

### Peptides Having Affinity for a Body Surface

Single chain peptides lacking an immunoglobulin fold that are capable of binding to at least one body surface are referred to as “body surface-binding peptides” (BSBPs) and may include, for example, peptides that bind to hair, skin, or nail. Peptides that have been identified to bind to at least human hair are also referred to as “hair-binding peptides (HBP).” Peptides that have been identified to bind to at least human skin are also referred to as “skin-binding peptides (SBP).” Peptides that have been identified to bind to at least human nail are also referred to as “nail-binding peptides (NBP).” Short single chain body surface-binding peptides may be empirically generated (e.g., positively charged polypeptides targeted to negatively charged surfaces) or generated using biopanning against a target body surface.

Short peptides having strong affinity for various body surfaces have been reported (U.S. Pat. Nos. 7,220,405; 7,309,482; 7,285,264 and 7,807,141; U.S. Patent Application Publication Nos. 2005-0226839; 2007-0196305; 2006-0199206; 2007-0065387; 2008-0107614; 2007-0110686; 2006-0073111; 2010-0158846 and 2010-0158847; and published PCT applications WO2008/054746; WO2004/048399, and WO2008/073368). The body surface-binding peptides have been used to construct peptide-based reagents capable of binding benefit agents to a target body surface. However, the use of these peptides to couple an active perhydrolase to the target body surface (i.e., “targeted perhydrolases”) for the production of a peracid benefit agent has not been described.

A non-limiting list of body surface-binding peptides having affinity for at least one body surface are provided herein including those having affinity for hair (hair-binding peptides having an amino acid sequence selected from the group consisting of SEQ ID NOs: 65-221, 271, 290, and 291), skin (skin-binding peptides comprise an amino acid sequence selected from the group consisting of SEQ ID NOs: 217-269), and nail (nail-binding peptides comprise an amino acid sequence selected from the group consisting of SEQ ID NOs: 270-271). In some embodiments, body surface-binding domains are comprised of body surface-binding peptides that are up to about 60 amino acids in length. In one embodiment, the body surface-binding peptides are 5 to 60 amino acids in length. In other embodiments, body surface-binding peptides are 7 to 50 amino acids in length or 7 to 30 amino acids in length. In still other embodiments are those body surface-binding peptides that are 7 to 27 amino acids in length.

While fusion peptides comprising body surface-binding peptides comprising a single hair-, skin-, nail-binding peptides are certain embodiments of the invention, in other embodiments of the invention, it may be advantageous to use multiple body surface-binding peptides. The inclusion of multiple, i.e., two or more, body surface-binding peptides can provide a peptidic component that is, for example, even more durable than those binding elements including a single body surface-binding. In some embodiments, the body surface-binding domains includes from 2 to about 50 or 2 to about 25 body surface-binding peptides. Other embodiments include those body surface-binding domains including 2 to about 10 or 2 to 5 body surface-binding peptides.

Multiple binding elements (i.e., body surface-binding peptides or body surface-binding domains) can be linked directly together or they can be linked together using peptide spacers. Certain peptide spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

Body surface-binding domains, and the shorter body surface-binding peptides of which they are comprised, can be identified using any number of methods known to those skilled in the art, including, for example, any known biopanning techniques such as phage display, bacterial display, yeast display, ribosome display, mRNA display, and combinations thereof. Typically a random or substantially random (in the event bias exists) library of peptides is biopanned against the target body surface to identify peptides within the library having affinity for the target body surface.

The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500); ribosome display (U.S. Pat. No. 5,643,768; U.S. Pat. No. 5,658,754; and U.S. Pat. No. 7,074,557), and mRNA display technology (PROFUSION™; see U.S. Pat. Nos. 6,258,558; 6,518,018; 6,281,344; 6,214,553; 6,261,804; 6,207,446; 6,846,655; 6,312,927; 6,602,685; 6,416,950; 6,429,300; 7,078,197; and 6,436,665).

### Binding Affinity

The peptidic component having affinity for the body surface comprises a binding affinity for human hair, skin, or nail or of 10⁻⁵ molar (M) or less. In certain embodiments, the peptidic component is one or more body surface-binding peptides and/or binding domain(s) having a binding affinity for human hair, skin, or nail of 10⁻⁵ molar (M) or less. In some embodiments, the binding peptides or domains will have a binding affinity value of 10⁻⁵ M or less in the presence of at least about 50-500 mM salt. The term “binding affinity” refers to the strength of the interaction of a binding peptide with its respective substrate, in this case, human hair, skin, or nail. Binding affinity can be defined or measured in terms of the binding peptide's dissociation constant (“K_{D}”), or “MB₅₀.”

“K_{D}” corresponds to the concentration of peptide at which the binding site on the target is half occupied, i.e., when the concentration of target with peptide bound (bound target material) equals the concentration of target with no peptide bound. The smaller the dissociation constant, the more tightly the peptide is bound. For example, a peptide with a nanomolar (nM) dissociation constant binds more tightly than a peptide with a micromolar (μM) dissociation constant. Certain embodiments of the invention will have a K_{D} value of 10⁻⁵ or less.

“MB₅₀” refers to the concentration of the binding peptide that gives a signal that is 50% of the maximum signal obtained in an ELISA-based binding assay. See, e.g., Example 3 of U.S. Patent Application Publication 2005/022683; hereby incorporated by reference. The MB₅₀ provides an indication of the strength of the binding interaction or affinity of the components of the complex. The lower the value of MB₅₀, the stronger, i.e., “better,” the interaction of the peptide with its corresponding substrate. For example, a peptide with a nanomolar (nM) MB₅₀ binds more tightly than a peptide with a micromolar (μM) MB₅₀. Certain embodiments of the invention will have a MB₅₀ value of 10⁻⁵ M or less.

In some embodiments, the peptidic component having affinity for a body surface may have a binding affinity, as measured by K_{D} or MB₅₀ values, of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, or less than or equal to about 10⁻¹⁰ M.

In some embodiments, the body surface-binding peptides and/or body surface-binding domains may have a binding affinity, as measured by K_{D} or MB₅₀ values, of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, or less than or equal to about 10⁻¹⁰ M.

As used herein, the term “strong affinity” will refer to a binding affinity having a K_{D} or MB₅₀ value of less than or equal to about 10⁻⁵ M, preferably less than or equal to about 10⁻⁶ M, more preferably less than or equal to about 10⁻⁷ M, more preferably less than or equal to about 10⁻⁸ M, less than or equal to about 10⁻⁹ M, or most preferably less than or equal to about 10⁻¹⁰ M.

### Multicomponent Peroxycarboxylic Acid Generation Systems

The design of systems and means for separating and combining multiple active components are known in the art and generally will depend upon the physical form of the individual reaction components. For example, multiple active fluids (liquid-liquid) systems typically use multi-chamber dispenser bottles or two-phase systems (e.g., U.S. Patent Application Publication No. 2005/0139608; U.S. Pat. No. 5,398,846; U.S. Pat. No. 5,624,634; U.S. Pat. No. 6,391,840; E.P. Patent 0807156B1; U.S. Patent Application. Pub. No. 2005/0008526; and PCT Publication No. WO 00/61713) such as found in some bleaching applications wherein the desired bleaching agent is produced upon mixing the reactive fluids. Other forms of multicomponent systems used to generate peroxycarboxylic acid may include, but are not limited to, those designed for one or more solid components or combinations of solid-liquid components, such as powders (e.g., U.S. Pat. No. 5,116,575), multi-layered tablets (e.g., U.S. Pat. No. 6,210,639), water dissolvable packets having multiple compartments (e.g., U.S. Pat. No. 6,995,125) and solid agglomerates that react upon the addition of water (e.g., U.S. Pat. No. 6,319,888).

In another embodiment, the carboxylic acid ester in the first component is selected from the group consisting of monoacetin, diacetin, triacetin, and combinations thereof. In another embodiment, the carboxylic acid ester in the first component is an acetylated saccharide. In another embodiment, the enzyme catalyst in the first component may be a particulate solid. In another embodiment, the first reaction component may be a solid tablet or powder

Peroxycarboxylic acids are quite reactive and generally decrease in concentration over time. This is especially true for commercial pre-formed peroxycarboxylic acid compositions that often lack long term stability. Aqueous solutions of pre-formed peroxycarboxylic acids may also present handling and/or shipping difficulties, especially when shipping large containers and/or highly concentrated peroxycarboxylic acid solutions over longer distances. Further, pre-formed peroxycarboxylic acid solutions may not be able to provide the desired concentration of peroxycarboxylic acid for a particular target application. As such, it is highly desirable to keep the various reaction components separated, especially for liquid formulations.

The use of multi-component peroxycarboxylic acid generation systems comprising two or more components that are combined to produce the desired peroxycarboxylic acid has been reported. The individual components should be safe to handle and stable for extended periods of time (i.e., as measured by the concentration of peroxycarboxylic acid produced upon mixing). In one embodiment, the storage stability of a multi-component enzymatic peroxycarboxylic acid generation system may be measured in terms of enzyme catalyst stability.

Personal care products comprising a multi-component peroxycarboxylic acid generation formulation are provided herein that use an enzyme catalyst to rapidly produce an aqueous peracid solution having a desired peroxycarboxylic acid concentration. The mixing may occur immediately prior to use and/or at the site (in situ) of application. In one embodiment, the personal care product formulation will be comprised of at least two components that remain separated until use. Mixing of the components rapidly forms an aqueous peracid solution. Each component is designed so that the resulting aqueous peracid solution comprises an efficacious peracid concentration suitable for the intended end use (e.g., peracid-based depilation, peracid-based reduction in hair tensile strength, peracid-enhanced hair removal for use with other depilatory products (such as thioglycolate-based hair removal products), hair bleaching, hair dye pretreatment (oxidative hair dyes), hair curling, hair conditioning, skin whitening, skin bleaching, skin conditioning, reducing the appearance of skin wrinkles, skin rejuvenation, reducing dermal adhesions, reducing or eliminating body odors, nail bleaching, or nail disinfecting. The composition of the individual components should be designed to (1) provide extended storage stability and/or (2) provide the ability to enhance formation of a suitable aqueous reaction formulation comprised of peroxycarboxylic acid.

The multi-component formulation may be comprised of at least two substantially liquid components. In one embodiment, the multi-component formulation may be a two component formulation comprises a first liquid component and a second liquid component. The use of the terms “first” or “second” liquid component is relative provided that two different liquid components comprising the specified ingredients remain separated until use. At a minimum, the multi-component peroxycarboxylic acid formulation comprises (1) at least one enzyme catalyst having perhydrolysis activity, (2) a carboxylic acid ester substrate, and (3) a source of peroxygen and water wherein the formulation enzymatically produces the desired peracid upon combining the components.

The type and amount of the various ingredients used within two component formulation should to be carefully selected and balanced to provide (1) storage stability of each component, especially the perhydrolysis activity of the enzyme catalyst and (2) physical characteristics that enhance solubility and/or the ability to effectively form the desired aqueous peroxycarboxylic acid solution (e.g., ingredients that enhance the solubility of the ester substrate in the aqueous reaction mixture and/or ingredients that modify the viscosity and/concentration of at least one of the liquid components [i.e., at least one cosolvent that does not have a significant, adverse effect on the enzymatic perhydrolysis activity]).

Various methods to improve the performance and/or catalyst stability of enzymatic peracid generation systems have been disclosed. U.S. Patent Application Publication No. 2010-0048448 A1 describes the use of at least one cosolvent to enhance solubility and/or the mixing characteristics of certain ester substrates. The present personal care compositions and methods may also use a cosolvent. In one embodiment, the component comprising the carboxylic acid ester substrate and the perhydrolase catalyst comprises an organic solvent having a Log P value of less than about 2, wherein Log P is defined as the logarithm of the partition coefficient of a substance between octanol and water, expressed as P=[solute]_{octanol}/[solute]_{water}. Several cosolvents having a log P value of 2 or less that do not have a significant adverse impact on enzyme activity are described. In another embodiment, the cosolvent is about 20 wt % to about 70 wt % within the reaction component comprising the carboxylic acid ester substrate and the enzyme. The reaction component comprising the carboxylic acid ester substrate and the enzyme may optionally comprise one or more buffers (e.g., sodium and/or potassium salts of bicarbonate, citrate, acetate, phosphate, pyrophosphate, methylphosphonate, succinate, malate, fumarate, tartrate, and maleate).

U.S. Patent Application Publication No. 2010-0086534 A1 describes the use of a two component system wherein the first component comprises a formulation of a liquid carboxylic acid ester and solid enzyme powder; wherein said enzyme powder comprises a formulation of (a) at least one CE-7 esterase having perhydrolysis activity and (b) at least one oligosaccharide excipient; and the second component comprises water having a source of peroxygen and a hydrogen peroxide stabilizer. The present personal care compositions and methods may use a two component formulation similar to the system described in US 2010-0086534 A1. As such, an oligosaccharide excipient may be used to help stabilize enzyme activity. In one embodiment, the oligosaccharide excipient may have a number average molecular weight of at least about 1250 and a weight average molecular weight of at least about 9000. In another embodiment, the oligosaccharide excipient has have a number average molecular weight of at least about 1700 and a weight average molecular weight of at least about 15000. In another embodiment, the oligosaccharide is maltodextrin.

U.S. Patent Application Publication No. 2010-0086535-A1 also describes a two component system wherein the first component comprises a formulation of a liquid carboxylic acid ester and solid enzyme powder, said formulation comprising (a) an enzyme powder comprising at least one CE-7 esterase having perhydrolysis activity and at least one oligosaccharide excipient and at least one surfactant; and (b) at least one buffer, where in a preferred embodiment the buffer is added as a separate (i.e. separate from the enzyme powder) insoluble component to the carboxylic acid ester substrate; and the second component comprises water having a source of peroxygen and a hydrogen peroxide stabilizer. The present personal care compositions and methods may use a two component formulation similar to the system described in US 2010-0086535 A1. In one embodiment, the excipient may be an oligosaccharide excipient that has a number average molecular weight of at least about 1250 and a weight average molecular weight of at least about 9000. In another embodiment, the oligosaccharide excipient may have a number average molecular weight of at least about 1700 and a weight average molecular weight of at least about 15000. In another embodiment, the oligosaccharide is maltodextrin. In a further embodiment, the optional pH buffer is a bicarbonate buffer. In yet a further embodiment, the hydrogen peroxide stabilizer is TURPINAL® SL.

### Enzyme Powders

In some embodiments, the personal care compositions may use an enzyme catalyst in form of a stabilized enzyme powder. Methods to make and stabilize formulations comprising an enzyme powder are described in U.S. Patent Application Publication Nos. 2010-0086534 and 2010-0086535.

In one embodiment, the enzyme may be in the enzyme powder in an amount in a range of from about 5 weight percent (wt %) to about 75 wt % based on the dry weight of the enzyme powder. A preferred weight percent range of the enzyme in the enzyme powder/spray-dried mixture is from about 10 wt % to 50 wt %, and a more preferred weight percent range of the enzyme in the enzyme powder/spray-dried mixture is from about 20 wt % to 33 wt %

In one embodiment, the enzyme powder may further comprise an excipient. In one aspect, the excipient is provided in an amount in a range of from about 95 wt % to about 25 wt % based on the dry weight of the enzyme powder. A preferred wt % range of excipient in the enzyme powder is from about 90 wt % to 50 wt %, and a more preferred wt % range of excipient in the enzyme powder is from about 80 wt % to 67 wt %.

In one embodiment, the excipient used to prepare an enzyme powder may be an oligosaccharide excipient. In one embodiment, the oligosaccharide excipient has a number average molecular weight of at least about 1250 and a weight average molecular weight of at least about 9000. In some embodiments, the oligosaccharide excipient has a number average molecular weight of at least about 1700 and a weight average molecular weight of at least about 15000. Specific oligosaccharides may include, but are not limited to, maltodextrin, xylan, mannan, fucoidan, galactomannan, chitosan, raffinose, stachyose, pectin, insulin, levan, graminan, amylopectin, sucrose, lactulose, lactose, maltose, trehalose, cellobiose, nigerotriose, maltotriose, melezitose, maltotriulose, raffinose, kestose, and mixtures thereof. In a preferred embodiment, the oligosaccharide excipient is maltodextrin. Oligosaccharide-based excipients may also include, but are not limited to, water-soluble non-ionic cellulose ethers, such as hydroxymethyl-cellulose and hydroxypropylmethylcellulose, and mixtures thereof. In yet a further embodiment, the excipient may be selected from, but not limited to, one or more of the following compounds: trehalose, lactose, sucrose, mannitol, sorbitol, glucose, cellobiose, α-cyclodextrin, and carboxymethylcellulose.

The formulations may comprise at least one optional surfactant, where the presence of at least one surfactant is preferred. Surfactants may include, but are not limited to, ionic and nonionic surfactants or wetting agents, such as ethoxylated castor oil, polyglycolyzed glycerides, acetylated monoglycerides, sorbitan fatty acid esters, poloxamers, polyoxyethylene sorbitan fatty acid esters, polyoxyethylene derivatives, monoglycerides or ethoxylated derivatives thereof, diglycerides or polyoxyethylene derivatives thereof, sodium docusate, sodium laurylsulfate, cholic acid or derivatives thereof, lecithins, phospholipids, block copolymers of ethylene glycol and propylene glycol, and non-ionic organosilicones. Preferably, the surfactant is a polyoxyethylene sorbitan fatty acid ester, with polysorbate 80 being more preferred.

In one embodiment, suitable nonionic surfactants may include cetomacrogol 1000 (polyoxyethylene(20) cetyl ether), cetostearyl alcohol, cetyl alcohol, coco-betaine, cocamide DEA, cocamide MEA, cocoglycerides, coco-glucoside, decyl glucoside, glyceryl laurate, glyceryl oleate, isoceteth-20, lauryl glucoside, narrow range ethoxylates, NONIDET® P-40, nonoxynol-9, nonoxynols, NP-40, octaethylene glycol monododecyl ether, octyl glucoside, oleyl alcohol, pentaethylene glycol monododecyl ether, Poloxamer, Poloxamer 407, polyglycerol polyricinoleate, polyglyceryl-10 laurate, polysorbate, polysorbate 20, polysorbate 80, sodium coco-sulfate, sorbitan monostearate, sorbitan tristearate, stearyl alcohol, sucrose laurate, TRITON® X-100, TWEEN®-20, and TWEEN®-80.

When the formulation comprises an enzyme powder, the surfactant used to prepare the powder may be present in an amount ranging of from about 5 wt % to 0.1 wt % based on the weight of protein present in the enzyme powder, preferably from about 2 wt % to 0.5 wt % based on the weight of protein present in the enzyme powder.

The enzyme powder may additionally comprise one or more buffers (e.g., sodium and/or potassium salts of bicarbonate, citrate, acetate, phosphate, pyrophosphate, methylphosphonate, succinate, malate, fumarate, tartrate, and maleate), and an enzyme stabilizer (e.g., ethylenediaminetetraacetic acid, (1-hydroxyethylidene)bisphosphonic acid)).

Spray drying of the formulation to form the enzyme powder is carried out, for example, as described generally in Spray Drying Handbook, 5^{th} ed., K. Masters, John Wiley & Sons, Inc., NY, N.Y. (1991), and in PCT Patent Publication Nos. WO 97/41833 and WO 96/32149 to Platz, R. et al.

In general spray drying consists of bringing together a highly dispersed liquid, and a sufficient volume of hot air to produce evaporation and drying of the liquid droplets. Typically the feed is sprayed into a current of warm filtered air that evaporates the solvent and conveys the dried product to a collector. The spent air is then exhausted with the solvent. Those skilled in the art will appreciate that several different types of apparatus may be used to provide the desired product. For example, commercial spray dryers manufactured. by Buchi Ltd. (Postfach, Switzerland) or GEA Niro Corp. (Copenhagen, Denmark) will effectively produce particles of desired size. It will further be appreciated that these spray dryers, and specifically their atomizers, may be modified or customized for specialized applications, such as the simultaneous spraying of two solutions using a double nozzle technique. More specifically, a water-in-oil emulsion can be atomized from one nozzle and a solution containing an anti-adherent such as mannitol can be co-atomized from a second nozzle. In other cases it may be desirable to push the feed solution though a custom designed nozzle using a high pressure liquid chromatography (HPLC) pump. Provided that microstructures comprising the correct morphology and/or composition are produced the choice of apparatus is not critical and would be apparent to the skilled artisan in view of the teachings herein.

The temperature of both the inlet and outlet of the gas used to dry the sprayed material is such that it does not cause degradation of the enzyme in the sprayed material. Such temperatures are typically determined experimentally, although generally, the inlet temperature will range from about 50° C. to about 225° C., while the outlet temperature will range from about 30° C. to about 150° C. Preferred parameters include atomization pressures ranging from about 20-150 psi (0.14 MPa-1.03 MPa), and preferably from about 30-40 to 100 psi (0.21-0.28 MPa to 0.69 MPa). Typically the atomization pressure employed will be one of the following (MPa) 0.14, 0.21, 0.28, 0.34, 0.41, 0.48, 0.55, 0.62, 0.69, 0.76, 0.83 or above.

When using an enzyme powder, the enzyme powder or a formulation of the enzyme powder in carboxylic acid ester may be required to substantially retain its enzymatic activity for an extended period of time when stored at ambient temperature. The enzyme powder or a formulation of the enzyme powder in carboxylic acid ester substantially retains its enzymatic activity at elevated temperatures for short periods of time. In one embodiment, “substantially retains its enzymatic activity” is meant that the enzyme powder or a formulation of the enzyme powder in carboxylic acid ester retains at least about 75 percent of the enzyme activity of the enzyme in the enzyme powder or a formulation of the enzyme powder after an extended storage period at ambient temperature and/or after a short storage period at an elevated temperature (above ambient temperature) in a formulation comprised of a carboxylic acid ester and the enzyme powder as compared to the initial enzyme activity of the enzyme powder prior to the preparation of a formulation comprised of the carboxylic acid ester and the enzyme powder. The extended storage period is a period of time from about one year to about two years at ambient temperature. In one embodiment, the short storage period is at an elevated temperature is a period of time from when the formulation comprised of a carboxylic acid ester and the enzyme powder is produced at 40° C. to about eight weeks at 40° C. In another embodiment, the elevated temperature is in a range of from about 30° C. to about 52° C. In a preferred embodiment, the elevated temperature is in a range of from about 30° C. to about 40° C.

In some embodiments, the enzyme powder retains at least 75 percent of the enzyme activity after eight weeks storage at 40° C. in a formulation comprised of a carboxylic acid ester and the enzyme powder as compared to the initial enzyme activity of the enzyme powder prior to the preparation of a formulation comprised of the carboxylic acid ester and the enzyme powder at 40° C. In other embodiments, the enzyme powder retains at least 76, 77, 78, 79, 80, 81, 82, 83, 84, 85, 86, 87, 88, 89, 90, 91, 92, 93, 94, 95, 96, 97, 98, 99, or 100 percent of the enzyme activity of the at least one enzyme after eight weeks storage at 40° C. in a formulation comprised of a carboxylic acid ester and the enzyme powder as compared to the initial enzyme activity of the enzyme powder prior to the preparation of a formulation comprised of the carboxylic acid ester and the enzyme powder at 40° C. Preferably, perhydrolysis activity is measured as described in Examples 8-13 of U.S. Patent Application Publication No. 2010-0086510; but any method of measuring perhydrolysis activity may be used.

A further improvement in enzyme activity over the stated periods of time may be achieved by adding a buffer having a buffering capacity in a pH range of from about 5.5 to about 9.5 to the formulation comprised of the carboxylic acid ester and the spray-dried enzyme powder as described in U.S. Patent Application Publication No. 2010-0086534. A suitable buffer may include, but is not limited to, sodium salt, potassium salt, or mixtures of sodium or potassium salts of bicarbonate, pyrophosphate, phosphate, methylphosphonate, citrate, acetate, malate, fumarate, tartrate maleate or succinate. Preferred buffers for use in the formulation comprised of the carboxylic acid ester and the spray-dried enzyme powder include the sodium salt, potassium salt, or mixtures of sodium or potassium salts of bicarbonate, pyrophosphate, phosphate, methylphosphonate, citrate, acetate, malate, fumarate, tartrate maleate or succinate. In preferred embodiment, the buffer comprises the sodium and/or potassium salts of bicarbonate.

In embodiments where a buffer may be present in the carboxylic acid ester and enzyme powder formulation, the buffer may be present in an amount in a range of from about 0.01 wt % to about 50 wt % based on the weight of carboxylic acid ester in the formulation comprised of carboxylic acid ester and enzyme powder. The buffer may be present in a more preferred range of from about 0.10% to about 10% based on the weight of carboxylic acid ester in the formulation comprised of carboxylic acid ester and enzyme powder. Further, in these embodiments, the comparison between perhydrolysis activity of the enzyme is determined as between an enzyme powder which retains at least 75 percent of the perhydrolysis activity of the at least one enzyme after eight weeks storage at 40° C. in a formulation comprised of a carboxylic acid ester, a buffer having a buffering capacity in a pH range of from about 5.5 to about 9.5, and the enzyme powder as compared to the initial perhydrolysis activity of the enzyme powder prior to the preparation of a formulation comprised of the carboxylic acid ester, the buffer having a buffering capacity in a pH range of from about 5.5 to about 9.5, and the enzyme powder.

It is intended that the dried enzyme powder be stored as a formulation in the organic compound that is a substrate for the at least one enzyme, such as triacetin. In the absence of added hydrogen peroxide, triacetin is normally hydrolyzed in aqueous solution by a perhydrolytic enzyme to produce diacetin and acetic acid, and the production of acetic acid results in a decrease in the pH of the reaction mixture. One requirement for long term storage stability of the enzyme in triacetin is that there is not a significant reaction of the triacetin with any water that might be present in the triacetin; the specification for water content in one commercial triacetin (supplied by Tessenderlo Group, Brussels, Belgium) is 0.03 wt % water (300 ppm). Any hydrolysis of triacetin that occurs during storage of the enzyme in triacetin would produce acetic acid, which could result in a decrease in activity or inactivation of the perhydrolases; the CE-7 perhydrolases are typically inactivated at or below a pH of 5.0 (see U.S. Patent Application Publication No. 2009-0005590 to DiCosimo, R., et al.). The excipient selected for use in the present application must provide stability of the enzyme in the organic substrate for the enzyme under conditions where acetic acid might be generated due to the presence of low concentrations of water in the formulation. The dried enzyme powder may be stored as a formulation in the organic compound that is a substrate for the at least one enzyme, where the formulation additionally comprises an excipient and one or more buffers (e.g., sodium and/or potassium salts of bicarbonate, citrate, acetate, phosphate, pyrophosphate, methylphosphonate, succinate, malate, fumarate, tartrate, and maleate).

Suitable Reaction Conditions for the Enzyme-Catalyzed Preparation of Peracids from Carboxylic Acid Esters and Hydrogen Peroxide

One or more enzymes having perhydrolytic activity may be used to generate an efficacious concentration of the desired peracid(s) in the present personal care compositions and methods. The desired peroxycarboxylic acid may be prepared by reacting carboxylic acid esters with a source of peroxygen including, but not limited to, hydrogen peroxide, sodium perborate or sodium percarbonate, in the presence of an enzyme catalyst having perhydrolysis activity.

The perhydrolytic enzyme within the targeted perhydrolase may be any perhydrolytic enzyme and may include lipases, proteases, esterases, acyl transferases, aryl esterases, carbohydrate esterases, and combinations so long as the enzyme has perhydrolytic activity for one or more of the present substrates. Examples may include, but are not limited to perhydrolytic proteases (subtilisin variant; U.S. Pat. No. 7,510,859), perhydrolytic esterases (Pseudomonas fluorescens; U.S. Pat. No. 7,384,787; SEQ ID NO: 315 [L29P variant] and SEQ ID NO: 339 [wild type]), perhydrolytic aryl esterases (Mycobacterium smegmatis; U.S. Pat. No. 7,754,460; WO2005/056782; and EP1689859 B1; SEQ ID NOs: 314 [S54V variant] and 338 [wild type]).

In one embodiment, the enzyme catalyst comprises at least one enzyme having perhydrolase activity, wherein said enzyme is structurally classified as a member of the CE-7 carbohydrate esterase family (CE-7; see Coutinho, P. M., and Henrissat, B., supra). In another embodiment, the perhydrolase catalyst is structurally classified as a cephalosporin C deacetylase. In another embodiment, the perhydrolase catalyst is structurally classified as an acetyl xylan esterase.

In one embodiment, the perhydrolase catalyst comprises an enzyme having perhydrolysis activity and a CE-7 signature motif comprising:
a) an RGQ motif that aligns with amino acid residues 118-120 of SEQ ID NO: 2;
b) a GXSQG motif that aligns with amino acid residues 179-183 of SEQ ID NO: 2; and
c) an HE motif that aligns with amino acid residues 298-299 of SEQ ID NO: 2.

In a preferred embodiment, the alignment to reference SEQ ID NO: 2 is performed using CLUSTALW.

In a further embodiment, the CE-7 signature motif additional may comprise and additional (i.e., fourth) motif defined as an LXD motif at amino acid residues 267-269 when aligned to reference sequence SEQ ID NO:2 using CLUSTALW.

In another embodiment, the perhydrolase catalyst comprises an enzyme having perhydrolase activity, said enzyme having an amino acid sequence selected from the group consisting of SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 299, 301, 303, 305, 307, 309, and 311.

In another embodiment, the perhydrolase catalyst comprises an enzyme having perhydrolase activity, said enzyme having an amino acid sequence selected from the group consisting of SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, 64, 293, 297, 299, 301, 303, 305, 307, 309, and 311 wherein said enzyme may have one or more additions, deletions, or substitutions so long as the signature motif is conserved and perhydrolase activity is retained.

As described above, the CE-7 perhydrolase may be a fusion protein having a first portion comprising CE-7 perhydrolase and a second portion comprising a peptidic component having affinity for a target body surface such as that perhydrolase is “targeted” to the desired body surface. In one embodiment, any CE-7 perhydrolase (as defined by the presence of the CE-7 signature motifs) may be fused to any peptidic component/binding element capable of targeting the enzyme to a body surface. In one aspect, the peptidic component having affinity for hair may include antibodies, antibody fragments (F_{ab}), as well as single chain variable fragments (scFv; a fusion of the variable regions of the heavy (V_{H}) and light chains (V_{L}) of immunoglobulins), single domain camelid antibodies, scaffold display proteins, and single chain affinity peptides lacking immunoglobulin folds. The compositions comprising antibodies, antibodies fragments and other immunoglobulin-derived binding elements, as well as large scaffold display proteins, are often not economically viable. As such, and in a preferred aspect, the peptidic component/binding element is a single chain affinity peptide lacking an immunoglobulin fold and/or immunoglobulin domain. Short single chain body surface-binding peptides may be empirically generated (e.g., positively charged polypeptides targeted to negatively charged surfaces) or generated using biopanning against a target body surface. Methods to identify/obtain affinity peptides using any number of display techniques (e.g., phage display, yeast display, bacterial display, ribosome display, and mRNA display) are well known in the art. Individual hair-binding peptides may be coupled together, via optional spacers/linkers, to form larger binding “domains” (also referred to herein as binding “hands”) to enhance attachment/localization of the perhydrolytic enzyme to hair.

The fusion proteins may also include one or more peptide linkers/spacers separating the CE-7 perhydrolase enzyme and the hair-binding domain and/or between different hair-binding peptides (e.g., when a plurality of hair-binding peptides are coupled together to form a larger target hair-binding domain). A non-limiting list of exemplary peptide spacers are provided by the amino acid sequences of SEQ ID NOs: 290, 291, 312, and 313.

Suitable peptides having affinity for hair are described herein, supra. Methods to identify additional hair-binding peptides using any of the above “display” techniques are well known and can be used to identify additional hair-binding peptides.

Suitable carboxylic acid ester substrates may include esters having the following formula:
(a) one or more esters having the structure

[X]ₘR₅
wherein
X is an ester group of the formula R₆C(O)O;
R₆ is a C1 to C7 linear, branched or cyclic hydrocarbyl moiety, optionally substituted with a hydroxyl group or C1 to C4 alkoxy group, wherein R₆ optionally comprises one or more ether linkages where R₆ is C2 to C7;
R₅ is a C1 to C6 linear, branched, or cyclic hydrocarbyl moiety or a five-membered cyclic heteroaromatic moiety or six-membered cyclic aromatic or heteroaromatic moiety optionally substituted with a hydroxyl group; wherein each carbon atom in R₅ individually comprises no more than one hydroxyl group or no more than one ester group or carboxylic acid group, and wherein R₅ optionally comprises one or more ether linkages;
m is an integer ranging from 1 to the number of carbon atoms in R₅,
said one or more esters having solubility in water of at least 5 ppm at 25° C.; or
(b) one or more glycerides having the structure

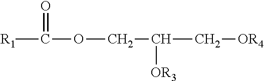

wherein R₁ is a C1 to C7 straight chain or branched chain alkyl optionally substituted with an hydroxyl or a C1 to C4 alkoxy group and R₃ and R₄ are individually H or R₁C(O); or
(c) one or more esters of the formula

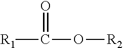

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; or
(d) one or more acetylated monosaccharides, acetylated disaccharides, or acetylated polysaccharides; or
(e) any combination of (a) through (d).

Suitable substrates may also include one or more acylated saccharides selected from the group consisting of acylated mono-, di-, and polysaccharides. In another embodiment, the acylated saccharides are selected from the group consisting of acetylated xylan; fragments of acetylated xylan; acetylated xylose (such as xylose tetraacetate); acetylated glucose (such as α-D-glucose pentaacetate; β-D-glucose pentaacetate; 1-thio-β-D-glucose-2,3,4,6-tetraacetate); β-D-galactose pentaacetate; sorbitol hexaacetate; sucrose octaacetate; β-D-ribofuranose-1,2,3,5-tetraacetate; β-D-ribofuranose-1,2,3,4-tetraacetate; tri-O-acetyl-D-galactal; tri-O-acetyl-D-glucal; β-D-xylofuranose tetraacetate, α-D-glucopyranose pentaacetate; β-D-glucopyranose-1,2,3,4-tetraacetate; β-D-glucopyranose-2,3,4,6-tetraacetate; 2-acetamido-2-deoxy-1,3,4,6-tetraacetyl-β-D-glucopyranose; 2-acetamido-2-deoxy-3,4,6-triacetyl-1-chloride-α-D-glucopyranose; α-D-mannopyranose pentaacetate, and acetylated cellulose. In a preferred embodiment, the acetylated saccharide is selected from the group consisting of β-D-ribofuranose-1,2,3,5-tetraacetate; tri-O-acetyl-D-galactal; tri-O-acetyl-D-glucal; sucrose octaacetate; and acetylated cellulose.

In another embodiment, additional suitable substrates may also include 5-acetoxymethyl-2-furaldehyde; 3,4-diacetoxy-1-butene; 4-acetoxybenzoic acid; vanillin acetate; propylene glycol methyl ether acetate; methyl lactate; ethyl lactate; methyl glycolate; ethyl glycolate; methyl methoxyacetate; ethyl methoxyacetate; methyl 3-hydroxybutyrate; ethyl 3-hydroxybutyrate; and triethyl 2-acetyl citrate.

In another embodiment, suitable substrates are selected from the group consisting of: monoacetin; diacetin; triacetin; monopropionin; dipropionin; tripropionin; monobutyrin; dibutyrin; tributyrin; glucose pentaacetate; xylose tetraacetate; acetylated xylan; acetylated xylan fragments; β-D-ribofuranose-1,2,3,5-tetraacetate; tri-O-acetyl-D-galactal; tri-O-acetyl-D-glucal; monoesters or diesters of 1,2-ethanediol; 1,2-propanediol; 1,3-propanediol; 1,2-butanediol; 1,3-butanediol; 2,3-butanediol; 1,4-butanediol; 1,2-pentanediol; 2,5-pentanediol; 1,5-pentanediol; 1,6-pentanediol; 1,2-hexanediol; 2,5-hexanediol; 1,6-hexanediol; and mixtures thereof. In another embodiment, the substrate is a C1 to C6 polyol comprising one or more ester groups. In a preferred embodiment, one or more of the hydroxyl groups on the C1 to C6 polyol are substituted with one or more acetoxy groups (such as 1,3-propanediol diacetate; 1,2-propanediol diacetate; 1,4-butanediol diacetate; 1,5-pentanediol diacetate, etc.). In a further embodiment, the substrate is propylene glycol diacetate (PGDA), ethylene glycol diacetate (EGDA), or a mixture thereof.

In a further embodiment, suitable substrates are selected from the group consisting of monoacetin, diacetin, triacetin, monopropionin, dipropionin, tripropionin, monobutyrin, dibutyrin, and tributyrin. In yet another aspect, the substrate is selected from the group consisting of diacetin and triacetin. In a most preferred embodiment, the suitable substrate comprises triacetin.

In a preferred embodiment, the carboxylic acid ester is a liquid substrate selected from the group consisting of monoacetin, diacetin, triacetin, and combinations (i.e., mixtures) thereof. The carboxylic acid ester is present in the reaction formulation at a concentration sufficient to produce the desired concentration of peroxycarboxylic acid upon enzyme-catalyzed perhydrolysis. The carboxylic acid ester need not be completely soluble in the reaction formulation, but has sufficient solubility to permit conversion of the ester by the perhydrolase catalyst to the corresponding peroxycarboxylic acid. The carboxylic acid ester is present in the reaction formulation at a concentration of 0.05 wt % to 40 wt % of the reaction formulation, preferably at a concentration of 0.1 wt % to 20 wt % of the reaction formulation, and more preferably at a concentration of 0.5 wt % to 10 wt % of the reaction formulation.

The peroxygen source may include, but is not limited to, hydrogen peroxide, hydrogen peroxide adducts (e.g., urea-hydrogen peroxide adduct (carbamide peroxide)) perborate salts and percarbonate salts. The concentration of peroxygen compound in the reaction formulation may range from 0.0033 wt % to about 50 wt %, preferably from 0.033 wt % to about 40 wt %, more preferably from 0.1 wt % to about 30 wt %.

The peroxygen source (i.e., hydrogen peroxide) may also be generated enzymatically using enzyme capable of producing and effective amount of hydrogen peroxide. For example, various oxidases can be used in the present compositions and methods to produce an effective amount of hydrogen peroxide including, but not limited to glucose oxidase, lactose oxidases, carbohydrate oxidase, alcohol oxidase, ethylene glycol oxidase, glycerol oxidase, and amino acid oxidase.

Many perhydrolase catalysts (whole cells, permeabilized whole cells, and partially purified whole cell extracts) have been reported to have catalase activity (EC 1.11.1.6). Catalases catalyze the conversion of hydrogen peroxide into oxygen and water. In one aspect, the perhydrolysis catalyst lacks catalase activity. In another aspect, a catalase inhibitor may be added to the reaction formulation. One of skill in the art can adjust the concentration of catalase inhibitor as needed. The concentration of the catalase inhibitor typically ranges from 0.1 mM to about 1 M; preferably about 1 mM to about 50 mM; more preferably from about 1 mM to about 20 mM.

In another embodiment, the enzyme catalyst lacks significant catalase activity or may be engineered to decrease or eliminate catalase activity. The catalase activity in a host cell can be down-regulated or eliminated by disrupting expression of the gene(s) responsible for the catalase activity using well known techniques including, but not limited to, transposon mutagenesis, RNA antisense expression, targeted mutagenesis, and random mutagenesis. In a preferred embodiment, the gene(s) encoding the endogenous catalase activity are down-regulated or disrupted (i.e., knocked-out). As used herein, a “disrupted” gene is one where the activity and/or function of the protein encoded by the modified gene is no longer present. Means to disrupt a gene are well-known in the art and may include, but are not limited to, insertions, deletions, or mutations to the gene so long as the activity and/or function of the corresponding protein is no longer present. In a further preferred embodiment, the production host is an E. coli production host comprising a disrupted catalase gene selected from the group consisting of katG and katE (see U.S. Patent Application Publication No. 2008-0176299). In another embodiment, the production host is an E. coli strain comprising a down-regulation and/or disruption in both katG and a katE catalase genes.

The concentration of the catalyst in the aqueous reaction formulation depends on the specific catalytic activity of the catalyst, and is chosen to obtain the desired rate of reaction. The weight of catalyst in perhydrolysis reactions typically ranges from 0.0001 mg to 10 mg per mL of total reaction volume, preferably from 0.001 mg to 2.0 mg per mL. The catalyst may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997. The use of immobilized catalysts permits the recovery and reuse of the catalyst in subsequent reactions. The enzyme catalyst may be in the form of whole microbial cells, permeabilized microbial cells, microbial cell extracts, partially-purified or purified enzymes, and mixtures thereof.

In one aspect, the concentration of peroxycarboxylic acid generated by the combination of chemical perhydrolysis and enzymatic perhydrolysis of the carboxylic acid ester is sufficient to provide an effective concentration of peroxycarboxylic acid for the chosen personal care application. In another aspect, the present methods provide combinations of enzymes and enzyme substrates to produce the desired effective concentration of peroxycarboxylic acid, where, in the absence of added enzyme, there is a significantly lower concentration of peroxycarboxylic acid produced. Although there may in some cases be substantial chemical perhydrolysis of the enzyme substrate by direct chemical reaction of inorganic peroxide with the enzyme substrate, there may not be a sufficient concentration of peroxycarboxylic acid generated to provide an effective concentration of peroxycarboxylic acid in the desired applications, and a significant increase in total peroxycarboxylic acid concentration is achieved by the addition of an appropriate perhydrolase catalyst to the reaction formulation.

The concentration of peroxycarboxylic acid generated (e.g. peracetic acid) by the perhydrolysis of at least one carboxylic acid ester is at least about 0.1 ppm, preferably at least 0.5 ppm, 1 ppm, 5 ppm, 10 ppm, 20 ppm, 100 ppm, 200 ppm, 300 ppm, 500 ppm, 700 ppm, 1000 ppm, 2000 ppm, 5000 ppm or 10,000 ppm of peracid within 10 minutes, preferably within 5 minutes, of initiating the perhydrolysis reaction. The product formulation comprising the peroxycarboxylic acid may be optionally diluted with water, or a solution predominantly comprised of water, to produce a formulation with the desired lower concentration of peroxycarboxylic acid base on the target application. Clearly one of skill in the art can adjust the reaction components and/or dilution amounts to achieve the desired peracid concentration for the chosen personal care product.

In one aspect, the reaction time required to produce the desired concentration of peracid is not greater than about two hours, preferably not greater than about 30 minutes, more preferably not greater than about 10 minutes, and most preferably in about 5 minutes or less. In other aspects, the surface comprising hair is contacted with the peroxycarboxylic acid formed in accordance with the processes described herein within 5 minutes of combining the reaction components. In one embodiment, the target body surface is contacted with the peroxycarboxylic acid produced with the processes described herein within about 5 minutes to about 168 hours of combining said reaction components, or within about 5 minutes to about 48 hours, or within about 5 minutes to 2 hours of combining said reaction components, or any such time interval therein.

The peracid formed in accordance with the processes describe herein is used in a personal care product/application wherein the peracid is contacted with a target body surface to provide a peracid-based benefit, such as hair removal (a peracid depilatory agent), decrease hair tensile strength, a hair pretreatment used to enhance other depilatory products (such as thioglycolate-based hair removal products), hair bleaching, hair dye pretreatment (oxidative hair dyes), hair curling, hair conditioning, skin whitening, skin bleaching, skin conditioning, reducing the appearance of skin wrinkles, skin rejuvenation, reducing dermal adhesions, reducing or eliminating body odors, nail bleaching, or nail disinfecting. In one embodiment, the process to produce a peracid for a target body surface is conducted in situ.

The temperature of the reaction may be chosen to control both the reaction rate and the stability of the enzyme catalyst activity. Clearly for certain personal care applications the temperature of the target body surface may be the temperature of the reaction. The temperature of the reaction may range from just above the freezing point of the reaction formulation (approximately 0° C.) to about 95° C., with a preferred range of 5° C. to about 75° C., and a more preferred range of reaction temperature of from about 5° C. to about 55° C.

The pH of the final reaction formulation containing peroxycarboxylic acid is from about 2 to about 9, preferably from about 3 to about 8, more preferably from about 5 to about 8, even more preferably about 5.5 to about 8, and yet even more preferably about 6.0 to about 7.5. The pH of the reaction, and of the final reaction formulation, may optionally be controlled by the addition of a suitable buffer including, but not limited to, phosphate, pyrophosphate, bicarbonate, acetate, or citrate. The concentration of buffer, when employed, is typically from 0.1 mM to 1.0 M, preferably from 1 mM to 300 mM, most preferably from 10 mM to 100 mM.

In another aspect, the enzymatic perhydrolysis reaction formulation may contain an organic solvent that acts as a dispersant to enhance the rate of dissolution of the carboxylic acid ester in the reaction formulation. Such solvents include, but are not limited to, propylene glycol methyl ether, acetone, cyclohexanone, diethylene glycol butyl ether, tripropylene glycol methyl ether, diethylene glycol methyl ether, propylene glycol butyl ether, dipropylene glycol methyl ether, cyclohexanol, benzyl alcohol, isopropanol, ethanol, propylene glycol, and mixtures thereof.

Single Step Vs. Multi-Step Application Methods

Typically the minimum set of reaction components to enzymatically produce a peracid benefit agent will include (1) at least one enzyme having perhydrolytic activity as described herein, such as a CE-7 perhydrolase (optionally in the form of a targeted fusion protein), (2) at least one suitable carboxylic acid ester substrate, and (3) a source of peroxygen.

The peracid-generating reaction components of the personal care composition may remain separated until use. In one embodiment, the peracid-generating components are combined and then contacted with the target body surface whereby the resulting peracid-based benefit agent provides a benefit to the body surface. The components may be combined and then contacted with the target body surface or may be combined on the targeted body surface. In one embodiment, the peracid-generating components are combined such that the peracid is produced in situ.

A multi-step application may also be used. One or two of the individual components of the peracid-generating system (i.e., a sequential application on the body surface of at least one of the three basic reaction components) composition may be contacted with hair prior to applying the remaining components required for enzymatic peracid production. In one embodiment, the perhydrolytic enzyme is contacted with the hair prior to contacting the hair with the carboxylic acid ester substrate and/or the source of peroxygen (i.e., a “two-step application”).

In one embodiment, the enzyme having perhydrolytic activity is a targeted perhydrolase that is applied to hair prior to combining the remaining components necessary for enzymatic peracid production. In one embodiment, the step of contacting the body surface comprising hair with the targeted perhydrolase lasts less than 1 hour, preferably less than 30 minutes, and most preferably 5 seconds to 5 minutes. In one embodiment, the enzyme is applied to the hair in the form of a body wash comprising at least one surfactant. In a preferred embodiment, the surfactants used are non-ionic. Targeted perhydrolases that do not durably bind to the hair may be removed by washing/rinsing with an aqueous solution (e.g., tap water in an shower or bath) and then optionally drying the hair prior to contacting the durably bound targeted perhydrolases (bound to hair) with the remaining reaction components (a source of hydrogen peroxide and an ester substrate). The conditions of the washing/rinsing step used to remove the subpopulation of enzymes that did not durably bind to the hair may be adjusted such that the hair receives an effective amount of the durably bound targeted perhydrolytic enzyme(s). The hair may be optionally dried after rinsing away the non-durably about targeted perhydrolases. In one embodiment, the remaining reaction components (a source of hydrogen peroxide and the carboxylic acid ester substrate) are contacted with the durably bound targeted perhydrolases for a period of time from 10 seconds to 24 hours. In a further aspect, the source of hydrogen peroxide and the ester substrate are in the form of a hair/skin moisturizer or body lotion (i.e., a water or a water-in-oil mixture) comprising dermally acceptable ingredients.

In a preferred embodiment, the enzyme having perhydrolytic activity is a “targeted CE-7 perhydrolase” CE-7 fusion protein) that is applied to hair prior to combining the remaining components necessary for enzymatic peracid production (i.e., a two-step application method). The targeted perhydrolase is contacted with the hair under suitable conditions to promote non-covalent bonding of the fusion protein to the hair surface. An optional rinsing step may be used to remove excess and/or unbound fusion protein prior to combining the remaining reaction components.

In a further embodiment, the perhydrolytic enzyme (optionally in the form of a fusion protein targeted to a hair surface) and the carboxylic acid ester are applied to the hair prior to the addition of the source of peroxygen.

In a further embodiment, the perhydrolytic enzyme (optionally in the form of a fusion protein targeted to hair) and source of peroxygen (e.g., an aqueous solution comprising hydrogen peroxide) are applied to the hair prior to the addition of the carboxylic acid ester substrate.

In a further embodiment, the carboxylic acid ester substrate and the source of peroxygen (e.g., an aqueous solution comprising hydrogen peroxide) are applied to the hair prior to the addition of the perhydrolytic enzyme (optionally in the form of a fusion protein targeted to hair).

In yet another embodiment, any of the compositions or methods described herein can be incorporated into a kit for practicing the invention. The kits may comprise materials and reagents to facilitate enzymatic production of peracid. An exemplary kit comprises a substrate, a source of peroxygen, and an enzyme catalyst having perhydrolytic activity, wherein the enzyme catalyst can be optionally targeted to hair or a body surface comprising hair. Other kit components may include, without limitation, one or more of the following: sample tubes, solid supports, instruction material, and other solutions or other chemical reagents useful in enzymatically producing peracids, such as acceptable components or carriers.

### Dermatologically Acceptable Components/Carriers/Medium

The compositions and methods described herein may further comprise one or more dermatologically or cosmetically acceptable components known or otherwise effective for use in hair care, skin care, nail care or other personal care products, provided that the optional components are physically and chemically compatible with the essential components described herein, or do not otherwise unduly impair product stability, aesthetics, or performance. Non-limiting examples of such optional components are disclosed in International Cosmetic Ingredient Dictionary, Ninth Edition, 2002, and CTFA Cosmetic Ingredient Handbook, Tenth Edition, 2004.

In one embodiment, the dermatologically acceptable carrier may comprise from about 10 wt % to about 99.9 wt %, alternatively from about 50 wt % to about 95 wt %, and alternatively from about 75 wt % to about 95 wt %, of a dermatologically acceptable carrier. Carriers suitable for use with the composition(s) may include, for example, those used in the formulation of hair sprays, mousses, tonics, gels, skin moisturizers, lotions, and leave-on conditioners. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters (with the understanding that the choice of ester(s) may be dependent on whether or not it may act as a carboxylic acid ester substrates for the perhydrolases) of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one embodiment, the carrier comprises water, fatty alcohols, volatile organic alcohols, and mixtures thereof.

The composition(s) of the present invention further may comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ SC91 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers. Preferred gelling agents include water-soluble unneutralized crosslinked ethylene/maleic anhydride copolymers, water-soluble unneutralized crosslinked carboxylic acid polymers, water-soluble hydrophobically modified nonionic cellulose polymers and surfactant/fatty alcohol gel networks such as those suitable for use in hair conditioning products.

### Hair Care Compositions

The peracid generation components can be incorporated into hair care compositions and products to generate an efficacious concentration of at least one peracid. The perhydrolase used to generate the desired amount of peracid may be used in the form of a fusion protein where the first portion of the fusion protein comprises the perhydrolase a second portion having affinity for hair.

The peracid produced provides a benefit to hair (i.e., a “peracid-based benefit agent”). The peracid may be used as a depilatory agent, a hair treatment agent to reduce the tensile strength of hair, a hair pretreatment agent used to enhance the performance of other depilatory products (such as thioglycolate-based hair removal products), a hair bleaching agent, a hair dye pretreatment agent (oxidative hair dyes), a hair curling/styling agent, and as a component in hair conditioning products.

In addition to the peracid-based benefit agent, hair care products and formulations may also include any number of additional components commonly found in hair care products. The additional components may help to improve the appearance, texture, color, and sheen of hair as well as increasing hair body or suppleness.

Hair conditioning agents are well known in the art, see for example Green et al. (WO 0107009), incorporated herein by reference, and are available commercially from various sources. Suitable examples of hair conditioning agents include, but are not limited to, cationic polymers, such as cationized guar gum, diallyl quaternary ammonium salt/acrylamide copolymers, quaternized polyvinylpyrrolidone and derivatives thereof, and various polyquaternium-compounds; cationic surfactants, such as stearalkonium chloride, centrimonium chloride, and sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and nanoparticles, such as silica nanoparticles and polymer nanoparticles.

The hair care products may also include additional components typically found in cosmetically acceptable media. Non-limiting examples of such components are disclosed in International Cosmetic Ingredient Dictionary, Ninth Edition, 2002, and CTFA Cosmetic Ingredient Handbook, Tenth Edition, 2004. A non-limiting list of components often included in a cosmetically acceptable medium for hair care are also described by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including but not limited to, antioxidants, preserving agents, fillers, surfactants, UVA and/or UVB sunscreens, fragrances, thickeners, gelling agents, wetting agents and anionic, nonionic or amphoteric polymers, and dyes or pigments.

The hair care compositions and methods may also include at least one coloring agents such as any dye, lake, pigment, and the like that may be used to change the color of hair, skin, or nails. Hair coloring agents are well known in the art (see for example Green et al. supra, CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Suitable hair coloring agents include, but are not limited to dyes, such as 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, titanium dioxide nanoparticles, zinc oxide, barium oxide, ultramarine blue, bismuth citrate, and carbon black particles. In one embodiment, the hair coloring agents are D&C Yellow 1 and 3, HC Yellow 6 and 8, D&C Blue 1, HC Blue 1, HC Brown 2, HC Red 5,2-nitro-paraphenylenediamine, N,N-hydroxyethyl-2-nitro-phenylenediamine, 4-nitro-indole, and carbon black. Metallic and semiconductor nanoparticles may also be used as hair coloring agents due to their strong emission of light (U.S. Patent Application Publication No. 2004-0010864 to Vic et al.).

Hair care compositions may include, but not limited to shampoos, conditioners, lotions, aerosols, gels, mousses, and hair dyes.

In one embodiment, a hair care composition is provided comprising:

a) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
i) an RGQ motif at positions corresponding to positions 118-120 of SEQ ID NO:2;
ii) a GXSQG motif at positions corresponding to positions 179-183 of SEQ ID NO:2; and
iii) an HE motif at positions corresponding to positions 298-299 of SEQ ID NO:2; and

b) at least one substrate selected from the group consisting of:
i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and

wherein said esters have a solubility in water of at least 5 ppm at 25° C.;
ii) glycerides having the structure

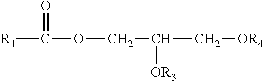

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

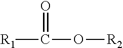

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides;

c) a source of peroxygen; and

d) a dermally acceptable carrier medium; wherein the composition comprises peracid when (a), (b), and (c) are combined.

In another embodiment, the perhydrolytic enzyme used in the hair care composition is a fusion protein comprising

a) a first portion comprising the enzyme having perhydrolytic activity and

b) a second portion having affinity for hair.

In one embodiment, the peracid formed in the hair care composition is peracetic acid.

The components of the hair care composition may remain separated until use. In one embodiment, the peracid-generating components are combined and then contacted with the hair surface whereby the resulting peracid-based benefit agent provides a benefit selected from the group consisting of hair removal, hair weakening (as measured by a decrease in the tensile strength of hair), hair bleaching, hair dye pretreating (oxidative hair dyes), hair curling, and hair conditioning (i.e., a one-step application method). In another embodiment, the peracid-generating components are combined such that the peracid is produced in situ. The relative amount of the ingredients in the hair care composition may be varied according to the desired effect.

In one embodiment a single-step hair treatment method is provided comprising:
1) providing a set of reaction components comprising:
   a) at least one substrate selected from the group consisting of:
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

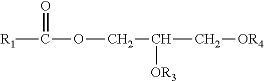

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

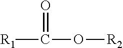

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides;
b) a source of peroxygen; and
c) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
   i) an RGQ motif at positions corresponding to positions 118-120 of SEQ ID NO:2;
   ii) a GXSQG motif at positions corresponding to positions 179-183 of SEQ ID NO:2; and
iii) an HE motif at positions corresponding to positions 298-299 of SEQ ID NO:2; and
2) combining the reaction components of (1), whereby at least one peracid is produced; and
3) contacting hair with said peracid; whereby the resulting peracid-based benefit agent provides a benefit selected from the group consisting of hair removal, hair weakening, hair bleaching, hair dye pretreating, hair curling, and hair conditioning; wherein one or more components of a cosmetically acceptable media may be present.

One or two of the individual components of the peracid generating system (i.e., sequential application on the hair surface) composition may be contacted with the hair surface prior to applying the remaining components required for enzymatic peracid production. In one embodiment, the perhydrolytic enzyme is contacted with the hair prior to the substrate and the source of peroxygen (i.e., a “two-step application”). In a preferred embodiment, the enzyme having perhydrolytic activity is a targeted perhydrolase (i.e., fusion protein) that is applied to the hair surface prior to the remaining components necessary for enzymatic peracid production (i.e., a two-step application method).

In another embodiment, a method is provided comprising
1) contacting hair with a fusion protein comprising;
   a) a first portion comprising an enzyme having perhydrolytic activity, wherein said enzyme having a CE-7 signature motif that aligns with a reference sequence SEQ ID NO: 2 using CLUSTALW, said signature motif comprising:
      i) an RGQ motif at positions corresponding to positions 118-120 of SEQ ID NO:2;
      ii) a GXSQG motif at positions corresponding to positions 179-183 of SEQ ID NO:2; and
      iii) an HE motif at positions corresponding to positions 298-299 of SEQ ID NO:2; and
   b) a second portion comprising a peptidic component having affinity for hair; whereby the fusion peptide binds to the hair;
2) optionally rinsing the hair with an aqueous solution to remove unbound fusion peptide;
3) contacting the hair comprising bound fusion peptide with
   a) at least one substrate selected from the group consisting of:
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

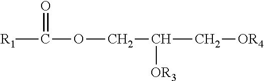

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

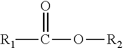

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iii) acetylated saccharides selected from the group consisting of acetylated monosaccharides, acetylated disaccharides, and acetylated polysaccharides; and
b) a source of peroxygen; whereby upon combining the fusion peptide with the substrate and the source of peroxygen a peracid is produced; whereby the resulting peracid provides a benefit selected from the group consisting of hair removal, hair weakening, hair bleaching, hair dye pretreating, hair curling, and hair conditioning.

In a preferred embodiment, the above peracid-based hair care methods are used to remove hair and/or weaken the tensile strength of hair. The hair care methods directed to hair removal or tensile strength reduction may optionally include a reducing agent (to reduce disulfide linkages that join pairs of cysteines), such as a thioglycolate, to enhance the weakening and/or removal of the hair from the surface comprising the hair targeted for removal.

In a further embodiment, the above hair depilatory methods may be used as a pre-treatment for subsequence application of a commercial hair removal product comprising at least one reducing agent, such as a thioglycolate-based hair removal product. As such, the above method may include the step of contacting the peracid treated hair with a reducing agent. Preferably the reducing agent is a thioglycolate, such as sodium thioglycolate or potassium thioglycolate (e.g., an active ingredient often used in hair removal products such as NAIR®).

### Hair Removal Using a Daily Use Product

In one embodiment, a personal care product comprising a low concentration of a peracid may be used to remove or weaken the tensile strength of hair. The concentration of peracid may be controlled such that other body surfaces (i.e., skin) are not adversely affected by the repeated application of the peracid composition. The peracid used in this specific embodiment (i.e., a multi-application, daily use product comprising a low concentration of peracid) may be enzymatically generated, chemically generated, a pre-formed peracid or any combination thereof.

In one embodiment, the personal care daily use product comprises at least one enzymatically generated peracid; preferably generated using a CE-7 perhydrolase. The concentration of peracid is controlled such that hair removal requires multiple applications of the product. Preferably the product may be applied once a month, once every 2 weeks, once a week, more than once per week or daily.

In one embodiment, a method to remove hair is provided comprising
a) providing a composition comprising 0.001 to 4 wt % peracid;
b) contacting a body surface comprising hair with said peracid under suitable conditions to form peracid-treated hair;
c) optionally rinsing the peracid-treated hair with an aqueous solution;
d) optionally drying the hair after step (b) or step (c);
e) repeating steps (a) through (d) from until the hair is removed from the body surface.

In one embodiment, the body surface comprising hair may optionally be wetted, cleaned with a body wash, or combination thereof prior to contacting the hair with peracid.

In yet another embodiment, a two-step application method may be used wherein the targeted perhydrolase is applied prior to the remaining components necessary for enzymatic peracid production.

In another embodiment, a method to decrease the tensile strength of hair is provided comprising
a) providing a composition comprising 0.01 to 4 wt % peracid;
b) contacting a body surface comprising hair characterized by an initial tensile strength with the composition comprising peracid under suitable conditions to form peracid-treated hair;
c) optionally rinsing the peracid-treated hair with an aqueous solution;
d) optionally drying the hair after step (b) or step (c); and
e) repeating steps (a) through (d) whereby the initial tensile strength of the hair is reduced.

In another embodiment, the above hair removal/tensile strength reduction method may optionally include contacting the hair with a swelling agent (i.e., a chemical used to swell or temporarily open up a hair fiber), such as up to 25 wt % urea, before, during or after step (b).

In another embodiment, the above methods may further include contacting the peracid-treated hair with at least one reducing agent. The reducing agent may include any reducing agent capable of reducing the disulfide bonds within the hair.

The reducing agent may include thioglycolates, sulphites, bisulphites, cysteine, glyceryl monothioglycolate, cysteamine, or any combination thereof. In a preferred embodiment, the reducing agent comprises at least one thioglycolate (e.g., potassium thioglycolate, calcium thioglycolate, etc.) in an amount suitable to enhance hair removal or weakening. Preferably the reducing agent is applied at a concentration of less than 25 wt %.

In another embodiment, any of the above hair removal/weakening methods is repeated up to 30 times prior to achieving the desired effect (hair removal/tensile strength reduction). Preferably the peracid concentration and application conditions are selected such that the hair removal/weakening product can be safely applied at least once per month, preferably at least once per week, more preferably once every other day, and most preferably at least once per day. In another embodiment, the hair removal/weakening method is repeated twice per day, for up to 30 days.

The peracid used to remove/weaken hair in the above method may be pre-formed, produced by chemical perhydrolysis, and/or enzymatically-produced using a perhydrolytic enzyme. Preferably the perhydrolytic enzyme is a CE-7 carbohydrate esterase having perhydrolytic activity.

In one embodiment, the composition comprising 0.001 to 4 wt % peracetic acid is produced non-enzymatically by chemically reacting a source of hydrogen peroxide and a suitable carboxylic acid ester substrate to produce the peracetic acid. In one aspect, the peracid precursor used to chemically generate the peracetic acid is coupled through a peptidic component having affinity to hair prior to chemically producing the peracetic acid. In another embodiment, the peracid precursor is TAED (tetraacetylethylenediamine) or a suitable carboxylic acid ester substrate as described herein.

In another embodiment, the composition comprising 0.001 to 4 wt % peracetic acid is produced enzymatically by combining a perhydrolytic enzyme, a source of peroxygen, and a carboxylic acid ester substrate prior to contacting the body surface.

In one embodiment, the enzyme having perhydrolytic activity is selected from the group lipases, proteases, esterases, acyl transferases, aryl esterases, carbohydrate esterases, and combinations thereof.

In another embodiment, the enzyme having perhydrolytic activity comprises an amino acid sequence having at least 95% identity to SEQ ID NOs: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

In one embodiment, the suitable perhydrolases may include enzymes comprising an amino acid sequence having at least 30%, 33%, 40%, 50%, 60%, 70%, 80%, 85%, 90%, 91%, 92%, 93%, 94%, 95%, 96%, 97%, 98%, or 99% amino acid identity to SEQ ID NO: 314, 315, 338, and 339.

In one embodiment, the suitable perhydrolase comprises an amino acid sequence having at least 95% identity to SEQ ID NO: 314.

In a further embodiment, the perhydrolases are CE-7 carbohydrate esterases having perhydrolytic activity, each enzyme having a CE-7 signature motif that aligns with a reference sequence SEQ ID NO: 2 using CLUSTALW, said signature motif comprising:
a) an RGQ motif at positions corresponding to positions 118-120 of SEQ ID NO:2;
b) a GXSQG motif at positions corresponding to positions 179-183 of SEQ ID NO:2; and
c) an HE motif at positions corresponding to positions 298-299 of SEQ ID NO:2.

A hair care product for hair removal and/or hair weakening is also provided comprising:

a) 0.1 to 50 wt % of at least one substrate selected from the group consisting of:
i) esters having the structure

[X]ₘR₅
wherein X=an ester group of the formula R₆C(O)O;
R₆=a C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups, wherein each carbon atom in R₅ individually comprises no more than one hydroxyl group or no more than one ester group or carboxylic acid group;
wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

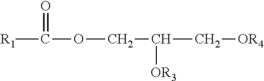

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

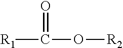

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides;

b) 0.1 to 15 wt % a source of hydrogen peroxide;

c) 0.001 to 1 wt % of an enzyme catalyst comprising at least one CE-7 carbohydrate esterase having perhydrolytic activity; and

d) up to 98 wt % of the balance of the personal care composition comprising a dermally acceptable carrier medium; whereby upon mixing the reaction components a peracid is produce. In one embodiment, the hair care product may comprise up to 25 wt % of a hair swelling agent. In a further embodiment, the hair swelling agent comprises urea.

The hair care product above may further comprise up to 25 wt % of a reducing agent capable of reducing disulfide bonds within hair keratin. In a preferred embodiment, the reducing agent comprises at least one thioglycolate. The reducing agent may be supplied as a separate component that is contacted with the peracid-treated hair.

### Skin Care Compositions

The CE-7 peracid generation components can be incorporated into skin care formulations and products to generate an efficacious concentration of at least one peracid. The CE-7 perhydrolase used to generate the desired amount of peracid may be used in the form of a fusion protein where the first portion of the fusion protein comprises the CE-7 perhydrolase a second portion having affinity for skin.

Examples of skin-binding peptides previously identified are provided herein. Method to identify additional peptides having affinity for skin are well known in the art any may include any number of peptide display techniques such as phage display, ribosome display, and mRNA-display, to name a few. Additionally, skin-binding peptide may be empirically prepared to have an electrostatic attraction to skin.

The peracid produced provides a benefit to skin. The peracid may be used for skin whitening, skin bleaching, skin conditioning, reducing the appearance of skin wrinkles, skin rejuvenation, reducing dermal adhesions, and reducing or eliminating body odors. The present skin care product may be used as a pre-treatment for non-peracid based skin care products

Any number of dermatologically-acceptable materials commonly used in skin care product may also be incorporated into the present skin care compositions such as skin conditioning agents and skin colorants.

Skin conditioning agents as herein defined include, but are not limited to astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Skin conditioning agents are well known in the art, see for example Green et al. (WO01/07009), and are available commercially from various sources. Suitable examples of skin conditioning agents include, but are not limited to, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils and triglyceride esters. The skin conditioning agents may include polysalicylates, propylene glycol (CAS No. 57-55-6, Dow Chemical, Midland, Mich.), glycerin (CAS No. 56-81-5, Proctor & Gamble Co., Cincinnati, Ohio), glycolic acid (CAS No. 79-14-1, DuPont Co., Wilmington, Del.), lactic acid (CAS No. 50-21-5, Alfa Aesar, Ward Hill, Mass.), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, Del.). Polysalicylates may be prepared by the method described by White et al. in U.S. Pat. No. 4,855,483, incorporated herein by reference. Glucaric acid may be synthesized using the method described by Merbouh et al. (Carbohydr. Res. 336:75-78 (2001). The 3-hydroxyvaleric acid may be prepared as described by Bramucci in published international patent application number WO 02/012530.

The cosmetically acceptable medium may contain a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

Skin coloring agents may include the following dyes: eosin derivatives such as D&C Red No. 21 and halogenated fluorescein derivatives such as D&C Red No. 27, D&C Red Orange No. 5 in combination with D&C Red No. 21 and D&C Orange No. 10, and the pigments: titanium dioxide, titanium dioxide nanoparticles, zinc oxide, D&C Red No. 36 and D&C Orange No. 17, the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of D&C Red No. 27, of D&C Red No. 21, of FD&C Blue No. 1, iron oxides, manganese violet, chromium oxide, ultramarine blue, and carbon black. The coloring agent may also be a sunless tanning agent, such as dihydroxyacetone, that produces a tanned appearance on the skin without exposure to the sun.

In one embodiment, a skin care composition is provided comprising:

a) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
i) an RGQ motif at positions corresponding to positions 118-120 of SEQ ID NO:2;
ii) a GXSQG motif at positions corresponding to positions 179-183 of SEQ ID NO:2; and
iii) an HE motif at positions corresponding to positions 298-299 of SEQ ID NO:2; and

b) at least one substrate selected from the group consisting of:
i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
   wherein said esters have a solubility in water of at least 5 ppm at 25° C.;
ii) glycerides having the structure

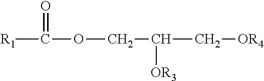

wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula

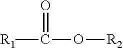

wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides;

c) a source of peroxygen; and

d) a dermally acceptable carrier medium for skin; wherein the composition comprises peracid when (a), (b), and (c) are combined.

In another embodiment, the perhydrolytic enzyme used in the skin care composition is a fusion protein comprising

a) a first portion comprising the enzyme having perhydrolytic activity; and

b) a second portion having affinity for skin.

In one embodiment, the peracid formed for use in a skin care product is peracetic acid. The relative amount of the ingredients in the skin care composition may be varied according to the desired effect.

The components of the skin care composition may remain separated until use. In one embodiment, the peracid-generating components are combined and then contacted with the skin surface whereby the peracid-based benefit agent provides a beneficial effect to the skin (i.e., a one-step application method). In another embodiment, the peracid-generating components are combined such that the peracid is produced in situ.

In one embodiment, a single step method is provided comprising:
1) providing a set of reaction components comprising:
   a) at least one substrate selected from the group consisting of:
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

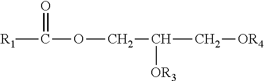

wherein R₁=C1 to C7 straight chain or branched chain alkyl optionally substituted with an hydroxyl or a C1 to C4 alkoxy group and R₃ and R₄ are individually H or R₁C(O); and
iii) acetylated saccharides selected from the group consisting of acetylated monosaccharides, acetylated disaccharides, and acetylated polysaccharides;
b) a source of peroxygen; and
c) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
   i) an RGQ motif at positions corresponding to positions 118-120 of SEQ ID NO:2;
   ii) a GXSQG motif at positions corresponding to positions 179-183 of SEQ ID NO:2; and
iii) an HE motif at positions corresponding to positions 298-299 of SEQ ID NO:2; and
2) combining the reaction components of (1), whereby at least one peracid is produced; and
3) contacting skin with said peracid; whereby the peracid treatment provides a benefit selected from the group consisting of skin whitening, skin bleaching, skin conditioning, reducing the appearance of skin wrinkles, skin rejuvenation, reducing dermal adhesions, and reducing or eliminating body odors; wherein one or more components of a cosmetically acceptable media may be present.

One or two of the individual components of the peracid generating system (i.e., sequential application on the skin surface) composition may be contacted with the skin surface prior to applying the remaining components required for enzymatic peracid production. In one embodiment, the perhydrolytic enzyme is contacted with the skin prior to the substrate and the source of peroxygen (i.e., a “two-step application”). In a preferred embodiment, the enzyme having perhydrolytic activity is a targeted perhydrolase (i.e., fusion protein) that is applied to the skin surface prior to the remaining components necessary for enzymatic peracid production (i.e., a two-step application method).

In another embodiment, a method is provided comprising
1) contacting skin with a fusion protein comprising;
   a) a first portion comprising an enzyme having perhydrolytic activity, wherein said enzyme having a CE-7 signature motif that aligns with a reference sequence SEQ ID NO: 2 using CLUSTALW, said signature motif comprising:
      i) an RGQ motif at positions corresponding to positions 118-120 of SEQ ID NO:2;
      ii) a GXSQG motif at positions corresponding to positions 179-183 of SEQ ID NO:2; and
      iii) an HE motif at positions corresponding to positions 298-299 of SEQ ID NO:2; and
   b) a second portion comprising a peptidic component having affinity for skin; whereby the fusion peptide binds to the skin;
2) optionally rinsing the skin with an aqueous solution to remove unbound fusion peptide;
3) contacting the skin comprising bound fusion peptide with
   a) at least one substrate selected from the group consisting of:
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

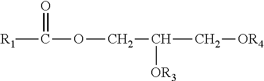

wherein R₁=C1 to C7 straight chain or branched chain alkyl optionally substituted with an hydroxyl or a C1 to C4 alkoxy group and R₃ and R₄ are individually H or R₁C(O); and
iii) acetylated saccharides selected from the group consisting of acetylated monosaccharides, acetylated disaccharides, and acetylated polysaccharides; and
b) a source of peroxygen; whereby upon combining the fusion peptide with the substrate and the source of peroxygen a peracid is produced; whereby the skin receives a benefit selected from the group consisting of skin whitening, skin bleaching, skin conditioning, reducing the appearance of skin wrinkles, skin rejuvenation, reducing dermal adhesions, and reducing or eliminating body odors.

### Nail Care Compositions

The CE-7 peracid generation components may be incorporated into nail care formulations and products to generate an efficacious concentration of at least one peracid. The CE-7 perhydrolase used to generate the desired amount of peracid may be used in the form of a fusion protein where the first portion of the fusion protein comprises the CE-7 perhydrolase a second portion having affinity for nail.

Provided here are peptides having affinity for nail. Method to identify additional peptides having affinity for nail are well known in the art any may include any number of peptide display techniques such as phage display, ribosome display, and mRNA-display, to name a few. Additionally, nail-binding peptide may be empirically prepared to have an electrostatic attraction to nail.

The peracid produced provides a benefit to nail. The peracid may be used for nail bleaching, nail disinfecting, nail resurfacing or as a pre-treatment for other nail care products, such as a nail polish.

Any number of dermatologically acceptable materials commonly used in nail care product may also be incorporated into the present nail care compositions such as nail conditioning agents and nail colorants.

Nail colorants may include D&C Red Nos. 8, 10, 30 and 36, the barium lakes of D&C Red Nos. 6, 9 and 12, the calcium lakes of D&C Red Nos. 7, 11, 31 and 34, the strontium lake of D&C Red No. 30 and D&C Orange No. 17 and D&C Blue No. 6.

The nail colorants may be used in nail polish compositions for coloring fingernails and toenails. Nail polish compositions are compositions for the treatment and coloring of nails, comprising an effective amount of at least one nail colorants in a cosmetically acceptable medium. Components of a cosmetically acceptable medium for nail polishes are described by Philippe et al., supra. The nail polish composition typically contains a solvent and a film forming substance, such as cellulose derivatives, polyvinyl derivatives, acrylic polymers or copolymers, vinyl copolymers and polyester polymers. Additionally, the nail polish may contain a plasticizer, such as tricresyl phosphate, benzyl benzoate, tributyl phosphate, butyl acetyl ricinoleate, triethyl citrate, tributyl acetyl citrate, dibutyl phthalate or camphor.

In one embodiment, a nail care composition is provided comprising:

a) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
i) an RGQ motif at positions corresponding to positions 118-120 of SEQ ID NO:2;
ii) a GXSQG motif at positions corresponding to positions 179-183 of SEQ ID NO:2; and
iii) an HE motif at positions corresponding to positions 298-299 of SEQ ID NO:2; and

b) at least one substrate selected from the group consisting of:
i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
   wherein said esters have a solubility in water of at least 5 ppm at 25° C.;
ii) glycerides having the structure

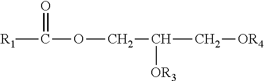

wherein R₁=C1 to C7 straight chain or branched chain alkyl optionally substituted with an hydroxyl or a C1 to C4 alkoxy group and R₃ and R₄ are individually H or R₁C(O); and
iii) acetylated saccharides selected from the group consisting of acetylated monosaccharides, acetylated disaccharides, and acetylated polysaccharides;

c) a source of peroxygen; and

d) a cosmetically acceptable carrier medium for nail; wherein the composition comprises peracid when (a), (b), and (c) are combined.

In another embodiment, the perhydrolytic enzyme used in the nail care composition is a fusion protein comprising

a) a first portion comprising the enzyme having perhydrolytic activity and

b) a second portion having affinity for nail.

In one embodiment, the peracid formed for the nail care composition is peracetic acid. The relative amount of the ingredients in the nail care composition may be varied according to the desired effect.

The components of the nail care composition may remain separated until use. In one embodiment, the peracid-generating components are combined and then contacted with the nail surface whereby the peracid-based benefit agent provides a beneficial effect to the nail (i.e., a one-step application method). In another embodiment, the peracid-generating components are combined such that the peracid is produced in situ.

In one embodiment, a single step method for treating nails is provided comprising:
1) providing a set of reaction components comprising:
   a) at least one substrate selected from the group consisting of:
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii) glycerides having the structure

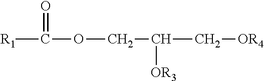

wherein R₁=C1 to C7 straight chain or branched chain alkyl optionally substituted with an hydroxyl or a C1 to C4 alkoxy group and R₃ and R₄ are individually H or R₁C(O); and
iii) acetylated saccharides selected from the group consisting of acetylated monosaccharides, acetylated disaccharides, and acetylated polysaccharides;
b) a source of peroxygen; and
c) an enzyme catalyst having perhydrolytic activity, wherein said enzyme catalyst comprises an enzyme having a CE-7 signature motif that aligns with a reference sequence SEQ ID NO: 2 using CLUSTALW, said signature motif comprising:
   i) an RGQ motif at positions corresponding to positions 118-120 of SEQ ID NO:2;
   ii) a GXSQG motif at positions corresponding to positions 179-183 of SEQ ID NO:2; and
   iii) an HE motif at positions corresponding to positions 298-299 of SEQ ID NO:2; and
2) combining the reaction components of (1), whereby at least one peracid is produced; and
3) contacting nail with said peracid; whereby the peracid treatment provides a benefit selected from the group consisting of nail bleaching, nail resurfacing, and nail disinfecting.

One or two of the individual components of the peracid generating system (i.e., sequential application on the nail surface) composition may be contacted with the nail surface prior to applying the remaining components required for enzymatic peracid production. In one embodiment, the perhydrolytic enzyme is contacted with the nail prior to the substrate and the source of peroxygen (i.e., a “two-step application”). In a preferred embodiment, the enzyme having perhydrolytic activity is a targeted perhydrolase (i.e., fusion protein) that is applied to the nail surface prior to the remaining components necessary for enzymatic peracid production (i.e., a two-step application method).

In another embodiment, a method is provided comprising
1) contacting nail with a fusion protein comprising;
   a) a first portion comprising an enzyme having perhydrolytic activity, wherein said enzyme having a CE-7 signature motif that aligns with a reference sequence SEQ ID NO: 2 using CLUSTALW, said signature motif comprising:
      i) an RGQ motif at positions corresponding to positions 118-120 of SEQ ID NO:2;
      ii) a GXSQG motif at positions corresponding to positions 179-183 of SEQ ID NO:2; and
      iii) an HE motif at positions corresponding to positions 298-299 of SEQ ID NO:2; and
   b) a second portion comprising a peptidic component having affinity for nail; whereby the fusion peptide binds to the nail;
2) optionally rinsing the nail with an aqueous solution to remove unbound fusion peptide;
3) contacting the nail comprising bound fusion peptide with
   a) at least one substrate selected from the group consisting of:
      i) esters having the structure

[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have a solubility in water of at least 5 ppm at 25° C.;
ii). glycerides having the structure

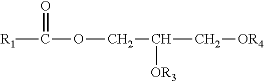

wherein R₁=C1 to C7 straight chain or branched chain alkyl optionally substituted with an hydroxyl or a C1 to C4 alkoxy group and R₃ and R₄ are individually H or R₁C(O); and
iii) acetylated saccharides selected from the group consisting of acetylated monosaccharides, acetylated disaccharides, and acetylated polysaccharides; and
b) a source of peroxygen; whereby upon combining the fusion peptide with the substrate and the source of peroxygen a peracid is produced; whereby the nail receives a benefit selected from the group consisting of nail bleaching, nail resurfacing, and nail disinfecting.
   HPLC Assay Method for Determining the Concentration of Peroxycarboxylic acid and Hydrogen Peroxide

A variety of analytical methods can be used in the present methods to analyze the reactants and products including, but not limited to, titration, high performance liquid chromatography (HPLC), gas chromatography (GC), mass spectroscopy (MS), capillary electrophoresis (CE), the analytical procedure described by U. Pinkernell et al., (Anal. Chem., 69(17):3623-3627 (1997)), and the 2,2′-azino-bis(3-ethylbenzothiazoline)-6-sulfonate (ABTS) assay (U. Pinkernell et. al. Analyst, 122: 567-571 (1997) and Dinu et. al. Adv. Funct. Mater., 20: 392-398 (2010)) as described in the present examples.

### Determination of Minimum Biocidal Concentration of Peroxycarboxylic Acids

Certain personal care applications may be associated with the removal of unwanted microbes, such as those associated with body odor and fungal infections, to name a few. As such, one may want to measure the minimum biocidal concentration for the target personal care application. The method described by J. Gabrielson, et al. (J. Microbiol. Methods 50: 63-73 (2002)) can be employed for determination of the Minimum Biocidal Concentration (MBC) of peroxycarboxylic acids, or of hydrogen peroxide and enzyme substrates. The assay method is based on XTT reduction inhibition, where XTT ((2,3-bis[2-methoxy-4-nitro-5-sulfophenyl]-5-[(phenylamino)carbonyl]-2H-tetrazolium, inner salt, monosodium salt) is a redox dye that indicates microbial respiratory activity by a change in optical density (OD) measured at 490 nm or 450 nm. However, there are a variety of other methods available for testing the activity of disinfectants and antiseptics including, but not limited to, viable plate counts, direct microscopic counts, dry weight, turbidity measurements, absorbance, and bioluminescence (see, for example Brock, Semour S., Disinfection, Sterilization, and Preservation, 5^{th} edition, Lippincott Williams & Wilkins, Philadelphia, Pa., USA; 2001).

### Recombinant Microbial Expression

The genes and gene products of the instant sequences may be produced in heterologous host cells, particularly in the cells of microbial hosts. Preferred heterologous host cells for expression of the instant genes and nucleic acid molecules are microbial hosts that can be found within the fungal or bacterial families and which grow over a wide range of temperature, pH values, and solvent tolerances. For example, it is contemplated that any of bacteria, yeast, and filamentous fungi may suitably host the expression of the present nucleic acid molecules. The perhydrolase may be expressed intracellularly, extracellularly, or a combination of both intracellularly and extracellularly, where extracellular expression renders recovery of the desired protein from a fermentation product more facile than methods for recovery of protein produced by intracellular expression. Transcription, translation and the protein biosynthetic apparatus remain invariant relative to the cellular feedstock used to generate cellular biomass; functional genes will be expressed regardless. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, bacterial host strains include Escherichia, Bacillus, Kluyveromyces, and Pseudomonas. In a preferred embodiment, the bacterial host cell is Bacillus subtilis or Escherichia coli.

Large-scale microbial growth and functional gene expression may use a wide range of simple or complex carbohydrates, organic acids and alcohols or saturated hydrocarbons, such as methane or carbon dioxide in the case of photosynthetic or chemoautotrophic hosts, the form and amount of nitrogen, phosphorous, sulfur, oxygen, carbon or any trace micronutrient including small inorganic ions. The regulation of growth rate may be affected by the addition, or not, of specific regulatory molecules to the culture and which are not typically considered nutrient or energy sources.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell and/or native to the production host, although such control regions need not be so derived.

Initiation control regions or promoters which are useful to drive expression of the present cephalosporin C deacetylase coding region in the desired host cell are numerous and familiar to those skilled in the art. Virtually any promoter capable of driving these genes is suitable for the present invention including but not limited to, CYC1, HIS3, GAL1, GAL10, ADH1, PGK, PHO5, GAPDH, ADC1, TRP1, URA3, LEU2, ENO, TP1 (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred host cell. In one embodiment, the inclusion of a termination control region is optional. In another embodiment, the chimeric gene includes a termination control region derived from the preferred host cell.

### Industrial Production

A variety of culture methodologies may be applied to produce the perhydrolase catalyst. For example, large-scale production of a specific gene product over-expressed from a recombinant microbial host may be produced by batch, fed-batch, and continuous culture methodologies. Batch and fed-batch culturing methods are common and well known in the art and examples may be found in Thomas D. Brock in Biotechnology: A Textbook of Industrial Microbiology, Second Edition, Sinauer Associates, Inc., Sunderland, Mass. (1989) and Deshpande, Mukund V., Appl. Biochem. Biotechnol., 36:227-234 (1992).

Commercial production of the desired perhydrolase catalyst may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Recovery of the desired perhydrolase catalysts from a batch fermentation, fed-batch fermentation, or continuous culture, may be accomplished by any of the methods that are known to those skilled in the art. For example, when the enzyme catalyst is produced intracellularly, the cell paste is separated from the culture medium by centrifugation or membrane filtration, optionally washed with water or an aqueous buffer at a desired pH, then a suspension of the cell paste in an aqueous buffer at a desired pH is homogenized to produce a cell extract containing the desired enzyme catalyst. The cell extract may optionally be filtered through an appropriate filter aid such as celite or silica to remove cell debris prior to a heat-treatment step to precipitate undesired protein from the enzyme catalyst solution. The solution containing the desired enzyme catalyst may then be separated from the precipitated cell debris and protein by membrane filtration or centrifugation, and the resulting partially-purified enzyme catalyst solution concentrated by additional membrane filtration, then optionally mixed with an appropriate carrier (for example, maltodextrin, phosphate buffer, citrate buffer, or mixtures thereof) and spray-dried to produce a solid powder comprising the desired enzyme catalyst.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

### General Methods

The following examples are provided to demonstrate preferred aspects of the invention. It should be appreciated by those of skill in the art that the techniques disclosed in the examples follow techniques to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the presently disclosed methods and examples.

All reagents and materials were obtained from DIFCO Laboratories (Detroit, Mich.), GIBCO/BRL (Gaithersburg, Md.), TCI America (Portland, Oreg.), Roche Diagnostics Corporation (Indianapolis, Ind.) or Sigma/Aldrich Chemical Company (St. Louis, Mo.), unless otherwise specified.

The following abbreviations in the specification correspond to units of measure, techniques, properties, or compounds as follows: “sec” or “s” means second(s), “min” means minute(s), “h” or “hr” means hour(s), “4” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “g” means gravity, “gf” means maximum grams force, “den” means denier, “N” means Newtons, “tex” means basic tex unit in mass of yard/fiber in grams per 1000 meters length, “HPLC” means high performance liquid chromatography, “dd H₂O” means distilled and deionized water, “dcw” means dry cell weight, “ATCC” or “ATCC®” means the American Type Culture Collection (Manassas, Va.), “U” means unit(s) of perhydrolase activity, “rpm” means revolution(s) per minute, “Tg” means glass transition temperature, “Ten.” means tenacity, “TS” means tensile strength, and “EDTA” means ethylenediaminetetraacetic acid.

### Single Fiber Tensile Strength Test

### Hair Sample Selection for Testing

Since the quality and diameter of the hair samples are variables, hair sample pre-selection is used to achieve reasonable standard deviation in the tensile strength test. Any hair fiber with visible defect or significantly thinner or thicker than most fibers was manually removed. To prevent contamination, gloves were used while handing hair fiber samples.

### Hair Sample Preparation

Plastic transfer pipette tubes were cut to 2^{7/8} inches (approximately 7.3 cm) by cutting off the bulb end. A hair bundle with 7 to 20 hair strands was carefully inserted into the tube keeping the hair fibers together by taping one end together and attached to the outside top of the wider end of the transfer pipette. This also helped to keep the strands in place once in the tube.

### Application Procedure

Solution/cream (200 μL) was added to a 2.0-mL microfuge tube. The hair sample was placed into tube by inserting the smaller end of transfer pipette inside tube with hair protruding out of the tip such that the hair strands formed a U shape and the free end of the strands hung loosely outside of the microfuge tube.

After certain application, hair samples were dried and equilibrated in the temperature and humidity control room for at least 12 hr, preferably 24 hr, before tensile strength test.

### Hair Fiber Tensile Strength Testing Procedure

Hair samples were cut into pieces 4-inches in length. The denier number (liner mass density) of each hair fiber was measured by Textechno Vibromat Instrument (Textechno Herbert Stein GmbH & Co. KG; Germany). The hair fibers were tested according to ASTM D 3822-01 (“Tensile Properties of Single Textile Fibers”; ASTM International, West Conshohocken, Pa., 2001), on an Instron testing system (Instron Model 1122 Tester; Instron, MA) at about 70° F. (approximately 21° C.) and 65% relative humidity. The treated area of the hair sample was placed in the middle of the gauge and both ends were gripped on the test heads. Both fiber ends were attached on a piece of tape, respectively, for easy handling and gripping on the test heads. The test used 2-inch (5.1 cm) gauge length, 500 mg load cell, and 1.2 inch/min (3 cm/min) pulling rate. The force-strain curve of the fiber was recorded during the test. The tenacity at maximum (gf/den) was calculated by dividing maximum force (gf) by denier number (den). Note that (gf/den)/11.33=Newtons/tex (N/tex).

### HPLC Peracetic Acid Assay

Determination of the peracetic acid (PAA) concentration in the reaction mixtures was performed according to the method described by Pinkernell et al., Anal. Chem. 1997, 69(17):3623-3627. Aliquots (0.040 mL) of the reaction mixture were removed at predetermined times and mixed with 0.960 mL of 5 mM phosphoric acid in water; adjustment of the pH of the diluted sample to less than pH 4 immediately terminated the reaction. The resulting solution was filtered using an ULTRAFREE® MC-filter unit (30,000 Normal Molecular Weight Limit (NMWL), Millipore cat #UFC3LKT 00) by centrifugation for 2 min at 12,000 rpm. An aliquot (0.100 mL) of the resulting filtrate was transferred to 1.5-mL screw cap HPLC vial (Agilent Technologies, Palo Alto, Calif.; #5182-0715) containing 0.300 mL of deionized water, then 0.100 mL of 20 mM MTS (methyl-p-tolyl-sulfide) in acetonitrile was added, the vials capped, and the contents briefly mixed prior to a 10 min incubation at ca. 25° C. in the absence of light. To each vial was then added 0.400 mL of acetonitrile and 0.100 mL of a solution of triphenylphosphine (TPP, 40 mM) in acetonitrile, the vials re-capped, and the resulting solution mixed and incubated at ca. 25° C. for 30 min in the absence of light. To each vial was then added 0.100 mL of 10 mM N,N-diethyl-m-toluamide (DEET; HPLC external standard) and the resulting solution analyzed by HPLC (Waters Alliance 2695, Waters Corporation; MA).

### HPLC Method

Supelco Discovery C8 column (10 cm×4.0-mm, 5 μm) (cat. #569422-U) w/precolumn Supelco Supelguard Discovery C8 (Sigma-Aldrich; cat #59590-U); 10 microliter injection volume; gradient method with CH₃CN (Sigma-Aldrich; #270717) and deionized water at 1.0 mL/min and ambient temperature:

TABLE 1
HPLC Gradient

| Time (min:sec) | (% CH₃CN) |
|---|---|
| 0:00 | 40 |
| 3:00 | 40 |
| 3:10 | 100 |
| 4:00 | 100 |
| 4:10 | 40 |
| 7:00 (stop) | 40 |

Expression Vector pLD001

Plasmid pLD001 (SEQ ID NO: 292) has been previous reported as a suitable expression vector for E. coli (see U.S. Patent Application Publication No. 2010-0158823 A1 to Wang et al.; incorporated herein by reference). The vector pLD001 was derived from the commercially available vector pDEST17 (Invitrogen, Carlsbad, Calif.). It includes sequences derived from the commercially available vector pET31b (Novagen, Madison, Wis.) and encodes a fragment of the enzyme ketosteroid isomerase (KSI).

Using standard recombinant DNA methods, the coding sequences for the various hydrolases/perhydrolases bounded by NdeI and BamHI sites may be ligated between NdeI and BamHI sites of pLD001 replacing the KSI fragment. Similarly the coding sequences of the binding domains bounded by the BamHI and AscI sites may be ligated between BamHI and AscI sites of pLD001.

### Example 1

### Setting the Target Level of Hair Weakening Efficacy Using a Commercial Depilatory Product

The purpose of this example is to establish a target level of hair weakening efficacy using a commercial depilatory product.

Tenacity at maximum (tensile strength) of each hair sample was used to compare the integrity of the hair sample. A lower value of the tenacity at maximum is indicative of an improved weakening efficacy. A commercial depilatory cream, NAIR® (an alkali/potassium thioglycolate-based hair removal product from Church and Dwight Co., Inc., Princeton, N.J.), was used to set a target level for hair weakening. Based on the NAIR® product instruction, the recommended treatment time is 3 min to 10 min. Therefore, the tenacity at maximum of a hair sample treated with NAIR® between 3 min to 10 min was used to determine the target level. During hair treatment, about 200 μL of NAIR® cream was added to a 2-mL microfuge tube and hair samples were soaked in the cream for 3, 5 and 10 min. Hair samples were then rinsed with tape water to remove all the cream and then were dried under a stream of N₂. Table 2 shows that the average tensile strength of hair strands treated with NAIR® for 3 min to 10 min was in the range from about 1.3 gf/den to 0.8 gf/den. Therefore, the desired level of hair weakening efficacy was targeted in the range from 0.8 gf/den to 1.3 gf/den under similar testing conditions.

TABLE 2
Target Level of Hair Weakening Efficacy

| | | Tenacity at | |
|---|---|---|---|
| | | Maximum | Standard |
| Sample | Time (min) | (gf/den) | Deviation |
| 1 | 0 | 1.7 | 0.5 |
| 2 | 0 | 1.6 | 0.2 |
| 3 | 3 | 1.2 | 0.5 |
| 4 | 3 | 1.3 | 0.3 |
| 5 | 5 | 1.2 | 0.2 |
| 6 | 5 | 1.1 | 0.3 |
| 7 | 10 | 0.8 | 0.2 |
| 8 | 10 | 0.9 | 0.1 |

### Example 2

### Identification of the Lowest Concentration of Peracetic Acid in a Single Application that Achieves the Target Level

The purpose of this example is to identify the lowest concentration of peracetic acid (PAA) to achieve in a single application the targeted level of hair weakening efficacy.

Peracetic acid was added in 50 mM phosphate buffer to prepare solutions with different PAA concentrations at pH 6 and pH 8. A PAA solution (200 μL) was added to a 2-mL microfuge tube and hair samples were inserted for 2 hours. Hair samples were then rinsed with deionized water and dried under N₂. Table 3 shows that the PAA concentration needs to be greater than 0.6 wt % to achieve the targeted hair weakening level under the testing conditions.

TABLE 3
Effect of PAA Concentration and pH on Hair Weakening

| | | | | Ten. @ | |
|---|---|---|---|---|---|
| | [PAA] | Soaking | | Max | Standard |
| Samples | (wt %) | Time (hr) | pH | (gf/den) | Deviation |
| 1 | 0.00 | 2 | 6 | 1.8 | 0.5 |
| 2 | 0.02 | 2 | 6 | 1.7 | 0.2 |
| 3 | 0.20 | 2 | 6 | 1.5 | 0.2 |
| 4 | 0.60 | 2 | 6 | 1.3 | 0.2 |
| 5 | 2.00 | 2 | 6 | 0.9 | 0.1 |
| 6 | 0.00 | 2 | 8 | 1.6 | 0.2 |
| 7 | 0.20 | 2 | 8 | 1.6 | 0.4 |
| 8 | 0.60 | 2 | 8 | 1.3 | 0.3 |
| 9 | 2.00 | 2 | 8 | 0.8 | 0.1 |

In order to lower the concentration of PAA, a longer soaking time and the inclusion of urea (a hair swelling agent) were used. Table 4 indicates that using longer soaking time and 20% urea helped to lower the effective PAA concentration to about 0.6 wt %. The effect of longer soaking time and urea were not significant when PAA concentration was less than 0.6 wt %.

TABLE 4
Effect of Treatment Time and Urea on Hair Weakening

| | | | | Ten. @ | |
|---|---|---|---|---|---|
| | [PAA] | Soaking | Urea | Max | Standard |
| Sample | (wt %) | Time (hr) | (wt %) | (gf/den) | Deviation |
| 1 | 0.02 | 2 | 0 | 1.6 | 0.1 |
| 2 | 0.02 | 4 | 20 | 1.7 | 0.1 |
| 3 | 0.20 | 2 | 20 | 1.6 | 0.2 |
| 4 | 0.20 | 4 | 20 | 1.5 | 0.5 |
| 5 | 0.60 | 2 | 0 | 1.3 | 0.3 |
| 6 | 0.60 | 4 | 0 | 1.2 | 0.1 |
| 7 | 0.60 | 2 | 20 | 1.4 | 0.2 |
| 8 | 0.60 | 4 | 20 | 0.8 | 0.2 |

### Example 3

### Multiple Treatments of Peracid

The purpose of this example is to show the advantage of multiple, repeated treatments of peracid versus a single peracid treatment with equal exposure time.

Solutions of PAA at various concentrations were prepared in 50 mM phosphate pH 8 containing 20 wt % urea. The hair samples were soaked in the solutions for 30 min and then rinsed and dried as described above. The treatment was repeated for 7 times or 15 times, i.e., 4 hours and 8 hours of total exposure time, respectively. The results are provided in Table 5.

TABLE 5
Effect of Multiple Treatments on Hair Weakening

| | | | | Ten. @ | |
|---|---|---|---|---|---|
| | [PAA] | Application | Urea | Max | Standard |
| Sample | (wt %) | Schedule | (wt %) | (gf/den) | Deviation |
| 1 | 0.00 | 30 min × 8 | 20 | 1.8 | 0.2 |
| 3 | 0.20 | 30 min × 8 | 20 | 1.3 | 0.3 |
| 4 | 0.20 | 30 min × 16 | 20 | 0.6 | 0.1 |
| 5 | 0.60 | 30 min × 8 | 20 | 0.4 | 0.1 |
| 6 | 0.60 | 30 min × 16 | 20 | NA | NA |

NA: Not applicable as all hair strains in sample 6 lost their integrity before the 16^{th} application and could not be analyzed.

Comparing Table 4 to Table 5, repeated treatment effectively improved the hair weakening efficacy for 0.6 wt % PAA containing 20 wt % urea solutions. The data in Table 5 indicated that the effective PAA concentration to achieve target hair weakening level can be lowered to 0.2 wt % while using multiple treatments up to 16 times.

### Example 4

### Hair Weakening Efficacy of Other Hair Bleaching Agents

The purpose of this example is to compare the hair weakening efficacy of other hair bleaching agents.

The same multiple treatment as in Example 3 was applied with two commercially available hydrogen peroxide-based bleaching creams (SALLY HANSEN® bleach cream for face and SALLY HANSEN® extra strength bleach cream for face & body; Del Laboratories, Inc., Uniondale, N.Y.) and 6 wt % hydrogen peroxide (H₂O₂) in a similar pH 8, 20% urea, phosphate solution. The color of hair samples treated with two bleaching creams was significantly lighter after the first treatment. However, the tenacity at maximum values of treated hair strands did not significantly decrease compared to untreated hair strand. No change was observed in color and tenacity value for hair strands treated with the 6 wt % H₂O₂ solution (Table 6).

TABLE 6
Hair Weakening Efficacy of other Hair Bleaching Agents.

| | | Ten. @ | |
|---|---|---|---|
| | Application | Max | Standard |
| Sample | Schedule | (gf/den) | Deviation |
| Bleach Cream | 30 min × 8 | 1.5 | 0.1 |
| Extra Strength Bleach Cream | 30 min × 8 | 1.6 | 0.2 |
| 6 wt % H₂O₂, 20 wt % urea | 30 min × 8 | 1.7 | 0.1 |

### Example 5

### Hair Weakening Efficacy with Lower Concentrations of Peracetic Acid Plus Urea

The purpose of this example is to demonstrate that similar hair weakening efficacy can be achieved with a lower PAA concentration and urea in an amount lower than that shown in Example 3.

In another experiment, different amounts of PAA and urea were mixed into 50 mM phosphate buffer pH 8 to make a 10 wt % urea solution with different concentrations of PAA. The hair samples were soaked in the solutions for 20 min and then rinsed and dried as describe above. The treatment was repeated for 15 times. Table 7 shows using the pH 8 solution containing 0.1 wt % PAA and 10 wt % urea can achieve target level of hair weakening efficacy under the testing conditions. In other word, treating hair fiber with a solution of 0.1 wt % PAA with 10 wt % urea at pH 8 for 16 times and 20 min per application could achieve better hair weakening efficacy than the hair sample treated with NAIR® for 5 min.

TABLE 7
Effect of PAA Concentration on Hair Weakening

| | | | | Ten. @ | |
|---|---|---|---|---|---|
| | [PAA] | Application | Urea | Max | Standard |
| Sample | (wt %) | Schedule | (wt %) | (gf/den) | Deviation |
| 1 | 0.01 | 20 min × 16 | 10 | 1.7 | 0.1 |
| 2 | 0.02 | 20 min × 16 | 10 | 1.7 | 0.1 |
| 3 | 0.05 | 20 min × 16 | 10 | 1.5 | 0.1 |
| 4 | 0.10 | 20 min × 16 | 10 | 1.1 | 0.2 |
| 5 | 0.20 | 20 min × 16 | 10 | 0.7 | 0.3 |

### Example 6

### Effect of Peracetic Acid Solutions on Wool Removal

The purpose of this example is to study the effect of PAA solution on removing wool from a pelt sample.

To evaluate the efficacy of PAA solution on removing the wool from a sheep pelt sample, the following experiment was carried out. Pelt strips (0.5 cm×3 cm) were cut and the wool fibers were trimmed to a length of 0.2 cm. Each solution (50 μL) was applied on separated pelt strip (half of the length treated and half of the length untreated). The pelt strip samples were allowed to stand for 10 min at room temperature (˜20° C.). Excess liquid was removed by blotting and the samples were put inside a plastic tube for 30 min for further treatment and then washed with 50 μL of a 2% sodium lauryl ether sulfate (SLES) solution, rinsed with tap water (excess liquid was removed by blotting), and then dried under N₂. The treatment was repeated until the wool fibers in treated area were all removed. Three different solutions were tested using this protocol, a) 0.6 wt % PAA, 15 wt % urea, with 0.5 wt % SLES in a pH 8, 50 mM phosphate buffer; b) 0.6 wt % PAA and 15 wt % urea in a pH 8, 50 mM phosphate buffer; c) 0.2 wt % PAA, 15 wt % urea, 0.5 wt % SLES in a pH 8, 50 mM phosphate buffer. At the 19^{th}, 23^{rd}, 24^{th} application, respectively, the wool fibers on the samples treated with solution (a), (b) and (c) were completely removed. The addition of 0.5 wt % SLES seems to accelerate the wool removal process.

### Example 7

### Sequentially-Applied Peracetic Acid Solution in Combination with a Reductive Solution

The purpose of this example is to demonstrate hair weakening efficacy when the hair samples were treated sequentially with PAA solution and a reductive solution.

Current hair removal products usually use reductants (chemical reducing agents) as active ingredients, such as potassium thioglycolate. To obtain necessary hair removal efficacy, a high pH and a high concentration of these reductants are typically required. For example, 15 wt % of potassium thioglycolate and pH 12 is used. However, these conditions may cause skin irritation. This example shows how sequentially using a peracetic acid (PAA) solution and a reductive solution can lower the pH and the concentration of active ingredients required for effective hair removal. In this experiment, different amounts of PAA and urea were added into 50 mM phosphate buffer pH 8 to make 10 wt % urea solution with different concentrations of PAA. A reductive solution was prepared comprising 5 wt % potassium thioglycolate, 10 wt % urea into 50 mM phosphate buffer pH 7.5. The hair samples were soaked in the PAA solutions for 20 min and then rinsed and dried as described above. The hair samples were then soaked in the above reductive solution for 20 min and then rinsed and dried. The whole treatment with both PAA solution and reductive solution was repeated for 15 times. In Table 8, sample 1 was not treated with PAA solution did not achieve the target level of hair weakening efficacy. When combining with 0.05 wt % PAA solution with the said reductive solution, the treatment significantly improve the hair weakening efficacy. Comparing Table 8 to Table 7, combining PAA solution with mild reductive solution showed better hair weakening efficacy than using PAA solution alone.

TABLE 8
Effect of Combining PAA Solution and
Reductive Solution on Hair Weakening.

| | | | | Ten. @ | |
|---|---|---|---|---|---|
| | [PAA] | Application | Urea | Max | Standard |
| Sample | (wt %) | Schedule | (wt %) | (gf/den) | Deviation |
| 1 | 0 | 20 min × 16 | 10 | 1.4 | 0.1 |
| 2 | 0.01 | 20 min × 16 | 10 | 1.2 | 0.2 |
| 3 | 0.02 | 20 min × 16 | 10 | 1.6 | 0.2 |
| 4 | 0.05 | 20 min × 16 | 10 | 0.8 | 0.3 |
| 5* | 0.10 | 20 min × 8 | 10 | 0.9 | 0.3 |
| 6* | 0.20 | 20 min × 8 | 10 | 0.7 | 0.2 |

*Samples 5 and 6 were stopped after 8^{th} application due to significant bleaching and morphology changes to the hair sample.

### Example 8

### Construction of Hair-Targeted Perhydrolase Fusions

The following example describes the design of an expression system for the production of perhydrolases targeted to hair via hair-binding sequences.

The genes (SEQ ID NO: 286 and SEQ ID NO: 287) encoding for fusions of an enzyme having perhydrolytic activity (a “perhydrolase”) to hair-binding domains (SEQ ID NO: 290 and SEQ ID NO: 291) were designed to have the polynucleotide sequence of the C277S variant of the Thermotoga maritima perhydrolase (SEQ ID NO: 293) fused at the 3′-end to the nucleotide sequence encoding a flexible linker; itself further fused to the hair-binding domains HC263 or HC1010 (SEQ ID NO: 290 and SEQ ID NO: 291; respectively). The genes were codon-optimized for expression in E. coli and synthesized by DNA2.0 (Menlo Park, Calif.). The genes were cloned behind the T7 promoter in the expression vector pLD001 (SEQ ID NO: 292) between the NdeI and AscI restriction sites yielding plasmids pLR1021 and pLR1022, respectively. To express the fusion protein, the plasmids were transferred to the E. coli strain BL21AI (Invitrogen, Carlsbad, Calif.) yielding strains LR3311 (perhydrolase fusion to HC263; SEQ ID NO: 288) and LR3312 (perhydrolase fusion to HC1010; SEQ ID NO: 289).

The non-targeted C277S variant of the Thermotoga maritima perhydrolase was cloned similarly. The preparation and recombinant expression of the Thermotoga maritima C277S variant has previously been reported by DiCosimo et al. in U.S. Patent Application Publication No. 2010-0087529; hereby incorporated by reference.

### Example 9

### Production of the Fusion Proteins

The following example describes the expression and purification of perhydrolases targeted to hair via a hair-binding domains.

Strains LR3311 and strain LR3312 were grown in 1 liter of autoinduction medium (10 g/L tryptone, 5 g/L yeast extract, 5 g/L NaCl, 50 mM Na₂HPO₄, 50 mM KH₂PO₄, 25 mM (NH₄)₂SO₄, 3 mM MgSO₄, 0.75% glycerol, 0.075% glucose and 0.05% arabinose) containing 50 mg/L spectinomycin at 37° C. for 20 hr under 200 rpm agitation. Production of the untargeted perhydrolase has been described previously in U.S. Patent Application Publication No. 2010-0087529 to DiCosimo et al.

The cells were harvested by centrifugation at 8000 rpm at 4° C. and washed by resuspending the cell pellets in 300 mL of ice chilled lysis buffer (50 mM Tris pH 7.5, 5 mM EDTA, 100 mM NaCl) using a tissue homogenizer (Brinkman Homogenizer model PCU11; Brinkmann Instruments, Mississauga, Canada) at 3500 rpm followed by centrifugation (8000 rpm, 4° C.). The cells were the lysed by resuspension in chilled lysis buffer containing 75 mg of chicken egg white lysozyme (Sigma) using the tissue homogenizer. The cell suspensions were allowed to rest on ice for 3 hr to allow the digestion of the cell wall by the lysozyme, with periodic homogenization with the tissue homogenizer. At this stage, care was taken to avoid any foaming of the extracts. The extracts were split (150 mL per 500-mL bottle) and frozen at −20° C. The frozen cell extracts were thawed at room temperature (˜22° C.), homogenized with the tissue homogenizer and disrupted by sonication using a sonicator (Branson Ultrasonics Corporation, Danbury, Conn.; Sonifier model 450) equipped with a 5 mm probe at 20% maximum output, 2 pulses per second for 1 min. The lysed cell extracts were transferred to 4×50-mL conical polypropylene centrifuge tubes and then centrifuged at 10,000 rpm for 10 min at 4° C. The pellet containing cell debris as well as unbroken cells was frozen. Aliquots of the lysate were transferred to 15-mL conical polypropylene tube (12×5-mL) and heated to 80° C. for 15 min, chilled on ice, and pooled into 4×50-mL conical polypropylene centrifuge tubes. The soluble fraction containing the thermostable enzyme and the precipitated E. coli proteins were separated by centrifugation at 10,000 rpm for 10 min at 4° C. If the cell disruption was incomplete after the sonication step, the frozen pellet was thawed again and subjected to a second round of sonication, centrifugation and heat treatment. The output of this purification protocol typically yielded 2-4 mg of protein per mL with a purity of the fusion perhydrolase between 90% and 75% of the protein as estimated by polyacrylamide gel electrophoresis (PAGE) analysis. Total protein was quantitated by the bicinchoninic acid (BCA) assay (Thermo Fisher Scientific, Rockford, Ill.) using a solution of Bovine Serum Albumin as a standard.

### Example 10

### Quantitation of the Enzyme Hydrolase Activity

The following example describes the method for the detection and quantitation of the perhydrolase via its hydrolase activity using a non-specific esterase substrate.

The hydrolase activity of the perhydrolase fusions was determined with pNPA (p-nitrophenyl acetyl ester). Typically the enzyme was diluted in hydrolase assay buffer (50 mM KH₂PO₄; pH 7.2) to concentration between 1 and 0.01 μg per mL. The reaction was initiated by addition of pNPA to a final concentration of 3 mM (30 μL/mL of 100 mM pNPA dissolved in acetonitrile) at 25° C. or 30° C. Absorption at 400 nm was recorded at chosen times. Due to a background level of non-enzymatic hydrolysis of pNPA, a no-enzyme control was included in the analysis. Activity was measured as Δ400/min (sample)−Δ400/min (no-enzyme control) and converted into μmol of pNPA hydrolyzed 1 mg of proteins×min (pNPA molar absorption: 10909 M⁻¹). The specific activity of the fusion proteins was typically between 10 and 30 μmol/mg×min.

### Example 11

### Binding of the Hair-Targeted Perhydrolase Fusion to Hair

The following example describes the binding of the perhydrolase to hair in a manner dependent on the fusion of hair-binding sequences to the perhydrolase (PAH).

For hair binding experiments brown hair tresses (International Hair Importers and Products, Glensdale N.Y.) were used. The hair was washed with 2% SLES, rinsed extensively with deionized water and air dried.

Around 20 mg of 1 cm brown hair fragments was added in a 1.8-mL microfuge tube. Hydrolase assay buffer (1.2 mL) as added to the hair followed by the addition of the perhydrolase enzymes to the solution. The enzymes were allowed to bind the hair for 30 min with gentle agitation (24 rpm) on an Adams Nutator (model 1105, Becton Dickinson, Franklin Lakes, N.J.). No enzyme controls, with hair and without hair, were included in the binding experiment to account for non-enzymatic hydrolysis of the pNPA hydrolase reagent. After the binding step, a 1.0-mL aliquot of the binding buffer was transferred to a new tube to quantitate the amount of unbound enzyme. Additional binding buffer was removed and the hair fragments were washed 4 times with 1 mL of 1% TWEEN®-20 in hydrolase buffer, followed by 2 washes with 1 mL each in hydrolase buffer. The hair fragments were then resuspended in 1 mL of hydrolase assay and the hydrolase activity that remained bound to the hair was measured. The C277S (“PAH”) variant of Thermotoga maritima perhydrolase (SEQ ID NO: 293) was used as a control for an un-targeted perhydrolase. The results are provided in Table 9.

TABLE 9
Retention of Perhydrolase on Hair.

| | | Activity in | Activity |
|---|---|---|---|
| | | the first | retained on hair |
| | Activity^{a} | TWEEN ®-20 | after 4 TWEEN ®- |
| Enzyme | unbound (%) | wash (%) | 20 washes (%) |
| Untargeted | 103 | 5 | 1 |
| T. maritima C277S | | | |
| (SEQ ID NO: 293) | | | |
| C277S-HC263 | 52 | 9 | 54 |
| (SEQ ID NO: 288) | | | |
| C277S-HC1010 | 20 | 20 | 41 |
| (SEQ ID NO: 289) | | | |

^{a}= The retention of perhydrolase on hair was detected by its hydrolase activity. 100% of activity is the hydrolase activity added to a tube containing ~20 mg of hair but not subjected to washes. For each enzyme, the 100% activity was: untargeted PAH, 148 μmol/min; C277S-HC263, 53 μmol/min; and C277S-HC1010, 125 μmol/min.

The data in Table 9 demonstrates that the perhydrolase fusions targeted to hair were retained on hair after extensive washes in 1% TWEEN®-20 whereas the untargeted perhydrolase was not.

### Example 12

### Binding Specificity of the Fusion Perhydrolase on Hair

The following example describes the binding of the hair-targeted perhydrolase predominantly on hair over skin in a manner dependent on the hair-binding sequences.

To assess the targeting of the perhydrolase to hair provides some level of binding specificity to hair vs skin, a visual detection of the hydrolase activity of the perhydrolase was implemented using an general esterase histological stain kit (Sigma Aldrich, catalog number: 91A-1 KT). This kit provides a colorless substrate that, upon hydrolysis yields a product that forms an insoluble brown precipitate thus allowing for a visual evaluation of the presence of hydrolase activity.

Hair and skin cells were deposited on D-Squame D-100 self-adhesive tape strips (CuDerm Corp Dallas, Tex.) as follows. A few strands of natural white hair (0.5 cm to 1 cm long) (International Hair Importers and Products, Glensdale N.Y.) were placed onto the sticky side of the tape strips and the strips were pressed for 10 seconds against the skin of the inside of an arm previously shaved. White hair was chosen as to maximize the visualization of brown dye deposition in the vicinity of the perhydrolase. Once the strips removed, they were coated with skin cells as well as white hairs. Each strip carrying hairs and skin cells was washed with gentle agitation with low speed (<40 rpm) in 2 mL of hydrolase assay buffer containing 1% TWEEN®-20 to wash hair/skin cells for 1-2 minutes inside well of a 6 multi-well cell culture polystyrene plate (Becton Dickinson). The buffer was removed and replaced with 2 mL of 50 mM KH₂PO₄; pH 7.2 (hydrolase assay buffer) and 10 μL of C277S-HC263 (1.4 μg/mL), 7.0 μL of C277S-HC1010 (2.0 μg/mL) or 1.7 μL of untargeted enzyme. Binding was allowed for 30 min at room temperature (˜22° C.) under gentle agitation after which the respective enzyme solution was removed and exchanged for hydrolase assay buffer containing 1% TWEEN®-20. This wash step was repeated three times followed by two washes in hydrolase assay buffer lacking TWEEN®-20. Each tape strips was then transferred to a new well. One milliliter of α-naphthyl acetate reagent (prepared freshly as described by the manufacturer) was added and the color was allowed to develop for 15 min. The stained tape strips were washed three times with hydrolase assay buffer. Visual observation of the straining of the hair and the skin cells are recorded in Table 10.

TABLE 10
Staining of Hair and Skin with a Hydrolase Histological Stain

| Enzyme | Color developed on | Color developed on |
|---|---|---|
| (SEQ ID NO:) | hair | skin |
| No enzyme | − | − |
| Untargeted PAH | − | −/+ |
| (T. maritima C277S) | | |
| (SEQ ID NO: 293) | | |
| C277S-HC263 | +++ | −/+ |
| (SEQ ID NO: 288) | | |
| C277S-HC1010 | +++ | −/+ |
| (SEQ ID NO: 287) | | |

This experiment demonstrates the retention of the perhydrolase on hair but not on skin for the targeted perhydrolase fusions.

### Example 13

### Hair Weakening Efficacy Using Perhydrolytic Enzymes in a One-Step Application

The purpose of this example is to show the hair weakening efficacy of PAA when enzymatically produced in a one-step application.

Two methods for hair removal I hair weakening were developed using an enzymatic in situ peracetic acid generation system comprising at least one CE-7 perhydrolase enzyme.

The first method (referred to herein as the “one-step approach”) comprises combining different amounts of at least one CE-7 perhydrolase with triacetin (an example of a suitable ester substrate) and hydrogen peroxide to generate peracetic acid. Hair bundles were inserted into “one step approach” solutions for 15 min immediately after mixing the enzyme solution with triacetin solution (final concentration was about 100 mM) and H₂O₂ solution (final concentration was about 250 mM). The hair samples were rinsed with water and dried. The method was repeated for another 15 times (total application was 16 times). In one experiment, two perhydrolase enzymes were used, i.e., untargeted T. maritima variant C277S (“C277S”; “PAH”; SEQ ID NO: 293) and “C277S-HC263” (SEQ ID NO: 288). The concentration of peracetic acid in solution after 15 min was evaluated by HPLC. The results are provided in Table 11.

TABLE 11
Hair Weakening Efficacy of Perhydrolase Systems.

| | [C277S- | | | | Ten. @ | |
|---|---|---|---|---|---|---|
| | HC263] | [C277S] | Urea | [PAA] | Max | Standard |
| Sample | (mg/mL) | (mg/mL) | (wt %) | (ppm) | (gf/den) | Deviation |
| 1 | 0.5 | — | 0 | 4132 | 0.7 | 0.1 |
| 2 | 0.25 | — | 0 | 3948 | 0.4^{#} | — |
| 3 | 0.05 | — | 0 | 1983 | 1.0 | 0.3 |
| 4 | — | 0.5 | 10 | 13656 | 0.6 | 0.2 |
| 5 | — | 0.25 | 10 | 14053 | 0.6^{#} | — |
| 6 | — | 0.05 | 10 | 6448 | 0.3 | 0.1 |
| 7 | 0 | 0 | 0 | 521 | 1.7 | 0.2 |
| 8 | 0 | 0 | 0 | 521 | 1.7 | 0.1 |
| 9 | 0 | 0 | 10 | 300 | 1.9 | 0.1 |
| 10 | 0 | 0 | 10 | 300 | 1.6 | 0.2 |

^{#}Tenacity value labeled with star was average of less than 3 strands since several strands broke during application.

In Table 11, the amount of PAA generated by enzymes increased with the amount of enzyme in solutions but leveled off after the enzyme concentration reached 0.25 mg/mL. The preparation of the fusion protein C277S-HC263 showed about 2-3 times lower perhydrolase activity than C277S under testing condition and on the same weight basis. The 0.05 mg/mL C277S-HC263 could generate about 0.2 wt % PAA and all other conditions generated more PAA. Table 11 also shows the tensile strength of the hair strands after each treatment. The hair samples treated with solutions containing 0.25 mg/mL enzyme showed lowest tensile strength and only two out of seven hair strands were not broken during the applications. Other samples had at least 5 strands left for tensile strength testing. The non-targeted version of the perhydrolase (C277S) showed better efficacy than C277S-HC263 (targeted fusion construct) since it generated more PAA. Using 0.25 mg/mL C277S-HC263 without using urea or 0.05 mg/mL of PAH with 10% urea could achieve better efficacy than NAIR® treatment for 10 min.

### Example 14

### Hair Weakening Efficacy Using Perhydrolytic Enzymes in a Two-Step Application

The purpose of this example is to demonstrate the hair weakening efficacy of perhydrolases systems in a two-step application.

In a two-step application, the hair strands are first treated with perhydrolase solutions to allow the enzyme to bind hair. The hair strands are then dried or rinsed with buffer and dried. The rinse step is intended to remove excess unbound enzyme from the hair. The hair strands are soaked in triacetin and H₂O₂ solutions. The application is repeated for multiple times.

One experiment was carried out with C277S-HC263 and untargeted T. maritima variant C277S using the following protocol:
1. Enzyme solutions comprising 0.5 mg/mL enzyme in 50 mM phosphate buffer (pH 8) were made with or without 10 wt % urea.
2. Seven (7) hair strands were soaked in 0.2 mL of an enzyme solution for 10 min and then dried under N₂ without prior rinsing.
3. The hair strands were then soaked in 0.2 mL of a pH 8 solution containing 100-mM triacetin and 250-mM H₂O₂ for 15 min, dried, then rinsed with DI water and then dried again.
4. Steps 2-3 were repeated up to 15 times.

The tensile strength results are shown in the Table 12. In the absence of a rinse step, enough of both C277S-HC263 and the untargeted C277S were retained on hair and achieved a hair-weakening target level. Adding 10 wt % urea appeared to enhance the untargeted C277S solution's ability to bleach hair faster, but decreased the hair weakening efficacy of C277S-HC263 system.

TABLE 12
Tensile Strength Results of Hair Strands
Treated with Perhydrolase Systems

| | | | Bleaching | Tenacity | |
|---|---|---|---|---|---|
| Sam- | | Urea | Start | @ max | Standard |
| ple | Enzyme | (wt %) | (cycle) | (gf/den) | Deviation |
| 1 | C277S-HC263 | 0 | 15th | 1.2 | 0.2 |
| | (SEQ ID NO: 288) | | | | |
| 2 | Untargeted C277S | 0 | 13th | 0.7 | 0.1 |
| | (SEQ ID NO: 293) | | | | |
| 3 | C277S- | 0 | 13th | 0.6 | 0.2 |
| | HC263:C277S | | | | |
| | (1:1) | | | | |
| 4 | Buffer | 0 | | 1.7 | 0.2 |
| 5 | C277S-HC263 | 10 | | 1.7 | 0.2 |
| | (SEQ ID NO: 288) | | | | |
| 6 | Untargeted C277S | 10 | 12th | 0.6 | 0.2 |
| | (SEQ ID NO: 293) | | | | |
| 7 | Buffer | 10 | | 1.8 | 0.2 |

Another experiment was carried out with C277S-HC263 (SEQ ID NO: 288), CPAH-HC263 (SEQ ID NO: 294), CPAH-HC1010 (SEQ ID NO: 295) and untargeted C277S using the following protocol:
1. Enzyme solutions comprising 1.0 mg/mL enzyme in 50 mM phosphate buffer (pH 6) were prepared. For solutions containing two enzymes, the concentration of each enzyme was approximately 0.5 mg/mL.
2. Seven (7) hair strands were soaked in 0.2 mL of an enzyme solution for 10 min, rinsed with 50 mM phosphate buffer (pH 6), and then dried under N₂.
3. The dried hair strands were then soaked into 0.2 mL of 50 mM phosphate buffer solution (pH 8) comprising 100 mM triacetin and 250 mM H₂O₂ for 15 min, dried, then rinsed with DI water and then dried again.
4. Step 2-3 were repeated for up to 13 times as some samples showed obvious morphology changes.
   The tensile strength results are provided in Table 13.

TABLE 13
Tensile Strength Results of Hair Strands
Treated with Perhydrolase Solutions

| | | Bleaching | | |
|---|---|---|---|---|
| | | Start | Tenacity @ | Standard |
| Sample | Enzyme | (cycle) | max (gf/den) | Deviation |
| 1 | C277S-HC263 | | 1.7 | 0.3 |
| | (SEQ ID NO: 288) | | | |
| 2 | C277S- | | 1.7 | 0.3 |
| | HC263:C277S | | | |
| | (1:1) | | | |
| 3 | Untargeted C277S | | 1.7 | 0.2 |
| | (SEQ ID NO: 293) | | | |
| 4 | Buffer | | 2.0 | 0.1 |
| 5 | CPAH-HC1010 | 12th | 1.2 | 0.3 |
| | (SEQ ID NO: 295) | | | |
| 6 | CPAH- | 10th | 1.0 | 0.3 |
| | HC1010:C277S | | | |
| | (1:1) | | | |
| 7 | CPAH-HC263 | 9th | 0.6 | 0.1 |
| | (SEQ ID NO: 294) | | | |
| 8 | CPAH- | 10th | 0.8 | 0.1 |
| | HC263:C277S | | | |
| | (1:1) | | | |

The perhydrolase solution comprising CPAH-HC263 (SEQ ID NO: 294) alone showed the best hair weakening efficacy. The perhydrolase solution containing both CPAH-HC263 (SEQ ID NO: 294) and untargeted C277S (SEQ ID NO: 293) had the second best performance. The perhydrolase solution containing CPAH-HC1010 also achieved the hair-weakening target level. However, the solutions using untargeted C277S, C277S-HC263 or C277S-HC263:C277S (1:1 mass ratio) did not show a significant hair-weakening effect. HPLC analysis on the perhydrolase activity indicated this preparation of C277S-HC263 has significantly lower perhydrolytic activity than other three perhydrolase enzymes. Therefore, it was difficult to tell whether the lower hair-weakening efficacy of C277S-HC263 resulted from the lower perhydrolase activity or the loss of C277S-HC263 due to the rinsing step. The untargeted C277S enzyme has similar perhydrolase activity compared to CPAH-HC263 and CPAH-HC1010 (SEQ ID NO: 295). Therefore, the difference in hair weakening efficacy is likely due to the fact that CPAH-HC263 and CPAH-HC1010 retained more on hair than untargeted C277S after the buffer rinsing step. This experiment demonstrated that targeted perhydrolase can achieve better hair weakening efficacy than non-targeted perhydrolase using two-step approach with appropriate rinsing challenge.

### Example 15

### Impact of Several Additives on Perhydrolytic Activity

The purpose of this example is to evaluate the impact of additives on the perhydrolase activity of several perhydrolase systems.

A peracetic acid indicator strip with detection range from 75 to 400 ppm was used to study the impact of additives on the perhydrolase activity of a perhydrolase. When the PAA concentration is at least 0.2 wt %, the dark color generated on the indicator strip will turn back to original white color over time.

A first solution (“solution A”) was prepared using 50 mM phosphate buffer (pH 8) and 10 wt % of one of the following additives: glycerin, ethanol, sorbitol, polyethylene glycol, propylene glycol in, respectively.

A second solution (“solution B”) was prepared by mixing 30% H₂O₂ and 50 mM phosphate buffer (pH 8) whereby the final concentration of H₂O₂ was 500 mM.

A third solution (“solution C”) was prepared comprising of CPAH-HC1010 (SEQ ID NO: 295) (0.2 mg/mL), triacetin (200 mM), 50 mM phosphate buffer (pH 8), and solution A (5 wt % additive). Immediately, added solution B in a 1:1 volume ratio. After 1 to 5 min, a 30-μL sample of the resulting solution was removed and immediately placed on a PAA indicator strip.

This process was repeated for each of the additives listed above. Under the testing conditions, none of the above mentioned additives significantly decreased the perhydrolase activity of CPAH-HC1010 (SEQ ID NO: 295) below 2000 ppm.

### Example 16

### Compatibility of a Commercial Moisturizer Lotion on the Perhydrolytic Activity of Perhydrolase Enzyme

The purpose of this example is to evaluate the compatibility of a perhydrolase enzyme with a commercial moisturizer lotion.

Lotion “A” was prepared by mixing 0.7667 g of SUAVE® Advanced Therapy Moisturizer (Lot#12148JU41, Unilever, CT) with 40 μL of perhydrolase fusion C277S-HC263 (10 mg/mL) in a 2-mL microfuge using an overhead mixer at 200 rpm for about 3 min. The concentration of C277S-HC263 (SEQ ID NO: 288) was about 0.1 mg/mL in lotion A.

SUAVE® Advanced Therapy Moisturizer is a commercial skin care lotion comprising the following ingredients: H₂O, glycerin, stearic acid, glycol stearate, retinyl palmitate, tocophenyl acetate, glyceryl stearate, cetyl alcohol, petrolatum, fragrance, dimethicone, magnesium aluminum silicate, isopropyl palmitate, triethanolamine, carbomer, DMDM hydantoin, methylparaben, iodopropynyl butylcarbamate, and titanium dioxide.

Two control samples, i.e., A-control1 and A-control2, were prepared by replacing perhydrolase (C277S-HC263; SEQ ID NO: 288) with same volume of DI water and 50 mM phosphate buffer (pH 8), respectively.

Solution “B” was made by combining 44 μL of triacetin, 899 μL of 50 mM, pH 8, phosphate buffer, and 57 μL of H₂O₂ (30 wt %). Solution B was freshly made every time just before running the PAA indicator strip test.

After a predetermined time, 50 μL of lotion “A” and 50 μL of solution “B” were mixed gently for about 1 min. PAA indicator strips were used to test for the presence of PAA in the mixture as described in Example 10. The results show that perhydrolase C277S-HC263 (SEQ ID NO: 288) retained certain perhydrolase activity in SUAVE® moisturizer for at least 30 days at room temperature (˜21° C.).

### Example 17

### Hair Weakening Efficacy of a Mixture of Triacetin and Hydrogen Peroxide

The purpose of this example is to show the hair weakening efficacy using a concentrated mixture triacetin and hydrogen peroxide (H₂O₂) applied to hair using multiple applications.

In this experiment, the hair strands are firstly treated with 50 mM phosphate buffer (pH 8) for 10 min, then dried under N₂. The hair strands were then soaked in a solution comprising 900 mM triacetin and 500 mM H₂O₂ with or without 20 wt % urea for 15 min. The hair strands were dried with a nitrogen purge, rinsed with deionized water, and dried again with N₂. The application was repeated an additional 15 times. The results are provided in Table 14.

TABLE 14
Hair Weakening Using Concentrated
Mixture of Triacetin and H₂O₂

| | | | Ten. @ | |
|---|---|---|---|---|
| | Application | Urea | Max | Standard |
| Sample | Schedule | (wt %) | (gf/den) | Deviation |
| 1 | 15 min × 16 | 0 | 1.0 | 0.2 |
| 2 | 15 min × 16 | 20 | 0.4 | 0.2 |

Table 14 shows the hair-weakening efficacy targeted can be achieved by using a relatively higher concentration of triacetin and H₂O₂ than the conditions used in Examples 13 to 15. Using 20% urea in the application appears to accelerate the hair weakening effect.

### Example 18

### Prophetic

### Construction and Production of a Skin-Targeted CE-7 Perhydrolase

Targeted perhydrolases having affinity for skin can be prepared to produce a peracid benefit agent for skin. Examples of peptides having affinity for skin are provided by the amino acid sequences of SEQ ID NOs 217-269. Additional skin-biding peptides may be identified using phage display or mRNA display. Examples of CE-7 perhydrolases are provided by the amino acid sequences of SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, and 64.

Briefly, a fusion protein comprising a first portion having at least one CE-7 perhydrolase and a second portion having affinity for skin can be constructed, produced, and assayed using the similar methods to those used to make the hair-targeted fusion peptides described in Examples 8 through 11 by substituting peptides having affinity for skin or by using an empirically-generated peptide having affinity for skin. The fusion peptide may be constructed using the general method described in Example 8. Production of the fusion protein can follow the general method described in Example 9. Example 10 may be used to quantify the amount of active fusion protein while the methodology of Example 11 may be used to test for surface specificity.

The skin-targeted perhydrolases may be used in skin-care products to produce a peracid benefit agent for skin care. The application method may follow the one-step or two-step application methods as described in the present application.

### Example 19

### Prophetic

### Construction and Production of a Nail-Targeted CE-7 Perhydrolase

Targeted perhydrolases having affinity for nail (e.g. human fingernails, toenails) can be prepared to produce a peracid benefit agent for nail. Examples of peptides having affinity for nail are provided by the amino acid sequences of SEQ ID NOs 270-271. Additional nail-biding peptides may be identified using phage display or mRNA display. Examples of CE-7 perhydrolases are provided by the amino acid sequences of SEQ ID NOs: 2, 4, 6, 8, 10, 12, 14, 16, 18, 20, 22, 24, 26, 27, 28, 29, 30, 31, 32, 33, 34, 35, 36, 38, 40, 42, 44, 46, 48, 50, 52, 54, 56, 58, 60, 62, and 64.

Briefly, a fusion protein comprising a first portion having at least one CE-7 perhydrolase and a second portion having affinity for nail can be constructed, produced, and assayed using the similar methods to those used to make the hair-targeted fusion peptides described in Examples 8 through 11 by substituting peptides having affinity for nail or by using an empirically-generated peptide having affinity for nail. The fusion peptide may be constructed using the general method described in Example 8. Production of the fusion protein can follow the general method described in Example 9. Example 10 may be used to quantify the amount of active fusion protein while the methodology of Example 11 may be used to test for surface specificity.

The nail targeted perhydrolases may be used in nail-care products to produce a peracid benefit agent for nail care. The application method may follow the one-step or two-step application methods as described in the present application.

### Example 20

### Binding of Hair-Targeted Perhydrolase to Hair in the Presence of Surfactants

The following example demonstrates the deposition of hair-targeted perhydrolase on hair in the presence of surfactants.

Ten mg of human hair was submerged in 2 mL of a binding solution containing 50 μg/mL of the Thermotoga maritima perhydrolase targeted to hair via the hair binding peptide HC263 (C277S-HC263; SEQ ID NO: 288; also referred to herein as “HC1121”) in 50 mM pH 7.2 potassium phosphate buffer containing 5% of the test surfactant. The test surfactants were two nonionic surfactants: TWEEN®-20 (Sigma, St Louis, Mo.), and TRITON®-X 100 (Aldrich, St Louis, Mo.), and two ionic surfactants: CHEMBETAINE® CAD (Cocamidopropyl betaine; Lubrizol, Wickliffe, Ohio) and RHODAPEX® ES-2K (sodium laurel ether sulfate; Rhodia Novecare, Cranbury, N.J.).

Hair was incubated in the binding solution at room temperature (˜22° C.) with gentle agitation for 30 minutes, at which time the solution was removed by aspiration and the hair rinsed with 1% TWEEN®-20 in 50 mM pH 7.2 potassium phosphate buffer. The hair was removed from the tube, blotted dry with paper towel, and transferred to a new set of tubes. The hair was rinsed once with 1% TWEEN®-20 in 50 mM pH 7.2 potassium phosphate buffer and then rinsed three times with 50 mM pH 7.2 potassium phosphate buffer. Perhydrolase activity remaining bound to the hair was determined by the ABTS assay.

The ABTS assay was performed as follows: hair samples treated as described above were transferred to new 2-mL microcentrifuge tubes (Eppendorf Protein LoBind; Eppendorf North America, Hauppauge, N.Y.) To each tube was added 480 μL of PAH buffer (50 mM potassium phosphate, pH 7.2), 14 μL of 39% hydrogen peroxide (Sigma 3410), and 6 μL triacetin (Sigma-Aldrich W200700). The perhydrolase reaction was allowed to proceed for 10 minutes at room temperature (˜22-25° C.). The reaction was stopped by diluting aliquots 10×, 100× and 1000× with 10 mM phosphoric acid. For detection 50 μL of 1 M acetic acid, 50 μL of 40 mg/mL potassium iodide, and 50 μL of 4 mM ABTS (2,2′-azino-bis(3-ethylbenzothiazoline-6-sulphonic acid; Sigma, St Louis, Mo.) were added to 50 μL of the above dilutions. Color was allowed to develop for 10 minutes and read in a spectrophotometer at 405 nm. Values from no-enzyme controls were subtracted to yield values attributable only to enzyme activity. A standard curve of OD 405 nm vs PAA concentration in ppm yielded a conversion factor of 8.75, such that an OD 405 nm of 1.0 corresponded to approximately 8.75 ppm PAA. This value was then multiplied by the appropriate dilution to arrive at the PAA concentration in the perhydrolase reaction.

The results of the deposition of the targeted perhydrolase on hair in the presence of surfactants is given in Table 15. The values obtained for the non-ionic surfactants were 2.43 for TWEEN®-20, and 1.76 for TRITON®-X 100. The values for the ionic surfactants were 0.52 for CH EMBETAINE™ CAD and 0.36 for RHODAPEX® ES-2K. The results indicated substantially higher activity for enzyme bound in the presence of nonionic surfactants.

TABLE 15
Perhydrolase Activity Retained on Hair Following Deposition of the
Hair-targeted Enzyme Deposited in the Presence of Surfactants.

| Surfactant | ppm PAA |
|---|---|
| TWEEN ®-20 | 2126 |
| TRITON ®-X 100 | 1540 |
| CHEMBETAINE ™ CAD | 455 |
| RHODAPEX ® ES-2K | 315 |

This example demonstrates that the hair-targeted perhydrolase can be deposited in the presence of surfactants and the deposition is particularly effective in the presence of non-ionic surfactants

### Example 21

### Optimization of deposition as function of pH

The following example demonstrates that the pH of a formulation including a targeted perhydrolase can be used to optimize its deposition on hair.

The Thermotoga maritima perhydrolase targeted to hair via the hair binding peptide HC263 (C277S-HC263; SEQ ID NO: 288) was diluted to 50 μg/mL in a solution of 5% PEG-80 sorbitan laurate in 100 mM citrate-phosphate buffer adjusted to pH 4.9, pH 5.5, pH 6.0, or pH 7.0. Ten mg of human hair was added to 2 mL of the above formulations and incubated with gentle agitation for 5 minutes at room temperature to allow enzyme binding to hair. A no-enzyme control sample was included with each pH. After binding, the binding solution was removed by aspiration and the hair was washed with 2 mL of 1% TWEEN®-20 in 50 mM pH 7.2 potassium phosphate buffer. The hair was removed from the tube, blotted dry with paper towel, and transferred to a new set of tubes. The hair was rinsed once with 1% TWEEN®-20 in 50 mM pH 7.2 potassium phosphate buffer and then rinsed three time with 50 mM pH 7.2 potassium phosphate buffer. Perhydrolase activity remaining bound to the hair was determined by the ABTS assay. The results are given as ppm PAA produced at 10 minutes (after subtraction of the no-enzyme control values)

The results are reported in Table 16 and indicate that within the range of values tested, pH 5.5 provides the best binding condition.

TABLE 16
Perhydrolase Activity Deposited on Hair as a Function of pH.

| pH | ppm PAA |
|---|---|
| 4.9 | 437 |
| 5.5 | 1312 |
| 6.0 | 1137 |
| 7.0 | 385 |

Based on the results, the pH of a formulation can be used to control the amount of hair-targeted Thermotoga perhydrolase, and more generally that of other hair targeted perhydrolases.

### Example 22

### Rapid Binding of Hair from a Surfactant Solution

The following example demonstrates that the deposition of a hair targeted perhydrolase can be rapid and compatible with a skin care regimen.

The Thermotoga maritima perhydrolase targeted to hair via the hair binding peptide HC263 (C277S-HC263; SEQ ID NO: 288) was diluted to 50 μg/mL in a solution of 5% PEG-80 sorbitan laurate in 100 mM citrate-phosphate buffer adjusted to pH 6.0. Ten mg of human hair was added to 2 mL of the above formulations and incubated with gentle agitation for 30 seconds, 1 minute, 2 minutes and 5 minutes at room temperature (˜22° C.) to allow enzyme binding to hair. A no-enzyme control sample was also included. After binding, the binding solution was removed by aspiration and the hair was washed with 2 mL of 50 mM pH 7.2 potassium phosphate buffer. The hair was removed from the tube, blotted dry with paper towel, and transferred to a new set of tubes. The hair was rinsed one more times with 50 mM pH 7.2 potassium phosphate buffer. Perhydrolase activity remaining bound to the hair was determined by the ABTS assay. The results are given as OD 405 nm at 10 minutes (after subtraction of the no-enzyme control values). The results presented in Table 17 show that substantial binding has occurred within 30 seconds of contacting the hair with enzyme.

TABLE 17
Perhydrolase Activity Deposited on Hair
After Various Lengths of Binding Time.

| Time (min) | ppm PAA |
|---|---|
| 0.5 | 954 |
| 1 | 962 |
| 2 | 988 |
| 5 | 1093 |

This example demonstrates that significant deposition of a targeted enzyme occurs for binding times as short at 30 seconds.

### Example 23

### Hair Tress Tensile Strength Testing Procedure

The following example demonstrates a quantitative test to assess the weakening of hair by a depilatory product using tensile strength measurements.

The fibers testing protocol according to ASTM D 3822-01 (“Tensile Properties of Single Textile Fibers”; ASTM International, West Conshohocken, Pa., 2001) is a more precise method for determining the tensile strength of a single fiber. The downside of this testing protocol for use in the current system is the stringency of control of temperature and humidity conditions and the length of time required to ultimately reach the final results. An additional downside is the number of replicate samples that should be tested to get the meaningful data. In order to quickly compare the samples following the treatments samples containing multiple hair fibers were used for averaged effects. In addition, the samples were tested wet to simulate 100% humidity conditions. The sample consisted of a hair bundle of approximately 30-70 mg hair of 4 cm length, held together by a 1 mm thick, 2 mm wide and 5 mm long glue strip. Five mm of the free end of this tress was further glued using a fast drying glue (such as DUCO® CEMENT®, a nitro cellulose household cement). After drying the glue, any loose hair strands were cut off and the sample was weighed.

COM-TEN® Tensile Tester 95-VD (Com-Ten Industries, Pinellas Park, Fla.), fitted with a 100 lb. (˜45.4 kg) load-cell was used for tensile tests. In order to reduce sample slippage, 5 mm wide strips of industrial grade VELCRO® (Velcro USA, Manchester, N.H.) were attached to the inside edges of the clamps. Before testing the CALIBRATION was set to “off”, FORCE UNITS were set to “grams” and the distance between the clamps was adjusted to 3 cm. The test sample was soaked in water for 30 seconds. Excess moisture was removed by gentle absorption on a paper towel, leaving enough moisture in hair for it to qualify as being at 100% humidity level. The glued edges of the test sample were clamped at both upper and lower clamps in such a way that the VELCRO® strips held the hair just below the glue. Tester speed was set to ˜2.5 inches by adjusting the speed control knob. With the Force meter in RUN mode, TARE was set to ZERO to set the starting PEAK FORCE to 0. To start the test the DIRECTION toggle switch was pressed to UP position. At the conclusion of the test, when the sample failed, the DIRECTION switch was moved to STOP and the peak force was recorded. The hair was cut off along the edge of the clamps at both lower and upper clamps. The clamps were opened and the stubs were removed, dried in air and weighed. The difference in original sample weight and combined weights of the stubs was the weight of the hair undergoing tensile elongation, and this quantity was used to calculate the tensile strength.

### Calculations of Tensile Strength

For the purpose of comparisons of samples following the treatments, the tensile strengths were defined as follows:

Tensile Strength (N/mg hair)=Peak force (Newtons)/(Initial sample weight−weight of stubs)

### Benchmarking the Tensile Assay

The purpose of this example is to establish a target level of hair weakening efficacy using a commercial depilatory product.

Benchmarking the assay was achieved by measuring the tensile-strength (Hair-weakening) of hair-tresses after treatment with a commercially available depilatory product, NAIR® Lotion with Cocoa Butter (Church & Dwight Co., Inc., Princeton, N.J.). Based on the NAIR® product instruction, the recommended treatment time is 5-10 min. Therefore, the tensile strength of a hair sample treated with NAIR® between 5 min to 10 min was used to determine the target level. Test hair sample consisted of a hair bundle of approximately 50 mg hair of 4 cm length, held together by a 1 mm thick, 2 mm wide and 5 mm long glue strip. The test-sample was placed on a glass plate. Approximately 1 mL of NAIR® lotion was applied to the tress with a gloved finger. The lotion was gently spread over and pressed into the tress to cover all hair fibers. After the desired treatment time at room temperature, the tress was rinsed thoroughly with tap water to remove all traces of the lotion. The sample was air-dried and tested for its tensile strength.

For these treatment times, the tensile strengths of the tresses (wet tress, 100% humidity) were found to be between ˜0.2 N/mg hair for 10 min and between 0.7-1.4 N/mg hair for 5 min. The data is provided in Table 18. Given the variation in the tensile strength the desired level of hair weakening efficacy was targeted in the range from 1.0 N/mgH to 1.6 N/mgH under similar testing conditions.

TABLE 18
Result of Benchmarking Tensile Assay.

| | | | | Treatment | TS, |
|---|---|---|---|---|---|
| Experiment | Sample | Hair state | Humidity | time, min | N/mgH** |
| 1 | 1 | wet | 100% | 5 | 0.74 |
| 2 | 2 | wet | 100% | 5 | 1.00 |
| 3 | 3 | wet | 100% | 5 | 1.18 |
| 4 | 4 | wet | 100% | 5 | 1.42 |
| 5 | 5 | dry | 10-20% | 5 | 2.53 |
| 6 | 6 | wet | 100% | 10 | 0.17 |
| 7 | 7 | wet | 100% | 10 | 0.18 |
| 8 | 8 | wet | 100% | 10 | 0.18 |
| 9 | 8 | wet | 100% | 10 | 0.24 |
| 10 | 10 | dry | 10-20% | 10 | 1.15 |

**The tensile strength (TS) is average of measurement on 2 samples, expressed as Newton per milligram hair (N/mgH)

### Example 24

### Calculations for Delivery of Substrates from Lotion

### General Recipe for Making Substrate Formulations

The following example demonstrates a prototypical preparation for the delivery of substrates for a perhydrolase, triacetin and H₂O₂ from a moisturizing solution.

For most examples the substrates were delivered from a moisturizing formulation. Exact proportion of the formulation is specified separately for each example. As a general example, if a 10 mL final formulation required 0.75 molar (M) hydrogen peroxide and 1.0 M triacetin in 20% lotion, the following recipe may be used:

(a) 5 mL of 1.5 M H₂O₂ formulation contains:
0.9 mL of 8.8 M stock H₂O₂ (17 v/v %),
1 mL of moisturizing lotion (2 v/v %) and
3.1 mL of buffer (63 v/v %)

(b) 5 mL of 2.0 M triacetin formulation contains:
1.9 mL of stock triacetin (37.5 v/v %),
1 mL of moisturizing lotion (2 v/v %) and
2.1 mL of buffer (42.5 v/v %)
   When equal volumes of H₂O₂ and triacetin formulations are applied on hair, final concentrations of 0.75 M H₂O₂ and 1.0 M of triacetin are achieved on the test surface.

### Example 25

### Demonstration of Hair Weakening in Two-Step

### General Procedure for a 2-Step Treatment Protocol

The following example demonstrates a protocol for a two-step product: the first step is a deposition of a targeted perhydrolase on hair (excess enzyme is washed away) and the second step is the delivery of the remaining reaction components (ester substrate, source of hydrogen peroxide) to the hair-deposited perhydrolase, wherein the remaining reaction components are delivered from a moisturizing solution.

Medium brown hair was used in these examples. Stock hair swatches were custom made by International Hair Importers (Glendale, N.Y.). Each swatch had 1500 mg of 50% reversed hair, soft glued in the middle with glue thickness of 1 mm and width of 2 cm.

### Tress Preparation:

Test-hair-sample consisted of a hair bundle of approximately 50 mg hair of 4 cm length, held together by a 1 mm thick, 2 mm wide and 5 mm long glue strip. A sheet of BYTAC® (TYPE VF-81, Saint-Gobain Performance Plastics, Wayne, N.J.) was attached to a polystyrene cover-plate, providing a TEFLON®-coated test-surface for mounting and treatment of hair. A strip of double-sided scotch tape was applied on the BYTAC® surface, closer to the top edge of the test plate. The glued side of the test-hair-sample was pressed down on the exposed side of the tape, making sure that the unglued hair strands are completely clear of the tape. The glued parts of the tress and exposed double-sided tape surface were covered with a strip of clear single-sided tape. Although the method is described for a single test-hair-tress, 4 tresses were used for each test condition and were placed side-by-side during the entire treatment.

### Enzyme Deposition:

The test tress was wetted by tap water and the excess water was absorbed with a paper towel. The calculated volume of the enzyme formulation was applied and was worked in gently and evenly into hair strands with gloved fingers for ˜30 seconds and let the formulation soak in for the specified deposition time. The tress was then rinsed with tap water agitated through a peristaltic pump for 20 seconds, to completely remove the surfactant.

### Substrate Application:

Excess water was removed from the wetted tress from the previous step by absorption on a paper towel. The requisite volumes of the substrates (H₂O₂ and triacetin (TA)) were applied onto the test-tress. The mixture was rubbed into the hair strand firmly and evenly, with gloved finger for ˜1 min. The tress was left semi-covered for the specified treatment time.

### Final Wash, Also Used as Pretreatment (Before Enzyme Deposition):

Test-hair was washed with a diluted surfactant solution by rubbing the surfactant into hair with enough vigor to foam, for one minute to remove all traced of oils and residues arising during tress preparation or from previous cycles. Hair was rinsed thoroughly with tap water, agitated through a peristaltic pump for 35 seconds, while gently opening tresses with gloved fingers to ascertain that all adherents (surfactant or moisturizer) are dislodged. Excess moisture was absorbed by pressing down the paper towel over the tress. This would mark the end of one cycle. Treatment cycles were repeated starting from the enzyme-deposition step till the desired number of cycles was reached.

After every fourth cycle the tresses were dried under air and color measurements were made using X-RITE® SP64 spectrophotometer (X-Rite, Grandville, Mich.) with 4 mm port. Color numbers were measured at D65/10® from reflectance, according to CIELAB76. Hair tresses (all 4 replicates) were placed under a card paper with punched out holes, making sure that the background was not visible. The port-hole of the spectrophotometer was centered on the hole to scan the hair sample underneath. The tress-bundle was turned over and placed under the card and an additional measurement was made. Average L*, a*, b* (color according to CIELAB76) values were recorded.

ΔE of color loss was calculated according to the following formula:

ΔE={square root over (((L1*−L0)̂2+(a1*−a0)̂2+(b1*−b0)̂2))}

### Where,

L1*, a1* and b1* are L*, a* and b* values for a sample tress after treatment,
L0*, a0* and b0* are L*, a* and b* values for untreated hair

Two samples from each experiment were subjected to Tensile assay as described in Example 23, to determine the extent of hair-weakening.

### Example 26

### 2-Step Daily Application

### 16 Cycles (Days) of 24 Hours Treatment Per Cycle

The following example demonstrates the structural weakening of hair in a two-step treatment.

The following experiments followed the general method of treatment disclosed in Example 25. Solutions were made in 5% TWEEN®-20 surfactant in 50 mM citrate buffer at pH 6. Approximately 0.8 mL of the enzyme solution was applied on ˜200 mg hair samples (4 test tresses) for 10 minutes. H₂O₂ and triacetin, formulations were made according to Example 24, in 20% LUBRIDERM® lotion in 50 mM citrate buffer at pH 6. After application of the reagents, the treatment was allowed to continue for 24 hr, as in daily application. TWEEN®-20 (5% in 50 mM citrate buffer at pH 6) was used for the final surfactant wash. The treatment was repeated for 15 additional cycles, 16 days in all. The results are provided in Table 19.

Under these conditions, all samples exhibited or exceeded the benchmark hair-weakening efficacy. The data demonstrates that a two-step product formulation can deposit enough perhydrolase in each cycle to produce peracetic acid from hydrogen peroxide and triacetin to significantly weaken hair in sixteen 24 hour cycles.

TABLE 19
Result of Hair Weakening Experiment Using 2-step Daily Application.

| | C277S- | | deposition | | | Mixture | treatment | |
|---|---|---|---|---|---|---|---|---|
| | HC263 | E/H, | time | [H₂O₂], | [TA], | volume, | time/cycle | TS, |
| Experiment | (μg/mL) | μg/mgH* | (min) | Molar | Molar | (mL) | (hr) | N/mgH** |
| 1 | control*** | 0 | 10 | 0.5 | 0.5 | 0.4 | 24 | 1.23 |
| 2 | 750 | 3 | 10 | 0.5 | 0.5 | 0.4 | 24 | 0.25 |
| 3 | 1750 | 7 | 10 | 0.5 | 0.5 | 0.4 | 24 | 0.14 |
| 4 | control*** | 0 | 10 | 0.25 | 0.25 | 0.4 | 24 | 2.39 |
| 5 | 750 | 3 | 10 | 0.25 | 0.25 | 0.4 | 24 | 1.51 |
| 6 | 1750 | 7 | 10 | 0.25 | 0.25 | 0.4 | 24 | 0.77 |

*E/H is the enzyme to hair ratio, expressed as micrograms per milligram hair (μg/mgH)
**TS is average (of 2) tensile strength, expressed as Newton per milligram hair (N/mgH)
***Control sample has no enzyme, hair is treated with surfactant only

### Example 27

### 2-Step Daily Application

### Accelerated Testing

The following example demonstrates an accelerated testing method for determining the effects of a treatment on hair weakening.

The following experiments followed the general method of treatment disclosed in Example 26, except that after application of the reagents, the treatment was allowed to continue for shorter times per cycle rather than 24 hours per cycle. The treatment conditions and tensile strength results after 16 cycles of treatment are shown in Table 20.

A comparison of these results and the results for 24 hour/cycle treatments from Table 19 in Example 26 demonstrates that tensile strengths for 24 hr/cycle treatments (TS−24 hr) can be predicted from the tensile strengths for 20 min/cycle treatments (TS−20 m), by substituting the values in the following equation:

(TS−24 hr)=1.34×(TS−20 m)−2.34

A tensile strength value of less than or equal to 1.8 N/mgH for 16 cycles of 20 min/cycle treatments indicates that the hair will completely disintegrate in 16 cycles of 24 hr/cycle treatments. Values obtained between 2.5 and 2.9 N/mgH will predict hair weakening efficacy similar to the benchmark, i.e., 1.0-1.5 N/mgH in 16 one per day cycles.

TABLE 20
Results of the Accelerated Testing Assay for Hair Weakening.

| | C277S- | E/H, | Deposition | | | Mixture | treatment | |
|---|---|---|---|---|---|---|---|---|
| | HC263 | μg/mg | time | [H₂O₂], | [TA], | volume, | time/cycle | TS, |
| Experiment | (μg/mL) | H* | (min) | Molar | Molar | (mL) | (min) | N/mgH*** |
| 1 | control**** | 0 | 10 | 0.5 | 0.5 | 0.8 | 20 | 2.58 |
| 2 | 750 | 3 | 10 | 0.5 | 0.5 | 0.8 | 20 | 2.09 |
| 3 | 1750 | 7 | 10 | 0.5 | 0.5 | 0.8 | 20 | 1.90 |
| 4 | control*** | 0 | 10 | 0.25 | 0.25 | 0.8 | 20 | 3.65 |
| 5 | 750 | 3 | 10 | 0.25 | 0.25 | 0.8 | 20 | 2.64 |
| 6 | 1750 | 7 | 10 | 0.25 | 0.25 | 0.8 | 20 | 2.33 |

*E/H is the enzyme to hair ratio, expressed as micrograms per milligram hair (μg/mgH)
***TS is average (of 2 samples) tensile strength, expressed as Newton per milligram hair (N/mgH)
****Control sample has no enzyme, hair is treated with surfactant only

### Example 28

### Range of Enzyme Load

The following example demonstrates the effects of enzyme load on tensile strength and color loss. This example followed the general method of treatment disclosed in Example 25.

Enzyme solutions were made in 0.5% or 5% TWEEN®-20 surfactant in 50 mM citrate buffer at pH 6. Approximately 0.8 mL of an enzyme solution was applied on ˜200 mg hair samples (4 test tresses) for 10 minutes. H₂O₂ and triacetin, formulations were made according to Example 24, in 20% LUBRIDERM® lotion (Johnson & Johnson, New Brunswick, N.J.) in 50 mM citrate buffer at pH 6. After application of 400 μL each of the reagents, the treatment was allowed to continue for 20-40 min. The final concentrations of H₂O₂ and TA in 800 μL of the substrate mixture are shown in Table 21. TWEEN®-20 (5% in 50 mM citrate buffer at pH 6) was used for the final surfactant wash. Tensile strength results for the samples thus treated and the corresponding predicted values for 16 cycles of 24 hr/cycle treatment regimen are shown in Table 21.

Even at the lowest enzyme concentration used in this example, 750 μg/mL, the predicted tensile strength for 16 cycles of 24 hr/cycle treatment regimen will be ˜1 N/mgH, which is similar to (or better than) the benchmark. Under these conditions the hair is also bleached in proportion to the decreasing tensile strength, which could be used as an early indication of treatment efficacy.

TABLE 21
Results of Enzyme Load Experiment on Tensile Strength and Color Loss of Hair.

| | C277S- | | Deposition | | | Treatment | Color | | Predicted |
|---|---|---|---|---|---|---|---|---|---|
| | HC263 | E/H, | time | [H₂O₂], | [TA], | time/cycle | loss | TS, | TS for |
| Sample | (μg/mL) | μg/mgH* | (min) | Molar | Molar | (min) | ΔE | N/mgH** | 24 h/cycle |
| 1 | 2500 | 10 | 10 | 0.75 | 1 | 20 | 10 | 1.73 | −0.02(d) |
| 2 | 2500 | 10 | 10 | 0.75 | 1 | 40 | 9 | 1.67 | −0.10(d) |
| 3 | 1750 | 7 | 10 | 0.75 | 1 | 40 | 10 | 1.65 | −0.13(d) |
| 4 | 1500 | 5 | 10 | 0.75 | 1 | 20 | 7 | 2.47 | 0.97 |
| 5 | 750 | 3 | 10 | 0.75 | 1 | 40 | 8 | 2.49 | 1.00 |
| 6 | control*** | 0 | 10 | 0.75 | 1 | 40 | 5 | 3.10 | 1.81 |

*E/H is the enzyme to hair ratio, expressed as micrograms per milligram hair (μg/mgH)
**TS is tensile strength, expressed as Newton per milligram hair (N/mgH)
***Control sample has no enzyme, hair is treated with surfactant only
(d)Predicted disintegration of hair in 16 × 24 h/cycle treatment

### Example 29

### Demonstration of Greater Hair Damage and Increased Enzyme Binding with Increased Number of Cycles

The following example demonstrates how the amount of peracetic acid produced at each repeat cycle increases with increasing cycles. The experiment followed the general method of treatment disclosed in Example 25.

The enzyme used in the following example was HC1121 (T. maritima C277S-HC263; SEQ ID NO: 288) and concentration used was 2500 μg/mL (E/H=10*). Enzyme solution was made in 5% TWEEN®-20 surfactant in 50 mM citrate buffer at pH 6. Approximately 0.8 mL of the enzyme solution was applied on ˜200 mg hair samples (4 test tresses) for 10 minutes. H₂O₂ and triacetin, formulations were made according to Example 24, in 20% LUBRIDERM® lotion in 50 mM citrate buffer at pH 6. After application of 400 μL each of the reagents, the treatment was allowed to continue for 20 minutes after which the hair was rinsed with tap-water and allowed to dry. Before proceeding with the next cycle the sample was subjected to the surfactant wash with 5% TWEEN®-20 in 50 mM citrate buffer at pH 6 and rinsed with tap-water.

In order to quantify the hair-weakening, hair-bleaching and the enzyme activity (its propensity to produce peracetic acid, PAA), tests such as tensile strength measurements, color measurements and the assay for bound perhydrolase enzyme activity were performed.

### Assay for Bound Perhydrolase Enzyme Activity:

After treatment for a defined number of cycles, 10 mg of hair was cut from each tress and placed into 2 mL microcentrifuge tubes (Eppendorf Protein LoBind; Eppendorf North America, Hauppauge, N.Y.). To each tube was added 480 uL of PAH buffer (50 mM potassium phosphate pH 7.2), 14 μL of 30% hydrogen peroxide (Sigma 3410), and 6 μL triacetin (Sigma-Aldrich, W200700). The perhydrolase reaction was allowed to proceed for 10 minutes at 25° C. The reaction was stopped by diluting aliquots 10×, 100× and 1000× with 10 mM phosphoric acid. For detection, 50 μL of 1 M acetic acid, 50 μL of 40 mg/mL potassium iodide, and 50 μL of 4 mM ABTS (2,2′-azino-bis(3-ethylbenzothiazoline-6-sulphonic acid; Sigma, St Louis, Mo.) were added to 50 μL of the above dilutions. Color was allowed to develop for 10 minutes and read in a spectrophotometer at 405 nm. Values from no-enzyme controls were subtracted to yield values attributable only to enzyme activity. A standard curve of OD 405 nm vs PAA concentration in ppm yielded a conversion factor of 8.75, such that an OD 405 nm of 1.0 corresponded to approximately 8.75 ppm PAA. This value was then multiplied by the appropriate dilution to arrive at the PAA concentration in the perhydrolase reaction.

The results are shown in Table 22. As the number of treatment cycles increased, larger extent of hair bleaching and lowering of tensile strengths were observed. Increasing numbers of treatment cycles also made hair more susceptible to larger amount of enzyme binding, which resulted in larger quantity of peracetic acid produced for the same concentrations of substrates added during the assay.

TABLE 22
Results Showing the Effect of Treatment Cycles
on Enzyme Deposition and Retention.

| | | | ΔE of | TS, | | PAA |
|---|---|---|---|---|---|---|
| Sample | Treatment | | color loss | N/mgH | OD 405ₙₘ | (ppm) |
| 1 | 1 | cycle | 2 | 3.44 | 0.172 | 150.8 |
| 2 | 4 | cycles | 4 | 3.13 | 0.439 | 384.1 |
| 3 | 8 | cycles | 6 | 2.34 | 0.901 | 788.4 |
| 4 | 12 | cycles | 8 | 1.66 | 1.287 | 1126.1 |
| 5 | 16 | cycles | 11 | 0.95 | 1.894 | 1657 |

| | | | | | |
|---|---|---|---|---|---|
| 6 | Enzyme-free | 3 | 3.7 | 0 | — |
| | control—16 cycles | | | | |

* E/H is the enzyme to hair ratio, expressed as micrograms per milligram hair (μg/mgH)
** TS is tensile strength, expressed as Newton per milligram hair (N/mgH)
*** Control sample has no enzyme, hair is treated with surfactant only

This example demonstrates that with additional cycles of the two step process, more peracetic acid is produced and more weakening of the hair takes place. This observation leads support to the increased efficacy of a depilatory regimen that incorporate repeated cycles of perhydrolase deposition and peracetic acid production.

### Example 30

### Role of Hair-Targeting Domain for Weakening of Hair in Two-Step Process

The following example demonstrates that the hair-binding domain on the perhydrolase fusion improves efficacy in weakening hair in the tress assay,

The following experiments followed the general method of treatment disclosed in Example 25. The enzymes used in the following example were the Thermotoga maritima perhydrolase targeted to hair via the hair binding peptide HC263 (C277S-HC263; SEQ ID NO: 288) and untargeted perhydrolase (T. maritima C277S; SEQ ID NO: 293). Concentrations of enzymes were 1500 μg/mL (E/H=6*). Enzyme solutions were made in 5% TWEEN®-20 surfactant in 50 mM citrate buffer at pH 6. Approximately 0.8 mL of the enzyme solution was applied on ˜200 mg hair samples (4 test tresses) for 5 minutes. H₂O₂ and triacetin, formulations were made according to Example 24, in 20% LUBRIDERM® lotion in 50 mM citrate buffer at pH 6. After application of 400 μL each of the reagents, the treatment was allowed to continue for 2 hours. Before proceeding with the next cycle the sample was subjected to the surfactant wash with 5% TWEEN®-20 in 50 mM citrate buffer at pH 6 and rinsed with tap-water. The treatment conditions and tensile strength results are summarized in Table 23.

Tensile strength (TS) results show that the targeted perhydrolase enzyme treatment showed lower tensile strength than the untargeted. This indicates that the targeting provides a larger hair-weakening efficacy in a two-step daily application depilatory product. This example demonstrates that in the two-step protocol the targeting of the enzyme to hair is necessary for efficacy at weakening the structure of the hair.

TABLE 23
Tensile Strength Results.

| | | | | Deposition | | | Treatment | |
|---|---|---|---|---|---|---|---|---|
| | | [E] | E/H, | time | [H₂O₂], | [TA], | time/cycle | TS, |
| Sample | Enzyme | (μg/mL) | μg/mgH* | (min) | Molar | Molar | (hr) | N/mgH** |
| 1 | none | control*** | 0 | 5 | 0.5 | 1 | 2 | 2.93 |
| 2 | C277S | 1500 | 6 | 5 | 0.5 | 1 | 2 | 2.84 |
| 3 | C277S- | 1500 | 6 | 5 | 0.5 | 1 | 2 | 1.62 |
| | HC263 | | | | | | | |

*E/H is the enzyme to hair ratio, expressed as micrograms per milligram hair (μg/mgH)
**Tensile strength (TS) is the average (of 2 samples) tensile strength, expressed as Newton per milligram hair (N/mgH)
***Control sample has no enzyme, hair is treated with surfactant only

### Example 31

### Construction and Production of Other Perhydrolases Targeted to Hair

The following example demonstrates the design of expression systems for the production of additional perhydrolases targeted to hair. A summary of the constructs is provided in Table 24.

Briefly, the polynucleotide sequences (SEQ ID NOs: 9, 39, and 41) were designed to encode fusions of xylan esterases from Bacillus pumilus, Lactococcus lactis and Mesorhizobium loti (SEQ ID NOs 10, 40, and 42) to a 18 amino acid flexible linker (GPGSGGAGSPGSAGGPGS; SEQ ID NO: 285); itself fused to the hair-binding domains HC263 (SEQ ID NO 290). These enzymes belong to the CE-7 family of carbohydrate esterases as does the Thermotoga maritima perhydrolase.

The polynucleotide sequences (SEQ ID NOs: 322, 324, 326 and 328) were designed to encode fusions of the S54V variant of the aryl esterase from Mycobacterium smegmatis (SEQ ID NO: 314) to a 18 amino acid flexible linker (SEQ ID NO: 285); itself fused to the hair-binding domains HC263 (SEQ ID NO 290). The aryl esterase from Mycobacterium smegmatis belongs to a different class of hydrolytic enzyme than that of the Thermotoga maritima perhydrolase. The polynucleotide sequences (SEQ ID NOs: 330, 332, 334, and 336) were designed to encode fusions of the L29P variant of the hydrolase from Pseudomonas fluorescens (SEQ ID NO: 315) to a 18 amino acid flexible linker (SEQ ID NO: 285); itself fused to the hair-binding domains HC263 (SEQ ID NO: 290). The esterase from Pseudomonas fluorescens belongs to a different class of hydrolytic enzymes than that of the Thermotoga maritima perhydrolase or of Mycobacterium smegmatis.

The genes were codon-optimized for expression in E. coli and synthesized by DNA2.0 (Menlo Park, Calif.). The coding sequences were cloned in plasmids behind the T7 promoter or the pBAD promoter in a manner similar as that described in Example 8. The plasmids were transferred in an appropriate expression host: E. coli strain BL21AI (Invitrogen, Carlsbad, Calif.) for constructs under the T7 promoter or in an AraBAD derivative of E. coli MG1655 for constructs under the pBAD promoter.

TABLE 24
Description of Various Hydrolase/Perhydrolases Fused
to Targeting Sequences with Affinity for Hair.

| | | Nucleic Acid | |
|---|---|---|---|
| | | Sequence | Amino Acid |
| | | Encoding the | sequence of |
| | Targeting | Targeted | the Targeted |
| Organism source | Sequence | Perhydrolase | Perhydrolase |
| of perhydrolase | (SEQ ID NO:) | (SEQ ID NO:) | (SEQ ID NO:) |
| Bacillus | HC263 | 316 | 317 |
| pumilus | (SEQ ID NO: 290) | | |
| Lactococcus | HC263 | 318 | 319 |
| lactis | (SEQ ID NO: 290) | | |
| Mesorhizobium | HC263 | 320 | 321 |
| loti | (SEQ ID NO: 290) | | |
| Mycobacterium | HC263 | 322 | 323 |
| smegmatis | (SEQ ID NO: 290) | | |
| Mycobacterium | HC263KtoR | 324 | 325 |
| smegmatis | (SEQ ID NO: 312) | | |
| Mycobacterium | HC1010 | 326 | 327 |
| smegmatis | (SEQ ID NO: 291) | | |
| Mycobacterium | (GK)₅-His6 | 328 | 329 |
| smegmatis | (SEQ ID NO: 313) | | |
| Pseudomonas | HC263 | 330 | 331 |
| fluorescens | (SEQ ID NO: 290) | | |
| Pseudomonas | HC263KtoR | 332 | 333 |
| fluorescens | (SEQ ID NO: 312) | | |
| Pseudomonas | HC1010 | 334 | 335 |
| fluorescens | (SEQ ID NO: 291) | | |
| Pseudomonas | (GK)₅-His6 | 336 | 337 |
| fluorescens | (SEQ ID NO: 313) | | |

### Example 32

### Production of Fusion Proteins Comprising Alternative Esterase/Perhydrolase and a Hair-Binding Domain

The following example demonstrates the expression and purification of various alternative esterase/perhydrolase targeted to hair as described in Example 31.

Strains expressing the genes encoding fusions to the perhydrolases in Table 24 of Example 31 were grown in 1 L of autoinduction medium (10 g/L Tryptone, 5 g/L Yeast Extract, 5 g/L NaCl, 50 mM Na₂HPO₄, 50 mM KH₂PO₄, 25 mM (NH₄)₂SO₄, 3 mM MgSO₄, 0.75% glycerol, 0.075% glucose and 0.05% arabinose) containing 50 mg/L spectinomycin at 37° C. for 20 hours under 200 rpm agitation. All protein fusions expressed well in E. coli. The cells were harvested by centrifugation at 8000 rpm at 4° C. and washed by resuspending the cell pellets in 300 mL of ice chilled lysis buffer (50 mM Tris, pH 7.5, 100 mM NaCl) using a tissue homogenizer (Brinkman Homogenizer model PCU11) at 3500 rpm followed by centrifugation (8000 rpm, 4° C.). The cells were disrupted by two passes through a French pressure cell at 16,000 psi (110.32 MPa). The lysed cell extracts were transferred to 4×50-mL conical polypropylene centrifuge tubes and centrifuged at 10,000 rpm for 10 min at 4° C. The supernatant containing the enzymes were transferred to new tubes. The approximate amount of fusion protein in each extract was estimated by comparison to bands of Bovine Serum Albumin standard on a Coomassie stained PAGE gel.

Since the perhydrolases fusions are not thermophilic, they were purified using their C-terminal His6 by metal chelation chromatography using Co-NTA agarose (HisPur Cobalt Resin, Thermo Scientific). Typically, cell extracts were loaded to a 5 to 10 mL column of Co-NTA agarose equilibrated with 4 volume of equilibration buffer (10 mM Tris HCl pH 7.5, 10% glycerol, 1 mM Imidazole and 150 mM NaCl). The amount of each extract loaded on the column was adjusted to contain between 5 and 10 mg of perhydrolase fusion per mL of Co-NTA agarose beads. The resin was washed with two bed volumes of equilibration buffer and was eluted with two volume of elution buffer (10 mM Tris HCl pH 7.5, 10% glycerol, 150 mM Imidazole, 500 mM NaCl). Fractions were collected and the presence of the purified proteins was detected by PAGE. The eluted proteins were analyzed by PAGE. All these proteins could be purified by affinity chromatography. That fact indicates that the fusion proteins were produced in the full length form.

This example demonstrates the feasibility of producing fusion hydrolases/perhydrolases from different families with a variety of binding domains having affinity to hair.

### Example 33

### Perhydrolase Activity of Alternative Perhydrolases Fused to a Hair-Binding Domains

The following example demonstrates the activity of alternative perhydrolases targeted to hair.

The perhydrolase activity of the enzymes targeted to hair with a variety of targeting domains produced as described in Example 32 was measured with the ABTS assay. The results are reported in Table 25 and show that CE-7 (carbohydrate esterase family 7) as well as non-CE-7 hydrolases have perhydrolytic activity

TABLE 25
Perhydrolase Activity of Various Targeted Hydrolases.

| | | Targeted | |
|---|---|---|---|
| | | Perhydrolase | Specific |
| | Targeting | Amino Acid | perhydrolase |
| Organism source | Sequence | Sequence | activity |
| of perhydrolase | (SEQ ID NO:) | (SEQ ID NO:) | (μmol/mg/min) |
| Bacillus | HC263 | 317 | 40 |
| pumilus | (SEQ ID NO: 290) | | |
| Lactococcus | HC263 | 319 | 99 |
| lactis | (SEQ ID NO: 290) | | |
| Mesorhizobium | HC263 | 321 | 34 |
| loti | (SEQ ID NO: 290) | | |
| Mycobacterium | HC263 | 323 | 270 |
| smegmatis | (SEQ ID NO: 290) | | |
| Mycobacterium | HC263KtoR | 325 | 46 |
| smegmatis | (SEQ ID NO: 312) | | |
| Mycobacterium | HC1010 | 327 | 20 |
| smegmatis | (SEQ ID NO: 291) | | |
| Mycobacterium | (GK)₅-His6 | 329 | 264 |
| smegmatis | (SEQ ID NO: 313) | | |
| Pseudomonas | HC263 | 331 | 0.37 |
| fluorescens | (SEQ ID NO: 290) | | |
| Pseudomonas | HC263KtoR | 333 | 1.45 |
| fluorescens | (SEQ ID NO: 312) | | |
| Pseudomonas | HC1010 | 335 | 1.5 |
| fluorescens | (SEQ ID NO: 291) | | |
| Pseudomonas | (GK)₅-His6 | 337 | 2.65 |
| fluorescens | (SEQ ID NO: 313) | | |

Note:
The perhydrolase activity of the fusions of the Pseudomonas fluorescens hydrolase was assayed using 1 m Na acetate at pH 5.5 instead of triacetin at pH 7.5 Targeted Perhydrolases HC1121 (C277S-HC263; SEQ ID NO: 288) had no detectable perhydrolase activity with acetate as a substrate.

This example demonstrates that other hair-targeting fusions of hydrolase enzymes, from the CE-7 family or from other families, show perhydrolytic activity and could be used directly or after enzyme evolution in hair applications.

### Example 34

### Hair Binding of Other Perhydrolases Targeted to Hair

The following example demonstrates that various targeted perhydrolases (other than the CE-7 Thermotoga maritima perhydrolase) can bind to hair.

Targeted Perhydrolases HC1121 (C277S-HC263; SEQ ID NO: 288), HC1169 (ArE-HC263; SEQ ID NO: 323) and variants of P. fluorescens perhydrolase (SEQ ID NO:331) were diluted to 50 μg/mL in a solution of 5% PEG-80 sorbitan laurate in 100 mM citrate-phosphate buffer adjusted to pH 6.0. Ten mg of human hair was added to 2 mL of the above formulations and incubated with gentle agitation for 5 minutes at room temperature to allow enzyme binding to hair. A no-enzyme control sample was also included. After binding, the binding solution was removed by aspiration and the hair was washed with 2 mL of 1% TWEEN®-20 in 50 mM pH 7.2 potassium phosphate buffer. The hair was removed from the tube, blotted dry with paper towel, and transferred to a new set of tubes. The hair was rinsed two times with 1% TWEEN®-20 in 50 mM pH 7.2 potassium phosphate buffer and then rinsed three times with 50 mM pH 7.2 potassium phosphate buffer. The amount of enzyme remaining bound to the hair was determined by SDS-PAGE analysis by cutting the hair into 2-3 mm fragments. The fragments were placed into a 500 μL polypropylene microcentrifuge tube and covered with 80 μL of gel loading buffer (20 μL NuPAGE LDS sample buffer (Invitrogen NP0007), 8 μL of 500 mM DTT, and 52 μL 50 mM pH 7.2 potassium phosphate). The hair samples were heated to 90° C. for 10 minutes, then cooled to 4 degrees.

The supernatant (25 μL) was loaded onto a NuPAGE 4-12% Bis-tris polyacrylamide gel (Invitrogen NP0322) and run at 150 v for 40 min. The gel was washed 3 times with water and stained in 15 mL SIMPLYBLUE™ Safestain (Invitrogen, Carlsbad, Calif.; LC6060) for 1 hour, rinsed 3 times, and then destained for 3 hours in water. The results are reported as relative intensity of enzyme band on the gel and provided in Table 26.

TABLE 26
Relative Binding of Various Perhydrolase Fusions on Hair.

| | | Targeted | |
|---|---|---|---|
| | Targeting | Perhydrolase | Relative |
| Organism source | sequence | Sequence | intensity band |
| of perhydrolase | (SEQ ID NO:) | (SEQ ID NO) | on PAGE |
| Thermotoga | HC263 | 288 | +++ |
| maritima | (SEQ ID NO: 290) | | |
| Mycobacterium | HC263 | 323 | +++ |
| smegmatis | (SEQ ID NO: 290) | | |
| Mycobacterium | HC263KtoR | 325 | +++ |
| smegmatis | (SEQ ID NO: 312) | | |
| Mycobacterium | HC1010 | 327 | + |
| smegmatis | (SEQ ID NO: 291) | | |
| Mycobacterium | (GK)₅-His6 | 329 | +++ |
| smegmatis | (SEQ ID NO: 313) | | |
| Pseudomonas | HC263 | 331 | +++ |
| fluorescens | (SEQ ID NO: 290) | | |
| Pseudomonas | HC263KtoR | 333 | ++ |
| fluorescens | (SEQ ID NO: 312) | | |
| Pseudomonas | HC1010 | 335 | + |
| fluorescens | (SEQ ID NO: 291) | | |
| Pseudomonas | (GK)₅-His6 | 337 | ++ |
| fluorescens | (SEQ ID NO: 313) | | |

The data indicates that diverse perhydrolases from different hydrolase families can be targeted to hair and that hair binding sequences are functional in the context of fusions to perhydrolases other than the Thermotoga perhydrolase.

### Example 35

### Hair Binding of Targeted Perhydrolases with K to R Substitutions in the Hair-Binding Domain HC263

The following example demonstrates that variations of hair binding domain of the hair-targeted perhydrolase also bind hair.

To test the binding ability of mutant binding sequences in which 10 lysine residues of the HC263 hair binding domain were substituted by arginine residues, a number of variant enzymes were each diluted to 50 μg/mL in a solution of 5% PEG-80 sorbitan laurate in 100 mM citrate-phosphate buffer adjusted to pH 6.0. Ten mg of human hair was added to 2 mL of the above formulations and incubated with gentle agitation for 10 minutes at room temperature to allow enzyme binding to hair. A no-enzyme control sample was also included. After binding, the binding solution was removed by aspiration and the hair was washed with 2 mL of 1% TWEEN®-20 in 50 mM pH 7.2 potassium phosphate buffer. The hair was removed from the tube, blotted dry with paper towel, and transferred to a new set of tubes. The hair was rinsed once with 1% TWEEN®-20 in 50 mM pH 7.2 potassium phosphate buffer and then rinsed three times with 50 mM pH 7.2 potassium phosphate buffer. Perhydrolase activity remaining bound to the hair was determined by the ABTS assay. The results are given in Table 27 as ppm PAA at 10 minutes (after subtraction of the no-enzyme control values)

TABLE 27
Binding of Perhydrolase Activity.

| | Targeting | Targeted | |
|---|---|---|---|
| Organism source | sequence | Perhydrolase | |
| of perhydrolase | (SEQ ID NO:) | (SEQ ID NO:) | ppm PAA |
| Thermotoga maritima | HC263 | 288 | 581 |
| Thermotoga maritima | HC263KtoR | 341 | 958 |

### Example 36

### Dependence of Hair Targeting Sequence for the Binding of the Mycobacterium smegmatis Perhydrolase on Hair

The following example demonstrates that hair-targeted perhydrolases other than the CE-7-carbohydrate esterase-derived perhydrolases bind to hair and that a hair binding domain is required for binding to hair.

The S54V variant of Mycobacterium smegmatis aryl esterase targeted to hair (SEQ ID NO: 323) and its untargeted counterpart (SEQ ID NO: 314) were diluted to 50 μg/mL in a solution of 5% PEG-80 sorbitan laurate in 100 mM citrate-phosphate buffer adjusted to pH 6.0. Ten mg of human hair was added to 2 mL of the above formulations and incubated with gentle agitation for 1 min at room temperature (˜22° C.) to allow enzyme binding to hair. A no-enzyme control sample was also included. After binding, the binding solution was removed by aspiration and the hair was washed with 2 mL of 1% TWEEN®-20 in 50 mM pH 7.2 potassium phosphate buffer. The hair was removed from the tubes, blotted dry with paper towel, and then transferred to a new set of tubes. The hair was rinsed once more with 50 mM pH 7.2 potassium phosphate buffer and then rinsed twice with 50 mM pH 7.2 potassium phosphate buffer. Perhydrolase activity remaining bound to the hair was determined by the ABTS assay. The results are provided in Table 28 as OD 405 nm and ppm PAA at 10 minutes (after subtraction of the no-enzyme control values). They show that essentially no un-targeted aryl esterase bound to hair.

TABLE 28
Mycobacterium smegmatis Aryl Esterase
Peracid Production When Targeted to Hair.

| Mycobaterium | | Peracetic acid |
|---|---|---|
| perhydrolase | OD 405ₙₘ | (ppm) |
| Targeted | 0.237 | 207 |
| (SEQ ID NO: 323) | | |
| Untargeted | 0.003 | 2.60 |
| (SEQ ID NO: 314) | | |

This example demonstrates the need for a hair-targeting sequence to bind the Mycobacterium perhydrolase to hair as well as the functionality of the hair targeting sequence in the context of the fusion protein. More generally this exemplifies that diverse perhydrolases from different hydrolase families can be targeted to hair.

### Example 37

### Improved Binding of a Targeted Perhydrolase to Hair Previously Damaged by PAA

The following example demonstrates that the binding of a hair targeted perhydrolase to hair is increased as the hair becomes damaged by peracetic acid (PAA).

To evaluate the effect on enzyme binding of PAA damage to hair, PAA-damaged human hair was prepared by soaking tresses of hair for 1 hr at room temperature (˜22° C.) in 0.2% PAA adjusted to pH 6.5. After soaking, the hair was rinsed three times with water and once with 50 mM pH 7.2 potassium phosphate buffer. Some tresses were treated as described twice. Targeted aryl esterase HC1169 (ArE-HC263; SEQ ID NO: 323) was diluted to 50 μg/mL in a solution of 5% PEG-80 sorbitan laurate in 100 mM citrate-phosphate buffer adjusted to pH 6.0. Ten mg of human hair (untreated, PAA-treated once, PAA-treated twice) was each added to 2 mL of the above formulation and incubated with gentle agitation for 1 minute at room temperature to allow enzyme binding to hair. A no-enzyme control sample was also included. After binding, the binding solution was removed by aspiration and the hair was washed with 2 mL of 50 mM pH 7.2 potassium phosphate buffer. The hair was removed from the tubes, blotted dry with paper towel, and transferred to a new set of tubes. The hair was rinsed once with 50 mM pH 7.2 potassium phosphate buffer. Perhydrolase activity remaining bound to the hair was determined by the ABTS assay. The results are reported in Table 29 as ppm PAA at 10 minutes (after subtraction of the no-enzyme control values). The results clearly show an increase perhydrolase activity retained on the hair corresponding to an increased amount with increasing PAA damage.

TABLE 29
Improvement of Binding of Targeted Perhydrolase
on Hair Treated with Peracetic Acid.

| Hair sample | ppm PAA |
|---|---|
| No PAA treatment | 269 |
| One PAA treatment | 526 |
| Two PAA treatments | 702 |

This example demonstrates that as the more damaged the hair is by peracetic acid, the more perhydrolase binds. This observation leads support to the increased efficacy of a depilatory regimen that incorporate repeated cycles of perhydrolase deposition and peracetic acid production.

### Example 38

### Weakening of Hair in a Two-Step Process Using a Perhydrolase from Mycobacterium Smegmatis

The following example demonstrates that other perhydrolases than the CE-7 carbohydrate esterase-derived perhydrolases can be used in a two-step process to weaken hair.

The following experiment followed the general method of treatment disclosed in Example 25. The enzymes used in this example a non-CE-7 enzyme obtained from Mycobacterium Smegmatis (aryl esterase S54V variant; SEQ ID NO: 314) with targeting hair-binding domain HC263 (SEQ ID NO: 290). The concentration of the enzyme was 1500 μg/mL (E/H=6*). The concentrations of reagents and test conditions were same as in Example 30. The treatment conditions and tensile strength results are summarized in Table 30 and were compared with previous results for the enzyme-free control.

Tensile strength (TS) results show that under these application conditions the hair-targeted Mycobacterium smegmatis aryl esterase S54V variant (ArE-HC263; SEQ ID NO: 323) enzyme showed lower tensile strength than the control and therefore, provided the hair-weakening efficacy in a two-step daily application depilatory product.

TABLE 30
Weakening of Hair in a Two-step Process.

| | | | | Deposition | | | treatment | |
|---|---|---|---|---|---|---|---|---|
| | | [E] | E/H, | time | [H₂O₂], | [TA], | time/cycle | TS, |
| Sample | Enzyme | μg/mL | μg/mgH* | (min) | Molar | Molar | (hr) | N/mgH** |
| 1 | none | control*** | 0 | 5 | 0.5 | 1 | 2 | 2.93 |
| 2 | ArE-HC263 | 1500 | 6 | 5 | 0.5 | 1 | 2 | 0.28 |
| | (HC1169) | | | | | | | |
| | (SEQ ID | | | | | | | |
| | NO: 323) | | | | | | | |

*E/H is the enzyme to hair ratio, expressed as micrograms per milligram hair (μg/mgH)
**TS is the average (of 2 samples) tensile strength, expressed as Newton per milligram hair (N/mgH)
***Control sample has no enzyme, hair is treated with surfactant only

### Example 39

### Use of Different Perhydrolases and Different Substrates to Generate Peracetic Acid

The following example demonstrates that an effective amount of PAA suitable for a depilatory application can be generated with different perhydrolases with different substrates.

HC1121 is a CE-7 class carbohydrate esterase from Thermotoga maritima (C277S-HC263; SEQ ID NO: 288), and HC1169 is an aryl esterase from M. smegmatis (ArE-HC263; SEQ ID NO: 323). Both enzymes were tested for their perhydrolytic activity with substrates triacetin or propylene glycol diacetate (PGDA, Aldrich 528072) and hydrogen peroxide between pH 5 and pH 7.2. The concentration of enzyme, substrate and buffer, and reaction time are listed in Table 31. Enzyme free reactions for some reaction conditions were run to determine the non-enzymatic generation of peracetic acid as well. At the end of reaction, the reaction was quenched by acidification 10 or 25 fold with 100 mM H₃PO₄. The quenched samples were filtered using a NANOSEP® MF centrifugal device (30K Molecular Weight Cutoff (MWCO), Pall Life Sciences, Ann Arbor, Mich., PIN OD030C35) by centrifugation for 6 min at 12,000 rpm. The filtrates were assayed by HPLC Karst assay in duplicates to determine the amount of peracetic acid (PAA) generated at those reaction conditions.

In the first group of tests (100 mM triacetin and 200 mM H₂O₂ were used in different buffers), without enzyme, triacetin and hydrogen peroxide generated very low amount of PAA (110 ppm PAA or less) in 5 min; while addition of 50 μg/mL HC1121 generated about 277 ppm to 4832 ppm PAA in 5 min depending on pH. The higher the pH, the more PAA was generated.

In the second group of tests (250 mM triacetin and 100 mM H₂O₂ were used in 20% LUBRIDERM® lotion in 100 mM, pH 7.2 phosphate buffer), without enzyme, triacetin and hydrogen peroxide generated 332 ppm PAA in 60 min, while addition of 10 μg/mL of HC1169 generated 3433 ppm PAA in 60 min, and addition of 10 μg/mL of HC1121 generated 4451 ppm PAA in 60 min.

In the third group of tests (250 mM triacetin and 100 mM H₂O₂ were used in different buffers), 20 μg/mL of HC1169 generated 3680 ppm to 4812 ppm PAA in 30 min, showing little dependence on pH.

In the fourth group of tests (250 mM PGDA and 100 mM H₂O₂ were used in different buffers), 10 μg/mL and 20 μg/mL of HC1169 generated 4140 ppm-4726 ppm PAA in 30 min, again showing little dependence on pH. In addition, 10 μg/mL HC1169 already saturated the reaction with the provided substrates, and 20 μg/mL HC1169 didn't show additional gain on PAA generation.

TABLE 31
PAA Generation using Different Perhydrolases and
Different Substrates at Different pH.

| Test | Perhydrolase | | H₂O₂ | | Reaction | PAA |
|---|---|---|---|---|---|---|
| Group | concentration | | (mM) | Buffer | time (min) | (ppm) |
| | | Triacetin | | | | |
| | | (mM) | | | | |
| Group 1 | No enzyme | 100 | 250 | pH 5, 50 mM | 5 | 51 |
| | | | | citrate buffer | | |
| | No enzyme | 100 | 250 | pH 5.6, 50 mM | 5 | 62 |
| | | | | citrate buffer | | |
| | No enzyme | 100 | 250 | pH 6, 50 mM | 5 | 49 |
| | | | | citrate buffer | | |
| | No enzyme | 100 | 250 | pH 6.6, 50 mM | 5 | 62 |
| | | | | citrate-phosphate | | |
| | | | | buffer | | |
| | No enzyme | 100 | 250 | pH 7, 50 mM | 5 | 110 |
| | | | | pyrophosphate | | |
| | | | | buffer | | |
| | 50 μg/mL | 100 | 250 | pH 5, 50 mM | 5 | 277 |
| | HC1121 | | | citrate buffer | | |
| | 50 μg/mL | 100 | 250 | pH 5.6, 50 mM | 5 | 1222 |
| | HC1121 | | | citrate buffer | | |
| | 50 μg/mL | 100 | 250 | pH6, 50 mM | 5 | 2350 |
| | HC1121 | | | citrate buffer | | |
| | 50 μg/mL | 100 | 250 | pH 6.6, 50 mM | 5 | 4067 |
| | HC1121 | | | citrate-phosphate | | |
| | | | | buffer | | |
| | 50 μg/mL | 100 | 250 | pH 7, 50 mM | 5 | 4832 |
| | HC1121 | | | pyrophosphate | | |
| | | | | buffer | | |
| Group 2 | No enzyme | 250 | 100 | 20% | 60 | 332 |
| | | | | LUBRIDERM ® in | | |
| | | | | pH 7.2, 100 mM | | |
| | | | | phosphate buffer | | |
| | 10 μg/mL | 250 | 100 | 20% | 60 | 3433 |
| | HC1169 | | | LUBRIDERM ® in | | |
| | | | | pH 7.2, 100 mM | | |
| | | | | phosphate buffer | | |
| | 10 μg/mL | 250 | 100 | 20% | 60 | 4451 |
| | HC1121 | | | LUBRIDERM ® in | | |
| | | | | pH 7.2, 100 mM | | |
| | | | | phosphate buffer | | |
| Group 3 | 20 μg/mL | 250 | 100 | pH 5, 100 mM | 30 | 3680 |
| | HC1169 | | | citrate buffer | | |
| | 20 μg/mL | 250 | 100 | pH 5.6, 100 mM | 30 | 4429 |
| | HC1169 | | | citrate buffer | | |
| | 20 μg/mL | 250 | 100 | pH 6, 100 mM | 30 | 4616 |
| | HC1169 | | | citrate buffer | | |
| | 20 μg/mL | 250 | 100 | pH 6.6, 100 mM | 30 | 4488 |
| | HC1169 | | | phosphate buffer | | |
| | 20 μg/mL | 250 | 100 | pH 7.2, 100 mM | 30 | 4812 |
| | HC1169 | | | phosphate buffer | | |
| | | PGDA | | | | |
| | | (mM) | | | | |
| Group 4 | 10 μg/mL | 250 | 100 | pH 5, 100 mM | 30 | 4158 |
| | HC1169 | | | citrate buffer | | |
| | 10 μg/mL | 250 | 100 | pH 5.6, 100 mM | 30 | 4482 |
| | HC1169 | | | citrate buffer | | |
| | 10 μg/mL | 250 | 100 | pH 6, 100 mM | 30 | 4647 |
| | HC1169 | | | citrate buffer | | |
| | 10 μg/mL | 250 | 100 | pH 6.6, 100 mM | 30 | 4608 |
| | HC1169 | | | phosphate buffer | | |
| | 10 μg/mL | 250 | 100 | pH 7.2, 100 mM | 30 | 4450 |
| | HC1169 | | | phosphate buffer | | |
| | 20 μg/mL | 250 | 100 | pH 5, 100 mM | 30 | 4140 |
| | HC1169 | | | citrate buffer | | |
| | 20 μg/mL | 250 | 100 | pH 5.6, 100 mM | 30 | 4403 |
| | HC1169 | | | citrate buffer | | |
| | 20 μg/mL | 250 | 100 | pH 6, 100 mM | 30 | 4609 |
| | HC1169 | | | citrate buffer | | |
| | 20 μg/mL | 250 | 100 | pH 6.6, 100 mM | 30 | 4526 |
| | HC1169 | | | phosphate buffer | | |
| | 20 μg/mL | 250 | 100 | pH 7.2, 100 mM | 30 | 4726 |
| | HC1169 | | | phosphate buffer | | |

This example demonstrate that significant levels of peracetic acid can be generated from H₂O₂ and triacetin or propylene glycol diacetate using different perhydrolases under various pH concentrations and buffer solutions as well as in moisturizer formulations.

### Example 40

### Hair Weakening Efficacy Using Perhydrolase in One-Step Procedure

### Effect of Enzyme Concentration and pH

The following example demonstrates that hair can be weakened in a one-step procedure where enzyme and substrates are combined in one-step at low concentrations. Effects of enzyme concentration and pH were examined.

The hair tresses in these examples were cut from the stock medium brown hair swatches that were custom made by International Hair Importers (Glendale, N.Y.). Each hair tress was glued at one end, and cut at 5 mm width and 4 cm long (excluding the glued portion), with 100+/−20 mg net hair weight. For each test condition, triplicates of hair tresses were used. In the one-step procedure, each hair tress was placed in a clean plastic weighing tray (VWR, Cat. #12577-053), then 1 mL of the perhydrolase HC1121 solution in a buffer was applied to the hair tress, and was rubbed into the hair tress with an applicator. Then calculated amount of triacetin and 30% H₂O₂ were applied onto each hair tress and rubbed into the hair tress which gave 200 mM triacetin and 100 mM H₂O₂. The hair tress sat in this reaction mixture for 30 min, before being washed thoroughly with tap water followed by paper towel dry. This completed a 30 min treatment cycle. The treatment cycle was repeated 16 times. Hair tress became lighter-colored and weakened during the treatment. After final rinse and air-drying, tensile strength tests as described in Example 25 were conducted on each hair tress to quantify hair weakening. In addition, L*, a*, b* color measurements were taken for each hair sample to quantify hair color loss, and L*, a*, b* color measurements were also taken for untreated hair as a reference for ΔE color difference calculations. ΔE was calculated in the standard way as ΔE=((L*−L*_{ref})²+(a*−a*_{ref})²+(b*−b*_{ref})²)^{0.5}. The treatment conditions are provided in table 32. The tensile strength test results shown in Table 33 indicated that hair got more weakened at higher enzyme concentration. At pH 6, when 30 μg/mL HC1121 was used which gave 0.3 μg/mg hair E/H ratio, the tensile strength of treated hair tress reduced to 1.9 N/mg hair, approaching 1.5 N/mg hair, the benchmark strength of hair treated with NAIR® cream for 5 min. At pH 7, when 30 μg/mL HC1121 was used, the tensile strength of treated hair tress was 0.3 N/mg hair, much less than the 1.5 N/mg hair benchmark. Therefore, proper concentration of enzyme at proper pH could weaken hair effectively. The measured hair color loss for treated hair tresses correlated well with the weakening of hair: the greater hair weakening, the greater hair color loss.

TABLE 32
Hair Treatment Conditions.

| | | | | | | Treatment | |
|---|---|---|---|---|---|---|---|
| | | | | | | time/ | |
| Treatment | HC1121 | | E/H | Triacetin | H₂O₂ | cycle | Treatment |
| condition | (μg/mL) | Buffer | (μg/mg H) | (mM) | (mM) | (min) | cycles |
| 1 | 5 | pH 6, 50 mM | 0.05 | 200 | 100 | 30 | 16 |
| | | citrate | | | | | |
| 2 | 15 | pH 6, 50 mM | 0.15 | 200 | 100 | 30 | 16 |
| | | citrate | | | | | |
| 3 | 30 | pH 6, 50 mM | 0.3 | 200 | 100 | 30 | 16 |
| | | citrate | | | | | |
| 4 | 30 | pH 7, 50 mM | 0.3 | 200 | 100 | 30 | 16 |
| | | phosphate | | | | | |

* E/H is the enzyme to hair ratio, expressed as micrograms per milligram hair (μg/mgH)

TABLE 33
Hair Tensile Strength Test Results and Hair Color Loss.

| | | | | | Hair color loss | |
|---|---|---|---|---|---|---|
| Treatment | Strength N/mg Hair | | | | avg | st dev |
| condition | replicate 1 | replicate 2 | replicate 3 | average | ΔE | ΔE |
| NAIR ® | 1.5 | | | | | |
| 5 min | | | | | | |
| treatment | | | | | | |
| 5 μg/mL | 2.89 | 3.16 | 3.03 | 3.03 | 5.2 | 0.6 |
| HC1121, | | | | | | |
| pH 6 | | | | | | |
| 15 μg/mL | 3.58 | 2.54 | 2.72 | 2.95 | 6.8 | 0.4 |
| HC1121, | | | | | | |
| pH 6 | | | | | | |
| 30 μg/mL | 1.77 | 1.86 | 1.95 | 1.86 | 8.7 | 0.9 |
| HC1121, | | | | | | |
| pH 6 | | | | | | |
| 30 μg/mL | 0.31 | 0.33 | 0.33 | 0.32 | 16.6 | 0.1 |
| HC1121, | | | | | | |
| pH 7 | | | | | | |

This example demonstrate that hair weakening can be obtained at low concentrations of enzyme and substrates in a one-step procedure

### Example 41

### Hair Weakening Efficacy Using Perhydrolase in One-Step Procedure

### Effect of Buffer Concentration and Treatment Time

The following example demonstrates that hair may be weakened effectively in a one-step procedure when using higher buffer concentration and longer treatment cycle time.

Hair tresses were treated using the same procedure as described in Example 40, except that SLES (sodium laurel ether sulfate, a common soap ingredient) wash step was added before tap water rinse step each cycle to imitate daily shower in real life, where 1 mL 1% SLES (“RHODAPEX ES 2K” by Rhodia Inc., Cranbury, N.J.) was transferred onto hair tress and the hair tress was rubbed with gloved hand on the palm for 30 seconds. The treatment parameters for this example (enzyme concentration, pH and buffer concentration, and treatment cycle time) were shown in Table 34. For the 24 hr treatment cycle, the hair tress sat in the mixture for 1 hr before being taken out to a dry dish. The hair tress sat dry for 23 hr before being washed with 1 mL 1% SLES followed by tap water rinse and paper towel dry. For the 30 min treatment cycles, the treatment cycle was repeated 16 times. For the 24 hr treatment cycles, the treatment was stopped at the completion of cycle 7 as visually the hair tresses treated with 24 hr cycles showed similar degree of damages as the hair tresses treated with 16 cycles of 30 min treatment. After final rinse and air-drying for each hair tress, tensile strength test and color measurement as described in Example 40 were conducted on each hair tress to quantify hair weakening and hair color loss. The tensile strength results shown in Table 35 indicated that at pH 6.6, with 30 μg/mL HC1121, 200 mM triacetin and 100 mM H₂O₂, hair tresses were significantly weakened with either 16 cycles of 30 min treatment or 7 cycles of 24 hr treatment. Hair tensile strength ranged from 0.3 to 0.8 N/mg hair, much less than the 1.5 N/mg hair tensile strength benchmark. Stronger hair weakening occurred at higher buffer concentration and longer treatment time. Same trend was observed for hair color loss: the greater hair weakening, the more hair color loss. Therefore, it was anticipated that even lower enzyme concentration could be used to reach benchmark hair weakening at slightly higher pH (pH 6.6 compared to pH 6 in Example 40), higher buffer concentration and longer treatment time.

TABLE 34
Hair Treatment Conditions.

| | | | | | | Treatment | |
|---|---|---|---|---|---|---|---|
| | | | | | | time/ | |
| Treatment | HC1121 | | E/H | Triacetin | H₂O₂ | cycle | Treatment |
| condition | (μg/mL) | Buffer | (μg/mg H) | (mM) | (mM) | (hr) | cycles |
| 1 | 30 | pH 6.6, 50 mM | 0.1 | 200 | 100 | 0.5 | 16 |
| | | phosphate | | | | | |
| 2 | 30 | pH 6.6, 100 mM | 0.1 | 200 | 100 | 0.5 | 16 |
| | | phosphate | | | | | |
| 3 | 30 | pH 6.6, 50 mM | 0.1 | 200 | 100 | 24 | 7 |
| | | phosphate | | | | | |

* E/H is the enzyme to hair ratio, expressed as micrograms per milligram hair

TABLE 35
Hair Tensile Strength Test Results and Hair Color Loss.

| | | | | | Hair color | |
|---|---|---|---|---|---|---|
| | | | | | loss | |
| Treatment | Strength N/mg Hair | | | | avg | stdev |
| condition | replicate 1 | replicate 2 | replicate 3 | average | ΔE | ΔE |
| NAIR ® | 1.5 | | | | | |
| 5 min | | | | | | |
| treatment | | | | | | |
| pH 6.6, | 0.59 | 0.50 | 0.85 | 0.65 | 17.7 | 0.52 |
| 50 mM | | | | | | |
| buffer, | | | | | | |
| 30 min/ | | | | | | |
| cycle × | | | | | | |
| 16 cycles | | | | | | |
| pH 6.6, | 0.22 | 0.34 | 0.35 | 0.30 | 24.2 | 1.35 |
| 100 mM | | | | | | |
| buffer, | | | | | | |
| 30 min/ | | | | | | |
| cycle × | | | | | | |
| 16 cycles | | | | | | |
| pH 6.6, | 0.86 | 0.94 | 0.66 | 0.82 | 15.6 | 0.37 |
| 50 mM | | | | | | |
| buffer, | | | | | | |
| 24 hr/cycle × | | | | | | |
| 7 cycles | | | | | | |

### Example 42

### Hair Weakening Efficacy Using Perhydrolase in One-Step Procedure

### Range of Substrate Concentrations

The following example demonstrates the range of substrate concentrations where hair could be weakened in one-step procedure.

Hair tresses were treated using the same procedure for 24 hr treatment cycles as described in Example 41 using 10 μg/mL HC1121 at pH 6.6 and substrates at various concentrations. The treatment parameters for this example (enzyme concentration, pH and buffer concentration, substrate concentrations and treatment cycle time) were shown in Table 36. Eight cycles of the 24 hr treatment cycle were repeated for all hair tresses. After final rinse and air-drying for each hair tress, tensile strength test and color measurement as described in Example 40 were conducted on each hair tress to quantify hair weakening and hair color loss. The tensile strength results shown in Table 37 indicated that at pH 6.6, with 10 μg/mL HC1121, 200-500 mM triacetin and 100-500 mM H₂O₂, hair tresses were significantly weakened with 8 cycles of 24 hr treatment. The higher the substrate concentrations were used, the greater hair weakening and hair color loss were obtained. At lowest substrate concentrations tested (200 mM triacetin and 100 mM H₂O₂), hair tensile strength reduced to 1.2 N/mg hair, substantially lower than the 1.5 N/mg hair benchmark. Once the H₂O₂ concentration was over 300 mM, hair tensile strength reduced to about 0.5 N/mg hair and leveled off. Therefore, with proper pH and treatment time, hair could be weakened using E/H ratio as low as 0.1 μg/mg hair, and substrate concentrations as low as 200 mM triacetin and 100 mM H₂O₂.

TABLE 36
Hair Treatment Conditions.

| | | | | | | Treatment | |
|---|---|---|---|---|---|---|---|
| | | | | | | time/ | |
| Treatment | HC1121 | | E/H | Triacetin | H₂O₂ | cycle | Treatment |
| condition | (μg/mL) | Buffer | (μg/mg H) | (mM) | (mM) | (hr) | cycles |
| 1 | 10 | pH 6.6, 50 mM | 0.1 | 200 | 100 | 24 | 8 |
| | | phosphate | | | | | |
| 2 | 10 | pH 6.6, 50 mM | 0.1 | 200 | 200 | 24 | 8 |
| | | phosphate | | | | | |
| 3 | 10 | pH 6.6, 50 mM | 0.1 | 200 | 300 | 24 | 8 |
| | | phosphate | | | | | |
| 4 | 10 | pH 6.6, 50 mM | 0.1 | 200 | 500 | 24 | 8 |
| | | phosphate | | | | | |
| 5 | 10 | pH 6.6, 50 mM | 0.1 | 500 | 500 | 24 | 8 |
| | | phosphate | | | | | |

* E/H is the enzyme to hair ratio, expressed as micrograms per milligram hair

TABLE 37
Hair tensile strength test results and hair color loss

| | | | | | Hair color | |
|---|---|---|---|---|---|---|
| | | | | | loss | |
| | Strength N/mg Hair | | | | avg | stdev |
| Treatment condition | replicate 1 | replicate 2 | replicate 3 | average | ΔE | ΔE |
| NAIR ® 5 min treatment | 1.5 | | | | | |
| 200 mM TA/100 mM H₂O₂ | 1.08 | 1.40 | 1.09 | 1.19 | 13.8 | 1.0 |
| 200 mM TA/200 mM H₂O₂ | 1.04 | 0.57 | 0.99 | 0.86 | 16.7 | 0.9 |
| 200 mM TA/300 mM H₂O₂ | 0.58 | 0.50 | 0.57 | 0.55 | 20.1 | 1.2 |
| 200 mM TA/500 mM H₂O₂ | 0.39 | 0.38 | 0.58 | 0.45 | 21.8 | 0.7 |
| 500 mM TA/500 mM H₂O₂ | 0.53 | 0.55 | 0.49 | 0.53 | 22.3 | 0.8 |

### Example 43

### Non-Enzymatic Depilatory Product with Hydrogen Peroxide and a Suitable Carboxylic Acid Ester Substrate to Produce the Peracetic Acid

The general method of treatment disclosed in Example 25 was followed, except there was no enzyme deposition step. H₂O₂ and triacetin, formulations were made according to Example 24, in 20% LUBRIDERM® lotion in 50 mM citrate buffer at pH 6. 200 microliters of each reagent was applied on ˜200 mg hair samples (4 test tresses). After application of the reagents, the treatment was allowed to continue for 24 hr, as in daily application. TWEEN®-20 (5% in 50 mM citrate buffer at pH 6) was used for the final surfactant wash. The treatment was repeated for 15 additional cycles, 16 days in all. The results are provided in Table 38.

This example illustrates that by tuning the concentration of H₂O₂ and triacetin, in a gentle moisturizing medium, the desired amount of hair-weakening can be achieved, in ˜15-16 days by following the daily application regimen. The product has potential utility in depilatory as well as in hair-lightening applications.

TABLE 38
Result of Hair Weakening Experiment using 2-step Daily Application.

| | | | Mixture | treatment | Color | |
|---|---|---|---|---|---|---|
| | [H₂O₂], | [TA], | volume, | time/cycle | loss | TS, |
| Experiment | Molar | Molar | (mL) | (hr) | ΔE | N/mgH* |
| 1 | 0.5 | 0.5 | 0.4 | 24 | 20 | 1.23 |
| 2 | 0.3 | 0.5 | 0.4 | 24 | 18 | 1.78 |
| 3 | 0.2 | 0.5 | 0.4 | 24 | 16 | 2.18 |
| 4 | 0.25 | 0.25 | 0.4 | 24 | 15 | 2.39 |
| 5 | 0.1 | 0.5 | 0.4 | 24 | 13 | 2.67 |

*TS is average (of 2) tensile strength, expressed as Newton per milligram hair (N/mgH)

What is claimed is:

1. A method to provide a peracid-based benefit agent preferentially to hair and not skin comprising:
a) providing a composition comprising a population of enzymes having perhydrolytic activity; said enzymes having at least one binding domain having affinity for hair;
b) contacting a body surface comprising hair and skin with the composition of step a), whereby a first fraction of the population of enzymes binds durably to hair and a second fraction of the population of enzymes does not durably bind to hair;
c) rinsing the body surface to remove the second fraction of enzymes not durably bound to hair;
d) optionally drying the rinsed body surface;
e) contacting said enzymes durably bound to hair with an aqueous solution comprising hydrogen peroxide and at least one carboxylic acid ester substrate; whereby a peracid benefit agent is generated, preferentially providing a peracid-based benefit to hair and not skin; and
f) optionally repeating steps (a) through (e).

2. The method of claim 1 wherein the peracid-based benefit is selected from group consisting of hair removal, hair weakening, hair bleaching, hair styling, hair curling, hair conditioning, hair pretreating prior to application of a non-peracid-based benefit agent, and combinations thereof.

3. The method of claim 2 wherein the non-peracid-based benefit agent is a depilatory agent, a hair dye, a hair conditioning agent or any combination thereof.

4. The method of claim 1 wherein an effective amount of peracid is generated within 60 minutes of performing contacting step (e), said effective amount ranging from 0.001 wt % to 4 wt %.

5. The method of claim 4 wherein the enzymatically produced peracid is peracetic acid.

6. The method of claim 1 wherein the enzymes having perhydrolytic activity are selected from the group consisting of lipases, proteases, esterases, aryl esterases, acyl transferases, carbohydrate esterases, and combinations thereof.

7. The method of claim 6 wherein the aryl esterase comprises an amino acid sequence having at least 95% identify to SEQ ID NO: 314.

8. The method of claim 6 wherein the enzyme having perhydrolytic activity comprises an amino acid sequence having at least 95% identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

9. The method of claim 6 wherein the carbohydrate esterases are CE-7 carbohydrate esterases, each having a CE-7 signature motif that aligns with a reference sequence SEQ ID NO: 2 using CLUSTALW, said signature motif comprising:
a) an RGQ motif at positions corresponding to positions 118-120 of SEQ ID NO: 2;
b) a GXSQG motif at positions corresponding to positions 179-183 of SEQ ID NO: 2; and
c) an HE motif at positions corresponding to positions 298-299 of SEQ ID NO: 2.

10. The method of claim 1 wherein the carboxylic acid ester substrate is selected from the group consisting of:
a) esters having the structure
[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have solubility in water of at least 5 ppm at 25° C.;
b) glycerides having the structure 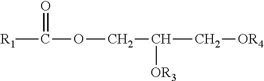
wherein R₁=C1 to C7 straight chain or branched chain alkyl optionally substituted with an hydroxyl or a C1 to C4 alkoxy group and R₃ and R₄ are individually H or R₁C(O);
c) one or more esters of the formula 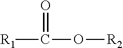
wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
d) acetylated saccharides selected from the group consisting of acetylated monosaccharides, acetylated disaccharides, and acetylated polysaccharides; and
e) mixtures thereof.

11. The method according to any one of claims 1 through 10 wherein the composition comprising a population of enzymes having perhydrolytic activity of step (a) of claim 1 is in the form of a body wash comprising at least one surfactant.

12. The method of claim 11 wherein the aqueous solution comprising hydrogen peroxide and at least one carboxylic acid ester substrate in contacting step (e) of claim 1 is in the form of a moisturizer or lotion comprising dermally acceptable ingredients.

13. The method of claim 12 wherein the moisturizer or lotion in step (e) is contacted with the body surface for a period of time from 10 seconds to 24 hours.

14. The method of claim 1 wherein steps (a) through (e) are repeated whereby the amount of durably bound enzyme having perhydrolytic activity increases with repeated treatment.

15. The method of claim 1 wherein contacting step (b) occurs for 5 seconds to 5 minutes.

16. A method to provide a peracid benefit agent to hair comprising:
a) providing a set of reaction components comprising:
1) at least one enzyme having perhydrolytic activity;
2) a source of peroxygen; and
3) a substrate selected form the group consisting of
i) esters having the structure
[X]ₘR₅
wherein X=an ester group of the formula R₆C(O)O R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have solubility in water of at least 5 ppm at 25° C.;
ii) glycerides having the structure 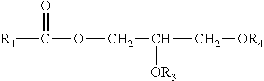
wherein R₁=C1 to C7 straight chain or branched chain alkyl optionally substituted with an hydroxyl or a C1 to C4 alkoxy group and R₃ and R₄ are individually H or R₁C(O);
iii) one or more esters of the formula 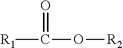
wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
iv) acetylated saccharides selected from the group consisting of acetylated monosaccharides, acetylated disaccharides, and acetylated polysaccharides; and
v) mixtures thereof;
b) contacting a body surface comprising hair with an effective amount of an enzymatically generated peracid is obtained by combining the set of reaction components; whereby a peracid-based benefit is provided to the body surface comprising hair;
c) rinsing the body surface;
d) optionally drying the rinsed body surface;
e) optionally repeating steps (a) through (d).

17. The method of claim 16 wherein the peracid-based benefit is selected from group consisting of hair removal, hair weakening, hair bleaching, hair styling, hair curling, hair conditioning, hair pretreating prior to application of a non-peracid-based benefit agent, and combinations thereof.

18. The method of claim 17 wherein the non-peracid-based benefit agent is a depilatory agent, a hair dye, a hair conditioning agent, and combinations thereof.

19. The method of claim 16 wherein the effective amount of peracid ranges from 0.001 wt % to 4 wt %.

20. The method of claim 19 wherein the peracid is peracetic acid.

21. The method of claim 16 wherein the set of reaction components are combined on the body surface.

22. The method of claim 16 wherein the set of reaction components are combined prior to contacting the body surface.

23. The method of claim 1, claim 6, claim 7, claim 8, claim 9 or claim 16 wherein the enzyme having perhydrolytic activity is a fusion protein having the following general structure:
PAH-[L]_{y}-HSBD
or
HSBD-[L]_{y}-PAH
wherein
PAH is the enzyme having perhydrolytic activity;
HSBD is a peptidic component having affinity for hair;
L is an optional peptide linker ranging from 1 to 100 amino acids in length; and
y is 0 or 1.

24. The method of claim 23 wherein the peptidic component having affinity for hair is an antibody, an F_{ab} antibody fragment, a single chain variable fragment (scFv) antibody, a Camelidae antibody, a scaffold display protein or a single chain polypeptide lacking an immunoglobulin fold.

25. The method of claim 24 wherein the single chain polypeptide lacking an immunoglobulin fold comprises a K_{D} value or an MB₅₀ value of 10⁻⁵ M or less for hair.

26. The method of claim 24 wherein the single chain polypeptide lacking an immunoglobulin fold comprises 2 to 50 hair-binding peptides wherein the hair-binding peptides are independently and optionally separated by a polypeptide spacer ranging from 1 to 100 amino acids in length.

27. The method of claim 23 wherein the peptidic component having affinity for hair comprises a net positive charge.

28. The method of claim 1 or claim 16 where the peracid is produced at a concentration of 0.001% to 4% within 60 minutes of combining the peracid—generating reaction components.

29. The method of claim 28 wherein the peracid is contacted with the hair for less than 1 hour.

30. A hair care product comprising:
a) an enzyme catalyst having perhydrolytic activity,
b) at least one substrate selected from the group consisting of:
1) esters having the structure
[X]ₘR₅
wherein X=an ester group of the formula R₆C(O)O
R₆=C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have solubility in water of at least 5 ppm at 25° C.;
2) glycerides having the structure 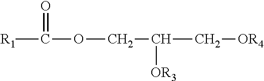
wherein R₁=C1 to C7 straight chain or branched chain alkyl optionally substituted with an hydroxyl or a C1 to C4 alkoxy group and R₃ and R₄ are individually H or R₁C(O);
3) one or more esters of the formula 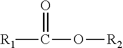
wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and 3) acetylated saccharides selected from the group consisting of acetylated monosaccharides, acetylated disaccharides, and acetylated polysaccharides;
c) a source of peroxygen; and
d) a dermally acceptable carrier medium suitable for use in a hair care product.

31. The hair care product of claim 30 wherein the enzyme having perhydrolytic activity is in the form of a fusion protein comprising:
a) a first portion comprising the enzyme having perhydrolytic activity; and
b) a second portion having a peptidic component having affinity for human hair.

32. The hair care product of claim 30 or claim 31 wherein the enzyme having perhydrolytic activity is selected from the group lipases, proteases, esterases, acyl transferases, aryl esterases, carbohydrate esterases, and combinations thereof.

33. The hair care product of claim 32 wherein the aryl esterase comprises an amino acid sequence having at least 95% identify to SEQ ID NO: 314.

34. The hair care product of claim 32 wherein the enzyme having perhydrolytic activity comprises an amino acid sequence having at least 95% identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

35. The hair care product of claim 32 wherein the carbohydrate esterases are CE-7 carbohydrate esterases, each having a CE-7 signature motif that aligns with a reference sequence SEQ ID NO: 2 using CLUSTALW, said signature motif comprising:
a) an RGQ motif at positions corresponding to positions 118-120 of SEQ ID NO:2;
b) a GXSQG motif at positions corresponding to positions 179-183 of SEQ ID NO:2; and
c) an HE motif at positions corresponding to positions 298-299 of SEQ ID NO:2.

36. The hair care product of claim 31 wherein the second portion having a peptidic component having affinity for human hair is a single chain peptide comprising at least one hair-binding peptide.

37. The hair care product of claim 36 wherein the hair-binding peptides ranges from 5 to 60 amino acids in length.

38. The hair care product of claim 30 or claim 31 wherein the hair care product is in the form of a powder, paste, gel, liquid, oil, ointment, spray, foam, tablet, a hair shampoo, a hair conditioner rinse or any combination thereof.

39. The hair care product of claim 30 wherein the enzyme catalyst remains separated from the carboxylic acid ester substrate, the source of peroxygen or both the carboxylic acid ester substrate and the source of peroxygen prior to use of the hair care product.

40. A hair care product for removing or weakening hair comprising a set of reaction components comprising:
a) 0.1 to 50 wt % of at least one substrate selected from the group consisting of:
1) esters having the structure
[X]ₘR₅
wherein X=an ester group of the formula R₆C(O)O;
R₆=a C1 to C7 linear, branched or cyclic hydrocarbyl moiety, optionally substituted with hydroxyl groups or C1 to C4 alkoxy groups, wherein R₆ optionally comprises one or more ether linkages for R₆=C2 to C7;
R₅=a C1 to C6 linear, branched, or cyclic hydrocarbyl moiety or a five-membered cyclic heteroaromatic moiety or six-membered cyclic aromatic or heteroaromatic moiety optionally substituted with hydroxyl groups; wherein each carbon atom in R₅ individually comprises no more than one hydroxyl group or no more than one ester group or carboxylic acid group; wherein R₅ optionally comprises one or more ether linkages;
m is an integer ranging from 1 to the number of carbon atoms in R₅; and
wherein said esters have solubility in water of at least 5 ppm at 25° C.;
2) glycerides having the structure 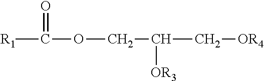
wherein R₁=C1 to C7 straight chain or branched chain alkyl optionally substituted with an hydroxyl or a C1 to C4 alkoxy group and R₃ and R₄ are individually H or R₁C(O);
3) one or more esters of the formula 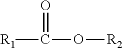
wherein R₁ is a C1 to C7 straight chain or branched chain alkyl optionally substituted with an hydroxyl or a C1 to C4 alkoxy group and R₂ is a C1 to C10 straight chain or branched chain alkyl, alkenyl, alkynyl, aryl, alkylaryl, alkylheteroaryl, heteroaryl, (CH₂CH₂O)ₙ, or (CH₂CH(CH₃)—O)ₙH and n is 1 to 10; and
4) acetylated saccharides selected from the group consisting of acetylated monosaccharides, acetylated disaccharides, and acetylated polysaccharides;
b) 0.1 to 15 wt % a source of hydrogen peroxide;
c) 0.001 to 1 wt % of an enzyme catalyst comprising at least one CE-7 carbohydrate esterase having perhydrolytic activity; and
d) up to 98 wt % of the balance of the hair care composition comprising a dermally acceptable carrier medium; whereby upon mixing the reaction components a peracid is produced.

41. The hair care product of claim 40 wherein said hair care product further comprises up to 25 wt % of a swelling agent.

42. The hair care product of claim 41 wherein the swell agent comprises urea.

43. The hair care product of claim 40 further comprising up to 25 wt % of a reducing agent.

44. The hair care product of claim 43 wherein the reducing agent is a thioglycolate.

45. A method to remove hair comprising
a) providing a composition comprising 0.001 to 4 wt % peracetic acid;
b) contacting a body surface comprising hair with said peracetic acid under suitable conditions to form peracid-treated hair;
c) optionally rinsing the peracetic acid-treated hair with an aqueous solution;
d) optionally drying the hair after step (b) or step (c); and
e) repeating steps (a) through (d) until the hair is removed from the body surface.

46. A method to decrease the tensile strength of hair comprising
a) providing a composition comprising 0.001 to 4 wt % peracetic acid;
b) contacting a body surface comprising hair characterized by an initial tensile strength with the composition comprising peracetic acid under suitable conditions to form peracetic acid-treated hair;
c) optionally rinsing the peracetic acid-treated hair with an aqueous solution;
d) optionally drying the hair after step (b) or step (c); and
e) repeating steps (a) through (d) whereby the initial tensile strength of the hair is reduced.

47. The method of claim 45 or claim 46 wherein the peracetic acid-treated hair is contacted with at least one reducing agent to enhance hair removal or hair weakening.

48. The method of claim 47 wherein the reducing agent is at least one thioglycolate.

49. The method of claim 45 or claim 46 wherein the composition comprising 0.001 to 4 wt % peracetic acid is produced non-enzymatically by reacting a source of hydrogen peroxide and a suitable carboxylic acid ester substrate to produce the peracetic acid.

50. The method of claim 45 or claim 46 wherein the composition comprising 0.001 to 4 wt % peracetic acid is produced enzymatically by combining a perhydrolytic enzyme, a source of peroxygen, and a carboxylic acid ester substrate prior to contacting the body surface.

51. The method of claim 50 wherein the enzyme having perhydrolytic activity is selected from the group lipases, proteases, esterases, acyl transferases, aryl esterases, carbohydrate esterases, and combinations thereof.

52. The method of claim 51 wherein the aryl esterase comprises an amino acid sequence having at least 95% identity to SEQ ID NO: 314.

53. The method of claim 51 wherein the enzyme having perhydrolytic activity comprises an amino acid sequence having at least 95% identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 293, 297, 299, 301, 303, 305, 307, 309, 311, 314, 315, 338, and 339.

54. The method of claim 51 wherein the carbohydrate esterases are CE-7 carbohydrate esterases, each having a CE-7 signature motif that aligns with a reference sequence SEQ ID NO: 2 using CLUSTALW, said signature motif comprising:
a) an RGQ motif at positions corresponding to positions 118-120 of SEQ ID NO:2;
b) a GXSQG motif at positions corresponding to positions 179-183 of SEQ ID NO:2; and
c) an HE motif at positions corresponding to positions 298-299 of SEQ ID NO:2.

55. A method for the production of a fusion protein comprising a perhydrolytic enzyme coupled to at least one hair binding domain comprising:
a) providing a recombinant microbial host cell comprising an expressible genetic construct encoding a fusion protein, said fusion protein comprising an enzyme having perhydrolytic activity coupled to a peptidic component having affinity for hair;
b) growing the recombinant microbial host cell under suitable conditions whereby the fusion protein is produced; and
c) optionally recovering the fusion protein.

56. The method of claim 55 wherein the recombinant microbial host cell is Escherichia coli or Bacillus subtilis.
